(19) United States
(12) Patent Application Publication
(10) Pub. No.: US 2026/0356955 A1
(43) Pub. Date: Nov. 12, 2026

(54) KLRB1 BINDING AGENTS AND METHODS OF USE THEREOF

(71) Applicant: The Brigham and Women's Hospital, Inc., Boston, MA (US)

(72) Inventor: Steven A. Greenberg, Boston, MA (US)

(21) Appl. No.: 19/123,478

(22) PCT Filed: Oct. 24, 2023
(86) PCT No.: PCT/US2023/077607

§ 371 Date: Apr. 23, 2025

Related U.S. Application Data
(60) Provisional application No. 63/419,201, filed on Oct. 25, 2022.
(60) Provisional application No. 63/419,186, filed on Oct. 25, 2022.

(51) Int. Cl. C07K 16/28

(52) U.S. Cl. CPC C07K 16/2851; C07K 2317/24; C07K 2317/33; C07K 2317/53; C07K 2317/565; C07K 2317/732; C07K 2317/734; C07K 2317/76; C07K 2317/92

(57) ABSTRACT

KLRB1 binding agents (in particular anti-KLRB1-antibodies and antigen binding portion thereof) and compositions thereof, as well as therapeutic methods of using the agents, e.g., for depleting cells or inhibiting cells or activating cells, (in particular, Th17, Th17.1, ex-Th17, Tc17, MAIT, INKT, peTh2, ILC2, ILC3, NK cells, and/or neoplastic T or NK cells in vivo), for the treatment of autoimmune disease, allergic diseases, transplant rejection, hematologic malignancies, and cancer.

### CLAIM OF PRIORITY

This application claims the benefit of U.S. Provisional Application Ser. No. 63/419,201, filed on Oct. 25, 2022, and No. 63/419,186, filed on Oct. 25, 2022. The entire contents of the foregoing are incorporated herein by reference.

### STATEMENT REGARDING FEDERALLY SPONSORED RESEARCH

This invention was made with government support under HL119145 awarded by the National Institutes of Health. The government has certain rights in the invention.

### TECHNICAL FIELD

This disclosure generally relates to KLRB1 binding agents, in particular anti-KLRB1-antibodies as well as therapeutic methods of using the agents for autoimmune disease, allergic diseases, transplant rejection, hematologic malignancies, and cancer.

### BACKGROUND

The expression of killer cell lectin-like receptor subfamily B, member 1 (KLRB1; also known as CD161) defines a unique population of immune cells implicated in a variety of autoimmune and allergic diseases. For example, T-helper IL-17 secreting (Th17) cells express KLRB1 (Maggi et al. 2010). Th17 cells, and the cytokine IL-17 they produce, are implicated in psoriasis, psoriatic arthritis, ankylosing spondylitis, and inflammatory bowel disease (Yang et al. 2014), among other autoimmune diseases. Monoclonal antibody therapeutics targeting IL-17, or the upstream cytokine IL-23 that stimulates IL-17 producing cells, are FDA approved and marketed for use in psoriasis (e.g., secukinumab and ixekizumab) and ankylosing spondylitis (e.g., secukinumab). However, therapeutic developments based upon targeting these molecules remain limited and there remains a need for new therapeutics utilizing these targets for various indications including autoimmune disease, allergic disease, inflammatory diseases, and cancer.

### SUMMARY OF THE DISCLOSURE

Described herein are KLRB1-binding antibodies (and antigen-binding fragments thereof) that have numerous uses, including therapeutic and diagnostic uses. For example, the antibodies can be used for treating, and in some cases preventing, various diseases associated with KLRB1 expressing cells (i.e., by depleting the KLRB1 expressing cells), e.g., for treating, and in some cases preventing (i.e., reducing the risk of developing), autoimmune diseases including psoriasis, psoriatic arthritis, ankylosing spondylitis, palmoplantar pustulosis, hidradenitis suppurativa, and inflammatory bowel disease; allergic diseases including asthma and atopic dermatitis; transplant rejection; hematologic malignancies, and cancer.

Provided herein are antibodies or antigen binding portions thereof that specifically bind to killer cell lectin-like receptor subfamily B, member 1, (KLRB1; optionally SEQ ID NO:1, SEQ ID NO:2, or SEQ ID NO:3), wherein the antibody or antigen binding portion thereof comprises or consists of at least one of (a) a heavy chain variable region (VH) comprising a VH complementarity determining region (CDR) 1 comprising a sequence that is at least 95% identical to a VH CDR1 amino acid sequence set forth in one of Table 1 to 18, preferably Table 9, 8, or 18; a VH CDR2 comprising a sequence that is at least 95% identical to a VH CDR2 amino acid sequence set forth in one of Table 1 to 18, preferably Table 9, 8, or 18, and a VH CDR3 comprising a sequence that is at least 95% identical to a VH CDR3 amino acid sequence set forth in one of Table 1 to 18, preferably Table 9, 8, or 18; and (b) a light chain variable region (VL) comprising a VL CDR1 comprising a sequence that is at least 95% identical to a VL CDR1 amino acid sequence set forth in one of Table 1 to 18, preferably Table 9, 8, or 18, a VL CDR2 comprising a sequence that is at least 95% identical to a VL CDR2 amino acid sequence set forth in one of Table 1 to 18, preferably Table 9, 8, or 18, and a VL CDR3 comprising a sequence that is at least 95% identical to a VL CDR3 amino acid sequence set forth in one of Table 1 to 18, preferably Table 9, 8, or 18.

In some embodiments, the VH comprises or consists of an VH amino acid sequence set forth in Table 9, 8, or 18. In some embodiments, the VL comprises or consists of an VL amino acid sequence set forth in Table 9, 8, or 18. In some embodiments, the VH comprises or consists of the VH amino acid sequence set forth in Table 3, and the VL comprises or consists of the VH amino acid sequence set forth in Table 3. In some embodiments, the VH comprises or consists of the VH amino acid sequence set forth in Table 5, and the VL comprises or consists of the VH amino acid sequence set forth in Table 5. In some embodiments, the VH comprises or consists of the VH amino acid sequence set forth in Table 8, and the VL comprises or consists of the VH amino acid sequence set forth in Table 8. In some embodiments, the VH comprises or consists of the VH amino acid sequence set forth in Table 9, and the VL comprises or consists of the VH amino acid sequence set forth in Table 9. In some embodiments, the VH comprises or consists of the VH amino acid sequence set forth in Table 18, and the VL comprises or consists of the VH amino acid sequence set forth in Table 18. In some embodiments, the VH comprises or consists of an amino acid sequence having at least 95% sequence identity to the VH amino acid sequence set forth in Table C, and the VL comprises or consists of an amino acid sequence having at least 95% sequence identity to the VL amino acid sequence set forth in Table C. In some embodiments, the VH comprises or consists of an amino acid sequence having at least 95% sequence identity to the VH amino acid sequence set forth in one of Tables 9, 8, or 18, and the VL comprises or consists of an amino acid sequence having at least 95% sequence identity to the amino acid sequence set forth in one of Tables 9, 8, or 18, preferably wherein the VH and VL are from the same Table.

In some embodiments, the antibodies or antigen binding portions thereof comprise a constant region heavy chain and light chain, wherein the constant region heavy chain and/or light chain comprises or consists of an amino acid sequence set forth in one of Table A.

Additionally, provided herein are antibodies or antigen binding portions thereof that specifically bind to human killer cell lectin-like receptor subfamily B, member 1, (KLRB1; e.g., SEQ ID NO:1), comprising or consisting of a variable region heavy chain consisting of the VH amino acid sequence set forth in Table 3 and a variable region light chain consisting of the VL amino acid sequence set forth in Table 3, and optionally a constant region, optionally comprising a sequence that is at least 95% identical to a sequence set forth in Table A.

Also provided herein are antibodies or antigen binding portions thereof that specifically bind to human killer cell lectin-like receptor subfamily B, member 1, (KLRB1; e.g., SEQ ID NOs: 1-3), comprising or consisting of a variable region heavy chain consisting of the VH amino acid sequence set forth in Table 5 and a variable region light chain consisting of the VL amino acid sequence set forth in Table 5, and optionally a constant region, optionally comprising a sequence that is at least 95% identical to a sequence set forth in Table A.

Additionally, provided herein are antibodies or antigen binding portions thereof that specifically bind to human killer cell lectin-like receptor subfamily B, member 1, (KLRB1; e.g., SEQ ID NO:1), comprising or consisting of a variable region heavy chain consisting of the VH amino acid sequence set forth in Table 8, and a variable region light chain consisting of the VL amino acid sequence set forth in Table 8, and optionally a constant region, optionally comprising a sequence that is at least 95% identical to a sequence set forth in Table A.

Further, provided herein are antibodies or antigen binding portions thereof that specifically bind to human killer cell lectin-like receptor subfamily B, member 1, (KLRB1; e.g., SEQ ID NO:1), comprising or consisting of a variable region heavy chain consisting of the VH amino acid sequence set forth in Table 9, and a variable region light chain consisting of the VL amino acid sequence set forth in Table 9, and optionally a constant region, optionally comprising a sequence that is at least 95% identical to a sequence set forth in Table A.

Further, provided herein are antibodies or antigen binding portions thereof that specifically bind to human killer cell lectin-like receptor subfamily B, member 1, (KLRB1; e.g., SEQ ID NO:1), comprising or consisting of a variable region heavy chain consisting of the VH amino acid sequence set forth in Table 18, and a variable region light chain consisting of the VL amino acid sequence set forth in Table 18, and optionally a constant region, optionally comprising a sequence that is at least 95% identical to a sequence set forth in Table A.

In some embodiments, the constant region comprises or consists of a sequence as set forth in Table A.

In some embodiments, the antibodies comprise or consist of a heavy chain variable and/or light chain variable sequence that is at least 95% identical to a sequence set forth in Table B.

In some embodiments, the antibodies comprise or consist of a complete heavy chain and/or light chain sequence that is at least 95% identical to a sequence set forth in Table C.

Also provided herein are antibodies or antigen binding portions thereof that specifically bind to human KLRB1, comprising CDRs from different tables herein, or heavy/light chain pairs from different tables herein. In some embodiments, the antibodies or antigen binding portions thereof comprise a heavy chain constant region hinge region and Fc domain.

In some embodiments, the antibody or antigen binding portion thereof is an antibody that comprises a heavy chain constant region comprising an amino acid sequence having at least 80%, 90%, 95%, or 97% sequence identity to a heavy chain constant region amino acid sequence set forth in Table A.

In some embodiments, the antibody or antigen binding portion thereof is a monoclonal antibody.

In some embodiments, the antibody or antigen binding portion thereof is a chimeric, humanized, or human antibody, and/or comprises one or more mutations (e.g., in a CDR) that remove Asn (N)-glycosylation sites or remove Cys, Asp, Met, Trp or Lys.

In some embodiments, the antibody or antigen binding portion thereof is an immunoglobulin G (IgG) subtype IgG1 antibody, an IgG2 antibody, or an IgG4 antibody.

In some embodiments, the antibody or antigen binding portion thereof is an antibody that comprises an Fc region, preferably human IgG1, that binds to Fc gamma receptors (FcγRs) and induces antibody dependent cell-mediated cytotoxicity (ADCC) to deplete cells expressing KLRB1, or that binds to C1q and induce complement dependent cytotoxicity (CDC).

In some embodiments, the antibody or antigen binding portion thereof is conjugated to a cytotoxic agent.

In some embodiments, the antibody or antigen binding portion thereof comprises an Fc region that is afucosylated.

As CD161 is an inhibitory receptor when bound to ligand CLEC2D (LLT1), modulation of this interaction can therefore affect immune cell activity. Thus, inhibiting the CD161/CLEC2D interaction could enhance T cell function, a typical mechanism of action of the class of immuno-oncology therapeutics, while augmenting the CD161/CLEC2D interaction could act as immunosuppression for autoimmune and allergic diseases. In some embodiments, antibodies disclosed herein block CD161/CLEC2D interaction thereby activating T or NK cells to attack tumor cells in a variety of cancers, typical of an immuno-oncology checkpoint inhibition mechanism.

In some embodiments, antibodies disclosed herein augment (e.g., increase) CD161/CLEC2D interaction thereby suppressing T or NK cells providing immunosuppression applicable for treating a variety of autoimmune, allergic, and inflammatory diseases.

In some embodiments, a KLRB1-binding antibody disclosed herein achieves better immune cell activation, for example demonstrates improvements in any one or more of the activities of activating T cells to produce cytokines, activating T cells to kill tumor cells, activating NK cells (e.g., increased expression of CD107a), activating NK cells to produce cytokines or cytotoxic molecules such as granzymes, and activating NK cells to kill tumor cells when compared to the immune cell activation of one or more of prior antibodies (e.g., B199.2, HP-3G10, OTI1D8, 14F1F11, 702228, B-D51, 2F3, EP7169, DX1, DX12, 191B8, Ab9, KW1.2.1, KW7.3.7, or JNH25G2G22).

In some embodiments, an antibody disclosed herein that has an N-terminal glutamine or glutamate may be post-translationally modified at such N-terminus to form a pyroglutamate (or pyrrolidone carboxylic acid).

Also provided herein are polynucleotides comprising a nucleic acid sequence encoding an antibody or antigen binding portion thereof as described herein. In some embodiments, the nucleic acid sequence is operably linked to a promoter.

Additionally provided are vectors comprising the polynucleotides described herein, as well as host cells comprising the polynucleotides or the vectors, and optionally expressing an antibody or antigen binding portion thereof as described herein. Also provided herein are methods for making the antibodies or antigen binding portions thereof as described herein. The methods can include culturing the host cells under conditions sufficient to express the antibody or antigen binding portion thereof and isolating the antibody or antigen binding portion thereof. In some embodiments, the methods include formulating the antibody as a pharmaceutical composition.

Further, provided herein are pharmaceutical compositions that comprise an antibody or antigen binding portion thereof of as described herein (e.g., comprises or consists of the antibody or antigen binding portion thereof as an active agent), and a pharmaceutically acceptable carrier or diluent.

In some embodiments, the antibody or antigen binding portion thereof is not B199.2 (Invitrogen), HP-3G10 (Invitrogen), OTI1D8 (OriGene), 14F1F11 (OriGene), 702228 (R&D Systems), B-D51 (Cell Sciences), 2F3 (Novus Biologics), EP7169 (Abcam), DX1 (Thermo Fisher), DX12 (BD Biosciences), 191B8 (Miltenyi Biotec), Ab9 (PCT publication WO2023028501A1), KW1.2.1 (United States patent publication US20210122826A1), KW7.3.7 (United States patent publication US20210122826A1), or JNH25G2G22 (Creative Diagnostics).

Also provided herein are methods for treating one or more of an autoimmune disease, an allergic disease, a transplant rejection, and a hematologic malignancy in a subject in need thereof, the method comprising administering to the subject an antibody that binds KLRB1, preferably an antibody or antigen binding portion thereof, polynucleotide, vector, pharmaceutical composition, or host cell that expresses the antibody or antigen binding portion thereof, as described herein. Also provided are an antibody that binds KLRB1, preferably an antibody or antigen binding portion thereof, polynucleotide, vector, pharmaceutical composition, or host cell that expresses the antibody or antigen binding portion thereof, as described herein, for use in a method of treating one or more of an autoimmune disease, an allergic disease, a transplant rejection, and a hematologic malignancy in a subject in need thereof. In some embodiments, the autoimmune disease is rheumatoid arthritis, Sjogren's syndrome, inclusion body myositis (IBM), discoid lupus, psoriasis, idiopathic pulmonary fibrosis, diabetes, alopecia universalis, primary biliary cholangitis, multiple sclerosis, lymphocytic colitis, palmoplantar pustulosis, or hidradenitis suppurativa. In some embodiments, the allergic disease is asthma, allergic eosinophilic asthma, allergy, atopic dermatitis, nasal polyposis, eosinophilic gastrointestinal disorder, or hypereosinophilic syndrome. In some embodiments, the transplant rejection can be a rejection of a kidney, lung, heart, liver, limb, skin, or multi-organ transplant. In some embodiments, the hematological malignancy is a leukemia, e.g., T cell leukemia, NK cell leukemia, T cell prolymphocytic leukemia (T-PLL), or large granular lymphocytic leukemia (LGLL). In some embodiments, the hematological malignancy is a lymphoma, e.g., hepatosplenic T cell lymphoma, NK/T cell lymphoma, mycosis fungoides, Sezary syndrome, peripheral T cell lymphoma, angioimmunoblastic T cell lymphoma (AITL), or peripheral T cell lymphoma not otherwise specified (PTCL-NOS).

In some embodiments, the antibody or antigen binding portion thereof, the polynucleotide, the vector, the antibody binds to and depletes Th17, Th17.1, ex-Th17, Tc17, mucosal-associated invariant T cells (MAIT), invariant NK-T cells (INKT), innate lymphoid cells types 2 and 3 (ILC2, and ILC3), pathogenic effector Th2 (peTh2) cells, and/or NK cells expressing KLRB1.

In some embodiments of each of the aforementioned aspects and embodiments, as well as other aspects and embodiments described herein, the subject is human.

Without wishing to be being bound by theory, Applicant believes that the antibodies disclosed herein will demonstrate superiority to previously described antibodies, e.g., commercially available antibodies, in one or more of the following activities:
increased binding potency (lower binding EC₅₀ to cell expressed KLRB1 or to soluble KLRB1 extracellular domain);
increased ADCC mediated depletion potency (lower EC50 for depletion of CHO-KLRB1+ cells or lower EC₅₀ for reporter cell line ADCC assay);
increased binding affinity kinetics (lower KD or lower K_{off});
increased blocking potency (lower IC₅₀ for inhibition of CLEC2D binding to cell expressed KLRB1); and/or
increased augmentation potency (lower EC₅₀ for augmenting (increasing) CLEC2D binding to cell expressed KLRB1).

In some embodiments, the antibodies disclosed herein have a higher production yield, lower immunogenicity (due to the presence of humanized variable regions and/or human Fc sequences), and/or improved biophysical parameters (e.g., higher melting temperature, greater freeze-thaw stability, lower isomerization, reduced or absence of deamidation, and/or less susceptibility to oxidation) when compared to previously described antibodies, e.g., commercially available antibodies. Examples of previously described antibodies include B199.2, HP-3G10, OTI1D8, 14F1F11, 702228, B-D51, 2F3, EP7169, DX1, DX12, 191B8, Ab9, KW1.2.1, KW7.3.7, or JNH25G2G22.

Where aspects or embodiments of the disclosure are described in terms of a Markush group or other grouping of alternatives, the present disclosure encompasses not only the entire group listed as a whole, but also each member of the group individually and all possible subgroups of the main group, and also the main group absent one or more of the group members. The present disclosure also envisages the explicit exclusion of one or more of any of the group members in the claimed disclosure.

These and other advantages of the present technology will be apparent when reference is made to the accompanying drawings and the following description.

### BRIEF DESCRIPTION OF THE DRAWINGS

FIG. 1 shows KLRB1 expression marks Th17, Th17.1, ex-Th17, Tc17, iNKTs, IL2, ILC3, peTh2, and a subset of NK cells.

FIG. 2 shows body-wide KLRB1 expression profiling data indicating that KLRB1 has no significant expression on any cell type other than immune cells

FIG. 3 shows KLRB1 is overexpressed in rheumatoid arthritis synovium compared to osteoarthritis and normal synovium.

FIG. 4 shows KLRB1 is overexpressed in rheumatoid arthritis synovium compared to osteoarthritis and crystal-induced arthritis synovium.

FIG. 5 shows KLRB1 is overexpressed in advanced and moderate Sjogren's syndrome salivary gland compared to normal.

FIG. 6 shows KLRB1 is overexpressed in Sjogren's syndrome parotid gland compared to normal.

FIG. 7 shows KLRB1 is overexpressed in muscle tissue from patients with inclusion body myositis.

FIG. 8 shows KLRB1 is overexpressed in skin tissue from patients with discoid lupus.

FIG. 9 shows KLRB1 is overexpressed in skin tissue from patients with psoriasis.

FIG. 10 shows KLRB1 is overexpressed in lung tissue from patients with idiopathic pulmonary fibrosis.

FIG. 11 shows KLRB1 is overexpressed in pancreas tissue from patients with diabetes.

FIG. 12 shows KLRB1 is overexpressed in scalp tissue from patients with alopecia universalis.

FIG. 13 shows KLRB1 is overexpressed in liver tissue from patients with primary biliary cholangitis.

FIG. 14 shows KLRB1 is overexpressed in brain tissue from patients with multiple sclerosis.

FIG. 15 shows KLRB1 is overexpressed in colon tissue from patients with lymphocytic colitis.

FIG. 16 shows KLRB1 is overexpressed in kidney tissue from patients with renal transplants.

FIG. 17 shows KLRB1 is overexpressed in broncheoalveolar lavage fluid from patients with lung transplants.

FIG. 18 shows KLRB1 is overexpressed in skin tissue from patients with atopic dermatitis.

FIG. 19 shows KLRB1 is overexpressed in skin tissue from patients with palmoplantar pustulosis.

FIG. 20 shows KLRB1 is overexpressed in skin tissue from patients with hidradenitis suppurativa.

FIG. 21 shows KLRB1 is overexpressed in airway brushings from patients with asthma.

FIGS. 22A-D shows KLRB1 is expressed in various T and NK cell lymphomas and leukemias. FIG. 22A shows increased expression of KLRB1 in tumor cells from 4/4 patients with hepatosplenic T cell lymphoma (HSTCL), 7/19 patients with NK/T cell lymphoma (NKTCL), and 2/2 patients with mycosis fungoides. FIG. 22B shows expression of KLRB1 in various forms of peripheral T cell lymphoma (PTCL) including angioimmunoblastic T cell lymphoma (AITL), anaplastic large cell lymphoma (ALCL; both ALK-positive and ALK-negative), NK/T cell lymphoma (NKTCL), peripheral T cell lymphoma not otherwise specified (PTCL-NOS), and T cell prolymphocytic leukemia (T-PLL). FIG. 22C shows KLRB1 increased expression in spleen tumor cells compared to healthy spleen and from tumor cells compared to healthy CD4 T cells in 4 patients each with HSTCL. FIG. 22D shows increased expression of KLRB1 an HSTCL cell line (DERL-2) and from aggressive NK cell leukemia (ANKL) cell lines (KHYG-1 and NKL).

FIGS. 23A-C shows binding of the murine antibodies 9C10G11, 57E2E4, 75B10F12, 7B6C7, 52G9B12B5, 13H2D11, 5A11D10, 5B1B11, 50A8F2, 47A5H2, 66G9C11, 33F11A6, 37G6A7, 39C5D6, 62H1D6, 92E12F2, and 22F10G3 against human KLRB1 by ELISA. Human KLRB1 was coated on ELISA plates and incubated with the indicated antibodies. Secondary anti-mouse IgG was used to detect antibodies bound to KLRB1 on the ELISA plate. FIG. 23A shows binding of 9C10G11, 57E2E4, 75B10F12, 7B6C7, 52G9B12B5, 13H2D11, and 5A11D10. FIG. 23B shows binding of 5B1B11, 50A8F2, 47A5H2, 66G9C11, 33F11A6, 37G6A7, 39C5D6, 62H1D6, and 92E12F2. FIG. 23C shows binding of 22F10G3.

FIGS. 24A-E shows binding of the murine antibodies 9C10G11, 57E2E4, 75B10F12, 7B6C7, 52G9B12B5, 5B1B11, 50A8F2, 47A5H2, 66G9C11, 33F11A6, 37G6A7, 39C5D6, 62H1D6, 92E12F2, and 22F10G3 to CHO-K1 cells expressing human or cynomolgus KLRB1 (CHO-hum-KLRB1 or CHO-cyno-KLRB1). Binding was detected by FACS. Secondary anti-mouse IgG was used to detect antibodies bound to CHO-hum-KLRB1 or CHO-cyno-KLRB1. FIG. 24A shows binding of 9C10G11, 57E2E4, 75B10F12, 7B6C7, and 52G9B12B5 to CHO-hum-KLRB1. FIG. 24B shows binding of 9C10G11, 57E2E4, 75B10F12, 7B6C7, and 52G9B12B5 to CHO-cyno-KLRB1. FIG. 24C shows binding of 5B1B11, 50A8F2, 47A5H2, 66G9C11, 33F11A6, 37G6A7, 39C5D6, 62H1D6, and 92E12F2 to CHO-hum-KLRB1. FIG. 24D shows binding of 22F10G3 to CHO-hum-KLRB1. FIG. 24E shows binding of 22F10G35 to CHO-cyno-KLRB1.

FIGS. 25A-B shows an antibody dependent cell-mediated cytotoxicity (ADCC) assay measuring target cell lysis of CHO-K1 target cells expressing human KLRB1 (CHO-hum-KLRB1) incubated with human peripheral blood mononuclear cells (PBMCs) and the following antibodies: (25A) mouse/human IgG1-kappa (wild-type) chimeric antibodies 57E2E4-Chimeric, 7B6C7-Chimeric, 52G9B12B5-Chimeric, 5B1B11-Chimeric, 50A8F2-Chimeric, 47A5H2-Chimeric; 52G9B12B5-Chimeric-EN (a mouse/human chimeric KLRB1-binding antibody with 52G9B12B5 variable regions and human IgG1-kappa mutant L234A/L235A/G237A mutation; used as a negative control); and human IgG1 (negative control); or (25B) mouse/human IgG1-kappa (wild-type) chimeric antibody 22F10G3-chimeric, and human IgG1 (negative control), in an ADCC cell lysis assay. The assay used LDH release and detection of formazan salt (Genscript, SC1544). The figures show % target cell lysis for each antibody at the various concentrations indicated.

FIGS. 26A-E show that some anti-KLRB1 binding antibodies may compete with the natural KLRB1 ligand CLEC2D for binding to KLRB1 on CHO-K1 cells expressing human KLRB1 (CHO-hum-KLRB1) and thereby block the ability of CLEC2D to bind to KLRB1. CHO-humKLRB1 cells were incubated with a CLEC2D-Fc-biotin fusion protein and with increasing concentrations of antibodies 9C10G11, 57E2E4, 75B10F12, 7B6C7, 52G9B12B5, 5B1B11, 50A8F2, 47A5H2, 66G9C11, 33F11A6, 37G6A7, 39C5D6, 62H1D6, 92E12F2, 13H2D11, 5A11D10, 22F10G3, and control mouse IgG. Binding of CLEC2D was determined by addition of streptavidin-AF488. The graphs show mean fluorescent intensity (MFI) over the indicated concentration range. FIG. 26A shows competition of 9C10G11, 57E2E4, 75B10F12, 7B6C7, 52G9B12B5, 5B1B11, and mouse IgG with CLEC2D. FIG. 26B shows competition of 47A5H2, 66G9C11, 33F11A6, 37G6A7, 39C5D6, 62H1D6, 92E12F2, 13H2D11, 5A11D10, and mouse IgG with CLEC2D. FIG. 26C shows competition of 50A8F2 and mouse IgG with CLEC2D, demonstrating that 50A8F2 enhances binding of CLEC2D to KLRB1. FIG. 26D shows competition in a second repeated experiment with a greater range of concentrations of 50A8F2, 47A5H2, and mouse IgG with CLEC2D, demonstrating in a second experiment that 50A8F2 enhances binding of CLEC2D to KLRB1. FIG. 26E shows competition of 22F10G3 and mouse IgG with CLEC2D.

FIGS. 27A-F show the binding of 47A5H2 chimeric and humanized antibodies and other KLRB1 binding antibodies to human KLRB1 extracellular protein by ELISA. FIG. 27A shows binding of Hu47A5H2-01, Hu47A5H2-02, Hu47A5H2-03, and Hu47A5H2-04. FIG. 27B shows binding of Hu47A5H2-05, Hu47A5H2-06, Hu47A5H2-07, and Hu47A5H2-08. FIG. 27C shows binding of Hu47A5H2-09, Hu47A5H2-10, Hu47A5H2-11, and Hu47A5H2-12. FIG. 27D shows binding of Hu47A5H2-13, Hu47A5H2-14, Hu47A5H2-15, Hu47A5H2-16, and Hu47A5H2-chimeric. FIG. 27E shows binding of Hu47A5H2-11, Hu50A8F2-04, Ab9, KW1.2.1, and KW7.3.7. FIG. 27F shows binding of Hu47A5H2-11, HP-3G10, DX12, and 191B8.

FIGS. 28A-F show the binding of 47A5H2 chimeric and humanized antibodies and other KLRB1 binding antibodies to cynomolgus KLRB1 extracellular protein by ELISA. FIG. 28A shows binding of Hu47A5H2-01, Hu47A5H2-02, Hu47A5H2-03, and Hu47A5H2-04. FIG. 28B shows binding of Hu47A5H2-05, Hu47A5H2-06, Hu47A5H2-07, and Hu47A5H2-08. FIG. 28C shows binding of Hu47A5H2-09, Hu47A5H2-10, Hu47A5H2-11, and Hu47A5H2-12. FIG. 28D shows binding of Hu47A5H2-13, Hu47A5H2-14, Hu47A5H2-15, Hu47A5H2-16, and Hu47A5H2-chimeric. FIG. 28E shows binding of Hu47A5H2-11, Hu50A8F2-04, Ab9, KW1.2.1, and KW7.3.7. Hu50A8F2-04 did not bind. FIG. 28F shows binding of Hu47A5H2-11, HP-3G10, and DX12. 191B8 did not bind.

FIGS. 29A-F show the binding of 47A5H2 chimeric and humanized antibodies and other KLRB1 binding antibodies to CHO-hum-KLRB1 cells measured by FACS. FIG. 29A shows binding of Hu47A5H2-01, Hu47A5H2-02, Hu47A5H2-03, and Hu47A5H2-04. FIG. 29B shows binding of Hu47A5H2-05, Hu47A5H2-06, Hu47A5H2-07, and Hu47A5H2-08. FIG. 29C shows binding of Hu47A5H2-09, Hu47A5H2-10, Hu47A5H2-11, and Hu47A5H2-12. FIG. 29D shows binding of Hu47A5H2-13, Hu47A5H2-14, Hu47A5H2-15, Hu47A5H2-16, and Hu47A5H2-chimeric. FIG. 29E shows binding of Hu47A5H2-11, Hu50A8F2-04, Ab9, KW1.2.1, and KW7.3.7. FIG. 29F shows binding of Hu47A5H2-11, HP-3G10, DX12, and 191B8.

FIGS. 30A-F show the binding of 47A5H2 chimeric and humanized antibodies and other KLRB1 binding antibodies to CHO-cyno-KLRB1 cells measured by FACS. FIG. 30A shows binding of Hu47A5H2-01, Hu47A5H2-02, Hu47A5H2-03, and Hu47A5H2-04. FIG. 30B shows binding of Hu47A5H2-05, Hu47A5H2-06, Hu47A5H2-07, and Hu47A5H2-08. FIG. 30C shows binding of Hu47A5H2-09, Hu47A5H2-10, Hu47A5H2-11, and Hu47A5H2-12. FIG. 30D shows binding of Hu47A5H2-13, Hu47A5H2-14, Hu47A5H2-15, Hu47A5H2-16, and Hu47A5H2-chimeric. FIG. 30E shows binding of Hu47A5H2-11, Hu50A8F2-04, Ab9, KW1.2.1, and KW7.3.7. FIG. 30F shows binding of Hu47A5H2-11, HP-3G10, DX12, and 191B8.

FIGS. 31A-F show the blocking by 47A5H2 chimeric and humanized antibodies and other KLRB1 binding antibodies of CLEC2D binding to CHOK1-human KLRB1 cells measured by FACS. FIG. 31A shows blocking by Hu47A5H2-01, Hu47A5H2-02, Hu47A5H2-03, and Hu47A5H2-04. FIG. 31B shows blocking by Hu47A5H2-05, Hu47A5H2-06, Hu47A5H2-07, and Hu47A5H2-08. FIG. 31C shows blocking by Hu47A5H2-09, Hu47A5H2-10, Hu47A5H2-11, and Hu47A5H2-12. FIG. 31D shows blocking by Hu47A5H2-13, Hu47A5H2-14, Hu47A5H2-15, Hu47A5H2-16, and Hu47A5H2-chimeric. FIG. 31E shows blocking by Hu47A5H2-11, Ab9, KW1.2.1, and KW7.3.7. FIG. 31F shows blocking by Hu47A5H2-11, HP-3G10, DX12, and 191B8.

FIGS. 32A-D show the binding of 50A8F2 chimeric and humanized antibodies to human KLRB1 extracellular protein by ELISA. FIG. 32A shows binding of Hu50A8F2-01, Hu50A8F2-02, Hu50A8F2-03, and Hu50A8F2-04. FIG. 32B shows binding of Hu50A8F2-05, Hu50A8F2-06, Hu50A8F2-07, and Hu50A8F2-08. FIG. 32C shows binding of Hu50A8F2-09, Hu50A8F2-10, Hu50A8F2-11, and Hu50A8F2-12. FIG. 32D shows binding of Hu50A8F2-13, Hu50A8F2-14, Hu50A8F2-15, Hu50A8F2-16, and Hu50A8F2-chimeric.

FIGS. 33A-E show the binding of 50A8F2 chimeric and humanized antibodies to CHO-hum-KLRB1 cells and absence of binding to CHO-cyno-KLRB1 cells measured by FACS. FIG. 33A shows binding to CHO-hum-KLRB1 of Hu50A8F2-01, Hu50A8F2-02, Hu50A8F2-03, and Hu50A8F2-04. FIG. 33B shows binding to CHO-hum-KLRB1 of Hu50A8F2-05, Hu50A8F2-06, Hu50A8F2-07, and Hu50A8F2-08. FIG. 33C shows binding of Hu50A8F2-09, Hu50A8F2-10, Hu50A8F2-11, and Hu50A8F2-12. FIG. 33D shows binding to CHO-hum-KLRB1 of Hu50A8F2-13, Hu50A8F2-14, Hu50A8F2-15, Hu50A8F2-16, and Hu50A8F2-chimeric. FIG. 33E shows absence of binding to CHO-cyno-KLRB1 of the 16 humanized 50A8F2 antibodies Hu50A8F2-01 through Hu50A8F2-16 and the 50A8F2 chimeric, with binding (as a positive control) of Hu47A5H2 chimeric.

FIGS. 34A-D show that 50A8F2 chimeric and humanized antibodies augmented CLEC2D binding to CHO-hum-KLRB1 cells measured by FACS. FIG. 34A shows augmentation of CLEC2D/CHO-hum-KLRB1 binding by Hu50A8F2-01, Hu50A8F2-02, Hu50A8F2-03, and Hu50A8F2-04. FIG. 34B shows augmentation of CLEC2D/CHO-hum-KLRB1 binding by Hu50A8F2-05, Hu50A8F2-06, Hu50A8F2-07, and Hu50A8F2-08. FIG. 34C shows augmentation of CLEC2D/CHO-hum-KLRB1 binding by Hu50A8F2-09, Hu50A8F2-10, Hu50A8F2-11, and Hu50A8F2-12. FIG. 34D shows augmentation of CLEC2D/CHO-hum-KLRB1 binding by Hu50A8F2-13, Hu50A8F2-14, Hu50A8F2-15, Hu50A8F2-16, and Hu50A8F2 chimeric. Hu47A5H2 did not augment but rather inhibited CLEC2D/CHO-hum-KLRB1 binding.

FIG. 35 shows that 50A8F2 chimeric and humanized antibodies Hu50A8F2-01, Hu50A8F2-03, Hu50A8F2-04, and Hu50A8F2-06 augmented CLEC2D binding to CHOK1-human KLRB1 cells measured by FACS. In comparison, human IgG1 had no effect on CLEC2D binding to CHOK1-human KLRB1 cells and KW1.2.1, KW7.3.7, and 47A5H2 chimeric antibodies inhibited CLEC2D binding to CHOK1-human KLRB1 cells.

FIGS. 36A-B show 47A5H2 chimeric antibody increased NK cell activation measured by FACS. FIG. 36A shows NK cell CD107a expression was decreased with K562-CLEC2D compared to K562 cells, and then increased with 47A5H2 chimeric (47A5H2-chi) antibody. FIG. 36B shows NK cell IFN-gamma (IFNg) expression was decreased with K562-CLEC2D compared to K562 cells, and then increased with 47A5H2 chimeric antibody.

FIGS. 37A-B show NK cell activation was suppressed by CLEC2D and anti-KLRB1 blocking antibodies activated NK cells in the presence of CLEC2D suppression. Primary NK cells showed less activation with K562-CLEC2D single cell clone 1D1 than with K562 cells. Hu47A5H2-11 and other anti-KLRB1 antibodies enhanced NK cell activation in assays in which NK cells targeted K562-CLEC2D but not assays in which NK cells targeted K562 cells alone. FIG. 37A shows NK cell activation measured as CD107a expression. FIG. 37B shows NK cell activation measured as IFNg expression.

FIGS. 38A-B show Hu47A5H2-11 enhanced NK cell activation measured by CD107a expression by FACS, with NK cells targeting K562-CLEC2D clone 1D1 cells. Hu47A5H2-11 had superior potency (EC50 0.039 nM) compared to other tested antibodies. FIG. 38A shows Hu47A5H2-11 in comparison to Ab9 (EC50 0.118 nM), KW1.2.1 (EC50 4.946 nM), and KW7.3.7 (EC50 36.29 nM). FIG. 34B shows Hu47A5H2-11 in comparison to HP-3G10 (EC50 1.071 nM), DX12 (EC50 6.137 nM), and 191B8 (EC50 0.085 nM).

FIGS. 39A-B show Hu47A5H2-11 enhanced NK cell activation measured by IFN-gamma expression by FACS, with NK cells targeting K562-CLEC2D clone 1D1 cells. Hu47A5H2-11 had superior potency (EC50 0.131 nM) compared to other tested antibodies. FIG. 39A shows Hu47A5H2-11 in comparison to Ab9 (EC50 0.436 nM), and KW1.2.1 and KW7.3.7 (neither of which activated NK cell IFNg production). FIG. 39B shows Hu47A5H2-11 in comparison to HP-3G10 (EC50 2.510 nM), DX12 (EC50 7.956 nM), and 191B8 (EC50 0.2475 nM; more correctly, 0.383 nM given NK cell toxicity at 40 nM).

FIG. 40 shows that 47A5H2 chimeric and humanized antibodies enhanced NK cell killing in a primary NK cell vs Raji target cell assay, with superior potency to KW1.2.1 and KW7.3.7.

FIG. 41A-B shows that Hu47A5H2-11 enhanced NK cell killing in a primary NK cell vs Raji target cell assay, with superior potency to Ab9, KW1.2.1, KW7.3.7, HP-3G10, DX12, and 191B8. FIG. 41A shows Hu47A5H2-11 (EC50 0.036 nM) in comparison to Ab9 (EC50 0.055 nM), KW1.2.1 (EC50 0.112 nM), and KW7.3.7 (EC50 0.440 nM). FIG. 41B shows Hu47A5H2-11 (EC50 0.036 nM) in comparison to HP-3G10 (EC50 0.325 nM), DX12 (no effect), and 191B8 (EC50 0.064 nM).

FIG. 42 shows that 47A5H2-chimeric antibody enhanced T cell activation in Jurkat-NFAT-1G4TCR-KLRB1 vs K562-CLEC2D-HLA loaded with 0.005 μg/ml NY-ESO-1 peptide assay. 47A5H2-chimeric, but not 50A8F2 chimeric, increased T cell activation.

FIG. 43A-B shows that Hu47A5H2-11 enhanced T cell activation in Jurkat-NFAT-1G4TCR-KLRB1 vs K562-CLEC2D-HLA loaded with 0.005 μg/ml NY-ESO-1 peptide assay, with superior potency to Ab9, KW1.2.1, KW7.3.7, HP-3G10, DX12, and 191B8. FIG. 43A shows Hu47A5H2-11 (EC50 0.379 nM) in comparison to Ab9 (EC50 0.675 nM), KW1.2.1 (did not activate), KW7.3.7 (did not activate), and Hu50A8F2-04 (did not activate). FIG. 43B shows Hu47A5H2-11 (EC50 0.379 nM) in comparison to HP-3G10 (EC50 12.56 nM), DX12 (EC50 0.179 nM), and 191B8 (EC50 0.392 nM).

### DETAILED DESCRIPTION

KLRB1 expression marks a unique set of immune system cells implicated in a variety of autoimmune diseases. These include Th17, Th17.1, ex-Th17, Tc17, MAIT, INKT, ILC2, ILC3, peTh2, and/or NK cells.

KLRB1 is also expressed by neoplastic cells present in a number of T and NK cell malignancies. These include various peripheral T cell and NK cell lymphomas and leukemias.

The KLRB1-binding antibodies described herein can be used to preferentially target Th17, Th17.1, ex-Th17, Tc17, MAIT, INKT, ILC2, ILC3, peTh2, NK cells, and/or neoplastic T or NK cells for depletion. The population of KLRB1 expressing immune cells more abundantly expresses IL-17 than the population of total CD4 or CD8 T cells, and are more specific to Th17 and Tc17 T cells than CD4 or CD8, respectively. Thus described herein are methods for treating a subject by administering to a subject in need thereof an effective amount of a killer cell lectin-like receptor B1 (KLRB1) binding agent with cell depleting activity, thereby depleting Th17, Th17.1, ex-Th17, Tc17, MAIT, INKT, ILC2, ILC3, peTh2, NK cells, and/or neoplastic T or NK cells in vivo. These methods can be used to treat an autoimmune disease, allergic disease, transplant rejection, or hematologic malignancies, e.g., as described herein.

Additionally, because modulating KLRB1 has inhibitory or stimulatory effects on T and NK cells, thus described herein are methods for treating a subject by administering to a subject in need thereof an effective amount of a killer cell lectin-like receptor B1 (KLRB1) binding agent with KLRB1 receptor blocking activity (in which blocking of binding of CLEC2D (LLT1) to KLRB1 is achieved), thereby activating or inhibiting Th17, Th17.1, ex-Th17, Tc17, MAIT, iNKT, ILC2, ILC3, and/or peTh2 in vivo. These methods can be used to activate T and/or NK cells to enhance their activity against tumor cells to be used to treat cancer.

In some embodiments, the antibodies have antibody dependent cellular cytotoxicity (ADCC) effector activity or complement dependent cytotoxicity (CDC) effector activity. Administering to a subject in need thereof an effective amount of an anti-KLRB1 antibody that has ADCC or CDC effector function, or that is linked to a cytotoxic agent, can be used to eliminate or reduce the number of Th17, Th17.1, ex-Th17, Tc17, MAIT, INKT, ILC2, ILC3, peTh2, NK cells, and/or neoplastic T or NK cells. In some embodiments, the disclosure provides a killer cell lectin-like receptor B1 (KLRB1) binding agent as described herein that has ADCC or CDC activity or is conjugated to a cytotoxic agent. In various aspects, the disclosure provides an mRNA or cDNA encoding the binding agent. In various aspects, the disclosure provides a pharmaceutical composition comprising an effective amount of the binding agent.

Various features of the disclosure, including KLRB1 and its ligands, anti-KLRB1 antibodies or antigen-binding portions thereof, pharmaceutical compositions, treatment and administration, and illustrative examples are discussed, in turn, below. The following sections contain definitions of terms used in this disclosure. Unless otherwise defined herein, technical and scientific terms used in the present description have the meanings that are commonly understood by those of ordinary skill in the art. For purposes of interpreting this specification, the following description of terms will apply and whenever appropriate, terms used in the singular will also include the plural and vice versa. In the event that any description of a term set forth conflicts with any document incorporated herein by reference, the description of the term set forth below shall control.

### Killer Cell Lectin-Like Receptor B1 (KLRB1)

Killer cell lectin-like receptor B1 (KLRB1), also known as CD161, is a type II transmembrane protein. It is a receptor modulating the activity of T and NK cells and can act as either a stimulatory (Fergusson et al. 2014) or inhibitory (Aldemir et al. 2005, Mathewson et al. 2021) receptor for different types of T and NK cells. It is present on the surface of a variety of immune cells. The ligand for KLRB1 is LLT1 (also called CLEC2D) (Aldemir et al. 2005).

KLRB1 expression among T cells is limited to those with an ability to respond to IL-12 and IL-18 (Fergusson et al. 2014). Among CD4+T helper cells, KLRB1 expression uniquely marks Th17, Th17.1, and ex-Th17 cells (cells that no longer produce IL-17 but produce IFN-γ). KLRB1 expression distinguishes Th1 IFNG producing cells (which are KLRB1-negative) from ex-Th17 IFNG producing cells (which are KLRB1+) (Basdeo et al. 2017). Among CD8+T cytotoxic cells, KLRB1 expression uniquely marks Tc17 cells. Thus, KLRB1 marks T cells with capacity for IL-17 production or, in the case of ex-Th17 cells, interferon-gamma (IFNG) production. This cytokine production is undesired in the case of autoimmune disease.

Described herein are antibodies and antigen-binding fragments thereof that bind to KLRB1, preferably human KLRB1. An exemplary sequence of human KLRB1 is provided as

SEQ ID NO: 1:
MDQQAIYAELNLPTDSGPESSSPSSLPRDVCQGSPWHQFALKLSCAGIIL
LVLVVTGLSVSVTSLIQKSSIEKCSVDIQQSRNKTTERPGLLNCPIYWQQ
LREKCLLFSHTVNPWNNSLADCSTKESSLLLIRDKDELIHTQNLIRDKAI
LFWIGLNFSLSEKNWKWINGSFLNSNDLEIRGDAKENSCISISQTSVYSE
YCSTEIRWICQKELTPVRNKVYPDS.

In some embodiments, the KLRB1 is cynomolgus KLRB1 (e.g., AOA2K5WYIl from UniParc UPI0003ABB264); an exemplary sequence is provided as SEQ ID NO:2:

MDQQMMYAELTLPKDSGPESSSPSSLPRDVCQGSPWHQFALKLSCAGIIL
LVLVVTGLSLSVASLLQKPSIGKCSVDIQQNRTKTTERPDLLNCPIYWQQ
VQEKCLLFSHTVNPWNNSLADCSTKESSLLLIQDKDELTRTQNLIHDKAI
SFWIGLNFSLSEKNWKWINGSFLSSNDLKITGDAKENSCVYISQTSVYSE
YCSTEMKWICQKELTLVRNKVSPDSWL.

In some embodiments, the KLRB1 is cynomolgus KLRB1 (e.g., UniProt A0A7N9D796); an exemplary sequence is provided as SEQ ID NO:3:

MDQQMMYAELTLPKDSGPESSSPSSLPRDVCQGSPWHQFALKLSCAGIIL
LVLVVTGLSLSVASLLQKPSIGKCSVDIQQNRTKTTERPDLLNCPIYWKQ
VQEKCLLFSHTVNPWNNSLADCSTKESSLLLIQDKDELTRTQNLIHDKAI
SFWIGLNFSLSEKNWKWINGSFLSSNDLKITGDAKENSCVYISQTSVYSE
YCSTEMKWICQKELTLVRNKVSPDSWL.

### KLRB1—Binding Antibodies

Described herein are antibodies and antigen binding fragments thereof that bind to KLRB1. The term “antibody” refers to an immunoglobulin molecule or immunologically active portion thereof, i.e., an antigen-binding portion.

The term “monoclonal antibody” as used herein refers to an antibody obtained from a population of substantially homogeneous antibodies, e.g., the individual antibodies comprising the population are identical except for possible naturally-occurring mutations that may be present in minor amounts. An antibody can be monoclonal. An antibody can be a human or humanized antibody. The term “monoclonal antibody” encompasses intact and full-length monoclonal antibodies as well as antibody fragments (e.g., Fab, Fab′, F(ab′)₂, Fv), single chain antibodies (e.g., scFv), fusion proteins comprising an antibody fragment, and any other modified immunoglobulin molecule comprising at least one antigen-binding site. Furthermore, “monoclonal antibody” refers to such antibodies made by any number of techniques, including but not limited to, hybridoma production, phage library display, recombinant expression, and transgenic animals.

The term “chimeric antibody” refers to an antibody in which a portion of the heavy and/or light chain is derived from a first source or species, while the remainder of the heavy and/or light chain is derived from a different source or species.

The term “humanized antibody” as used herein refers to an antibody that comprises a human heavy chain variable region and a light chain variable region wherein the native CDR residues are replaced by residues from corresponding CDRs from a nonhuman antibody (e.g., mouse, rat, rabbit, or nonhuman primate), wherein the nonhuman antibody has the desired specificity, affinity, and/or activity. In some embodiments, one or more framework region residues of the human heavy chain or light chain variable regions are replaced by corresponding residues from nonhuman antibody. Furthermore, humanized antibodies can comprise residues that are not found in the human antibody or in the nonhuman antibody. In some embodiments, these modifications are made to further refine and/or optimize antibody characteristics. In some embodiments, the humanized antibody comprises at least a portion of an immunoglobulin constant region (e.g., CH1, CH2, CH3, Fc), typically that of a human immunoglobulin. Exemplary constant regions include those shown in Table A.

TABLE A
Sequences of Exemplary Constant Regions

| Antibody Fragment | Sequence |
|---|---|
| Human IgG1 | ASTKGPSVFPLAPSSKSTSGGTAALGCLVKDYFPEPVTVSWNSGALTSGVHTFPAVLQSSGLYS |
| Constant (IGHG1)/ | LSSVVTVPSSSLGTQTYICNVNHKPSNTKVDKKVEPKSCDKTHTCPPCPAPELLGGPSVFLFPPK |
| Glm(z) allele | PKDTLMISRTPEVTCVVVDVSHEDPEVKFNWYVDGVEVHNAKTKPREEQYNSTYRVVSVLTV |
| (K214/D356/L358) | LHQDWLNGKEYKCKVSNKALPAPIEKTISKAKGQPREPQVYTLPPSRDELTKNQVSLTCLVKG |
| | FYPSDIAVEWESNGQPENNYKTTPPVLDSDGSFFLYSKLTVDKSRWQQGNVFSCSVMHEALHN |
| | HYTQKSLSLSPGK (SEQ ID NO: 4) |
| Human IgG1 | ASTKGPSVFPLAPSSKSTSGGTAALGCLVKDYFPEPVTVSWNSGALTSGVHTFPAVLQSSGLYS |
| Constant (IGHG1) | LSSVVTVPSSSLGTQTYICNVNHKPSNTKVDKKVEPKSCDKTHTCPPCPAPEAAGAPSVFLFPPK |
| LALAGA/Glm(z) | PKDTLMISRTPEVTCVVVDVSHEDPEVKFNWYVDGVEVHNAKTKPREEQYNSTYRVVSVLTV |
| allele with | LHQDWLNGKEYKCKVSNKALPAPIEKTISKAKGQPREPQVYTLPPSRDELTKNQVSLTCLVKG |
| L234A/L235A/G237A | FYPSDIAVEWESNGQPENNYKTTPPVLDSDGSFFLYSKLTVDKSRWQQGNVFSCSVMHEALHN |
| | HYTQKSLSLSPGK (SEQ ID NO: 5) |
| Human IgG1 Fc | LGGPSVFLFPPKPKDTLMISRTPEVTCVVVDVSHEDPEVKFNWYVDGVEVHNAKTKPREEQYN |
| region/WT | STYRVVSVLTVLHQDWLNGKEYKCKVSNKALPAPIEKTISKAKGQPREPQVYTLPPSRDELTK |
| | NQVSLTCLVKGFYPSDIAVEWESNGQPENNYKTTPPVLDSDGSFFLYSKLTVDKSRW |
| | QQGNVFSCSVMHEALHNHYTQKSLSLSPGK (SEQ ID NO: 6) |
| Human IgK Constant | RTVAAPSVFIFPPSDEQLKSGTASVVCLLNNFYPREAKVQWKVDNALQSGNSQESVTEQDSKD |
| (IGKC)/Uniprot | STYSLSSTLTLSKADYEKHKVYACEVTHQGLSSPVTKSFNRGEC (SEQ ID NO: 7) |
| P01834 | |
| Mouse IgG1 | AKTTPPSVYPLAPGSAAQTNSMVTLGCLVKGYFPEPVTVTWNSGSLSSGVHTFPAVLQSDLYT |
| Constant (IGHG1 | LSSSVTVPSSPRPSETVTCNVAHPASSTKVDKKIVPRDCGCKPCICTVPEVSSVFIFPPKPKDVLTI |
| Mouse)/Uniprot | TLTPKVTCVVVDISKDDPEVQFSWFVDDVEVHTAQTQPREEQFNSTFRSVSELPIMHQDWLNG |
| P01868 | KEFKCRVNSAAFPAPIEKTISKTKGRPKAPQVYTIPPPKEQMAKDKVSLTCMITDFFPEDITVEW |
| | QWNGQPAENYKNTQPIMNTNGSYFVYSKLNVQKSNWEAGNTFTCSVLHEGLHNHHTEKSLSH |
| | SPGK (SEQ ID NO: 8) |
| Mouse IgG2a | AKTTAPSVYPLAPVCGDTTGSSVTLGCLVKGYFPEPVTLTWNSGSLSSGVHTFPAVLQSDLYTL |
| Constant (IGHG | SSSVTVTSSTWPSQSITCNVAHPASSTKVDKKIEPRGPTIKPCPPCKCPAPNLLGGPSVFIFPPKIK |
| Mouse)/Uniprot | DVLMISLSPIVTCVVVDVSEDDPDVQISWFVNNVEVHTAQTQTHREDYNSTLRVVSALPIQHQD |
| P01863 | WMSGKEFKCKVNNKDLPAPIERTISKPKGSVRAPQVYVLPPPEEEMTKKQVTLTCMVTDFMPE |
| | DIYVEWTNNGKTELNYKNTEPVLDSDGSYFMYSKLRVEKKNWVERNSYSCSVVHEGLHNHH |
| | TTKSFSRTPGK (SEQ ID NO: 9) |
| Mouse IgG2b | KTTPPSVYPLAPGCGDTTGSSVTLGCLVKGYFPESVTVTWNSGSLSSSVHTFPALLQSGLYTMS |
| Constant (IGG2B | SSVTVPSSTWPSQTVTCSVAHPASSTTVDKKLEPSGPISTINPCPPCKECHKCPAPNLEGGPSVFIF |
| Mouse)/Uniprot | PPNIKDVLMISLTPKVTCVVVDVSEDDPDVQISWFVNNVEVHTAQTQTHREDYNSTIRVVSTLP |
| P01867 | IQHQDWMSGKEFKCKVNNKDLPSPIERTISKIKGLVRAPQVYILPPPAEQLSRKDVSLTCLVVGF |
| | NPGDISVEWTSNGHTEENYKDTAPVLDSDGSYFIYSKLNMKTSKWEKTDSFSCNVRHEGLKNY |
| | YLKKTISRSPGLDLDDICAEAKDGELDGLWTTITIFISLFLLSVCYSASVTLFKVKWIFSSVVELK |
| | QKISPDYRNMIGQGA (SEQ ID NO: 10) |
| Mouse IgK Constant | RADAAPTVSIFPPSSEQLTSGGASVVCFLNNFYPKDINVKWKIDGSERQNGVLNSWTDQDSKDS |
| (IGKC Mouse)/ | TYSMSSTLTLTKDEYERHNSYTCEATHKTSTSPIVKSFNRNEC (SEQ ID NO: 11) |
| Uniprot P01837 | |

The term “human antibody” as used herein refers to an antibody that possesses an amino acid sequence that corresponds to an antibody produced by a human and/or an antibody that has been made using any of the techniques that are known to those of skill in the art for making human antibodies. These techniques include, but not limited to, phage display libraries, yeast display libraries, transgenic animals, recombinant protein production, and B-cell hybridoma technology.

“Antibody fragments” can include a portion of an intact antibody, preferably the antigen binding or variable region of the intact antibody. Examples of antibody fragments include Fab, Fab′, F(ab′)2, and Fv fragments; diabodies; linear antibodies; single-chain antibody molecules; and multispecific antibodies formed from antibody fragments.

The terms “epitope” and “antigenic determinant” are used interchangeably herein and refer to that portion of an antigen or target capable of being recognized and bound by a particular antibody. When the antigen or target is a polypeptide, epitopes can be formed both from contiguous amino acids and noncontiguous amino acids juxtaposed by tertiary folding of the protein. Epitopes formed from contiguous amino acids (also referred to as linear epitopes) are typically retained upon protein denaturing, whereas epitopes formed by tertiary folding (also referred to as conformational epitopes) are typically lost upon protein denaturing. An epitope typically includes at least 3, and more usually, at least 5, 6, 7, or 8-10 amino acids in a unique spatial conformation. Epitopes can be predicted using any one of a large number of software bioinformatic tools available on the internet. X-ray crystallography may be used to characterize an epitope on a target protein by analyzing the amino acid residue interactions of an antigen/antibody complex.

“Fv” includes the minimum antibody fragment which contains a complete antigen-recognition and binding site. This region consists of a dimer of one heavy- and one light-chain variable domain in tight, non-covalent association. It is in this configuration that the three CDRs of each variable domain interact to define an antigen-binding site on the surface of the VH-VL dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site. The Fab fragment also contains the constant domain of the light chain and the first constant domain (CH1) of the heavy chain. Fab fragments differ from Fab′ fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

Depending on the amino acid sequence of the constant domain of their heavy chains, immunoglobulins can be assigned to different classes. There are five major classes of immunoglobulins: IgA, IgD, IgE, IgG, and IgM, and several of these may be further divided into subclasses (isotypes), e.g., IgB1, IgG2, IgG3, IgG4, IgA, and IgA2. “Single-chain Fv” or “sFv” antibody fragments comprise the VH and VL domains of antibody, wherein these domains are present in a single polypeptide chain. Preferably, the Fv polypeptide further comprises a polypeptide linker between the VH and VL domains which enables the sFv to form the desired structure for antigen binding.

In various embodiments, the antibody or antigen binding fragment thereof comprises a human or humanized antibody. Humanized forms of non-human (e.g., murine) antibodies are chimeric immunoglobulins, immunoglobulin chains or fragments thereof (such as Fv, Fab, Fab′, F(ab′)2 or other antigen-binding subsequences of antibodies) which contain minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. Methods for humanizing non-human antibodies are well known in the art.

The KLRB1 antibodies described herein can be affinity matured, for example using selection and/or mutagenesis methods known in the art. In general, an “affinity matured” antibody is one with one or more alterations in one or more hyper variable regions thereof which result in an improvement in the affinity of the antibody for antigen, compared to a parent antibody which does not possess those alteration(s). In one embodiment, an affinity matured antibody has nanomolar or even picomolar affinities for the target antigen. Preferred affinity matured antibodies have an affinity that is five times, more preferably 10 times, even more preferably 20 or 30 times greater than the starting antibody (generally murine, humanized or human) from which the matured antibody is prepared.

An antibody that “binds to,” “specifically binds to,” or is “specific for” a particular polypeptide or an epitope on a particular polypeptide is one that binds to that particular polypeptide or epitope on a particular polypeptide without substantially binding to any other polypeptide or polypeptide epitope. The term “specifically binds” as used herein refers to a KLRB1 agent (e.g., an anti-KLRB1 antibody) that interacts more frequently, more rapidly, with greater duration, with greater affinity, or with some combination of the above to a particular antigen, epitope, protein, or target molecule than with alternative substances. A binding agent (e.g. antibody) that specifically binds an antigen can be identified, for example, by immunoassays, ELISAs, Surface Plasmon Resonance (SPR) assays (e.g., Biacore), or other techniques known to those of skill in the art. As such, described herein are functional equivalents to the specific anti-KLRB1 antibodies described. In some cases, the KLRB1 antibody may be cross reactive with various similar KLRB1 proteins (e.g., with highest affinity for one, such as human KLRB1, and lower affinity for others, such as mouse KLRB1). A binding agent that specifically binds an antigen binds the target antigen with a higher affinity than its affinity for a different antigen. The different antigen can be a related antigen. In some embodiments, a binding agent that specifically binds an antigen binds the target antigen with an affinity that is at least 20 times greater than its affinity for a different antigen, e.g., at least 30 times greater, at least 40 times greater, at least 50 times greater, at least 60 times greater, at least 70 times greater, at least 80 times greater, at least 90 times greater, or at least 100 times greater, than its affinity for a different antigen. In some embodiments, a binding agent that specifically binds a particular antigen binds a different antigen at such a low affinity that binding cannot be detected using an assay described herein or otherwise known in the art. In some embodiments, affinity is measured using SPR technology, e.g., in a Biacore system or other system known to those of skill in the art.

The terms “identical” or “percent identity” in the context of two or more polypeptides (e.g., two anti-KLRB1 antibodies), refer to two or more sequences or subsequences that are the same or have a specified percentage of amino acid residues that are the same, when compared and aligned (introducing gaps, if necessary) for maximum correspondence, not considering any conservative amino acid substitutions as part of the sequence identity. The percent identity may be measured using sequence comparison software or algorithms or by visual inspection. Various algorithms and software that may be used to obtain alignments of amino acid or nucleotide sequences are well-known in the art. These include, but are not limited to, BLAST, ALIGN, Megalign, BestFit, GCG Wisconsin Package, and variants thereof.

The percent identity between two sequences is a function of the number of identical positions shared by the sequences (i.e., % homology=#of identical positions/total #of positions×100), taking into account the number of gaps, and the length of each gap, which need to be introduced for optimal alignment of the two sequences. The comparison of sequences and determination of percent identity between two sequences can be accomplished using a mathematical algorithm. Such homology is well-represented in the art via local alignment tools and/or algorithms, and may include pairwise alignment, multiple sequence alignment methods, structural alignment methods, and/or phylogenetic analysis methods. Where sequences differ in conservative substitutions, the percent sequence identity may be, but not necessarily is, adjusted upwards to correct for the conservative nature of the substitution. Means for making this adjustment are well known to those of skill in the art. Typically, but not necessarily, this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1.

In some embodiments, two polypeptides (e.g., antibodies or antibody domains (e.g., VL, CL, VH, CH1, CH2, CH3 domains) thereof) of the disclosure are substantially identical, meaning they have at least 70%, at least 75%, at least 80%, at least 85%, at least 90%, and in some embodiments at least 95%, 96%, 97%, 98%, 99% amino acid residue identity, when compared and aligned for maximum correspondence, as measured using a sequence comparison algorithm or by visual inspection. In some embodiments, percent identity exists over a region of the sequences that is at least about 10, at least about 20, at least about 20-40, at least about 40-60 amino acid residues, at least about 60-80 nucleotides or amino acid residues in length or any integral value there between. In some embodiments, percent identity exists over a longer region than 60-80 amino acid residues, such as at least about 80-100 amino acid residues, and in some embodiments the sequences are substantially identical over the full length of the sequences being compared, for example, an amino acid sequence.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 80% of the length of the reference sequence, and in some embodiments is at least 90% or 100%. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. For example, the percent identity between two amino acid sequences can determined using the Needleman and Wunsch ((1970) J. Mol. Biol. 48:444-453) algorithm which has been incorporated into the GAP program in the GCG software package (available on the world wide web at gcg.com), using the default parameters, e.g., a Blossum 62 scoring matrix with a gap penalty of 12, a gap extend penalty of 4, and a frameshift gap penalty of 5.

The terms “conservative sequence modifications” or “conservative substitutions” as used herein may refer to amino acid modifications to a target epitope or antibodies and antigen-binding portions thereof of the disclosure that does not significantly affect or alter the binding characteristics of the anti-KLRB1 antibodies. “Conservative substitution” as used herein refers to a substitution in which one amino acid residue is replaced with another amino acid residue having a similar side chain. Families of amino acid residues having similar side chains have been generally defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). For example, substitution of a phenylalanine for a tyrosine is considered to be a conservative substitution. Methods of identifying amino acid conservative substitutions that do not eliminate binding are well-known in the art.

In various embodiments, the antibody is a blocking or antagonist binding agent. “Blocking” or “antagonist” means the agent (e.g., antibody or binding fragment thereof) is one that inhibits or reduces biological activity of the antigen it binds. Certain blocking agents or antagonist agents substantially or completely inhibit the biological activity of the antigen. For example, a KLRB1 binding agent can block KLRB1 signaling (e.g., thereby disrupting KLRB1 signaling and modulating Th17, Th17.1, ex-Th17, Tc17, MAIT, INKT, ILC2, ILC3, peTh2, NK cells, and/or neoplastic T or NK cells cells).

In some embodiments, the KLRB1 binding agent is an antibody that comprises: a. a full length antibody that binds KLRB1 and comprises an Fc domain that can bind Fcgamma receptors with effector function to trigger antibody-dependent cell-mediated cytotoxicity (ADCC); b. an antibody that binds KLRB1 and comprises an Fc domain that can bind complement protein 1q (C1q) with effector function to trigger complement dependent cytotoxicity (CDC); c. an antibody conjugate that binds KLRB1 and comprises a cytotoxic agent, e.g., antibody-drug conjugate (ADC); or d. a multispecific antibody (e.g., a bispecific or trispecific antibody), wherein the antibody binds to KLRB1 and another antigen.

In some embodiments, the KLRB1 antibodies described herein bind to the extracellular domain of human KLRB1; in some embodiments, the antibody cross-reacts with (binds to both of) the extracellular domains of human and cynomolgus KLRB1. In some embodiments, the antibody binds to an epitope of the extracellular domain of KLRB1, wherein the epitope is at least 90% identical in human and cynomolgus.

In some embodiments, the antibody binds to KLRB1 and is not a mouse antibody.

In some embodiments, the antibody described herein is not B199.2 (Invitrogen), HP-3G10 (Invitrogen), OTI1D8 (OriGene), 14F1F11 (OriGene), 702228 (R&D Systems), B-D51 (Cell Sciences), 2F3 (Novus Biologics), EP7169 (Abcam), 191B8 (Miltenyi), DX12 (BD Biosciences), or JNH25G2G22 (Creative Diagnostics).

In some embodiments, a KLRB1-binding agent is an antibody, e.g., a full length antibody comprising an Fc domain including at least one heavy chain. In some embodiments, the antibody is a recombinant antibody. In some embodiments, the antibody is a monoclonal antibody. In some embodiments, the antibody is a chimeric antibody. In some embodiments, the antibody is a humanized antibody. In some embodiments, the antibody is a human antibody. In some embodiments, the antibody is an IgA, IgD, IgE, IgG, or IgM antibody. In some embodiments, the antibody is an IgG1 antibody. In some embodiments, the antibody is an IgG2 antibody. In some embodiments, the antibody is an IgG3 antibody. In some embodiments, the antibody is an IgG4 antibody.

In some embodiments, the antibody is an antibody fragment comprising an antigen-binding site. In some embodiments, the antibody is a scFv. In some embodiments, the antibody is a disulfide-linked scFv. In some embodiments, the antibody is a bispecific antibody or a multispecific antibody. In some embodiments, the antibody is a monovalent antibody. In some embodiments, the antibody is a monospecific antibody. In some embodiments, the antibody is a bivalent antibody. In some embodiments, the antibody is isolated. In some embodiments, the antibody is substantially pure. In some embodiments, a KLRB1-binding agent is a polyclonal antibody. Polyclonal antibodies can be prepared by any method known to those of skill in the art. In some embodiments, polyclonal antibodies are produced by immunizing an animal (e.g., a rabbit, rat, mouse, goat, donkey) with an antigen of interest (e.g., a purified peptide fragment, a recombinant protein, or a fusion protein) using multiple subcutaneous or intraperitoneal injections. In some embodiments, the antigen is conjugated to a carrier such as keyhole limpet hemocyanin (KLH), serum albumin, bovine thyroglobulin, or soybean trypsin inhibitor. The antigen (with or without a carrier protein) is diluted in sterile saline and usually combined with an adjuvant (e.g., Complete or Incomplete Freund's Adjuvant) to form a stable emulsion. After a period of time, polyclonal antibodies are recovered from the immunized animal (e.g., from blood or ascites). In some embodiments, the polyclonal antibodies are purified from serum or ascites according to standard methods in the art including, but not limited to, affinity chromatography, ion-exchange chromatography, gel electrophoresis, and/or dialysis.

In some embodiments, a KLRB1-binding agent is a monoclonal antibody. Monoclonal antibodies can be prepared by any method known to those of skill in the art. In some embodiments, monoclonal antibodies are prepared using hybridoma methods known to one of skill in the art. For example, using a hybridoma method, a mouse, rat, rabbit, hamster, or other appropriate host animal, is immunized as described above. In some embodiments, lymphocytes are immunized in vitro. In some embodiments, the immunizing antigen is a human protein or a fragment thereof. In some embodiments, the immunizing antigen is a mouse protein or a fragment thereof.

Following immunization, lymphocytes are isolated and fused with a suitable myeloma cell line using, for example, polyethylene glycol. The hybridoma cells are selected using specialized media as known in the art and unfused lymphocytes and myeloma cells do not survive the selection process. Hybridomas that produce monoclonal antibodies directed specifically against a chosen antigen can be identified by a variety of methods including, but not limited to, immunoprecipitation, immunoblotting, and in vitro binding assays (e.g., flow cytometry, FACS, ELISA, SPR (e.g., Biacore), and radioimmunoassay). Once hybridoma cells that produce antibodies of the desired specificity, affinity, and/or activity are identified, the clones may be subcloned by limiting dilution techniques. The hybridomas can be propagated either in in vitro culture using standard methods or in vivo as ascites tumors in an animal. The monoclonal antibodies can be purified from the culture medium or ascites fluid according to standard methods in the art including, but not limited to, affinity chromatography, ion-exchange chromatography, gel electrophoresis, and dialysis.

In some embodiments, monoclonal antibodies are made using recombinant DNA techniques as known to one skilled in the art. For example, the polynucleotides encoding an antibody are isolated from mature B-cells or hybridoma cells, such as by RT-PCR using oligonucleotide primers that specifically amplify the genes encoding the heavy and light chains of the antibody, and their sequence is determined using standard techniques. The isolated polynucleotides encoding the heavy and light chains are then cloned into suitable expression vectors which produce the monoclonal antibodies when transfected into host cells such as E. coli, simian COS cells, Chinese hamster ovary (CHO) cells, or myeloma cells that do not otherwise produce immunoglobulin proteins.

In some embodiments, recombinant monoclonal antibodies are isolated from phage display libraries expressing variable domains or CDRs of a desired species. Screening of phage libraries can be accomplished by various techniques known in the art.

In some embodiments, a monoclonal antibody is modified by using recombinant DNA technology to generate alternative antibodies. In some embodiments, the constant domains of the light chain and heavy chain of a mouse monoclonal antibody are substituted for constant regions of a human antibody to generate a chimeric antibody. In some embodiments, the constant regions are truncated or removed to generate a desired antibody fragment of a monoclonal antibody. In some embodiments, site-directed or high-density mutagenesis of the variable region(s) is used to optimize specificity and affinity of a monoclonal antibody.

In some embodiments, a KLRB1-binding agent is a humanized antibody. Various methods for generating humanized antibodies are known in the art. In some embodiments, a humanized antibody comprises one or more amino acid residues that have been introduced into it from a source that is non-human. In some embodiments, humanization is performed by substituting one or more non-human CDR sequences for the corresponding CDR sequences of a human antibody. In some embodiments, the humanized antibodies are constructed by substituting all six CDRs of a non-human antibody (e.g., a mouse antibody) for the corresponding CDRs of a human antibody.

The choice of which human heavy chain variable region and/or light chain variable region is used for generating humanized antibodies can be made based on a variety of factors and by a variety of methods known in the art. In some embodiments, the “best-fit” method is used where the sequence of the variable region of a non-human (e.g., rodent) antibody is screened against the entire library of known human variable region sequences. The human sequence that is most similar to that of the non-human (e.g., rodent) sequence is selected as the human variable region framework for the humanized antibody. In some embodiments, a particular variable region framework derived from a consensus sequence of all human antibodies of a particular subgroup of light or heavy chains is selected as the variable region framework. In some embodiments, the variable region framework sequence is derived from the consensus sequences of the most abundant human subclasses. In some embodiments, human germline genes are used as the source of the variable region framework sequences.

Other methods for humanization include, but are not limited to, a method called “superhumanization” which is described as the direct transfer of CDRs to a human germline framework, a method termed Human String Content (HSC) which is based on a metric of “antibody humanness”, methods based on generation of large libraries of humanized variants (including phage, ribosomal, and yeast display libraries), and methods based on framework region shuffling.

In some embodiments, a KLRB1-binding agent is a human antibody. Human antibodies can be prepared using various techniques known in the art. In some embodiments, human antibodies are generated from immortalized human B lymphocytes immunized in vitro. In some embodiments, human antibodies are generated from lymphocytes isolated from an immunized individual. In any case, cells that produce an antibody directed against a target antigen can be generated and isolated. In some embodiments, a human antibody is selected from a phage library, where that phage library expresses human antibodies. Alternatively, phage display technology may be used to produce human antibodies and antibody fragments in vitro, from immunoglobulin variable region gene repertoires from unimmunized donors. Techniques for the generation and use of antibody phage libraries are well known in the art. Once antibodies are identified, affinity maturation strategies known in the art, including but not limited to, chain shuffling and site-directed mutagenesis, may be employed to generate higher affinity human antibodies. In some embodiments, human antibodies are produced in transgenic mice that contain human immunoglobulin loci. Upon immunization these mice are capable of producing the full repertoire of human antibodies in the absence of endogenous immunoglobulin production.

In some embodiments, a KLRB1-binding agent is a scFv antibody. ScFvs are molecules that comprise a variable heavy chain region and a variable light chain region linked to form a single polypeptide. ScFvs can be produced using recombinant technologies known in the art. In some embodiments, a scFv comprises a polypeptide linker between the heavy chain variable region and the light chain variable region. In some embodiments, the scFv comprises an orientation of (from N- to C-terminus) (i) heavy chain variable region, (ii) linker, and (iii) light chain variable region. In some embodiments, the scFv comprises an orientation (from N- to C-terminus) of (i) light chain variable region, (ii) linker, and (iii) heavy chain variable region. In some embodiments, the scFv is a disulfide-linked scFv (dsscFv), which is a scFv comprising an engineered disulfide bond between the light chain variable region and heavy chain variable region of the scFv. In some embodiments, the scFv (e.g., dsscFv) is attached (either directly or indirectly) to a half-life extending moiety such as, e.g., an Fc molecule, a CH3 domain of an immunoglobulin (e.g., CH3 of IgG1), polyethylene glycol (PEG) or a PEG mimetic, XTEN, serum albumin (e.g., human serum albumin), polysicalic acid, N-(2-hydroxypropyl) methacrylamide, or dextran, or is modified by, e.g., hyperglycosylation, to extend the half-life of the scFv (e.g., dsscFv).

A variety of suitable linkers are known to those of skill in the art and are not limited by any specific sequences disclosed herein. In some embodiments, the polypeptide linker is comprised of naturally, or non-naturally, occurring amino acids. In some embodiments, the linker comprises amino acids that allow for flexibility. In some embodiments, the linker comprises amino acids that allow for suitable solubility. In some embodiments, the linker comprises glycine amino acids. In some embodiments, the linker comprises glycine and serine amino acids. In certain embodiments, the linker comprises one or more sets of glycine/serine repeats. In some embodiments, the polypeptide linker is selected from the group consisting of: (GGGGS) n wherein n=1-4 (SEQ ID NO:399), GGGGS (SEQ ID NO:399), GGGGSGGGGS (SEQ ID NO:400), GGGGSGGGGSGGGGS (SEQ ID NO:401), GGGGGGGGSGGGGSGGGGS (SEQ ID NO:402), and (GGGGA) n wherein n=1-4 (SEQ ID NO: 403). In some embodiments, the linker comprises GGGGSGGGGSGGGGS (SEQ ID NO: 401).

In some embodiments, a KLRB1-binding agent is a Fv. A Fv comprises a heavy chain variable region and a light chain variable region. In some embodiments, the Fv is attached (either directly or indirectly) to a half-life extending moiety such as, e.g., an Fc molecule, a CH3 domain of an IgG (e.g., CH3 of IgG1), PEG or a PEG mimetic, XTEN, serum albumin (e.g., human serum albumin), polysicalic acid, N-(2-hydroxypropyl) methacrylamide, or dextran, or is modified, e.g., by hyperglycosylation, to extend the half-life of the Fv.

In some embodiments, a KLRB1-binding agent is a Fab. A Fab is one of the molecules that result from digestion of an immunoglobulin antibody with papain. Fabs are monovalent molecules that comprise a light chain, a heavy chain variable region, a CH1 region, and, optionally, a heavy chain constant region hinge region or a portion thereof. Fabs can be produced using recombinant technologies known in the art. In some embodiments, a Fab comprises a polypeptide linker between the light chain constant region and the heavy chain variable region. In some embodiments, a Fab comprises a polypeptide linker between the heavy chain constant region and the light chain variable region. A variety of suitable linkers are known to those of skill in the art and are not limited by any specific sequences disclosed herein. In certain embodiments, the linker is a linker described herein. In some embodiments, the Fab is attached (either directly or indirectly) to a half-life extending moiety such as, e.g., an Fc molecule, a CH3 domain of an IgG (e.g., CH3 of IgG1), PEG or a PEG mimetic, XTEN, serum albumin (e.g., human serum albumin), polysicalic acid, N-(2-hydroxypropyl) methacrylamide, or dextran, or is modified, e.g., by hyperglycosylation, to extend the half-life of the Fab.

In some embodiments, a Fab comprises a disulfide bond formed between the heavy chain variable region and the light chain variable region. In some embodiments, a Fab comprises a disulfide bond that increases stability of the Fab molecule. In some embodiments, a Fab comprises a disulfide bond that increases thermostability of the Fab molecule.

In some embodiments, a KLRB1-binding agent is a F(ab′)₂. A F(ab′)₂ is one of the molecules that results from digestion of an immunoglobulin antibody with pepsin. A F(ab′)₂ is a divalent molecule that comprises a first light chain in association with a first polypeptide comprising a first heavy chain variable region, a first CH1, and a first hinge region, and a second light chain in association with a second polypeptide comprising a second heavy chain variable region, a second CH1, and a second hinge region, wherein the first hinge region is linked to the second hinge region via at least one disulfide bond. F(ab′)_{2S} can be produced using recombinant technologies known in the art. In some embodiments, the F(ab′)₂ is attached (either directly or indirectly) to a half-life extending moiety such as, e.g., a CH3 domain of an IgG (e.g., CH3 of IgG1), PEG or a PEG mimetic, XTEN, serum albumin (e.g., human serum albumin), polysicalic acid, N-(2-hydroxypropyl) methacrylamide, or dextran, or is modified, e.g., by hyperglycosylation, to extend the half-life of the F(ab′)₂.

In some embodiments, a F(ab′)₂ comprises a disulfide bond formed between the heavy chain variable region and the light chain variable region. In some embodiments, a F(ab′)₂ comprises a disulfide bond that increases stability of the F(ab′)₂ molecule. In some embodiments, a F(ab′)₂ comprises a disulfide bond that increases thermostability of the F(ab′)₂ molecule.

In some embodiments, a KLRB1-binding agent is a F(ab′). A F(ab′) is a molecule that results from treatment of a F(ab′)₂ with beta-mercaptoethanol. A F(ab′) is a monovalent molecule that comprises a light chain in association with a polypeptide comprising a heavy chain variable region, a CH1, and a hinge region. In some embodiments, the F(ab′) is attached (either directly or indirectly) to a half-life extending moiety such as, e.g., an Fc molecule, a CH3 domain of an IgG (e.g., CH3 of IgG1), PEG or a PEG mimetic, XTEN, serum albumin (e.g., human serum albumin), polysicalic acid, N-(2-hydroxypropyl) methacrylamide, or dextran, or is modified, e.g., by hyperglycosylation, to extend the half-life of the F(ab′).

In some embodiments, a F(ab′) comprises a disulfide bond formed between the heavy chain variable region and the light chain variable region. In some embodiments, a F(ab′) comprises a disulfide bond that increases stability of the F(ab′) molecule. In some embodiments, a F(ab′) comprises a disulfide bond that increases thermostability of the F(ab′) molecule.

In some embodiments, a KLRB1-binding agent is a bispecific antibody. Bispecific antibodies are capable of recognizing and binding at least two different antigens or epitopes. The different epitopes can either be within the same molecule (e.g., two epitopes on KLRB1) or on different molecules (e.g., one epitope on KLRB1 and one epitope on a different target). In some embodiments, a bispecific antibody has enhanced potency as compared to an individual antibody or to a combination of more than one antibody. In some embodiments, a bispecific antibody has reduced toxicity as compared to an individual antibody or to a combination of more than one antibody. It is known to those of skill in the art that any therapeutic agent may have unique pharmacokinetics (PK) (e.g., circulating half-life). In some embodiments, a bispecific antibody has the ability to synchronize the PK of two active binding agents wherein the two individual binding agents have different PK profiles. In some embodiments, a bispecific antibody has the ability to concentrate the actions of two agents in a common area (e.g., tissue) in a subject (e.g., a human). In some embodiments, a bispecific antibody has the ability to concentrate the actions of two agents to a common target (e.g., a specific cell type). In some embodiments, a bispecific antibody has the ability to target the actions of two agents to more than one biological pathway or function. In some embodiments, a bispecific antibody has the ability to target two different cells and bring them closer together.

In some embodiments, a bispecific antibody has decreased toxicity and/or side effects. In some embodiments, a bispecific antibody has decreased toxicity and/or side effects as compared to a mixture of the two individual antibodies or the antibodies as single agents. In some embodiments, a bispecific antibody has an increased therapeutic index. In some embodiments, a bispecific antibody has an increased therapeutic index as compared to a mixture of the two individual antibodies or the antibodies as single agents.

Several techniques for making bispecific antibodies are known by those skilled in the art. In some embodiments, the bispecific antibodies comprise heavy chain constant regions with modifications in the amino acids that are part of the interface between the two heavy chains. These modifications are made to enhance heterodimer formation and generally reduce or eliminate homodimer formation. In some embodiments, the bispecific antibodies are generated using a knobs-into-holes (KIH) strategy. In some embodiments, the bispecific antibodies comprise variant hinge regions incapable of forming disulfide linkages between identical heavy chains (e.g., reduce homodimer formation). In some embodiments, the bispecific antibodies comprise heavy chains with changes in amino acids that result in altered electrostatic interactions. In some embodiments, the bispecific antibodies comprise heavy chains with changes in amino acids that result in altered hydrophobic/hydrophilic interactions.

Bispecific antibodies can be intact antibodies or antibody fragments comprising antigen-binding sites.

KLRB1-binding agents with more than two valencies are also contemplated. In some embodiments, trispecific or tetraspecific antibodies are generated.

In some embodiments, a KLRB1-binding agent is an anti-KLRB1 antibody that comprises one, two, three, four, five, and/or six CDRs of any one of the antibodies described herein. In some embodiments, an anti-KLRB1 antibody comprises (i) one, two, and/or three heavy chain CDRs from antibody 18G11F11, e.g., as shown in Table 1 and/or (ii) one, two, and/or three light chain CDRs from antibody 18G11F11, e.g., as shown in Table 1. In some embodiments, an anti-KLRB1 antibody comprises (i) three heavy chain CDRs from antibody 18G11F11, e.g., as shown in Table 1 (i.e., a CDR1, a CDR2, and a CDR3), and (ii) three light chain CDRs from antibody 18G11F11, e.g., as shown in Table 1. In some embodiments, an anti-KLRB1 antibody comprises (i) one, two, and/or three heavy chain CDRs from antibody 9C10G11, e.g., as shown in Table 2, and/or (ii) one, two, and/or three light chain CDRs from antibody 9C10G11, e.g., as shown in Table 2. In some embodiments, an anti-KLRB1 antibody comprises (i) three heavy chain CDRs from antibody 9C10G11, e.g., as shown in Table 2 (i.e., a CDR1, a CDR2, and a CDR3), and (ii) three light chain CDRs from antibody 9C10G11, e.g., as shown in Table 2. In some embodiments, an anti-KLRB1 antibody comprises (i) one, two, and/or three heavy chain CDRs from antibody 57E2E4, e.g., as shown in Table 3, and/or (ii) one, two, and/or three light chain CDRs from antibody 57E2E4, e.g., as shown in Table 3. In some embodiments, an anti-KLRB1 antibody comprises (i) three heavy chain CDRs from antibody 57E2E4, e.g., as shown in Table 3 (i.e., a CDR1, a CDR2, and a CDR3), and (ii) three light chain CDRs from antibody 57E2E4, e.g., as shown in Table 3. In some embodiments, an anti-KLRB1 antibody comprises (i) one, two, and/or three heavy chain CDRs from antibody 75B10F12, e.g., as shown in Table 4, and/or (ii) one, two, and/or three light chain CDRs from antibody 75B10F12, e.g., as shown in Table 4. In some embodiments, an anti-KLRB1 antibody comprises (i) three heavy chain CDRs from antibody 75B10F12, e.g., as shown in Table 4 (i.e., a CDR1, a CDR2, and a CDR3), and (ii) three light chain CDRs from antibody 75B10F12, e.g., as shown in Table 4. In some embodiments, an anti-KLRB1 antibody comprises (i) one, two, and/or three heavy chain CDRs from antibody 7B6C7, e.g., as shown in Table 5 and/or (ii) one, two, and/or three light chain CDRs from antibody 7B6C7, e.g., as shown in Table 5. In some embodiments, an anti-KLRB1 antibody comprises (i) three heavy chain CDRs from antibody 7B6C7, e.g., as shown in Table 5 (i.e., a CDR1, a CDR2, and a CDR3), and (ii) three light chain CDRs from antibody 7B6C7, e.g., as shown in Table 5. In some embodiments, an anti-KLRB1 antibody comprises (i) one, two, and/or three heavy chain CDRs from antibody 52G9B12B5, e.g., as shown in Table 6 and/or (ii) one, two, and/or three light chain CDRs from antibody 52G9B12B5, e.g., as shown in Table 6. In some embodiments, an anti-KLRB1 antibody comprises (i) three heavy chain CDRs from antibody 52G9B12B5, e.g., as shown in Table 6 (i.e., a CDR1, a CDR2, and a CDR3), and (ii) three light chain CDRs from antibody 52G9B12B5, e.g., as shown in Table 6. In some embodiments, an anti-KLRB1 antibody comprises (i) one, two, and/or three heavy chain CDRs from antibody 5B1B11, e.g., as shown in Table 7 and/or (ii) one, two, and/or three light chain CDRs from antibody 5B1B11, e.g., as shown in Table 7. In some embodiments, an anti-KLRB1 antibody comprises (i) three heavy chain CDRs from antibody 5B1B11, e.g., as shown in Table 7 (i.e., a CDR1, a CDR2, and a CDR3), and (ii) three light chain CDRs from antibody 5B1B11, e.g., as shown in Table 7. In some embodiments, an anti-KLRB1 antibody comprises (i) one, two, and/or three heavy chain CDRs from antibody 50A8F2, e.g., as shown in Table 8 and/or (ii) one, two, and/or three light chain CDRs from antibody 50A8F2, e.g., as shown in Table 8. In some embodiments, an anti-KLRB1 antibody comprises (i) three heavy chain CDRs from antibody 50A8F2, e.g., as shown in Table 8 (i.e., a CDR1, a CDR2, and a CDR3), and (ii) three light chain CDRs from antibody 50A8F2, e.g., as shown in Table 8. In some embodiments, an anti-KLRB1 antibody comprises (i) one, two, and/or three heavy chain CDRs from antibody 47A5H2, e.g., as shown in Table 9 and/or (ii) one, two, and/or three light chain CDRs from antibody 47A5H2, e.g., as shown in Table 9. In some embodiments, an anti-KLRB1 antibody comprises (i) three heavy chain CDRs from antibody 47A5H2, e.g., as shown in Table 9 (i.e., a CDR1, a CDR2, and a CDR3), and (ii) three light chain CDRs from antibody 47A5H2, e.g., as shown in Table 9. In some embodiments, an anti-KLRB1 antibody comprises (i) one, two, and/or three heavy chain CDRs from antibody 66G9C11, e.g., as shown in Table 10 and/or (ii) one, two, and/or three light chain CDRs from antibody 66G9C11, e.g., as shown in Table 10. In some embodiments, an anti-KLRB1 antibody comprises (i) three heavy chain CDRs from antibody 66G9C11, e.g., as shown in Table 10 (i.e., a CDR1, a CDR2, and a CDR3), and (ii) three light chain CDRs from antibody 66G9C11, e.g., as shown in Table 10. In some embodiments, an anti-KLRB1 antibody comprises (i) one, two, and/or three heavy chain CDRs from antibody 33F11A6, e.g., as shown in Table 11 and/or (ii) one, two, and/or three light chain CDRs from antibody 33F11A6, e.g., as shown in Table 11. In some embodiments, an anti-KLRB1 antibody comprises (i) three heavy chain CDRs from antibody 33F11A6, e.g., as shown in Table 11 (i.e., a CDR1, a CDR2, and a CDR3), and (ii) three light chain CDRs from antibody 33F11A6, e.g., as shown in Table 11. In some embodiments, an anti-KLRB1 antibody comprises (i) one, two, and/or three heavy chain CDRs from antibody 37G6A7, e.g., as shown in Table 12 and/or (ii) one, two, and/or three light chain CDRs from antibody 37G6A7, e.g., as shown in Table 12. In some embodiments, an anti-KLRB1 antibody comprises (i) three heavy chain CDRs from antibody 37G6A7, e.g., as shown in Table 12 (i.e., a CDR1, a CDR2, and a CDR3), and (ii) three light chain CDRs from antibody 37G6A7, e.g., as shown in Table 12. In some embodiments, an anti-KLRB1 antibody comprises (i) one, two, and/or three heavy chain CDRs from antibody 39C5D6, e.g., as shown in Table 13 and/or (ii) one, two, and/or three light chain CDRs from antibody 39C5D6, e.g., as shown in Table 13. In some embodiments, an anti-KLRB1 antibody comprises (i) three heavy chain CDRs from antibody 39C5D6, e.g., as shown in Table 13 (i.e., a CDR1, a CDR2, and a CDR3), and (ii) three light chain CDRs from antibody 39C5D6 e.g., as shown in Table 13. In some embodiments, an anti-KLRB1 antibody comprises (i) one, two, and/or three heavy chain CDRs from antibody 62H1D6, e.g., as shown in Table 14 and/or (ii) one, two, and/or three light chain CDRs from antibody 62H1D6, e.g., as shown in Table 14. In some embodiments, an anti-KLRB1 antibody comprises (i) three heavy chain CDRs from antibody 62H1D6, e.g., as shown in Table 14 (i.e., a CDR1, a CDR2, and a CDR3), and (ii) three light chain CDRs from antibody 62H1D6, e.g., as shown in Table 14. In some embodiments, an anti-KLRB1 antibody comprises (i) one, two, and/or three heavy chain CDRs from antibody 92E12F2, e.g., as shown in Table 15 and/or (ii) one, two, and/or three light chain CDRs from antibody 92E12F2, e.g., as shown in Table 15. In some embodiments, an anti-KLRB1 antibody comprises (i) three heavy chain CDRs from antibody 92E12F2, e.g., as shown in Table 15 (i.e., a CDR1, a CDR2, and a CDR3), and (ii) three light chain CDRs from antibody 92E12F2, e.g., as shown in Table 15. In some embodiments, an anti-KLRB1 antibody comprises (i) one, two, and/or three heavy chain CDRs from antibody 13H2D11, e.g., as shown in Table 16 and/or (ii) one, two, and/or three light chain CDRs from antibody 13H2D11, e.g., as shown in Table 16. In some embodiments, an anti-KLRB1 antibody comprises (i) three heavy chain CDRs from antibody 13H2D11, e.g., as shown in Table 16 (i.e., a CDR1, a CDR2, and a CDR3), and (ii) three light chain CDRs from antibody 13H2D11, e.g., as shown in Table 16. In some embodiments, an anti-KLRB1 antibody comprises (i) one, two, and/or three heavy chain CDRs from antibody 5A11D10, e.g., as shown in Table 17 and/or (ii) one, two, and/or three light chain CDRs from antibody 5A11D10, e.g., as shown in Table 17. In some embodiments, an anti-KLRB1 antibody comprises (i) three heavy chain CDRs from antibody 5A11D10, e.g., as shown in Table 17 (i.e., a CDR1, a CDR2, and a CDR3), and (ii) three light chain CDRs from antibody 5A11D10, e.g., as shown in Table 17. In some embodiments, an anti-KLRB1 antibody comprises (i) one, two, and/or three heavy chain CDRs from antibody 22F10G3, e.g., as shown in Table 18 and/or (ii) one, two, and/or three light chain CDRs from antibody 22F10G3, e.g., as shown in Table 18. In some embodiments, an anti-KLRB1 antibody comprises (i) three heavy chain CDRs from antibody 22F10G3, e.g., as shown in Table 18 (i.e., a CDR1, a CDR2, and a CDR3), and (ii) three light chain CDRs from antibody 22F10G3, e.g., as shown in Table 18.

In some embodiments, a KLRB1-binding agent is a humanized version of an anti-KLRB1 antibody that comprises (i) one, two, and/or three heavy chain CDRs, and/or (ii) one, two, and/or three light chain CDRs from any one of Tables 1-18. In some embodiments, a KLRB1-binding agent is a humanized version of an anti-KLRB1 antibody that comprises (i) three heavy chain CDRs (i.e., a CDR1, a CDR2, and a CDR3) and (ii) three light chain CDRs from any one of Tables 1-18, i.e., wherein the heavy chain CDRs and light chain CDRs are from the same table.

TABLE 1
Antibody 18G11F11 Sequences

| 18G11F11 | Kabat | Chothia | AbM | Contact | IMGT |
|---|---|---|---|---|---|
| CDRH1 | DYGVN (SEQ ID | GFSLTDY (SEQ | GFSLTDYGVN | TDYGVN (SEQ | GFSLTDYG (SEQ |
| | NO: 12) | ID NO: 18) | (SEQ ID NO: 20) | ID NO: 22) | ID NO: 28) |
| CDRH2 | MIWGDGSTDYNSA | WGDGS (SEQ | MIWGDGSTD | WLGMIWGDGS | IWGDGST (SEQ |
| | LKS (SEQ ID NO: 13) | ID NO: 19) | (SEQ ID NO: 21) | TD (SEQ ID | ID NO: 29) |
| | | | | NO: 23) | |
| CDRH3 | TYNSYHYWYFDV | TYNSYHYWYF | TYNSYHYWYF | ARTYNSYHYW | ARTYNSYHYWY |
| | (SEQ ID NO: 14) | DV (SEQ ID | DV (SEQ ID | YFD (SEQ ID | FDV (SEQ ID |
| | | NO: 14) | NO: 14) | NO: 24) | NO: 30) |
| CDRL1 | RASGNIHNYLA | RASGNIHNYL | RASGNIHNYL | HNYLAWY (SEQ | GNIHNY (SEQ ID |
| | (SEQ ID NO: 15) | A (SEQ ID | A (SEQ ID | ID NO: 25) | NO: 31) |
| | | NO: 15) | NO: 15) | | |
| CDRL2 | NAETLAD (SEQ ID | NAETLAD (SEQ | NAETLAD (SEQ | LLVYNAETLA | NA (SEQ ID |
| | NO: 16) | ID NO: 16) | ID NO: 16) | (SEQ ID NO: 26) | NO: 32) |
| CDRL3 | QHFWSTPYT (SEQ | QHFWSTPYT | QHFWSTPYT | QHFWSTPY | QHFWSTPYT |
| | ID NO: 17) | (SEQ ID NO: 17) | (SEQ ID NO: 17) | (SEQ ID NO: 27) | (SEQ ID NO: 17) |

18G11F11-VH
QVQLKESGPGLVAPSQSLSITCTVSGFSLTDYGVNWVRQPPGKGLEWLGMIWGDGSTDYNSALKSRLTINKDNSK
SQVFLNMNSLHTDDTASYYCARTYNSYHYWYFDVWGAGTAVTVSS (SEQ ID NO: 33)
18G11F11-VL
DIQMTQSPASLSASVGETVTITCRASGNIHNYLAWYQQKQGKSPQLLVYNAETLADGVPSRFSGSGSGTQYSLKIN
SLQPEDFGSYYCQHFWSTPYTFGGGTKLEIK (SEQ ID NO: 34)

TABLE 2
Antibody 9C10G11 Sequences

| 9C10G11 | Kabat | Chothia | AbM | Contact | IMGT |
|---|---|---|---|---|---|
| CDRH1 | GYGVN (SEQ ID | GFSLTGY (SEQ | GFSLTGYGVN | TGYGVN (SEQ | GFSLTGYG (SEQ |
| | NO: 35) | ID NO: 38) | (SEQ ID NO: 39) | ID NO: 40) | ID NO: 43) |
| CDRH2 | MIWGDGSTDYNSA | WGDGS (SEQ | MIWGDGSTD | WLGMIWGDGST | IWGDGST (SEQ ID |
| | LKS (SEQ ID NO: 13) | ID NO: 19) | (SEQ ID NO: 21) | D (SEQ ID NO: 23) | NO: 29) |
| CDRH3 | TYNNYHYWYFDV | TYNNYHYWYF | TYNNYHYWYF | ARTYNNYHYW | ARTYNNYHYWY |
| | (SEQ ID NO: 36) | DV (SEQ ID | DV (SEQ ID | YFD (SEQ ID | FDV (SEQ ID |
| | | NO: 36) | NO: 36) | NO: 41) | NO: 44) |
| CDRL1 | RASGNIHNYLA | RASGNIHNYLA | RASGNIHNYLA | HNYLAWY (SEQ | GNIHNY (SEQ ID |
| | (SEQ ID NO: 15) | (SEQ ID NO: 15) | (SEQ ID NO: 15) | ID NO: 25) | NO: 31) |
| CDRL2 | NAKTLAD (SEQ ID | NAKTLAD | NAKTLAD | LLVYNAKTLA | NA (SEQ ID |
| | NO: 37) | (SEQ ID NO: 37) | (SEQ ID NO: 37) | (SEQ ID NO: 42) | NO: 32) |
| CDRL3 | QHFWSTPYT (SEQ | QHFWSTPYT | QHFWSTPYT | QHFWSTPY | QHFWSTPYT |
| | ID NO: 17) | (SEQ ID NO: 17) | (SEQ ID NO: 17) | (SEQ ID NO: 27) | (SEQ ID NO: 17) |

9C10G11-VH
QVQLKESGPGL VAPSQSLSITCTVSGFSLTGYGVNWVRQPPGKGLEWLGMIWGDGSTDYNSALKSRLSISKDNSK
SQVFLKMNSLQTDDTARYYCARTYNNYHYWYFDVWGAGTTVTVSS (SEQ ID NO: 45)
9C10G11-VL
DIQMTQSPASLSASVGETVTITCRASGNIHNYLAWYQQKQGKSPQLLVYNAKTLADGVPSRFSGSGSGTQYSLKI
NSLQPEDFGSYYCQHFWSTPYTFGGGTKLEIK (SEQ ID NO: 46)

TABLE 3
Antibody 57E2E4 Sequences

| 57E2E4 | Kabat | Chothia | AbM | Contact | IMGT |
|---|---|---|---|---|---|
| CDRH1 | SGYYWN (SEQ ID | GYSISSGY (SEQ ID | GYSISSGYYWN | SSGYYWN | GYSISSGYY |
| | NO: 47) | NO: 53) | (SEQ ID NO: 55) | (SEQ ID NO: 57) | (SEQ ID |
| | | | | | NO: 63) |
| CDRH2 | YISYDGSNNYNPS | SYDGS (SEQ ID | YISYDGSNN (SEQ | WMAYISYDGS | ISYDGSN |
| | LKN (SEQ ID | NO: 54) | ID NO: 56) | NN (SEQ ID | (SEQ ID |
| | NO: 48) | | | NO: 58) | NO: 64) |
| CDRH3 | GDFYGYVY (SEQ | GDFYGYVY (SEQ | GDFYGYVY (SEQ | TRGDFYGYV | TRGDFYGY |
| | ID NO: 49) | ID NO: 49) | ID NO: 49) | (SEQ ID NO: 59) | VY (SEQ ID |
| | | | | | NO: 65) |
| CDRL1 | RASKRVSTSDYSY | RASKRVSTSDYSY | RASKRVSTSDYSY | STSDYSYMHW | KRVSTSDYS |
| | MH (SEQ ID | MH (SEQ ID NO: 50) | MH (SEQ ID NO: 50) | Y (SEQ ID | Y (SEQ ID |
| | NO: 50) | | | NO: 60) | NO: 66) |
| CDRL2 | LASNLES (SEQ ID | LASNLES (SEQ ID | LASNLES (SEQ ID | LLIYLASNLE | LA (SEQ ID |
| | NO: 51) | NO: 51) | NO: 51) | (SEQ ID NO: 61) | NO: 67) |
| CDRL3 | QHSREFPRT (SEQ | QHSREFPRT (SEQ | QHSREFPRT (SEQ | QHSREFPR | QHSREFPRT |
| | ID NO: 52) | ID NO: 52) | ID NO: 52) | (SEQ ID NO: 62) | (SEQ ID |
| | | | | | NO: 52) |

57E2E4-VH
DVQLQESGPGLVKPSQSLSLTCSVTGYSISSGYYWNWIRQFPGNKLEWMAYISYDGSNNYNPSLKNRVSITRDTS
KNQFFLRLNSVTAEDTATYYCTRGDFYGYVYWGQGTTLTVSL (SEQ ID NO: 68)
57E2E4-VL
DIVLTQSPASLAVSLGQRATISCRASKRVSTSDYSYMHWYQQKPGQPPKLLIYLASNLESGVPARFSGSGSGTDFT
LNIHPVEEEDAATYYCQHSREFPRTFGGGTKLEIK (SEQ ID NO: 69)

TABLE 4
Antibody 75B10F12 Sequences

| 75B10F12 | Kabat | Chothia | AbM | Contact | IMGT |
|---|---|---|---|---|---|
| CDRH1 | SFWIE (SEQ ID | GYTFSSF (SEQ | GYTFSSFWIE | SSFWIE (SEQ ID | GYTFSSFW |
| | NO: 70) | ID NO: 76) | (SEQ ID NO: 78) | NO: 80) | (SEQ ID NO: 86) |
| CDRH2 | EIFPGSGRTNFNERF | FPGSGR (SEQ | EIFPGSGRTN | WIGEIFPGSGRT | IFPGSGRT |
| | KD (SEQ ID NO: 71) | ID NO: 77) | (SEQ ID NO: 79) | N (SEQ ID | (SEQ ID NO: 87) |
| | | | | NO: 81) | |
| CDRH3 | FNYDVRFDF (SEQ | FNYDVRFDF | FNYDVRFDF | ARFNYDVRFD | ARFNYDVRFD |
| | ID NO: 72) | (SEQ ID NO: 72) | (SEQ ID NO: 72) | (SEQ ID NO: 82) | F (SEQ ID |
| | | | | | NO: 88) |
| CDRL1 | RASENVYNNLA | RASENVYNNL | RASENVYNNL | YNNLAWY (SEQ | ENVYNN (SEQ |
| | (SEQ ID NO: 73) | A (SEQ ID | A (SEQ ID | ID NO: 83) | ID NO: 89) |
| | | NO: 73) | NO: 73) | | |
| CDRL2 | GATNLAD (SEQ ID | GATNLAD | GATNLAD | LLIYGATNLA | GA (SEQ ID |
| | NO: 74) | (SEQ ID NO: 74) | (SEQ ID NO: 74) | (SEQ ID NO: 84) | NO: 90) |
| CDRL3 | QHFWGTPWT (SEQ | QHFWGTPWT | QHFWGTPWT | QHFWGTPW | QHFWGTPWT |
| | ID NO: 75) | (SEQ ID NO: 75) | (SEQ ID NO: 75) | (SEQ ID NO: 85) | (SEQ ID NO: 75) |

75B10F12-VH
QVQLQQSGAELMKPGASVKISCKATGYTFSSFWIEWLKQRPGHGLEWIGEIFPGSGRTNFNERFKDKATFAADTS
SNTAYMQLRSLTSEDSAVYYCARFNYDVRFDFWGQGTTLTVSS (SEQ ID NO: 91)
75B10F12-VL
DIQMTQSPVSLSISVGETVTITCRASENVYNNLAWYQQKQGKSPQLLIYGATNLADGVPSRFSGSGSGTQFSLKIN
SLQSEDFGTYYCQHFWGTPWTFGGGTKLEIK (SEQ ID NO: 92)

TABLE 5
Antibody 7B6C7 Sequences

| 7B6C7 | Kabat | Chothia | AbM | Contact | IMGT |
|---|---|---|---|---|---|
| CDRH1 | DYGVN (SEQ ID | GFSLNDY (SEQ | GFSLNDYGVN | NDYGVN (SEQ | GFSLNDYG (SEQ |
| | NO: 12) | ID NO: 95) | (SEQ ID NO: 96) | ID NO: 97) | ID NO: 100) |
| CDRH2 | MIWGDGSTDYNS | WGDGS (SEQ | MIWGDGSTD | WLGMIWGDGST | IWGDGST (SEQ ID |
| | ALKS (SEQ ID | ID NO: 19) | (SEQ ID NO: 21) | D (SEQ ID NO: 23) | NO: 29) |
| | NO: 13) | | | | |
| CDRH3 | TYNYYHYWYFD | TYNYYHYWYF | TYNYYHYWYF | ARTYNYYHYW | ARTYNYYHYWY |
| | V (SEQ ID NO: 93) | DV (SEQ ID | DV (SEQ ID | YFD (SEQ ID | FDV (SEQ ID |
| | | NO: 93) | NO: 93) | NO: 98) | NO: 101) |
| CDRL1 | RASGNIHNYLS | RASGNIHNYLS | RASGNIHNYLS | HNYLSWY (SEQ | GNIHNY (SEQ ID |
| | (SEQ ID NO: 94) | (SEQ ID NO: 94) | (SEQ ID NO: 94) | ID NO: 99) | NO: 31) |
| CDRL2 | NAETLAD (SEQ | NAETLAD (SEQ | NAETLAD (SEQ | LLVYNAETLA | NA (SEQ ID |
| | ID NO: 16) | ID NO: 16) | ID NO: 16) | (SEQ ID NO: 26) | NO: 32) |
| CDRL3 | QHFWSTPYT | QHFWSTPYT | QHFWSTPYT | QHFWSTPY | QHFWSTPYT |
| | (SEQ ID NO: 17) | (SEQ ID NO: 17) | (SEQ ID NO: 17) | (SEQ ID NO: 27) | (SEQ ID NO: 17) |

7B6C7-VH
QVQLKESGPGLVAPSQSLSITCTVSGFSLNDYGVNWVRQPPGKALEWLGMIWGDGSTDYNSALKSRLTITKDNS
KSQVFLKMNSLHTDDTARYFCARTYNYYHYWYFDVWGAGTTVTVSS (SEQ ID NO: 102)
7B6C7-VL
DIQMTQSPASLSASVGETVTITCRASGNIHNYLSWYQQKQGKSPQLLVYNAETLADGVPSRFSGSGSGTQYSLKIN
NLQPEDFGSYYCQHFWSTPYTFGGGTKLEIK (SEQ ID NO: 103)

TABLE 6
Antibody 52G9B12B5 Sequences

| 52G9B12B5 | Kabat | Chothia | AbM | Contact | IMGT |
|---|---|---|---|---|---|
| CDRH1 | SFWME (SEQ ID | GYTFSSF (SEQ | GYTFSSFWME | SSFWME (SEQ | GYTFSSFW |
| | NO: 104) | ID NO: 76) | (SEQ ID | ID NO: 113) | (SEQ ID |
| | | | NO: 111) | | NO: 86) |
| CDRH2 | EIFPGSGSTNYNEKFK | FPGSGS (SEQ | EIFPGSGSTN | WIGEIFPGSGST | IFPGSGST |
| | G (SEQ ID NO: 105) | ID NO: 110) | (SEQ ID | N (SEQ ID | (SEQ ID |
| | | | NO: 112) | NO: 114) | NO: 119) |
| CDRH3 | LITTGLTY (SEQ ID | LITTGLTY (SEQ | LITTGLTY (SEQ | ATLITTGLT | ATLITTGLT |
| | NO: 106) | ID NO: 106) | ID NO: 106) | (SEQ ID NO: 115) | Y (SEQ ID |
| | | | | | NO: 120) |
| CDRL1 | KASQDVNTAVA | KASQDVNTAV | KASQDVNTAV | NTAVAWY | QDVNTA |
| | (SEQ ID NO: 107) | A (SEQ ID | A (SEQ ID | (SEQ ID NO: 116) | (SEQ ID |
| | | NO: 107) | NO: 107) | | NO: 121) |
| CDRL2 | WASTRHT (SEQ ID | WASTRHT (SEQ | WASTRHT (SEQ | LLIYWASTRH | WA (SEQ ID |
| | NO: 108) | ID NO: 108) | ID NO: 108) | (SEQ ID NO: 117) | NO: 122) |
| CDRL3 | QQHYRIPFT (SEQ ID | QQHYRIPFT | QQHYRIPFT | QQHYRIPF (SEQ | QQHYRIPFT |
| | NO: 109) | (SEQ ID | (SEQ ID | ID NO: 118) | (SEQ ID |
| | | NO: 109) | NO: 109) | | NO: 109) |

52G9B12B5-VH
QVQLQQSGAELMQPGTSVKIPCKATGYTFSSFWMEWVKQRPGHGLEWIGEIFPGSGSTNYNEKFKGKATFTADT
SSNTAYMQLSSLTSEDSAVYYCATLITTGLTYWGQGTLVTVSA (SEQ ID NO: 123)
52G9B12B5-VL
NIVMTQSHKFMSTSVGDRVSITCKASQDVNTAVAWYQQIPGRSPKLLIYWASTRHTGVPDRFTGSGSGTDYTLTIS
SVQAEDLAFYYCQQHYRIPFTFGAGTKLELK (SEQ ID NO: 124)

TABLE 7
Antibody 5B1B11 Sequences

| 5B1B11 | Kabat | Chothia | AbM | Contact | IMGT |
|---|---|---|---|---|---|
| CDRH1 | NYWMH (SEQ ID | GYTFTNY | GYTFTNYWM | TNYWMH (SEQ | GYTFTNYW (SEQ |
| | NO: 125) | (SEQ ID | H (SEQ ID | ID NO: 135) | ID NO: 141) |
| | | NO: 131) | NO: 133) | | |
| CDRH2 | EINPSNGRTNFNEKF | NPSNGR (SEQ | EINPSNGRTN | WIGEINPSNGRT | INPSNGRT (SEQ |
| | KS (SEQ ID NO: 126) | ID NO: 132) | (SEQ ID | N (SEQ ID | ID NO: 142) |
| | | | NO: 134) | NO: 136) | |
| CDRH3 | SYDSYYAAVDY | SYDSYYAAVD | SYDSYYAAVD | ARSYDSYYAAV | ARSYDSYYAAVD |
| | (SEQ ID NO: 127) | Y (SEQ ID | Y (SEQ ID | D (SEQ ID | Y (SEQ ID NO: 143) |
| | | NO: 127) | NO: 127) | NO: 137) | |
| CDRL1 | SASSSVSYMH (SEQ | SASSSVSYMH | SASSSVSYMH | SYMHWY (SEQ | SSVSY (SEQ ID |
| | ID NO: 128) | (SEQ ID | (SEQ ID | ID NO: 138) | NO: 144) |
| | | NO: 128) | NO: 128) | | |
| CDRL2 | TTSNLPS (SEQ ID | TTSNLPS (SEQ | TTSNLPS (SEQ | LLIYTTSNLP | TT (SEQ ID |
| | NO: 129) | ID NO: 129) | ID NO: 129) | (SEQ ID NO: 139) | NO: 145) |
| CDRL3 | HQWSTYPYT (SEQ | HQWSTYPYT | HQWSTYPYT | HQWSTYPY | HQWSTYPYT |
| | ID NO: 130) | (SEQ ID | (SEQ ID | (SEQ ID NO: 140) | (SEQ ID NO: 130) |
| | | NO: 130) | NO: 130) | | |

5B1B11-VH
QVQLQQPGAELVKPGASVKLSCKASGYTFTNYWMHWVKQRPGQGLEWIGEINPSNGRTNFNEKFKSKATLTVD
KSSSTAYMQLSSLTSEDSAVYYCARSYDSYYAAVDYWGQGTSVTVSS (SEQ ID NO: 146)
5B1B11-VL
QIVLTQSPAIMSASLGEEITLTCSASSSVSYMHWYQQKSGTSPKLLIYTTSNLPSGVPSRFSGSGSGTFYSLTISSVEA
EDAADYYCHQWSTYPYTFGGGTKLEIK (SEQ ID NO: 147)

TABLE 8
Antibody 50A8F2 Sequences with Humanized Sequence Variants

| 50A8F2 | Kabat | Chothia | AbM | Contact | IMGT |
|---|---|---|---|---|---|
| CDRH1 | DYYIN (SEQ ID | GYTFIDY (SEQ | GYTFIDYYIN | IDYYIN (SEQ ID | GYTFIDYY (SEQ |
| | NO: 148) | ID NO: 154) | (SEQ ID | NO: 158) | ID NO: 164) |
| | | | NO: 156) | | |
| CDRH2 | WIFPGSGHTKYNENF | FPGSGH (SEQ | WIFPGSGHTK | WIGWIFPGSGHT | IFPGSGHT (SEQ |
| | KG (SEQ ID NO: 149) | ID NO: 155) | (SEQ ID | K (SEQ ID | ID NO: 165) |
| | | | NO: 157) | NO: 159) | |
| CDRH3 | GDRSALGYFDY (SEQ | GDRSALGYFD | GDRSALGYFD | ARGDRSALGYF | ARGDRSALGYF |
| | ID NO: 150) | Y (SEQ ID | Y (SEQ ID | D (SEQ ID | DY (SEQ ID |
| | | NO: 150) | NO: 150) | NO: 160) | NO: 166) |
| CDRL1 | TASSSVSSTYLH (SEQ | TASSSVSSTYL | TASSSVSSTYL | SSTYLHWY | SSVSSTY (SEQ ID |
| | ID NO: 151) | H (SEQ ID | H (SEQ ID | (SEQ ID NO: 161) | NO: 167) |
| | | NO: 151) | NO: 151) | | |
| CDRL2 | STSNLAS (SEQ ID | STSNLAS (SEQ | STSNLAS (SEQ | LWIYSTSNLA | ST (SEQ ID |
| | NO: 152) | ID NO: 152) | ID NO: 152) | (SEQ ID NO: 162) | NO: 168) |
| CDRL3 | HQYHRSPLT (SEQ ID | HQYHRSPLT | HQYHRSPLT | HQYHR SPL | HQYHRSPLT |
| | NO: 153) | (SEQ ID | (SEQ ID | (SEQ ID NO: 163) | (SEQ ID NO: 153) |
| | | NO: 153) | NO: 153) | | |

50A8F2-VH
QIQLQQSGPELVKPGASVKISCKASGYTFIDYYINWVKQKPGQGLEWIGWIFPGSGHTKYNENFKGKATLTVDTSS
STAYMHLSSLTSEDTAVYFCARGDRSALGYFDYWGQGSTLTVSS (SEQ ID NO: 169)
50A8F2-VL
QIVLTQSPAIMSASLGERVTMTCTASSSVSSTYLHWYQQKPGSSPKLWIYSTSNLASGVPARFSGSGSGTSYSLTFS
SMEAEDAATYYCHQYHRSPLTFGAGTKLELK (SEQ ID NO: 170)
50A8F2-VH_g0
QVQLVQSGAEVKKPGASVKVSCKASGYTFIDYYINWVRQAPGQGLEWMGWIFPGSGHTKYNENFKGRVTMTRD
TSTSTVYMELSSLRSEDTAVYYCARGDRSALGYFDYWGQGTTVTVSS (SEQ ID NO: 355)
50A8F2-VH_g1
QVQLVQSGAEVKKPGASVKVSCKASGYTFIDYYINWVRQAPGQGLEWMGWIFPGSGHTKYNENFKGKVTMTV
DTSTSTVYMELSSLRSEDTAVYYCARGDRSALGYFDYWGQGTTVTVSS (SEQ ID NO: 356)
50A8F2-VH_g2
QVQLVQSGAEVKKPGASVKVSCKASGYTFIDYYINWVRQAPGQGLEWMGWIFPGSGHTKYNENFKGKVTLTVD
TSTSTVYMELSSLRSEDTAVYYCARGDRSALGYFDYWGQGTTVTVSS (SEQ ID NO: 357)
50A8F2-VH_g3
QVQLVQSGAEVKKPGASVKVSCKASGYTFIDYYINWVRQAPGQGLEWIGWIFPGSGHTKYNENFKGKVTLTVDT
STSTVYMELSSLRSEDTAVYFCARGDRSALGYFDYWGQGTTVTVSS (SEQ ID NO: 358)
50A8F2-Vκ_g0
DIQMTQSPSSLSASVGDRVTITCTASSSVSSTYLHWYQQKPGKAPKLLIYSTSNLASGVPSRFSGSGSGTDFTLTISS
LQPEDFATYYCHQYHRSPLTFGQGTKLEIK (SEQ ID NO: 359)
50A8F2-Vκ_g1
DIQMTQSPSSLSASVGDR VTITCTASSSVSSTYLHWYQQKPGKAPKLLIYSTSNLASGVPSRFSGSGSGTDYTLTISS
LQPEDFATYYCHQYHRSPLTFGQGTKLEIK (SEQ ID NO: 360)
50A8F2-Vκ_g2
DIQLTQSPSSLSASVGDRVTITCTASSSVSSTYLHWYQQKPGKSPKLLIYSTSNLASGVPSRFSGSGSGTDYTLTISSL
QPEDFATYYCHQYHRSPLTFGQGTKLEIK (SEQ ID NO: 361)
50A8F2-Vκ_g3
DIQLTQSPSSLSASVGDRVTITCTASSSVSSTYLHWYQQKPGKSPKLLIYSTSNLASGVPSRFSGSGSGTDYTLTISSL
QPEDAATYYCHQYHRSPLTFGQGTKLEIK (SEQ ID NO: 362)

TABLE 9
Antibody 47A5H2 Sequences with Humanized Sequence Variants

| 47A5H2 | Kabat | Chothia | AbM | Contact | IMGT |
|---|---|---|---|---|---|
| CDRH1 | GYWMH (SEQ ID | GYSFTGY (SEQ ID | GYSFTGYWMH | TGYWMH | GYSFTGYW |
| | NO: 171) | NO: 177) | (SEQ ID NO: 179) | (SEQ ID | (SEQ ID |
| | | | | NO: 181) | NO: 187) |
| CDRH2 | MIHPSDSETRLNQK | HPSDSE (SEQ ID | MIHPSDSETR | WIGMIHPSDS | IHPSDSET |
| | FKD (SEQ ID | NO: 178) | (SEQ ID NO: 180) | ETR (SEQ ID | (SEQ ID |
| | NO: 172) | | | NO: 182) | NO: 188) |
| CDRH3 | EGLRRGSFAY (SEQ | EGLRRGSFAY | EGLRRGSFAY | ASEGLRRGSF | ASEGLRRGSF |
| | ID NO: 173) | (SEQ ID NO: 173) | (SEQ ID NO: 173) | A (SEQ ID | AY (SEQ ID |
| | | | | NO: 183) | NO: 189) |
| CDRL1 | RASESVDNYGMTF | RASESVDNYGMT | RASESVDNYGMT | DNYGMTFMS | ESVDNYGMT |
| | MS (SEQ ID NO: 174) | FMS (SEQ ID | FMS (SEQ ID | WF (SEQ ID | F (SEQ ID |
| | | NO: 174) | NO: 174) | NO: 184) | NO: 190) |
| CDRL2 | AASNQGS (SEQ ID | AASNQGS (SEQ ID | AASNQGS (SEQ ID | LLIYAASNQG | AA (SEQ ID |
| | NO: 175) | NO: 175) | NO: 175) | (SEQ ID | NO: 191) |
| | | | | NO: 185) | |
| CDRL3 | QQSKEVPYT (SEQ | QQSKEVPYT (SEQ | QQSKEVPYT (SEQ | QQSKEVPY | QQSKEVPYT |
| | ID NO: 176) | ID NO: 176) | ID NO: 176) | (SEQ ID | (SEQ ID |
| | | | | NO: 186) | NO: 176) |

47A5H2-VH
QVQLQQPGAELVRPGASVKLSCKGSGYSFTGYWMHWVKQRPGQGLEWIGMIHPSDSETRLNQKFKDKATLTVD
KSSSTTYMQLSSPTSEDSAVYYCASEGLRRGSFAYWGQGTLVTVSA (SEQ ID NO: 192)
47A5H2-VL
DIVLTQSPASLAVSLGQRATISCRASESVDNYGMTFMSWFQQKPGQPPKLLIYAASNQGSGVPARFSGSGSGTDFS
LNIHPMEEDDTAMYFCQQSKEVPYTFGGGTKLEIK (SEQ ID NO: 193)
47A5H2-VH_g0
QVQLVQSGAEVKKPGASVKVSCKASGYSFTGYWMHWVRQAPGQGLEWMGMIHPSDSETRLNQKFKDRVTMT
RDTSTSTVYMELSSLRSEDTAVYYCASEGLRRGSFAYWGQGTLVTVSS (SEQ ID NO: 347)
47A5H2-VH_g1
QVQLVQSGAEVKKPGASVKVSCKASGYSFTGYWMHWVRQAPGQGLEWMGMIHPSDSETRLNQKFKDRVTMT
VDTSTSTVYMELSSLRSEDTAVYYCASEGLRRGSFAYWGQGTLVTVSS (SEQ ID NO: 348)
47A5H2-VH_g2
QVQLVQSGAEVKKPGASVKVSCKASGYSFTGYWMHWVRQAPGQGLEWMGMIHPSDSETRLNQKFKDRVTMT
VDKSTSTVYMELSSLRSEDTAVYYCASEGLRRGSFAYWGQGTLVTVSS (SEQ ID NO: 349)
47A5H2-VH_g3
QVQLVQSGAEVKKPGASVKVSCKGSGYSFTGYWMHWVRQAPGQGLEWMGMIHPSDSETRLNQKFKDKVTMT
VDKSTSTVYMELSSLRSEDTAVYYCASEGLRRGSFAYWGQGTLVTVSS (SEQ ID NO: 350)
47A5H2-Vκ1_g0
DIVMTQSPDSLAVSLGERATINCRASESVDNYGMTFMSWYQQKPGQPPKLLIYAASNQGSGVPDRFSGSGSGTDF
TLTISSLQAEDVAVYYCQQSKEVPYTFGQGTKLEIK (SEQ ID NO: 351)
47A5H2-Vκ1_g1
DIVLTQSPDSLAVSLGERATINCRASESVDNYGMTFMSWYQQKPGQPPKLLIYAASNQGSGVPDRFSGSGSGTDF
TLTISSLQAEDVAVYYCQQSKEVPYTFGQGTKLEIK (SEQ ID NO: 352)
47A5H2-Vκ1_g2
DIVLTQSPDSLAVSLGERATINCRASESVDNYGMTFMSWFQQKPGQPPKLLIYAASNQGSGVPDRFSGSGSGTDFT
LTISSLQAEDVAVYYCQQSKEVPYTFGQGTKLEIK (SEQ ID NO: 353)
47A5H2-Vκ2_g0
EIVLTQSPATLSLSPGERATLSCRASESVDNYGMTFMSWYQQKPGQAPRLLIYAASNQGSGIPARFSGSGSGTDFT
LTISSLEPEDFAVYYCQQSKEVPYTFGQGTKLEIK (SEQ ID NO: 354)

TABLE 10
Antibody 66G9C11 Sequences

| 66G9C11 | Kabat | Chothia | AbM | Contact | IMGT |
|---|---|---|---|---|---|
| CDRH1 | TFWMN (SEQ ID | GYIFFTF (SEQ | GYIFFTFWMN | FTFWMN (SEQ | GYIFFTFW |
| | NO: 194) | ID NO: 200) | (SEQ ID | ID NO: 204) | (SEQ ID |
| | | | NO: 202) | | NO: 210) |
| CDRH2 | QIFPASGSTNYNEMFE | FPASGS (SEQ | QIFPASGSTN | WIGQIFPASGST | IFPASGST |
| | G (SEQ ID NO: 195) | ID NO: 201) | (SEQ ID | N (SEQ ID | (SEQ ID |
| | | | NO: 203) | NO: 205) | NO: 211) |
| CDRH3 | LERAWFAY (SEQ ID | LERAWFAY | LERAWFAY | ASLERAWFA | ASLERAWFAY |
| | NO: 196) | (SEQ ID | (SEQ ID | (SEQ ID NO: 206) | (SEQ ID |
| | | NO: 196) | NO: 196) | | NO: 212) |
| CDRL1 | RASQSINNYLH (SEQ | RASQSINNYLH | RASQSINNYLH | NNYLHWY (SEQ | QSINNY (SEQ |
| | ID NO: 197) | (SEQ ID | (SEQ ID | ID NO: 207) | ID NO: 213) |
| | | NO: 197) | NO: 197) | | |
| CDRL2 | YASQSIS (SEQ ID | YASQSIS (SEQ | YASQSIS (SEQ | LLIKYASQSI | YA (SEQ ID |
| | NO: 198) | ID NO: 198) | ID NO: 198) | (SEQ ID NO: 208) | NO: 214) |
| CDRL3 | QQSNSWPPT (SEQ ID | QQSNSWPPT | QQSNSWPPT | QQSNSWPP (SEQ | QQSNSWPPT |
| | NO: 199) | (SEQ ID | (SEQ ID | ID NO: 209) | (SEQ ID |
| | | NO: 199) | NO: 199) | | NO: 199) |

66G9C11-VH
HVQLQQSGPELVRPGASVKLSCKASGYIFFTFWMNWVKQRPGQGLEWIGQIFPASGSTNYNEMFEGKATLTVDT
SSSTAYMQLSSLTSEDSAVYYCASLERAWFAYWGQGTLVTVSA (SEQ ID NO: 215)
66G9C11-VL
DIVLTQSPATLSVTPGDRVSLSCRASQSINNYLHWYQQKSHESPRLLIKYASQSISGIPSRFSGSGSGADFTLSINSVE
TEDFGMYFCQQSNSWPPTFGGGTKLEIK (SEQ ID NO: 216)

TABLE 11
Antibody 33F11A6 Sequences

| 33F11A6 | Kabat | Chothia | AbM | Contact | IMGT |
|---|---|---|---|---|---|
| CDRH1 | DYAVN (SEQ ID | GFSLTDY (SEQ | GFSLTDYAVN | TDYAVN (SEQ | GFSLTDYA (SEQ |
| | NO: 217) | ID NO: 18) | (SEQ ID | ID NO: 221) | ID NO: 224) |
| | | | NO: 220) | | |
| CDRH2 | MIWGDGSTDFNSP | WGDGS (SEQ | MIWGDGSTD | WLGMIWGDGS | IWGDGST (SEQ |
| | LKS (SEQ ID | ID NO: 19) | (SEQ ID NO: 21) | TD (SEQ ID | ID NO: 29) |
| | NO: 218) | | | NO: 23) | |
| CDRH3 | TYNDYHYWYFDV | TYNDYHYWYF | TYNDYHYWYF | ARTYNDYHYW | ARTYNDYHYWY |
| | (SEQ ID NO: 219) | DV (SEQ ID | DV (SEQ ID | YFD (SEQ ID | FDV (SEQ ID |
| | | NO: 219) | NO: 219) | NO: 222) | NO: 225) |
| CDRL1 | RASGNIHNYLA | RASGNIHNYLA | RASGNIHNYLA | HNYLAWY (SEQ | GNIHNY (SEQ ID |
| | (SEQ ID NO: 15) | (SEQ ID NO: 15) | (SEQ ID NO: 15) | ID NO: 25) | NO: 31) |
| CDRL2 | NAKTLAD (SEQ ID | NAKTLAD | NAKTLAD | LLIYNAKTLA | NA (SEQ ID |
| | NO: 37) | (SEQ ID NO: 37) | (SEQ ID NO: 37) | (SEQ ID NO: 223) | NO: 32) |
| CDRL3 | QHFWSTPYT (SEQ | QHFWSTPYT | QHFWSTPYT | QHFWSTPY | QHFWSTPYT |
| | ID NO: 17) | (SEQ ID NO: 17) | (SEQ ID NO: 17) | (SEQ ID NO: 27) | (SEQ ID NO: 17) |

33F11A6-VH
QVQLKESGPGLVAPSQSLSITCTVSGFSLTDYAVNWVRQPPGKGLEWLGMIWGDGSTDFNSPLKSRLSISKDNSKS
QVFLKMNSLQTDDTARYFCARTYNDYHYWYFDVWGAGTTVTVSS (SEQ ID NO: 226)
33F11A6-VL
DIQMTQSPASLSASVGETVTITCRASGNIHNYLAWYQQKQGKSPQLLIYNAKTLADGVPSRFSGSGSGTQYFLKIN
SLQPEDFGSYCCQHFWSTPYTFGGGTKLEIK (SEQ ID NO: 227)

TABLE 12
Antibody 37G6A7 Sequences

| 37G6A7 | Kabat | Chothia | AbM | Contact | IMGT |
|---|---|---|---|---|---|
| CDRH1 | DYGVN (SEQ ID | GFSLTDY (SEQ | GFSLTDYGVN | TDYGVN (SEQ | GFSLTDYG (SEQ |
| | NO: 12) | ID NO: 18) | (SEQ ID NO: 20) | ID NO: 22) | ID NO: 28) |
| CDRH2 | MIWGDGSTDYNSA | WGDGS (SEQ | MIWGDGSTD | WLGMIWGDGS | IWGDGST (SEQ |
| | LKS (SEQ ID NO: 13) | ID NO: 19) | (SEQ ID NO: 21) | TD (SEQ ID | ID NO: 29) |
| | | | | NO: 23) | |
| CDRH3 | TYGTYHYWYFDV | TYGTYHYWYF | TYGTYHYWYF | ARTYGTYHYW | ARTYGTYHYWY |
| | (SEQ ID NO: 228) | DV (SEQ ID | DV (SEQ ID | YFD (SEQ ID | FDV (SEQ ID |
| | | NO: 228) | NO: 228) | NO: 230) | NO: 232) |
| CDRL1 | RASGNIHNYLA | RASGNIHNYL | RASGNIHNYL | HNYLAWY (SEQ | GNIHNY (SEQ ID |
| | (SEQ ID NO: 15) | A (SEQ ID | A (SEQ ID | ID NO: 25) | NO: 31) |
| | | NO: 15) | NO: 15) | | |
| CDRL2 | IAKTLAD (SEQ ID | IAKTLAD (SEQ | IAKTLAD (SEQ | LLVYIAKTLA | IA (SEQ ID |
| | NO: 229) | ID NO: 229) | ID NO: 229) | (SEQ ID NO: 231) | NO: 233) |
| CDRL3 | QHFWSTPYT (SEQ | QHFWSTPYT | QHFWSTPYT | QHFWSTPY | QHFWSTPYT |
| | ID NO: 17) | (SEQ ID NO: 17) | (SEQ ID NO: 17) | (SEQ ID NO: 27) | (SEQ ID NO: 17) |

37G6A7-VH
QVQLKESGPGLVAPSQSLSITCTVSGFSLTDYGVNWVRQPPGKGLEWLGMIWGDGSTDYNSALKSRLTISKDNSK
SQVFLKMNSLQTDDTARYYCARTYGTYHYWYFDVWGAGTTVTVSS (SEQ ID NO: 234)
37G6A7-VL
DIQMTQSPASLSASVGETVTITCRASGNIHNYLAWYQQKQGKSPQLLVYIAKTLADGVPSRFSGSGSGTQYSLKIN
SLQPEDFGTYYCQHFWSTPYTFGGGTKLEIK (SEQ ID NO: 235)

TABLE 13
Antibody 39C5D6 Sequences

| 39C5D6 | Kabat | Chothia | AbM | Contact | IMGT |
|---|---|---|---|---|---|
| CDRH1 | DYGVN (SEQ ID | GFSLTDY (SEQ | GFSLTDYGVN | TDYGVN (SEQ | GFSLTDYG (SEQ |
| | NO: 12) | ID NO: 18) | (SEQ ID NO: 20) | ID NO: 22) | ID NO: 28) |
| CDRH2 | MIWGDGSTDYNLP | WGDGS (SEQ | MIWGDGSTD | WLGMIWGDGST | IWGDGST (SEQ ID |
| | LKS (SEQ ID | ID NO: 19) | (SEQ ID NO: 21) | D (SEQ ID NO: 23) | NO: 29) |
| | NO: 236) | | | | |
| CDRH3 | TYANYHYWYFDV | TYANYHYWYF | TYANYHYWYF | ARTYANYHYW | ARTYANYHYWY |
| | (SEQ ID NO: 237) | DV (SEQ ID | DV (SEQ ID | YFD (SEQ ID | FDV (SEQ ID |
| | | NO: 237) | NO: 237) | NO: 239) | NO: 241) |
| CDRL1 | RASGNIHNYLA | RASGNIHNYLA | RASGNIHNYLA | HNYLAWY (SEQ | GNIHNY (SEQ ID |
| | (SEQ ID NO: 15) | (SEQ ID NO: 15) | (SEQ ID NO: 15) | ID NO: 25) | NO: 31) |
| CDRL2 | NAKTLED (SEQ ID | NAKTLED (SEQ | NAKTLED (SEQ | LLVYNAKTLE | NA (SEQ ID |
| | NO: 238) | ID NO: 238) | ID NO: 238) | (SEQ ID NO: 240) | NO: 32) |
| CDRL3 | QHFWSTPYT (SEQ | QHFWSTPYT | QHFWSTPYT | QHFWSTPY | QHFWSTPYT |
| | ID NO: 17) | (SEQ ID NO: 17) | (SEQ ID NO: 17) | (SEQ ID NO: 27) | (SEQ ID NO: 17) |

39C5D6-VH
QVQLKESGPGLVAPSQSLSITCTVSGFSLTDYGVNWVRQTPGKGLEWLGMIWGDGSTDYNLPLKSRLSITKDNSK
SQVFLKMNSLQTDDTARYYCARTYANYHYWYFDVWGAGTTVTVSS (SEQ ID NO: 242)
39C5D6-VL
DIQMTQSPASLSASVGETVTITCRASGNIHNYLAWYQQKQGKSPQLLVYNAKTLEDGVPSRFSGSGSGTQYSLKIN
SLQPDDFGNYFCQHFWSTPYTFGGGTKLEIK (SEQ ID NO: 243)

TABLE 14
Antibody 62H1D6 Sequences

| 62H1D6 | Kabat | Chothia | AbM | Contact | IMGT |
|---|---|---|---|---|---|
| CDRH1 | DYAMH (SEQ ID | GYTFTDY (SEQ | GYTFTDY AMH | TDYAMH (SEQ | GYTFTDYA |
| | NO: 244) | ID NO: 250) | (SEQ ID NO: 252) | ID NO: 254) | (SEQ ID NO: 260) |
| CDRH2 | VISTYYGNTNYN | STYYGN (SEQ ID | VISTYYGNTN | WIGVISTYYGN | ISTYYGNT (SEQ |
| | QKFKG (SEQ ID | NO: 251) | (SEQ ID NO: 253) | TN (SEQ ID | ID NO: 261) |
| | NO: 245) | | | NO: 255) | |
| CDRH3 | EYGNYPYYYAM | EYGNYPYYYAM | EYGNYPYYYAM | AREYGNYPYY | AREYGNYPYYY |
| | DY (SEQ ID | DY (SEQ ID | DY (SEQ ID | YAMD (SEQ ID | AMDY (SEQ ID |
| | NO: 246) | NO: 246) | NO: 246) | NO: 256) | NO: 262) |
| CDRL1 | KSSQSLLNSSNQ | KSSQSLLNSSNQ | KSSQSLLNSSNQ | LNSSNQKNYL | QSLLNSSNQKN |
| | KNYLA (SEQ ID | KNYLA (SEQ ID | KNYLA (SEQ ID | AWY (SEQ ID | Y (SEQ ID |
| | NO: 247) | NO: 247) | NO: 247) | NO: 257) | NO: 263) |
| CDRL2 | FASTRES (SEQ ID | FASTRES (SEQ ID | FASTRES (SEQ ID | LLVYFASTRE | FA (SEQ ID |
| | NO: 248) | NO: 248) | NO: 248) | (SEQ ID | NO: 264) |
| | | | | NO: 258) | |
| CDRL3 | QQHYNTPFT | QQHYNTPFT | QQHYNTPFT | QQHYNTPF | QQHYNTPFT |
| | (SEQ ID NO: 249) | (SEQ ID NO: 249) | (SEQ ID NO: 249) | (SEQ ID | (SEQ ID NO: 249) |
| | | | | NO: 259) | |

62H1D6-VH
QVQLQQSGPELVRPGVSVKISCKGSGYTFTDYAMHWVKQSHAKSLEWIGVISTYYGNTNYNQKFKGRATMTVD
KSSSTAYMELARLTSEDSAIYYCAREYGNYPYYYAMDYWGQGTSVTVSS (SEQ ID NO: 265)
62H1D6-VL
DIVMTQSPSSLAMSVGQKVTMSCKSSQSLLNSSNQKNYLAWYQQKPGQSPKLLVYFASTRESGVPDRFIGSGSGT
DFTLTISSVQAEDLADYFCQQHYNTPFTFGSGTKLEIK (SEQ ID NO: 266)

TABLE 15
Antibody 92E12F2 Sequences

| 92E12F2 | Kabat | Chothia | AbM | Contact | IMGT |
|---|---|---|---|---|---|
| CDRH1 | SGYYWN (SEQ | GYSITSGY (SEQ | GYSITSGYYWN | TSGYYWN | GYSITSGYY |
| | ID NO: 47) | ID NO: 271) | (SEQ ID NO: 272) | (SEQ ID | (SEQ ID |
| | | | | NO: 273) | NO: 279) |
| CDRH2 | YISYDGSNNDNP | SYDGS (SEQ ID | YISYDGSNN (SEQ | WLGYISYDGS | ISYDGSN |
| | SLTN (SEQ ID | NO: 54) | ID NO: 56) | NN (SEQ ID | (SEQ ID |
| | NO: 267) | | | NO: 274) | NO: 64) |
| CDRH3 | GDFYGYVY | GDFYGYVY (SEQ | GDFYGYVY (SEQ | ARGDFYGYV | ARGDFYGYV |
| | (SEQ ID NO: 49) | ID NO: 49) | ID NO: 49) | (SEQ ID | Y (SEQ ID |
| | | | | NO: 275) | NO: 280) |
| CDRL1 | RASKSVSSSGYT | RASKSVSSSGYTY | RASKSVSSSGYTY | SSSGYTYLHW | KSVSSSGYT |
| | YLH (SEQ ID | LH (SEQ ID | LH (SEQ ID | Y (SEQ ID | Y (SEQ ID |
| | NO: 268) | NO: 268) | NO: 268) | NO: 276) | NO: 281) |
| CDRL2 | LTSNLDS (SEQ | LTSNLDS (SEQ ID | LTSNLDS (SEQ ID | LLIYLTSNLD | LT (SEQ ID |
| | ID NO: 269) | NO: 269) | NO: 269) | (SEQ ID | NO: 282) |
| | | | | NO: 277) | |
| CDRL3 | QHSRELPRT | QHSRELPRT (SEQ | QHSRELPRT (SEQ | QHSRELPR | QHSRELPRT |
| | (SEQ ID NO: 270) | ID NO: 270) | ID NO: 270) | (SEQ ID | (SEQ ID |
| | | | | NO: 278) | NO: 270) |

92E12F2-VH
DVQLQESGPGLVKPSQSLSLTCSVTGYSITSGYYWNWIRQFPGNKLEWLGYISYDGSNNDNPSLTNRISISRDTSR
NQFFLKFNSVTTDDSATYYCARGDFYGYVYWGQGTTLTVSS (SEQ ID NO: 283)
92E12F2-VL
DIVLTQSPASLAVSLGQRATISCRASKSVSSSGYTYLHWYQQKPGQPPKLLIYLTSNLDSGVPARFSGSGSGTDFTL
NIHPVEEEDAATYYCQHSRELPRTFGGGTKLEIK (SEQ ID NO: 284)

TABLE 16
Antibody 13H2D11 Sequences

| 13H2D11 | Kabat | Chothia | AbM | Contact | IMGT |
|---|---|---|---|---|---|
| CDRH1 | SYWMN (SEQ ID | GYSFTSY (SEQ ID | GYSFTSYWMN | TSYWMN (SEQ | GYSFTSYW |
| | NO: 285) | NO: 288) | (SEQ ID NO: 289) | ID NO: 290) | (SEQ ID |
| | | | | | NO: 293) |
| CDRH2 | MIHPSD SETRLNQK | HPSDSE (SEQ ID | MIHPSDSETR | WIGMIHPSDSE | IHPSDSET |
| | FKD (SEQ ID | NO: 178) | (SEQ ID NO: 180) | TR (SEQ ID | (SEQ ID |
| | NO: 172) | | | NO: 182) | NO: 188) |
| CDRH3 | EGFRRGSFAY (SEQ | EGFRRGSFAY | EGFRRGSFAY | ASEGFRRGSF | ASEGFRRGSF |
| | ID NO: 286) | (SEQ ID NO: 286) | (SEQ ID NO: 286) | A (SEQ ID | AY (SEQ ID |
| | | | | NO: 291) | NO: 294) |
| CDRL1 | RASESVDNYGISFM | RASESVDNY GISF | RASESVDNYGISF | DNYGISFMNW | ESVDNYGISF |
| | N (SEQ ID NO: 287) | MN (SEQ ID | MN (SEQ ID | F (SEQ ID | (SEQ ID |
| | | NO: 287) | NO: 287) | NO: 292) | NO: 295) |
| CDRL2 | AASNQGS (SEQ ID | AASNQGS (SEQ | AASNQGS (SEQ | LLIYAASNOG | AA (SEQ ID |
| | NO: 175) | ID NO: 175) | ID NO: 175) | (SEQ ID | NO: 191) |
| | | | | NO: 185) | |
| CDRL3 | QQSKEVPYT (SEQ | QQSKEVPYT | QQSKEVPYT | QQSKEVPY | QQSKEVPYT |
| | ID NO: 176) | (SEQ ID NO: 176) | (SEQ ID NO: 176) | (SEQ ID | (SEQ ID |
| | | | | NO: 186) | NO: 176) |

13H2D11-VH
QVQLQQPGAELVRPGASVKLSCKASGYSFTSYWMNWVKQRPGQGLEWIGMIHPSDSETRLNQKFKDKATLTVD
KSSSTAYMQLSSPTSEDSAVYYCASEGFRRGSFAYWGQGTLVTVSA (SEQ ID NO: 296)
13H2D11-VL
DIVLTQSPASLAVSLGQRATISCRASESVDNYGISFMNWFQQKPGQPPKLLIYAASNQGSGVPARFSGSGSGTDFSL
NIHPMEEDDTAMYFCQQSKEVPYTFGGGTKLEIK (SEQ ID NO: 297)

TABLE 17
Antibody 5A11D10 Sequences

| 5A11D10 | Kabat | Chothia | AbM | Contact | IMGT |
|---|---|---|---|---|---|
| CDRH1 | SDFAWN (SEQ ID | GYSLTSDF (SEQ | GYSLTSDFAWN | TSDFAWN | GYSLTSDFA |
| | NO: 298) | ID NO: 304) | (SEQ ID NO: 306) | (SEQ ID | (SEQ ID |
| | | | | NO: 308) | NO: 314) |
| CDRH2 | DIRYSGSTDYNPSL | RYSGS (SEQ ID | DIRYSGSTD | WMGDIRYSGS | IRYSGST (SEQ |
| | KT (SEQ ID NO: 299) | NO: 305) | (SEQ ID NO: 307) | TD (SEQ ID | ID NO: 315) |
| | | | | NO: 309) | |
| CDRH3 | GGYYGYDEGYPMD | GGYYGYDEGYP | GGYYGYDEGYP | ARGGYYGYDE | ARGGYYGYD |
| | Y (SEQ ID NO: 300) | MDY (SEQ ID | MDY (SEQ ID | GYPMD (SEQ | EGYPMDY |
| | | NO: 300) | NO: 300) | ID NO: 310) | (SEQ ID |
| | | | | | NO: 316) |
| CDRL1 | RASESVDVYGNSF | RASESVDVYGNS | RASESVDVYGN | DVYGNSFMHW | ESVDVYGNSF |
| | MH (SEQ ID NO: 301) | FMH (SEQ ID | SFMH (SEQ ID | Y (SEQ ID | (SEQ ID |
| | | NO: 301) | NO: 301) | NO: 311) | NO: 317) |
| CDRL2 | LAFNLKS (SEQ ID | LAFNLKS (SEQ ID | LAFNLKS (SEQ | LLIYLAFNLK | LA (SEQ ID |
| | NO: 302) | NO: 302) | ID NO: 302) | (SEQ ID | NO: 67) |
| | | | | NO: 312) | |
| CDRL3 | QQNNEDPFT (SEQ | QQNNEDPFT | QQNNEDPFT | QQNNEDPF | QQNNEDPFT |
| | ID NO: 303) | (SEQ ID NO: 303) | (SEQ ID NO: 303) | (SEQ ID | (SEQ ID |
| | | | | NO: 313) | NO: 303) |

5A11D10-VH
DVQLQESGPGQVKPSQSLSLTCTVTGYSLTSDFAWNWIRQFPGNKLEWMGDIRYSGSTDYNPSLKTRSSITRDTSK
NQFFLHLNSVTTEDTATYYCARGGYYGYDEGYPMDYWGQGTSVTVSS (SEQ ID NO: 318)
5A11D10-VL
NIVLTQSPASLAVSLGQRATISCRASESVDVYGNSFMHWYQQKAGQPPKLLIYLAFNLKSGVPARFSGSGSRTDFT
LTIDPVEADDAATYYCQQNNEDPFTFGSGTKLEIK (SEQ ID NO: 319)

TABLE 18
Antibody 22F10G3 Sequences and Humanized Versions

| 22F10G3 | Kabat | Chothia | AbM | Contact | IMGT |
|---|---|---|---|---|---|
| CDRH1 | SDFAWN (SEQ | GYSLTSDF (SEQ | GYSLTSDFAWN | TSDFAWN | GYSLTSDFA |
| | ID NO: 298) | ID NO: 304) | (SEQ ID NO: 306) | (SEQ ID | (SEQ ID |
| | | | | NO: 308) | NO: 314) |
| CDRH2 | DIRYSGSTDYNP | RYSGS (SEQ ID | DIRYSGSTD | WMGDIRYSGS | IRYSGST (SEQ |
| | SLKG (SEQ ID | NO: 305) | (SEQ ID NO: 307) | TD (SEQ ID | ID NO: 315) |
| | NO: 320) | | | NO: 309) | |
| CDRH3 | GGYYGYDEGYP | GGYYGYDEGYP | GGYYGYDEGYP | ARGGYYGYDE | ARGGYYGYD |
| | MDY (SEQ ID | MDY (SEQ ID | MDY (SEQ ID | GYPMD (SEQ | EGYPMDY |
| | NO: 300) | NO: 300) | NO: 300) | ID NO: 310) | (SEQ ID |
| | | | | | NO: 316) |
| CDRL1 | RASESVDVYGN | RASESVDVYGNS | RASESVDVYGN | DVYGNSFMHW | ESVDVYGNSF |
| | SFMH (SEQ ID | FMH (SEQ ID | SFMH (SEQ ID | Y (SEQ ID | (SEQ ID |
| | NO: 301) | NO: 301) | NO: 301) | NO: 311) | NO: 317) |
| CDRL2 | LAFNLKS (SEQ | LAFNLKS (SEQ ID | LAFNLKS (SEQ | LLIYLAFNLK | LA (SEQ ID |
| | ID NO: 302) | NO: 302) | ID NO: 302) | (SEQ ID | NO: 67) |
| | | | | NO: 312) | |
| CDRL3 | QQNNEDPFT | QQNNEDPFT | QQNNEDPFT | QQNNEDPF | QQNNEDPFT |
| | (SEQ ID NO: 303) | (SEQ ID NO: 303) | (SEQ ID NO: 303) | (SEQ ID | (SEQ ID |
| | | | | NO: 313) | NO: 303) |
| CDRL1-NA | RASESVDVYGN | RASESVDVYGNA | RASESVDVYGN | DVYGNAFMH | ESVDVYGNAF |
| | AFMH (SEQ ID | FMH (SEQ ID | AFMH (SEQ ID | WY (SEQ ID | (SEQ ID |
| | NO: 323) | NO: 323) | NO: 323) | NO: 324) | NO: 325) |
| CDRL1-QS | RASESVDVYGQ | RASESVDVYGQS | RASESVDVYGQ | DVYGQSFMHW | ESVDVYGQSF |
| | SFMH (SEQ ID | FMH (SEQ ID | SFMH (SEQ ID | Y (SEQ ID | (SEQ ID |
| | NO: 327) | NO: 327) | NO: 327) | NO: 328) | NO: 329) |

22F10G3-VH
DVQLQESGPGQVKPSQSLSLTCTVTGYSLTSDFAWNWIRQFPGNRLEWMGDIRYSGSTDYNPSLKGRSSITRDTSK
NQFFLQLNSVTTEDTATYYCARGGYYGYDEGYPMDYWGQGTSVTVSS (SEQ ID NO: 321)
22F10G3-VL
NIVLTQSPASLAVSLGQRATISCRASESVDVYGNSFMHWYQQKTGQPPKLLIYLAFNLKSGVPARFSGSGSRTDFT
LTIDPVEADDAATYYCQQNNEDPFTFGSGTKLEIK (SEQ ID NO: 322)
Hu22F10G3-1.1 -VH
QVQLQESGPGLVKPSQTLSLTCTVSGYSLTSDFAWNWIRQHPGKGLEWIGDIRYSGSTDYNPSLKGRVTISVDTSK
NQFSLKLSSVTAADTAVYYCARGGYYGYDEGYPMDYWGQGTLVTVSS (SEQ ID NO: 331)
Hu22F10G3-1.1 -VL
DIVMTQSPDSLAVSLGERATINCRASESVDVYGNSFMHWYQQKPGQPPKLLIYLAFNLKSGVPDRFSGSGSGTDFT
LTISSLQAEDVAVYYCQQNNEDPFTFGQGTKLEIK (SEQ ID NO: 335)
Hu22F10G3-2.3 VH
QVQLQESGPGLVKPSQTLSLTCTVSGYSLTSDFAWNWIRQHPGKGLEWMGDIRYSGSTDYNPSLKGRVTISRDTS
KNQFSLKLSSVTAADTAVYYCARGGYYGYDEGYPMDYWGQGTLVTVSS (SEQ ID NO: 332)
Hu22F10G3-2.3 VL
DIVMTQSPDSLAVSLGERATISCRASESVDVYGNSFMHWYQQKPGQPPKLLIYLAFNLKSGVPDRFSGSGSGTDFT
LTISSLQAEDVAVYYCQQNNEDPFTFGQGTKLEIK (SEQ ID NO: 337)
Hu22F10G3-2.3-NA VH
QVQLQESGPGLVKPSQTLSLTCTVSGYSLTSDFAWNWIRQHPGKGLEWMGDIRYSGSTDYNPSLKGRVTISRDTS
KNQFSLKLSSVTAADTAVYYCARGGYYGYDEGYPMDYWGQGTLVTVSS (SEQ ID NO: 332)
Hu22F10G3-2.3-NA VL
DIVMTQSPDSLAVSLGERATISCRASESVDVYGNAFMHWYQQKPGQPPKLLIYLAFNLKSGVPDRFSGSGSGTDFT
LTISSLQAEDVAVYYCQQNNEDPFTFGQGTKLEIK (SEQ ID NO: 341)
Hu22F10G3-2.3-QS VH
QVQLQESGPGLVKPSQTLSLTCTVSGYSLTSDFAWNWIRQHPGKGLEWMGDIRYSGSTDYNPSLKGRVTISRDTS
KNQFSLKLSSVTAADTAVYYCARGGYYGYDEGYPMDYWGQGTLVTVSS (SEQ ID NO: 332)
Hu22F10G3-2.3-QS VL
DIVMTQSPDSLAVSLGERATISCRASESVDVYGQSFMHWYQQKPGQPPKLLIYLAFNLKSGVPDRFSGSGSGTDFT
LTISSLQAEDVAVYYCQQNNEDPFTFGQGTKLEIK (SEQ ID NO: 345)
HU22F10G3-3.4 VH
QVQLQESGPGLVKPSQTLSLTCTVSGYSLTSDFAWNWIRQHPGKGLEWMGDIRYSGSTDYNPSLKGRSTISRDTSK
NQFSLKLSSVTAADTAVYYCARGGYYGYDEGYPMDYWGQGTLVTVSS (SEQ ID NO: 333)
Hu22F10G3-3.4 VL
DIVMTQSPDSLAVSLGERATISCRASESVDVYGNSFMHWYQQKPGQPPKLLIYLAFNLKSGVPARFSGSGSGTDFT
LTISSLQAEDVATYYCQQNNEDPFTFGQGTKLEIK (SEQ ID NO: 338)

In some embodiments, a KLRB1-binding agent comprises a heavy chain CDR1, CDR2, and CDR3 and/or a light chain CDR1, CDR2, CDR3 from an antibody described herein. In some embodiments, a KLRB1-binding agent comprises a humanized version or humanized variant of an antibody described herein. In some embodiments, a KLRB1-binding agent comprises a heavy chain CDR1, CDR2, and CDR3 and/or a light chain CDR1, CDR2, CDR3 from antibody 18G11F11 (Table 1) or a humanized version thereof. In some embodiments, a KLRB1-binding agent comprises a heavy chain CDR1, CDR2, and CDR3 and/or a light chain CDR1, CDR2, CDR3 from antibody 9C10G11 (Table 2) or a humanized version thereof. In some embodiments, a KLRB1-binding agent comprises a heavy chain CDR1, CDR2, and CDR3 and/or a light chain CDR1, CDR2, CDR3 from antibody 57E2E4 (Table 3) or a humanized version thereof. In some embodiments, a KLRB1-binding agent comprises a heavy chain CDR1, CDR2, and CDR3 and/or a light chain CDR1, CDR2, CDR3 from antibody 75B10F12 (Table 4) or a humanized version thereof. In some embodiments, a KLRB1-binding agent comprises a heavy chain CDR1, CDR2, and CDR3 and/or a light chain CDR1, CDR2, CDR3 from antibody 7B6C7 (Table 5) or a humanized version thereof. In some embodiments, a KLRB1-binding agent comprises a heavy chain CDR1, CDR2, and CDR3 and/or a light chain CDR1, CDR2, CDR3 from antibody 52G9B12B5 (Table 6) or a humanized version thereof. In some embodiments, a KLRB1-binding agent comprises a heavy chain CDR1, CDR2, and CDR3 and/or a light chain CDR1, CDR2, CDR3 from antibody 5B1B11 (Table 7) or a humanized version thereof. In some embodiments, a KLRB1-binding agent comprises a heavy chain CDR1, CDR2, and CDR3 and/or a light chain CDR1, CDR2, CDR3 from antibody 50A8F2 (Table 8) or a humanized version thereof. In some embodiments, a KLRB1-binding agent comprises a heavy chain CDR1, CDR2, and CDR3 and/or a light chain CDR1, CDR2, CDR3 from antibody 47A5H2 (Table 9) or a humanized version thereof. In some embodiments, a KLRB1-binding agent comprises a heavy chain CDR1, CDR2, and CDR3 and/or a light chain CDR1, CDR2, CDR3 from antibody 66G9C11 (Table 10) or a humanized version thereof. In some embodiments, a KLRB1-binding agent comprises a heavy chain CDR1, CDR2, and CDR3 and/or a light chain CDR1, CDR2, CDR3 from antibody 33F11A6 (Table 11) or a humanized version thereof. In some embodiments, a KLRB1-binding agent comprises a heavy chain CDR1, CDR2, and CDR3 and/or a light chain CDR1, CDR2, CDR3 from antibody 37G6A7 (Table 12) or a humanized version thereof. In some embodiments, a KLRB1-binding agent comprises a heavy chain CDR1, CDR2, and CDR3 and/or a light chain CDR1, CDR2, CDR3 from antibody 39C5D6 (Table 13) or a humanized version thereof. In some embodiments, a KLRB1-binding agent comprises a heavy chain CDR1, CDR2, and CDR3 and/or a light chain CDR1, CDR2, CDR3 from antibody 62H1D6 (Table 14) or a humanized version thereof. In some embodiments, a KLRB1-binding agent comprises a heavy chain CDR1, CDR2, and CDR3 and/or a light chain CDR1, CDR2, CDR3 from antibody 92E12F2 (Table 15) or a humanized version thereof. In some embodiments, a KLRB1-binding agent comprises a heavy chain CDR1, CDR2, and CDR3 and/or a light chain CDR1, CDR2, CDR3 from antibody 13H2D11 (Table 16) or a humanized version thereof. In some embodiments, a KLRB1-binding agent comprises a heavy chain CDR1, CDR2, and CDR3 and/or a light chain CDR1, CDR2, CDR3 from antibody 5A11D10 (Table 17) or a humanized version thereof. In some embodiments, a KLRB1-binding agent comprises a heavy chain CDR1, CDR2, and CDR3 and/or a light chain CDR1, CDR2, CDR3 from antibody 22F10G3 (Table 18) or a humanized version thereof.

In some embodiments, a KLRB1-binding agent comprises a humanized version or humanized variant of an antibody described herein. In some embodiments, a KLRB1-binding agent comprises a heavy chain CDR1, CDR2, and CDR3 and/or a light chain CDR1, CDR2, CDR3 shown in Tables 1-18 or a humanized version thereof.

CDRs are defined by a variety of methods/systems by those skilled in the art. These systems and/or definitions have been developed and refined over a number of years and include Kabat, Chothia, IMGT, AbM, and Contact. The Kabat definition is based on sequence variability and generally is the most commonly used. The Chothia definition is based on the location of the structural loop regions. The IMGT system is based on sequence variability and location within the structure of the variable domain. The AbM definition is a compromise between Kabat and Chothia. The Contact definition is based on analyses of the available antibody crystal structures. An Exemplary system is a combination of Kabat and Chothia. Software programs (e.g., abYsis (bioinf.org.uk/abysis/sequence_input/key_annotation/key_annotation.cgi)) are available and known to those of skill in the art for analysis of antibody sequences and determination of CDRs.

The specific CDR sequences defined herein are generally based on Kabat definitions. However, it will be understood that reference to a heavy chain CDR or CDRs and/or a light chain CDR or CDRs of a specific antibody will encompass all CDR definitions as known to those of skill in the art, e.g., as shown in the Tables herein. In some embodiments, CDR sequences used will all be identified using the same definitions, i.e., will all be Chothia, all Kabat, all IMGT, and so on.

In some embodiments, a KLRB1-binding agent is a variant of an agent described herein. In some embodiments, a KLRB1-binding agent (e.g., an antibody) comprises (a) a heavy chain CDR1, CDR2, CDR3 from a VH sequence presented herein (e.g., in Tables 1-18, Table B, or Table C), or a variant thereof comprising 1, 2, 3, or 4 amino acid substitutions; and/or a light chain CDR1, CDR2, and/or CDR3 from a VL sequence presented herein (e.g., in Tables 1-18, Table B, or Table C), or a variant thereof comprising 1, 2, 3, or 4 amino acid substitutions. In some embodiments, the amino acid substitutions are conservative substitutions. In some embodiments, a CDR comprises one conservative amino acid substitution. In some embodiments, a CDR comprises two conservative amino acid substitutions. In some embodiments, a CDR comprises three conservative amino acid substitutions. In some embodiments, a CDR comprises four conservative amino acid substitutions. In some embodiments, the CDR is a heavy chain CDR1. In some embodiments, the CDR is a heavy chain CDR2. In some embodiments, the CDR is a heavy chain CDR3. In some embodiments, the CDR is a light chain CDR1. In some embodiments, the CDR is a light chain CDR2. In some embodiments, the CDR is a light chain CDR3. In some embodiments, the substitutions are made as part of a humanization process. In some embodiments, the substitutions are made as part of a germline humanization process. In some embodiments, the substitutions are made as part of an affinity maturation process. In some embodiments, the substitutions are made as part of an optimization process.

In some embodiments, a KLRB1-binding agent (e.g., an antibody) comprises one or more heavy chain or light chain CDRs that have been modified, e.g., to reduce deamidation within the CDR sequence, to remove Asn (N)-glycosylation sites, to remove cysteines, or to remove Asp to reduce isomerization sites, to remove Met/Trp or Lys, e.g., to reduce the likelihood within a CDR sequence of asparagine (N)-glycosylation, cysteinylation, asparagine (Asn) deamidation, aspartate (Asp) isomerization, methionine/tryptophan (Met/Trp) oxidation, and non-enzymatic lysine (Lys) glycation (see, e.g., Haberger et al., MAbs. 2014 Mar. 1; 6 (2): 327-339; Lu et al., MAbs. 2019 January; 11 (1): 45-57). Deamidation is a chemical reaction in which an amide functional group in the side chain of the amino acids asparagine (N) or glutamine (Q) is removed or converted to another functional group. Generally, asparagine is converted to aspartic acid or isoaspartic acid and glutamine is converted to glutamic acid or polyglutamic acid. In some situations, deamidation may change the structure, function, and/or stability of a polypeptide, potentially resulting in decreased biological activity.

Exemplary human heavy chain variable region and light chain variable region sequences are provided in Table B.

TABLE B
Sequences of Exemplary Antibody Variable Sequences

| Name | Heavy Chain Variable Sequence | Light Chain Variable Sequence |
|---|---|---|
| 9C10G11 | QVQLKESGPGLVAPSQSLSITCTVSGFSLTGY | DIQMTQSPASLSASVGETVTITCRASGNIHNYLA |
| | GVNWVRQPPGKGLEWLGMIWGDGSTDYNS | WYQQKQGKSPQLLVYNAKTLADGVPSRFSGSGS |
| | ALKSRLSISKDNSKSQVFLKMNSLQTDDTAR | GTQYSLKINSLQPEDFGSYYCQHFWSTPYTFGGG |
| | YYCARTYNNYHYWYFDVWGAGTTVTVSS | TKLEIK (SEQ ID NO: 46) |
| | (SEQ ID NO: 45) | |
| 57E2E4 | DVQLQESGPGLVKPSQSLSLTCSVTGYSISSG | DIVLTQSPASLAVSLGQRATISCRASKRVSTSDYS |
| | YYWNWIRQFPGNKLEWMAYISYDGSNNYNP | YMHWYQQKPGQPPKLLIYLASNLESGVPARFSG |
| | SLKNRVSITRDTSKNQFFLRLNSVTAEDTATY | SGSGTDFTLNIHPVEEEDAATYYCQHSREFPRTF |
| | YCTRGDFYGYVYWGQGTTLTVSL (SEQ ID | GGGTKLEIK (SEQ ID NO: 69) |
| | NO: 68) | |
| 75B10F12 | QVQLQQSGAELMKPGASVKISCKATGYTFSS | DIQMTQSPVSLSISVGETVTITCRASENVYNNLA |
| | FWIEWLKQRPGHGLEWIGEIFPGSGRTNFNER | WYQQKQGKSPQLLIYGATNLADGVPSRFSGSGS |
| | FKDKATFAADTSSNTAYMQLRSLTSEDSAVY | GTQFSLKINSLQSEDFGTYYCQHFWGTPWTFGG |
| | YCARFNYDVRFDFWGQGTTLTVSS (SEQ ID | GTKLEIK (SEQ ID NO: 92) |
| | NO: 91) | |
| 7B6C7 | QVQLKESGPGLVAPSQSLSITCTVSGFSLNDY | DIQMTQSPASLSASVGETVTITCRASGNIHNYLS |
| | GVNWVRQPPGKALEWLGMIWGDGSTDYNS | WYQQKQGKSPQLLVYNAETLADGVPSRFSGSGS |
| | ALKSRLTITKDNSKSQVFLKMNSLHTDDTAR | GTQYSLKINNLQPEDFGSYYCQHFWSTPYTFGG |
| | YFCARTYNYYHYWYFDVWGAGTTVTVSS | GTKLEIK (SEQ ID NO: 103) |
| | (SEQ ID NO: 102) | |
| 52G9B12B5 | QVQLQQSGAELMQPGTSVKIPCKATGYTFSS | NIVMTQSHKFMSTSVGDRVSITCKASQDVNTAV |
| | FWMEWVKQRPGHGLEWIGEIFPGSGSTNYNE | AWYQQIPGRSPKLLIYWASTRHTGVPDRFTGSGS |
| | KFKGKATFTADTSSNTAYMQLSSLTSEDSAV | GTDYTLTISSVQAEDLAFYYCQQHYRIPFTFGAG |
| | YYCATLITTGLTYWGQGTLVTVSA (SEQ ID | TKLELK (SEQ ID NO: 124) |
| | NO: 123) | |
| 13H2D11 | QVQLQQPGAELVRPGASVKLSCKASGYSFTS | DIVLTQSPASLAVSLGQRATISCRASESVDNYGIS |
| | YWMNWVKQRPGQGLEWIGMIHPSDSETRLN | FMNWFQQKPGQPPKLLIYAASNQGSGVPARFSG |
| | QKFKDKATLTVDKSSSTAYMQLSSPTSEDSA | SGSGTDFSLNIHPMEEDDTAMYFCQQSKEVPYTF |
| | VYYCASEGFRRGSFAYWGQGTLVTVSA (SEQ | GGGTKLEIK (SEQ ID NO: 297) |
| | ID NO: 296) | |
| 5A11D10 | DVQLQESGPGQVKPSQSLSLTCTVTGYSLTSD | NIVLTQSPASLAVSLGQRATISCRASESVDVYGN |
| | FAWNWIRQFPGNKLEWMGDIRYSGSTDYNP | SFMHWYQQKAGQPPKLLIYLAFNLKSGVPARFS |
| | SLKTRSSITRDTSKNQFFLHLNSVTTEDTATY | GSGSRTDFTLTIDPVEADDAATYYCQQNNEDPFT |
| | YCARGGYYGYDEGYPMDYWGQGTSVTVSS | FGSGTKLEIK (SEQ ID NO: 319) |
| | (SEQ ID NO: 318) | |
| 5B1B11 | QVQLQQPGAELVKPGASVKLSCKASGYTFTN | QIVLTQSPAIMSASLGEEITLTCSASSSVSYMHWY |
| | YWMHWVKQRPGQGLEWIGEINPSNGRTNFN | QQKSGTSPKLLIYTTSNLPSGVPSRFSGSGSGTFY |
| | EKFKSKATLTVDKSSSTAYMQLSSLTSEDSAV | SLTISSVEAEDAADYYCHQWSTYPYTFGGGTKL |
| | YYCARSYDSYYAAVDYWGQGTSVTVSS | EIK (SEQ ID NO: 147) |
| | (SEQ ID NO: 146) | |
| 50A8F2 | QIQLQQSGPELVKPGASVKISCKASGYTFIDY | QIVLTQSPAIMSASLGER VTMTCTASSSVSSTYLH |
| | YINWVKQKPGQGLEWIGWIFPGSGHTKYNEN | WYQQKPGSSPKLWIYSTSNLASGVPARFSGSGSG |
| | FKGKATLTVDTSSSTAYMHLSSLTSEDTAVY | TSYSLTFSSMEAEDAATYYCHQYHRSPLTFGAG |
| | FCARGDRSALGYFDYWGQGSTLTVSS (SEQ | TKLELK (SEQ ID NO: 170) |
| | ID NO: 169) | |
| 47A5H2 | QVQLQQPGAELVRPGASVKLSCKGSGYSFTG | DIVLTQSPASLAVSLGQRATISCRASESVDNYGM |
| | YWMHWVKQRPGQGLEWIGMIHPSDSETRLN | TFMSWFQQKPGQPPKLLIYAASNQGSGVPARFS |
| | QKFKDKATLTVDKSSSTTYMQLSSPTSEDSA | GSGSGTDFSLNIHPMEEDDTAMYFCQQSKEVPY |
| | VYYCASEGLRRGSFAYWGQGTLVTVSA | TFGGGTKLEIK (SEQ ID NO: 193) |
| | (SEQ ID NO: 192) | |
| 66G9C11 | HVQLQQSGPELVRPGASVKLSCKASGYIFFTF | DIVLTQSPATLSVTPGDR VSLSCRASQSINNYLH |
| | WMNWVKQRPGQGLEWIGQIFPASGSTNYNE | WYQQKSHESPRLLIKYASQSISGIPSRFSGSGSGA |
| | MFEGKATLTVDTSSSTAYMQLSSLTSEDSAV | DFTLSINSVETEDFGMYFCQQSNSWPPTFGGGTK |
| | YYCASLERAWFAYWGQGTLVTVSA (SEQ ID | LEIK (SEQ ID NO: 216) |
| | NO: 215) | |
| 33F11A6 | QVQLKESGPGLVAPSQSLSITCTVSGFSLTDY | DIQMTQSPASLSASVGETVTITCRASGNIHNYLA |
| | AVNWVRQPPGKGLEWLGMIWGDGSTDFNSP | WYQQKQGKSPQLLIYNAKTLADGVPSRFSGSGS |
| | LKSRLSISKDNSKSQVFLKMNSLQTDDTARYF | GTQYFLKINSLQPEDFGSYCCQHFWSTPYTFGGG |
| | CARTYNDYHYWYFDVWGAGTTVTVSS (SEQ | TKLEIK (SEQ ID NO: 227) |
| | ID NO: 226) | |
| 37G6A7 | QVQLKESGPGLVAPSQSLSITCTVSGFSLTDY | DIQMTQSPASLSASVGETVTITCRASGNIHNYLA |
| | GVNWVRQPPGKGLEWLGMIWGDGSTDYNS | WYQQKQGKSPQLLVYIAKTLADGVPSRFSGSGS |
| | ALKSRLTISKDNSKSQVFLKMNSLQTDDTAR | GTQYSLKINSLQPEDFGTYYCQHFWSTPYTFGGG |
| | YYCARTYGTYHYWYFDVWGAGTTVTVSS | TKLEIK (SEQ ID NO: 235) |
| | (SEQ ID NO: 234) | |
| 39C5D6 | QVQLKESGPGLVAPSQSLSITCTVSGFSLTDY | DIQMTQSPASLSASVGETVTITCRASGNIHNYLA |
| | GVNWVRQTPGKGLEWLGMIWGDGSTDYNL | WYQQKQGKSPQLLVYNAKTLEDGVPSRFSGSGS |
| | PLKSRLSITKDNSKSQVFLKMNSLQTDDTAR | GTQYSLKINSLQPDDFGNYFCQHFWSTPYTFGG |
| | YYCARTYANYHYWYFDVWGAGTTVTVSS | GTKLEIK (SEQ ID NO: 243) |
| | (SEQ ID NO: 242) | |
| 62H1D6 | QVQLQQSGPELVRPGVSVKISCKGSGYTFTD | DIVMTQSPSSLAMSVGQKVTMSCKSSQSLLNSSN |
| | YAMHWVKQSHAKSLEWIGVISTYYGNTNYN | QKNYLAWYQQKPGQSPKLLVYFASTRESGVPDR |
| | QKFKGRATMTVDKSSSTAYMELARLTSEDSA | FIGSGSGTDFTLTISSVQAEDLADYFCQQHYNTPF |
| | IYYCAREYGNYPYYYAMDYWGQGTSVTVSS | TFGSGTKLEIK (SEQ ID NO: 266) |
| | (SEQ ID NO: 265) | |
| 92E12F2 | DVQLQESGPGLVKPSQSLSLTCSVTGYSITSG | DIVLTQSPASLAVSLGQRATISCRASKSVSSSGYT |
| | YYWNWIRQFPGNKLEWLGYISYDGSNNDNP | YLHWYQQKPGQPPKLLIYLTSNLDSGVPARFSGS |
| | SLTNRISISRDTSRNQFFLKFNSVTTDDSATYY | GSGTDFTLNIHPVEEEDAATYYCQHSRELPRTFG |
| | CARGDFYGYVYWGQGTTLTVSS (SEQ ID | GGTKLEIK (SEQ ID NO: 284) |
| | NO: 283) | |
| 18G11F11 | QVQLKESGPGLVAPSQSLSITCTVSGFSLTDY | DIQMTQSPASLSASVGETVTITCRASGNIHNYLA |
| | GVNWVRQPPGKGLEWLGMIWGDGSTDYNS | WYQQKQGKSPQLLVYNAETLADGVPSRFSGSGS |
| | ALKSRLTINKDNSKSQVFLNMNSLHTDDTAS | GTQYSLKINSLQPEDFGSYYCQHFWSTPYTFGGG |
| | YYCARTYNSYHYWYFDVWGAGTAVTVSS | TKLEIK (SEQ ID NO: 34) |
| | (SEQ ID NO: 33) | |
| Hu47A5H2- | QVQLVQSGAEVKKPGASVKVSCKASGYSFT | DIVMTQSPDSLAVSLGERATINCRASESVDNYG |
| 01 | GYWMHWVRQAPGQGLEWMGMIHPSDSETR | MTFMSWYQQKPGQPPKLLIYAASNQGSGVPDRF |
| | LNQKFKDRVTMTRDTSTSTVYMELSSLRSED | SGSGSGTDFTLTISSLQAEDVAVYYCQQSKEVPY |
| | TAVYYCASEGLRRGSFAYWGQGTLVTVSS | TFGQGTKLEIK (SEQ ID NO: 351) |
| | (SEQ ID NO: 347) | |
| Hu47A5H2- | QVQLVQSGAEVKKPGASVKVSCKASGYSFT | DIVLTQSPDSLAVSLGERATINCRASESVDNYGM |
| 02 | GYWMHWVRQAPGQGLEWMGMIHPSDSETR | TFMSWYQQKPGQPPKLLIYAASNQGSGVPDRFS |
| | LNQKFKDRVTMTRDTSTSTVYMELSSLRSED | GSGSGTDFTLTISSLQAEDVAVYYCQQSKEVPYT |
| | TAVYYCASEGLRRGSFAYWGQGTLVTVSS | FGQGTKLEIK (SEQ ID NO: 352) |
| | (SEQ ID NO: 347) | |
| Hu47A5H2- | QVQLVQSGAEVKKPGASVKVSCKASGYSFT | DIVLTQSPDSLAVSLGERATINCRASESVDNYGM |
| 03 | GYWMHWVRQAPGQGLEWMGMIHPSDSETR | TFMSWFQQKPGQPPKLLIYAASNQGSGVPDRFS |
| | LNQKFKDRVTMTRDTSTSTVYMELSSLRSED | GSGSGTDFTLTISSLQAEDVAVYYCQQSKEVPYT |
| | TAVYYCASEGLRRGSFAYWGQGTLVTVSS | FGQGTKLEIK (SEQ ID NO: 353) |
| | (SEQ ID NO: 347) | |
| Hu47A5H2- | QVQLVQSGAEVKKPGASVKVSCKASGYSFT | EIVLTQSPATLSLSPGERATLSCRASESVDNYGM |
| 04 | GYWMHWVRQAPGQGLEWMGMIHPSDSETR | TFMSWYQQKPGQAPRLLIYAASNQGSGIPARFSG |
| | LNQKFKDRVTMTRDTSTSTVYMELSSLRSED | SGSGTDFTLTISSLEPEDFAVYYCQQSKEVPYTFG |
| | TAVYYCASEGLRRGSFAYWGQGTLVTVSS | QGTKLEIK (SEQ ID NO: 354) |
| | (SEQ ID NO: 347) | |
| Hu47A5H2- | QVQLVQSGAEVKKPGASVKVSCKASGYSFT | DIVMTQSPDSLAVSLGERATINCRASESVDNYG |
| 05 | GYWMHWVRQAPGQGLEWMGMIHPSDSETR | MTFMSWYQQKPGQPPKLLIYAASNQGSGVPDRF |
| | LNQKFKDRVTMTVDTSTSTVYMELSSLRSED | SGSGSGTDFTLTISSLQAEDVAVYYCQQSKEVPY |
| | TAVYYCASEGLRRGSFAYWGQGTLVTVSS | TFGQGTKLEIK (SEQ ID NO: 351) |
| | (SEQ ID NO: 348) | |
| Hu47A5H2- | QVQLVQSGAEVKKPGASVKVSCKASGYSFT | DIVLTQSPDSLAVSLGERATINCRASESVDNYGM |
| 06 | GYWMHWVRQAPGQGLEWMGMIHPSDSETR | TFMSWYQQKPGQPPKLLIYAASNQGSGVPDRFS |
| | LNQKFKDRVTMTVDTSTSTVYMELSSLRSED | GSGSGTDFTLTISSLQAEDVAVYYCQQSKEVPYT |
| | TAVYYCASEGLRRGSFAYWGQGTLVTVSS | FGQGTKLEIK (SEQ ID NO: 352) |
| | (SEQ ID NO: 348) | |
| Hu47A5H2- | QVQLVQSGAEVKKPGASVKVSCKASGYSFT | DIVLTQSPDSLAVSLGERATINCRASESVDNYGM |
| 07 | GYWMHWVRQAPGQGLEWMGMIHPSDSETR | TFMSWFQQKPGQPPKLLIYAASNQGSGVPDRFS |
| | LNQKFKDRVTMTVDTSTSTVYMELSSLRSED | GSGSGTDFTLTISSLQAEDVAVYYCQQSKEVPYT |
| | TAVYYCASEGLRRGSFAYWGQGTLVTVSS | FGQGTKLEIK (SEQ ID NO: 353) |
| | (SEQ ID NO: 348) | |
| Hu47A5H2- | QVQLVQSGAEVKKPGASVKVSCKASGYSFT | EIVLTQSPATLSLSPGERATLSCRASESVDNYGM |
| 08 | GYWMHWVRQAPGQGLEWMGMIHPSD SETR | TFMSWYQQKPGQAPRLLIYAASNQGSGIPARFSG |
| | LNQKFKDRVTMTVDTSTSTVYMELSSLRSED | SGSGTDFTLTISSLEPEDFAVYYCQQSKEVPYTFG |
| | TAVYYCASEGLRRGSFAYWGQGTLVTVSS | QGTKLEIK (SEQ ID NO: 354) |
| | (SEQ ID NO: 348) | |
| Hu47A5H2- | QVQLVQSGAEVKKPGASVKVSCKASGYSFT | DIVMTQSPDSLAVSLGERATINCRASESVDNYG |
| 09 | GYWMHWVRQAPGQGLEWMGMIHPSDSETR | MTFMSWYQQKPGQPPKLLIYAASNQGSGVPDRF |
| | LNQKFKDRVTMTVDKSTSTVYMELSSLRSED | SGSGSGTDFTLTISSLQAEDVAVYYCQQSKEVPY |
| | TAVYYCASEGLRRGSFAYWGQGTLVTVSS | TFGQGTKLEIK (SEQ ID NO: 351) |
| | (SEQ ID NO: 349) | |
| Hu47A5H2- | QVQLVQSGAEVKKPGASVKVSCKASGYSFT | DIVLTQSPDSLAVSLGERATINCRASESVDNYGM |
| 10 | GYWMHWVRQAPGQGLEWMGMIHPSDSETR | TFMSWYQQKPGQPPKLLIYAASNQGSGVPDRFS |
| | LNQKFKDRVTMTVDKSTSTVYMELSSLRSED | GSGSGTDFTLTISSLQAEDVAVYYCQQSKEVPYT |
| | TAVYYCASEGLRRGSFAYWGQGTLVTVSS | FGQGTKLEIK (SEQ ID NO: 352) |
| | (SEQ ID NO: 349) | |
| Hu47A5H2- | QVQLVQSGAEVKKPGASVKVSCKASGYSFT | DIVLTQSPDSLAVSLGERATINCRASESVDNYGM |
| 11 | GYWMHWVRQAPGQGLEWMGMIHPSDSETR | TFMSWFQQKPGQPPKLLIYAASNQGSGVPDRFS |
| | LNQKFKDRVTMTVDKSTSTVYMELSSLRSED | GSGSGTDFTLTISSLQAEDVAVYYCQQSKEVPYT |
| | TAVYYCASEGLRRGSFAYWGQGTLVTVSS | FGQGTKLEIK (SEQ ID NO: 353) |
| | (SEQ ID NO: 349) | |
| Hu47A5H2- | QVQLVQSGAEVKKPGASVKVSCKASGYSFT | EIVLTQSPATLSLSPGERATLSCRASESVDNYGM |
| 12 | GYWMHWVRQAPGQGLEWMGMIHPSDSETR | TFMSWYQQKPGQAPRLLIYAASNQGSGIPARFSG |
| | LNQKFKDRVTMTVDKSTSTVYMELSSLRSED | SGSGTDFTLTISSLEPEDFAVYYCQQSKEVPYTFG |
| | TAVYYCASEGLRRGSFAYWGQGTLVTVSS | QGTKLEIK (SEQ ID NO: 354) |
| | (SEQ ID NO: 349) | |
| Hu47A5H2- | QVQLVQSGAEVKKPGASVKVSCKGSGYSFT | DIVMTQSPDSLAVSLGERATINCRASESVDNYG |
| 13 | GYWMHWVRQAPGQGLEWMGMIHPSDSETR | MTFMSWYQQKPGQPPKLLIYAASNQGSGVPDRF |
| | LNQKFKDKVTMTVDKSTSTVYMELSSLRSED | SGSGSGTDFTLTISSLQAEDVAVYYCQQSKEVPY |
| | TAVYYCASEGLRRGSFAYWGQGTLVTVSS | TFGQGTKLEIK (SEQ ID NO: 351) |
| | (SEQ ID NO: 350) | |
| Hu47A5H2- | QVQLVQSGAEVKKPGASVKVSCKGSGYSFT | DIVLTQSPDSLAVSLGERATINCRASESVDNYGM |
| 14 | GYWMHWVRQAPGQGLEWMGMIHPSDSETR | TFMSWYQQKPGQPPKLLIYAASNQGSGVPDRFS |
| | LNQKFKDKVTMTVDKSTSTVYMELSSLRSED | GSGSGTDFTLTISSLQAEDVAVYYCQQSKEVPYT |
| | TAVYYCASEGLRRGSFAYWGQGTLVTVSS | FGQGTKLEIK (SEQ ID NO: 352) |
| | (SEQ ID NO: 350) | |
| Hu47A5H2- | QVQLVQSGAEVKKPGASVKVSCKGSGYSFT | DIVLTQSPDSLAVSLGERATINCRASESVDNYGM |
| 15 | GYWMHWVRQAPGQGLEWMGMIHPSDSETR | TFMSWFQQKPGQPPKLLIYAASNQGSGVPDRFS |
| | LNQKFKDKVTMTVDKSTSTVYMELSSLRSED | GSGSGTDFTLTISSLQAEDVAVYYCQQSKEVPYT |
| | TAVYYCASEGLRRGSFAYWGQGTLVTVSS | FGQGTKLEIK (SEQ ID NO: 353) |
| | (SEQ ID NO: 350) | |
| Hu47A5H2- | QVQLVQSGAEVKKPGASVKVSCKGSGYSFT | EIVLTQSPATLSLSPGERATLSCRASESVDNYGM |
| 16 | GYWMHWVRQAPGQGLEWMGMIHPSDSETR | TFMSWYQQKPGQAPRLLIYAASNQGSGIPARFSG |
| | LNQKFKDKVTMTVDKSTSTVYMELSSLRSED | SGSGTDFTLTISSLEPEDFAVYYCQQSKEVPYTFG |
| | TAVYYCASEGLRRGSFAYWGQGTLVTVSS | QGTKLEIK (SEQ ID NO: 354) |
| | (SEQ ID NO: 350) | |
| Hu50A8F2- | QVQLVQSGAEVKKPGASVKVSCKASGYTFID | DIQMTQSPSSLSASVGDRVTITCTASSSVSSTYLH |
| 01 | YYINWVRQAPGQGLEWMGWIFPGSGHTKYN | WYQQKPGKAPKLLIYSTSNLASGVPSRFSGSGSG |
| | ENFKGRVTMTRDTSTSTVYMELSSLRSEDTA | TDFTLTISSLQPEDFATYYCHQYHRSPLTFGQGT |
| | VYYCARGDRSALGYFDYWGQGTTVTVSS | KLEIK (SEQ ID NO: 359) |
| | (SEQ ID NO: 355) | |
| Hu50A8F2- | QVQLVQSGAEVKKPGASVKVSCKASGYTFID | DIQMTQSPSSLSASVGDRVTITCTASSSVSSTYLH |
| 02 | YYINWVRQAPGQGLEWMGWIFPGSGHTKYN | WYQQKPGKAPKLLIYSTSNLASGVPSRFSGSGSG |
| | ENFKGRVTMTRDTSTSTVYMELSSLRSEDTA | TDYTLTISSLQPEDFATYYCHQYHRSPLTFGQGT |
| | VYYCARGDRSALGYFDYWGQGTTVTVSS | KLEIK (SEQ ID NO: 360) |
| | (SEQ ID NO: 355) | |
| Hu50A8F2- | QVQLVQSGAEVKKPGASVKVSCKASGYTFID | DIQLTQSPSSLSASVGDRVTITCTASSSVSSTYLH |
| 03 | YYINWVRQAPGQGLEWMGWIFPGSGHTKYN | WYQQKPGKSPKLLIYSTSNLASGVPSRFSGSGSG |
| | ENFKGRVTMTRDTSTSTVYMELSSLRSEDTA | TDYTLTISSLQPEDFATYYCHQYHRSPLTFGQGT |
| | VYYCARGDRSALGYFDYWGQGTTVTVSS | KLEIK (SEQ ID NO: 361) |
| | (SEQ ID NO: 355) | |
| Hu50A8F2- | QVQLVQSGAEVKKPGASVKVSCKASGYTFID | DIQLTQSPSSLSASVGDRVTITCTASSSVSSTYLH |
| 04 | YYINWVRQAPGQGLEWMGWIFPGSGHTKYN | WYQQKPGKSPKLLIYSTSNLASGVPSRFSGSGSG |
| | ENFKGRVTMTRDTSTSTVYMELSSLRSEDTA | TDYTLTISSLQPEDAATYYCHQYHRSPLTFGQGT |
| | VYYCARGDRSALGYFDYWGQGTTVTVSS | KLEIK (SEQ ID NO: 362) |
| | (SEQ ID NO: 355) | |
| Hu50A8F2- | QVQLVQSGAEVKKPGASVKVSCKASGYTFID | DIQMTQSPSSLSASVGDRVTITCTASSSVSSTYLH |
| 05 | YYINWVRQAPGQGLEWMGWIFPGSGHTKYN | WYQQKPGKAPKLLIYSTSNLASGVPSRFSGSGSG |
| | ENFKGKVTMTVDTSTSTVYMELSSLRSEDTA | TDFTLTISSLQPEDFATYYCHQYHRSPLTFGQGT |
| | VYYCARGDRSALGYFDYWGQGTTVTVSS | KLEIK (SEQ ID NO: 359) |
| | (SEQ ID NO: 356) | |
| Hu50A8F2- | QVQLVQSGAEVKKPGASVKVSCKASGYTFID | DIQMTQSPSSLSASVGDRVTITCTASSSVSSTYLH |
| 06 | YYINWVRQAPGQGLEWMGWIFPGSGHTKYN | WYQQKPGKAPKLLIYSTSNLASGVPSRFSGSGSG |
| | ENFKGKVTMTVDTSTSTVYMELSSLRSEDTA | TDYTLTISSLQPEDFATYYCHQYHRSPLTFGQGT |
| | VYYCARGDRSALGYFDYWGQGTTVTVSS | KLEIK (SEQ ID NO: 360) |
| | (SEQ ID NO: 356) | |
| Hu50A8F2- | QVQLVQSGAEVKKPGASVKVSCKASGYTFID | DIQLTQSPSSLSASVGDRVTITCTASSSVSSTYLH |
| 07 | YYINWVRQAPGQGLEWMGWIFPGSGHTKYN | WYQQKPGKSPKLLIYSTSNLASGVPSRFSGSGSG |
| | ENFKGKVTMTVDTSTSTVYMELSSLRSEDTA | TDYTLTISSLQPEDFATYYCHQYHRSPLTFGQGT |
| | VYYCARGDRSALGYFDYWGQGTTVTVSS | KLEIK (SEQ ID NO: 361) |
| | (SEQ ID NO: 356) | |
| Hu50A8F2- | QVQLVQSGAEVKKPGASVKVSCKASGYTFID | DIQLTQSPSSLSASVGDRVTITCTASSSVSSTYLH |
| 08 | YYINWVRQAPGQGLEWMGWIFPGSGHTKYN | WYQQKPGKSPKLLIYSTSNLASGVPSRFSGSGSG |
| | ENFKGKVTMTVDTSTSTVYMELSSLRSEDTA | TDYTLTISSLQPEDAATYYCHQYHRSPLTFGQGT |
| | VYYCARGDRSALGYFDYWGQGTTVTVSS | KLEIK (SEQ ID NO: 362) |
| | (SEQ ID NO: 356) | |
| Hu50A8F2- | QVQLVQSGAEVKKPGASVKVSCKASGYTFID | DIQMTQSPSSLSASVGDRVTITCTASSSVSSTYLH |
| 09 | YYINWVRQAPGQGLEWMGWIFPGSGHTKYN | WYQQKPGKAPKLLIYSTSNLASGVPSRFSGSGSG |
| | ENFKGKVTLTVDTSTSTVYMELSSLRSEDTA | TDFTLTISSLQPEDFATYYCHQYHRSPLTFGQGT |
| | VYYCARGDRSALGYFDYWGQGTTVTVSS | KLEIK (SEQ ID NO: 359) |
| | (SEQ ID NO: 357) | |
| Hu50A8F2- | QVQLVQSGAEVKKPGASVKVSCKASGYTFID | DIQMTQSPSSLSASVGDRVTITCTASSSVSSTYLH |
| 10 | YYINWVRQAPGQGLEWMGWIFPGSGHTKYN | WYQQKPGKAPKLLIYSTSNLASGVPSRFSGSGSG |
| | ENFKGKVTLTVDTSTSTVYMELSSLRSEDTA | TDYTLTISSLQPEDFATYYCHQYHRSPLTFGQGT |
| | VYYCARGDRSALGYFDYWGQGTTVTVSS | KLEIK (SEQ ID NO: 360) |
| | (SEQ ID NO: 357) | |
| Hu50A8F2- | QVQLVQSGAEVKKPGASVKVSCKASGYTFID | DIQLTQSPSSLSASVGDRVTITCTASSSVSSTYLH |
| 11 | YYINWVRQAPGQGLEWMGWIFPGSGHTKYN | WYQQKPGKSPKLLIYSTSNLASGVPSRFSGSGSG |
| | ENFKGKVTLTVDTSTSTVYMELSSLRSEDTA | TDYTLTISSLQPEDFATYYCHQYHRSPLTFGQGT |
| | VYYCARGDRSALGYFDYWGQGTTVTVSS | KLEIK (SEQ ID NO: 361) |
| | (SEQ ID NO: 357) | |
| Hu50A8F2- | QVQLVQSGAEVKKPGASVKVSCKASGYTFID | DIQLTQSPSSLSASVGDRVTITCTASSSVSSTYLH |
| 12 | YYINWVRQAPGQGLEWMGWIFPGSGHTKYN | WYQQKPGKSPKLLIYSTSNLASGVPSRFSGSGSG |
| | ENFKGKVTLTVDTSTSTVYMELSSLRSEDTA | TDYTLTISSLQPEDAATYYCHQYHRSPLTFGQGT |
| | VYYCARGDRSALGYFDYWGQGTTVTVSS | KLEIK (SEQ ID NO: 362) |
| | (SEQ ID NO: 357) | |
| Hu50A8F2- | QVQLVQSGAEVKKPGASVKVSCKASGYTFID | DIQMTQSPSSLSASVGDRVTITCTASSSVSSTYLH |
| 13 | YYINWVRQAPGQGLEWIGWIFPGSGHTKYNE | WYQQKPGKAPKLLIYSTSNLASGVPSRFSGSGSG |
| | NFKGKVTLTVDTSTSTVYMELSSLRSEDTAV | TDFTLTISSLQPEDFATYYCHQYHRSPLTFGQGT |
| | YFCARGDRSALGYFDYWGQGTTVTVSS (SEQ | KLEIK (SEQ ID NO: 359) |
| | ID NO: 358) | |
| Hu50A8F2- | QVQLVQSGAEVKKPGASVKVSCKASGYTFID | DIQMTQSPSSLSASVGDRVTITCTASSSVSSTYLH |
| 14 | YYINWVRQAPGQGLEWIGWIFPGSGHTKYNE | WYQQKPGKAPKLLIYSTSNLASGVPSRFSGSGSG |
| | NFKGKVTLTVDTSTSTVYMELSSLRSEDTAV | TDYTLTISSLQPEDFATYYCHQYHRSPLTFGQGT |
| | YFCARGDRSALGYFDYWGQGTTVTVSS (SEQ | KLEIK (SEQ ID NO: 360) |
| | ID NO: 358) | |
| Hu50A8F2- | QVQLVQSGAEVKKPGASVKVSCKASGYTFID | DIQLTQSPSSLSASVGDRVTITCTASSSVSSTYLH |
| 15 | YYINWVRQAPGQGLEWIGWIFPGSGHTKYNE | WYQQKPGKSPKLLIYSTSNLASGVPSRFSGSGSG |
| | NFKGKVTLTVDTSTSTVYMELSSLRSEDTAV | TDYTLTISSLQPEDFATYYCHQYHRSPLTFGQGT |
| | YFCARGDRSALGYFDYWGQGTTVTVSS (SEQ | KLEIK (SEQ ID NO: 361) |
| | ID NO: 358) | |
| Hu50A8F2- | QVQLVQSGAEVKKPGASVKVSCKASGYTFID | DIQLTQSPSSLSASVGDRVTITCTASSSVSSTYLH |
| 16 | YYINWVRQAPGQGLEWIGWIFPGSGHTKYNE | WYQQKPGKSPKLLIYSTSNLASGVPSRFSGSGSG |
| | NFKGKVTLTVDTSTSTVYMELSSLRSEDTAV | TDYTLTISSLQPEDAATYYCHQYHRSPLTFGQGT |
| | YFCARGDRSALGYFDYWGQGTTVTVSS (SEQ | KLEIK (SEQ ID NO: 362) |
| | ID NO: 358) | |
| 22F10G3 | DVQLQESGPGQVKPSQSLSLTCTVTGYSLTSD | NIVLTQSPASLAVSLGQRATISCRASESVDVYGN |
| | FAWNWIRQFPGNRLEWMGDIRYSGSTDYNPS | SFMHWYQQKTGQPPKLLIYLAFNLKSGVPARFS |
| | LKGRSSITRDTSKNQFFLQLNSVTTEDTATYY | GSGSRTDFTLTIDPVEADDAATYYCQQNNEDPFT |
| | CARGGYYGYDEGYPMDYWGQGTSVTVSS | FGSGTKLEIK (SEQ ID NO: 322) |
| | (SEQ ID NO: 321) | |
| 22F10G3- | QVQLQESGPGLVKPSQTLSLTCTVSGYSLTSD | NIVLTQSPASLAVSLGQRATISCRASESVDVYGN |
| NA | FAWNWIRQHPGKGLEWMGDIRYSGSTDYNP | AFMHWYQQKTGQPPKLLIYLAFNLKSGVPARFS |
| | SLKGRVTISRDTSKNQFSLKLSSVTAADTAVY | GSGSRTDFTLTIDPVEADDAATYYCQQNNEDPFT |
| | YCARGGYYGYDEGYPMDYWGQGTLVTVSS | FGSGTKLEIK (SEQ ID NO: 326) |
| | (SEQ ID NO: 332) | |
| 22F10G3- | DVQLQESGPGQVKPSQSLSLTCTVTGYSLTSD | NIVLTQSPASLAVSLGQRATISCRASESVDVYGQ |
| QS | FAWNWIRQFPGNRLEWMGDIRYSGSTDYNPS | SFMHWYQQKTGQPPKLLIYLAFNLKSGVPARFS |
| | LKGRSSITRDTSKNQFFLQLNSVTTEDTATYY | GSGSRTDFTLTIDPVEADDAATYYCQQNNEDPFT |
| | CARGGYYGYDEGYPMDYWGQGTSVTVSS | FGSGTKLEIK (SEQ ID NO: 330) |
| | (SEQ ID NO: 321) | |
| Hu22F10G3- | QVQLQESGPGLVKPSQTLSLTCTVSGYSLTSD | DIVMTQSPDSLAVSLGERATINCRASESVDVYGN |
| 1.1 | FAWNWIRQHPGKGLEWIGDIRYSGSTDYNPS | SFMHWYQQKPGQPPKLLIYLAFNLKSGVPDRFS |
| | LKGRVTISVDTSKNQFSLKLSSVTAADTAVY | GSGSGTDFTLTISSLQAEDVAVYYCQQNNEDPFT |
| | YCARGGYYGYDEGYPMDYWGQGTLVTVSS | FGQGTKLEIK (SEQ ID NO: 335) |
| | (SEQ ID NO: 331) | |
| Hu22F10G3- | QVQLQESGPGLVKPSQTLSLTCTVSGYSLTSD | DIVMTQSPDSLAVSLGERATINCRASESVDVYGN |
| 1.2 | FAWNWIRQHPGKGLEWIGDIRYSGSTDYNPS | SFMHWYQQKPGQPPKLLIYLAFNLKSGVPARFS |
| | LKGRVTISVDTSKNQFSLKLSSVTAADTAVY | GSGSGTDFTLTISSLQAEDVAVYYCQQNNEDPFT |
| | YCARGGYYGYDEGYPMDYWGQGTLVTVSS | FGQGTKLEIK (SEQ ID NO: 336) |
| | (SEQ ID NO: 331) | |
| Hu22F10G3- | QVQLQESGPGLVKPSQTLSLTCTVSGYSLTSD | DIVMTQSPDSLAVSLGERATISCRASESVDVYGN |
| 1.3 | FAWNWIRQHPGKGLEWIGDIRYSGSTDYNPS | SFMHWYQQKPGQPPKLLIYLAFNLKSGVPDRFS |
| | LKGRVTISVDTSKNQFSLKLSSVTAADTAVY | GSGSGTDFTLTISSLQAEDVAVYYCQQNNEDPFT |
| | YCARGGYYGYDEGYPMDYWGQGTLVTVSS | FGQGTKLEIK (SEQ ID NO: 337) |
| | (SEQ ID NO: 331) | |
| Hu22F10G3- | QVQLQESGPGLVKPSQTLSLTCTVSGYSLTSD | DIVMTQSPDSLAVSLGERATISCRASESVDVYGN |
| 1.4 | FAWNWIRQHPGKGLEWIGDIRYSGSTDYNPS | SFMHWYQQKPGQPPKLLIYLAFNLKSGVPARFS |
| | LKGRVTISVDTSKNQFSLKLSSVTAADTAVY | GSGSGTDFTLTISSLQAEDVATYYCQQNNEDPFT |
| | YCARGGYYGYDEGYPMDYWGQGTLVTVSS | FGQGTKLEIK (SEQ ID NO: 338) |
| | (SEQ ID NO: 331) | |
| Hu22F10G3- | QVQLQESGPGLVKPSQTLSLTCTVSGYSLTSD | DIVMTQSPDSLAVSLGERATINCRASESVDVYGN |
| 2.1 | FAWNWIRQHPGKGLEWMGDIRYSGSTDYNP | SFMHWYQQKPGQPPKLLIYLAFNLKSGVPDRFS |
| | SLKGRVTISRDTSKNQFSLKLSSVTAADTAVY | GSGSGTDFTLTISSLQAEDVAVYYCQQNNEDPFT |
| | YCARGGYYGYDEGYPMDYWGQGTLVTVSS | FGQGTKLEIK (SEQ ID NO: 335) |
| | (SEQ ID NO: 332) | |
| Hu22F10G3- | QVQLQESGPGLVKPSQTLSLTCTVSGYSLTSD | DIVMTQSPDSLAVSLGERATINCRASESVDVYGN |
| 2.2 | FAWNWIRQHPGKGLEWMGDIRYSGSTDYNP | SFMHWYQQKPGQPPKLLIYLAFNLKSGVPARFS |
| | SLKGRVTISRDTSKNQFSLKLSSVTAADTAVY | GSGSGTDFTLTISSLQAEDVAVYYCQQNNEDPFT |
| | YCARGGYYGYDEGYPMDYWGQGTLVTVSS | FGQGTKLEIK (SEQ ID NO: 336) |
| | (SEQ ID NO: 332) | |
| Hu22F10G3- | QVQLQESGPGLVKPSQTLSLTCTVSGYSLTSD | DIVMTQSPDSLAVSLGERATISCRASESVDVYGN |
| 2.3 | FAWNWIRQHPGKGLEWMGDIRYSGSTDYNP | SFMHWYQQKPGQPPKLLIYLAFNLKSGVPDRFS |
| | SLKGRVTISRDTSKNQFSLKLSSVTAADTAVY | GSGSGTDFTLTISSLQAEDVAVYYCQQNNEDPFT |
| | YCARGGYYGYDEGYPMDYWGQGTLVTVSS | FGQGTKLEIK (SEQ ID NO: 337) |
| | (SEQ ID NO: 332) | |
| Hu22F10G3- | QVQLQESGPGLVKPSQTLSLTCTVSGYSLTSD | DIVMTQSPDSLAVSLGERATISCRASESVDVYGN |
| 2.4 | FAWNWIRQHPGKGLEWMGDIRYSGSTDYNP | SFMHWYQQKPGQPPKLLIYLAFNLKSGVPARFS |
| | SLKGRVTISRDTSKNQFSLKLSSVTAADTAVY | GSGSGTDFTLTISSLQAEDVATYYCQQNNEDPFT |
| | YCARGGYYGYDEGYPMDYWGQGTLVTVSS | FGQGTKLEIK (SEQ ID NO: 338) |
| | (SEQ ID NO: 332) | |
| Hu22F10G3- | QVQLQESGPGLVKPSQTLSLTCTVSGYSLTSD | DIVMTQSPDSLAVSLGERATINCRASESVDVYGN |
| 3.1 | FAWNWIRQHPGKGLEWMGDIRYSGSTDYNP | SFMHWYQQKPGQPPKLLIYLAFNLKSGVPDRFS |
| | SLKGRSTISRDTSKNQFSLKLSSVTAADTAVY | GSGSGTDFTLTISSLQAEDVAVYYCQQNNEDPFT |
| | YCARGGYYGYDEGYPMDYWGQGTLVTVSS | FGQGTKLEIK (SEQ ID NO: 335) |
| | (SEQ ID NO: 333) | |
| Hu22F10G3- | QVQLQESGPGLVKPSQTLSLTCTVSGYSLTSD | DIVMTQSPDSLAVSLGERATINCRASESVDVYGN |
| 3.2 | FAWNWIRQHPGKGLEWMGDIRYSGSTDYNP | SFMHWYQQKPGQPPKLLIYLAFNLKSGVPARFS |
| | SLKGRSTISRDTSKNQFSLKLSSVTAADTAVY | GSGSGTDFTLTISSLQAEDVAVYYCQQNNEDPFT |
| | YCARGGYYGYDEGYPMDYWGQGTLVTVSS | FGQGTKLEIK (SEQ ID NO: 336) |
| | (SEQ ID NO: 333) | |
| Hu22F10G3- | QVQLQESGPGLVKPSQTLSLTCTVSGYSLTSD | DIVMTQSPDSLAVSLGERATISCRASESVDVYGN |
| 3.3 | FAWNWIRQHPGKGLEWMGDIRYSGSTDYNP | SFMHWYQQKPGQPPKLLIYLAFNLKSGVPDRFS |
| | SLKGRSTISRDTSKNQFSLKLSSVTAADTAVY | GSGSGTDFTLTISSLQAEDVAVYYCQQNNEDPFT |
| | YCARGGYYGYDEGYPMDYWGQGTLVTVSS | FGQGTKLEIK (SEQ ID NO: 337) |
| | (SEQ ID NO: 333) | |
| Hu22F10G3- | QVQLQESGPGLVKPSQTLSLTCTVSGYSLTSD | DIVMTQSPDSLAVSLGERATISCRASESVDVYGN |
| 3.4 | FAWNWIRQHPGKGLEWMGDIRYSGSTDYNP | SFMHWYQQKPGQPPKLLIYLAFNLKSGVPARFS |
| | SLKGRSTISRDTSKNQFSLKLSSVTAADTAVY | GSGSGTDFTLTISSLQAEDVATYYCQQNNEDPFT |
| | YCARGGYYGYDEGYPMDYWGQGTLVTVSS | FGQGTKLEIK (SEQ ID NO: 338) |
| | (SEQ ID NO: 333) | |
| Hu22F10G3- | QVQLQESGPGLVKPSQTLSLTCTVSGYSLTSD | DIVMTQSPDSLAVSLGERATINCRASESVDVYGN |
| 4.1 | FAWNWIRQFPGKGLEWMGDIRYSGSTDYNP | SFMHWYQQKPGQPPKLLIYLAFNLKSGVPDRFS |
| | SLKGRSTISRDTSKNQFFLKLSSVTAADTAVY | GSGSGTDFTLTISSLQAEDVAVYYCQQNNEDPFT |
| | YCARGGYYGYDEGYPMDYWGQGTLVTVSS | FGQGTKLEIK (SEQ ID NO: 335) |
| | (SEQ ID NO: 334) | |
| Hu22F10G3- | QVQLQESGPGLVKPSQTLSLTCTVSGYSLTSD | DIVMTQSPDSLAVSLGERATINCRASESVDVYGN |
| 4.2 | FAWNWIRQFPGKGLEWMGDIRYSGSTDYNP | SFMHWYQQKPGQPPKLLIYLAFNLKSGVPARFS |
| | SLKGRSTISRDTSKNQFFLKLSSVTAADTAVY | GSGSGTDFTLTISSLQAEDVAVYYCQQNNEDPFT |
| | YCARGGYYGYDEGYPMDYWGQGTLVTVSS | FGQGTKLEIK (SEQ ID NO: 336) |
| | (SEQ ID NO: 334) | |
| Hu22F10G3- | QVQLQESGPGLVKPSQTLSLTCTVSGYSLTSD | DIVMTQSPDSLAVSLGERATISCRASESVDVYGN |
| 4.3 | FAWNWIRQFPGKGLEWMGDIRYSGSTDYNP | SFMHWYQQKPGQPPKLLIYLAFNLKSGVPDRFS |
| | SLKGRSTISRDTSKNQFFLKLSSVTAADTAVY | GSGSGTDFTLTISSLQAEDVAVYYCQQNNEDPFT |
| | YCARGGYYGYDEGYPMDYWGQGTLVTVSS | FGQGTKLEIK (SEQ ID NO: 337) |
| | (SEQ ID NO: 334) | |
| Hu22F10G3- | QVQLQESGPGLVKPSQTLSLTCTVSGYSLTSD | DIVMTQSPDSLAVSLGERATISCRASESVDVYGN |
| 4.4 | FAWNWIRQFPGKGLEWMGDIRYSGSTDYNP | SFMHWYQQKPGQPPKLLIYLAFNLKSGVPARFS |
| | SLKGRSTISRDTSKNQFFLKLSSVTAADTAVY | GSGSGTDFTLTISSLQAEDVATYYCQQNNEDPFT |
| | YCARGGYYGYDEGYPMDYWGQGTLVTVSS | FGQGTKLEIK (SEQ ID NO: 338) |
| | (SEQ ID NO: 334) | |
| Hu22F10G3- | QVQLQESGPGLVKPSQTLSLTCTVSGYSLTSD | DIVMTQSPDSLAVSLGERATINCRASESVDVYGN |
| 1.1-NA | FAWNWIRQHPGKGLEWIGDIRYSGSTDYNPS | AFMHWYQQKPGQPPKLLIYLAFNLKSGVPDRFS |
| | LKGRVTISVDTSKNQFSLKLSSVTAADTAVY | GSGSGTDFTLTISSLQAEDVAVYYCQQNNEDPFT |
| | YCARGGYYGYDEGYPMDYWGQGTLVTVSS | FGQGTKLEIK (SEQ ID NO: 339) |
| | (SEQ ID NO: 331) | |
| Hu22F10G3- | QVQLQESGPGLVKPSQTLSLTCTVSGYSLTSD | DIVMTQSPDSLAVSLGERATINCRASESVDVYGN |
| 1.2-NA | FAWNWIRQHPGKGLEWIGDIRYSGSTDYNPS | AFMHWYQQKPGQPPKLLIYLAFNLKSGVPARFS |
| | LKGRVTISVDTSKNQFSLKLSSVTAADTAVY | GSGSGTDFTLTISSLQAEDVAVYYCQQNNEDPFT |
| | YCARGGYYGYDEGYPMDYWGQGTLVTVSS | FGQGTKLEIK (SEQ ID NO: 340) |
| | (SEQ ID NO: 331) | |
| Hu22F10G3- | QVQLQESGPGLVKPSQTLSLTCTVSGYSLTSD | DIVMTQSPDSLAVSLGERATISCRASESVDVYGN |
| 1.3-NA | FAWNWIRQHPGKGLEWIGDIRYSGSTDYNPS | AFMHWYQQKPGQPPKLLIYLAFNLKSGVPDRFS |
| | LKGRVTISVDTSKNQFSLKLSSVTAADTAVY | GSGSGTDFTLTISSLQAEDVAVYYCQQNNEDPFT |
| | YCARGGYYGYDEGYPMDYWGQGTLVTVSS | FGQGTKLEIK (SEQ ID NO: 341) |
| | (SEQ ID NO: 331) | |
| Hu22F10G3- | QVQLQESGPGLVKPSQTLSLTCTVSGYSLTSD | DIVMTQSPDSLAVSLGERATISCRASESVDVYGN |
| 1.4-NA | FAWNWIRQHPGKGLEWIGDIRYSGSTDYNPS | AFMHWYQQKPGQPPKLLIYLAFNLKSGVPARFS |
| | LKGRVTISVDTSKNQFSLKLSSVTAADTAVY | GSGSGTDFTLTISSLQAEDVATYYCQQNNEDPFT |
| | YCARGGYYGYDEGYPMDYWGQGTLVTVSS | FGQGTKLEIK (SEQ ID NO: 342) |
| | (SEQ ID NO: 331) | |
| Hu22F10G3- | QVQLQESGPGLVKPSQTLSLTCTVSGYSLTSD | DIVMTQSPDSLAVSLGERATINCRASESVDVYGN |
| 2.1-NA | FAWNWIRQHPGKGLEWMGDIRYSGSTDYNP | AFMHWYQQKPGQPPKLLIYLAFNLKSGVPDRFS |
| | SLKGRVTISRDTSKNQFSLKLSSVTAADTAVY | GSGSGTDFTLTISSLQAEDVAVYYCQQNNEDPFT |
| | YCARGGYYGYDEGYPMDYWGQGTLVTVSS | FGQGTKLEIK (SEQ ID NO: 339) |
| | (SEQ ID NO: 332) | |
| Hu22F10G3- | QVQLQESGPGLVKPSQTLSLTCTVSGYSLTSD | DIVMTQSPDSLAVSLGERATINCRASESVDVYGN |
| 2.2-NA | FAWNWIRQHPGKGLEWMGDIRYSGSTDYNP | AFMHWYQQKPGQPPKLLIYLAFNLKSGVPARFS |
| | SLKGRVTISRDTSKNQFSLKLSSVTAADTAVY | GSGSGTDFTLTISSLQAEDVAVYYCQQNNEDPFT |
| | YCARGGYYGYDEGYPMDYWGQGTLVTVSS | FGQGTKLEIK (SEQ ID NO: 340) |
| | (SEQ ID NO: 332) | |
| Hu22F10G3- | QVQLQESGPGLVKPSQTLSLTCTVSGYSLTSD | DIVMTQSPDSLAVSLGERATISCRASESVDVYGN |
| 2.3-NA | FAWNWIRQHPGKGLEWMGDIRYSGSTDYNP | AFMHWYQQKPGQPPKLLIYLAFNLKSGVPDRFS |
| | SLKGRVTISRDTSKNQFSLKLSSVTAADTAVY | GSGSGTDFTLTISSLQAEDVAVYYCQQNNEDPFT |
| | YCARGGYYGYDEGYPMDYWGQGTLVTVSS | FGQGTKLEIK (SEQ ID NO: 341) |
| | (SEQ ID NO: 332) | |
| Hu22F10G3- | QVQLQESGPGLVKPSQTLSLTCTVSGYSLTSD | DIVMTQSPDSLAVSLGERATISCRASESVDVYGN |
| 2.4-NA | FAWNWIRQHPGKGLEWMGDIRYSGSTDYNP | AFMHWYQQKPGQPPKLLIYLAFNLKSGVPARFS |
| | SLKGRVTISRDTSKNQFSLKLSSVTAADTAVY | GSGSGTDFTLTISSLQAEDVATYYCQQNNEDPFT |
| | YCARGGYYGYDEGYPMDYWGQGTLVTVSS | FGQGTKLEIK (SEQ ID NO: 342) |
| | (SEQ ID NO: 332) | |
| Hu22F10G3- | QVQLQESGPGLVKPSQTLSLTCTVSGYSLTSD | DIVMTQSPDSLAVSLGERATINCRASESVDVYGN |
| 3.1-NA | FAWNWIRQHPGKGLEWMGDIRYSGSTDYNP | AFMHWYQQKPGQPPKLLIYLAFNLKSGVPDRFS |
| | SLKGRSTISRDTSKNQFSLKLSSVTAADTAVY | GSGSGTDFTLTISSLQAEDVAVYYCQQNNEDPFT |
| | YCARGGYYGYDEGYPMDYWGQGTLVTVSS | FGQGTKLEIK (SEQ ID NO: 339) |
| | (SEQ ID NO: 333) | |
| Hu22F10G3- | QVQLQESGPGLVKPSQTLSLTCTVSGYSLTSD | DIVMTQSPDSLAVSLGERATINCRASESVDVYGN |
| 3.2-NA | FAWNWIRQHPGKGLEWMGDIRYSGSTDYNP | AFMHWYQQKPGQPPKLLIYLAFNLKSGVPARFS |
| | SLKGRSTISRDTSKNQFSLKLSSVTAADTAVY | GSGSGTDFTLTISSLQAEDVAVYYCQQNNEDPFT |
| | YCARGGYYGYDEGYPMDYWGQGTLVTVSS | FGQGTKLEIK (SEQ ID NO: 340) |
| | (SEQ ID NO: 333) | |
| Hu22F10G3- | QVQLQESGPGLVKPSQTLSLTCTVSGYSLTSD | DIVMTQSPDSLAVSLGERATISCRASESVDVYGN |
| 3.3-NA | FAWNWIRQHPGKGLEWMGDIRYSGSTDYNP | AFMHWYQQKPGQPPKLLIYLAFNLKSGVPDRFS |
| | SLKGRSTISRDTSKNQFSLKLSSVTAADTAVY | GSGSGTDFTLTISSLQAEDVAVYYCQQNNEDPFT |
| | YCARGGYYGYDEGYPMDYWGQGTLVTVSS | FGQGTKLEIK (SEQ ID NO: 341) |
| | (SEQ ID NO: 333) | |
| Hu22F10G3- | QVQLQESGPGLVKPSQTLSLTCTVSGYSLTSD | DIVMTQSPDSLAVSLGERATISCRASESVDVYGN |
| 3.4-NA | FAWNWIRQHPGKGLEWMGDIRYSGSTDYNP | AFMHWYQQKPGQPPKLLIYLAFNLKSGVPARFS |
| | SLKGRSTISRDTSKNQFSLKLSSVTAADTAVY | GSGSGTDFTLTISSLQAEDVATYYCQQNNEDPFT |
| | YCARGGYYGYDEGYPMDYWGQGTLVTVSS | FGQGTKLEIK (SEQ ID NO: 342) |
| | (SEQ ID NO: 333) | |
| Hu22F10G3- | QVQLQESGPGLVKPSQTLSLTCTVSGYSLTSD | DIVMTQSPDSLAVSLGERATINCRASESVDVYGN |
| 4.1-NA | FAWNWIRQFPGKGLEWMGDIRYSGSTDYNP | AFMHWYQQKPGQPPKLLIYLAFNLKSGVPDRFS |
| | SLKGRSTISRDTSKNQFFLKLSSVTAADTAVY | GSGSGTDFTLTISSLQAEDVAVYYCQQNNEDPFT |
| | YCARGGYYGYDEGYPMDYWGQGTLVTVSS | FGQGTKLEIK (SEQ ID NO: 339) |
| | (SEQ ID NO: 334) | |
| Hu22F10G3- | QVQLQESGPGLVKPSQTLSLTCTVSGYSLTSD | DIVMTQSPDSLAVSLGERATINCRASESVDVYGN |
| 4.2-NA | FAWNWIRQFPGKGLEWMGDIRYSGSTDYNP | AFMHWYQQKPGQPPKLLIYLAFNLKSGVPARFS |
| | SLKGRSTISRDTSKNQFFLKLSSVTAADTAVY | GSGSGTDFTLTISSLQAEDVAVYYCQQNNEDPFT |
| | YCARGGYYGYDEGYPMDYWGQGTLVTVSS | FGQGTKLEIK (SEQ ID NO: 340) |
| | (SEQ ID NO: 334) | |
| Hu22F10G3- | QVQLQESGPGLVKPSQTLSLTCTVSGYSLTSD | DIVMTQSPDSLAVSLGERATISCRASESVDVYGN |
| 4.3-NA | FAWNWIRQFPGKGLEWMGDIRYSGSTDYNP | AFMHWYQQKPGQPPKLLIYLAFNLKSGVPDRFS |
| | SLKGRSTISRDTSKNQFFLKLSSVTAADTAVY | GSGSGTDFTLTISSLQAEDVAVYYCQQNNEDPFT |
| | YCARGGYYGYDEGYPMDYWGQGTLVTVSS | FGQGTKLEIK (SEQ ID NO: 341) |
| | (SEQ ID NO: 334) | |
| Hu22F10G3- | QVQLQESGPGLVKPSQTLSLTCTVSGYSLTSD | DIVMTQSPDSLAVSLGERATISCRASESVDVYGN |
| 4.4-NA | FAWNWIRQFPGKGLEWMGDIRYSGSTDYNP | AFMHWYQQKPGQPPKLLIYLAFNLKSGVPARFS |
| | SLKGRSTISRDTSKNQFFLKLSSVTAADTAVY | GSGSGTDFTLTISSLQAEDVATYYCQQNNEDPFT |
| | YCARGGYYGYDEGYPMDYWGQGTLVTVSS | FGQGTKLEIK (SEQ ID NO: 342) |
| | (SEQ ID NO: 334) | |
| Hu22F10G3- | QVQLQESGPGLVKPSQTLSLTCTVSGYSLTSD | DIVMTQSPDSLAVSLGERATINCRASESVDVYGQ |
| 1.1-QS | FAWNWIRQHPGKGLEWIGDIRYSGSTDYNPS | SFMHWYQQKPGQPPKLLIYLAFNLKSGVPDRFS |
| | LKGRVTISVDTSKNQFSLKLSSVTAADTAVY | GSGSGTDFTLTISSLQAEDVAVYYCQQNNEDPFT |
| | YCARGGYYGYDEGYPMDYWGQGTLVTVSS | FGQGTKLEIK (SEQ ID NO: 343) |
| | (SEQ ID NO: 331) | |
| Hu22F10G3- | QVQLQESGPGLVKPSQTLSLTCTVSGYSLTSD | DIVMTQSPDSLAVSLGERATINCRASESVDVYGQ |
| 1.2-QS | FAWNWIRQHPGKGLEWIGDIRYSGSTDYNPS | SFMHWYQQKPGQPPKLLIYLAFNLKSGVPARFS |
| | LKGRVTISVDTSKNQFSLKLSSVTAADTAVY | GSGSGTDFTLTISSLQAEDVAVYYCQQNNEDPFT |
| | YCARGGYYGYDEGYPMDYWGQGTLVTVSS | FGQGTKLEIK (SEQ ID NO: 344) |
| | (SEQ ID NO: 331) | |
| Hu22F10G3- | QVQLQESGPGLVKPSQTLSLTCTVSGYSLTSD | DIVMTQSPDSLAVSLGERATISCRASESVDVYGQ |
| 1.3-QS | FAWNWIRQHPGKGLEWIGDIRYSGSTDYNPS | SFMHWYQQKPGQPPKLLIYLAFNLKSGVPDRFS |
| | LKGRVTISVDTSKNQFSLKLSSVTAADTAVY | GSGSGTDFTLTISSLQAEDVAVYYCQQNNEDPFT |
| | YCARGGYYGYDEGYPMDYWGQGTLVTVSS | FGQGTKLEIK (SEQ ID NO: 345) |
| | (SEQ ID NO: 331) | |
| Hu22F10G3- | QVQLQESGPGLVKPSQTLSLTCTVSGYSLTSD | DIVMTQSPDSLAVSLGERATISCRASESVDVYGQ |
| 1.4-QS | FAWNWIRQHPGKGLEWIGDIRYSGSTDYNPS | SFMHWYQQKPGQPPKLLIYLAFNLKSGVPARFS |
| | LKGRVTISVDTSKNQFSLKLSSVTAADTAVY | GSGSGTDFTLTISSLQAEDVATYYCQQNNEDPFT |
| | YCARGGYYGYDEGYPMDYWGQGTLVTVSS | FGQGTKLEIK (SEQ ID NO: 346) |
| | (SEQ ID NO: 331) | |
| Hu22F10G3- | QVQLQESGPGLVKPSQTLSLTCTVSGYSLTSD | DIVMTQSPDSLAVSLGERATINCRASESVDVYGQ |
| 2.1-QS | FAWNWIRQHPGKGLEWMGDIRYSGSTDYNP | SFMHWYQQKPGQPPKLLIYLAFNLKSGVPDRFS |
| | SLKGRVTISRDTSKNQFSLKLSSVTAADTAVY | GSGSGTDFTLTISSLQAEDVAVYYCQQNNEDPFT |
| | YCARGGYYGYDEGYPMDYWGQGTLVTVSS | FGQGTKLEIK (SEQ ID NO: 343) |
| | (SEQ ID NO: 332) | |
| Hu22F10G3- | QVQLQESGPGLVKPSQTLSLTCTVSGYSLTSD | DIVMTQSPDSLAVSLGERATINCRASESVDVYGQ |
| 2.2-QS | FAWNWIRQHPGKGLEWMGDIRYSGSTDYNP | SFMHWYQQKPGQPPKLLIYLAFNLKSGVPARFS |
| | SLKGRVTISRDTSKNQFSLKLSSVTAADTAVY | GSGSGTDFTLTISSLQAEDVAVYYCQQNNEDPFT |
| | YCARGGYYGYDEGYPMDYWGQGTLVTVSS | FGQGTKLEIK (SEQ ID NO: 344) |
| | (SEQ ID NO: 332) | |
| Hu22F10G3- | QVQLQESGPGLVKPSQTLSLTCTVSGYSLTSD | DIVMTQSPDSLAVSLGERATISCRASESVDVYGQ |
| 2.3-QS | FAWNWIRQHPGKGLEWMGDIRYSGSTDYNP | SFMHWYQQKPGQPPKLLIYLAFNLKSGVPDRFS |
| | SLKGRVTISRDTSKNQFSLKLSSVTAADTAVY | GSGSGTDFTLTISSLQAEDVAVYYCQQNNEDPFT |
| | YCARGGYYGYDEGYPMDYWGQGTLVTVSS | FGQGTKLEIK (SEQ ID NO: 345) |
| | (SEQ ID NO: 332) | |
| Hu22F10G3- | QVQLQESGPGLVKPSQTLSLTCTVSGYSLTSD | DIVMTQSPDSLAVSLGERATISCRASESVDVYGQ |
| 2.4-QS | FAWNWIRQHPGKGLEWMGDIRYSGSTDYNP | SFMHWYQQKPGQPPKLLIYLAFNLKSGVPARFS |
| | SLKGRVTISRDTSKNQFSLKLSSVTAADTAVY | GSGSGTDFTLTISSLQAEDVATYYCQQNNEDPFT |
| | YCARGGYYGYDEGYPMDYWGQGTLVTVSS | FGQGTKLEIK (SEQ ID NO: 346) |
| | (SEQ ID NO: 332) | |
| Hu22F10G3- | QVQLQESGPGLVKPSQTLSLTCTVSGYSLTSD | DIVMTQSPDSLAVSLGERATINCRASESVDVYGQ |
| 3.1-QS | FAWNWIRQHPGKGLEWMGDIRYSGSTDYNP | SFMHWYQQKPGQPPKLLIYLAFNLKSGVPDRFS |
| | SLKGRSTISRDTSKNQFSLKLSSVTAADTAVY | GSGSGTDFTLTISSLQAEDVAVYYCQQNNEDPFT |
| | YCARGGYYGYDEGYPMDYWGQGTLVTVSS | FGQGTKLEIK (SEQ ID NO: 343) |
| | (SEQ ID NO: 333) | |
| Hu22F10G3- | QVQLQESGPGLVKPSQTLSLTCTVSGYSLTSD | DIVMTQSPDSLAVSLGERATINCRASESVDVYGQ |
| 3.2-QS | FAWNWIRQHPGKGLEWMGDIRYSGSTDYNP | SFMHWYQQKPGQPPKLLIYLAFNLKSGVPARFS |
| | SLKGRSTISRDTSKNQFSLKLSSVTAADTAVY | GSGSGTDFTLTISSLQAEDVAVYYCQQNNEDPFT |
| | YCARGGYYGYDEGYPMDYWGQGTLVTVSS | FGQGTKLEIK (SEQ ID NO: 344) |
| | (SEQ ID NO: 333) | |
| Hu22F10G3- | QVQLQESGPGLVKPSQTLSLTCTVSGYSLTSD | DIVMTQSPDSLAVSLGERATISCRASESVDVYGQ |
| 3.3-QS | FAWNWIRQHPGKGLEWMGDIRYSGSTDYNP | SFMHWYQQKPGQPPKLLIYLAFNLKSGVPDRFS |
| | SLKGRSTISRDTSKNQFSLKLSSVTAADTAVY | GSGSGTDFTLTISSLQAEDVAVYYCQQNNEDPFT |
| | YCARGGYYGYDEGYPMDYWGQGTLVTVSS | FGQGTKLEIK (SEQ ID NO: 345) |
| | (SEQ ID NO: 333) | |
| Hu22F10G3- | QVQLQESGPGLVKPSQTLSLTCTVSGYSLTSD | DIVMTQSPDSLAVSLGERATISCRASESVDVYGQ |
| 3.4-QS | FAWNWIRQHPGKGLEWMGDIRYSGSTDYNP | SFMHWYQQKPGQPPKLLIYLAFNLKSGVPARFS |
| | SLKGRSTISRDTSKNQFSLKLSSVTAADTAVY | GSGSGTDFTLTISSLQAEDVATYYCQQNNEDPFT |
| | YCARGGYYGYDEGYPMDYWGQGTLVTVSS | FGQGTKLEIK (SEQ ID NO: 346) |
| | (SEQ ID NO: 333) | |
| Hu22F10G3- | QVQLQESGPGLVKPSQTLSLTCTVSGYSLTSD | DIVMTQSPDSLAVSLGERATINCRASESVDVYGQ |
| 4.1-QS | FAWNWIRQFPGKGLEWMGDIRYSGSTDYNP | SFMHWYQQKPGQPPKLLIYLAFNLKSGVPDRFS |
| | SLKGRSTISRDTSKNQFFLKLSSVTAADTAVY | GSGSGTDFTLTISSLQAEDVAVYYCQQNNEDPFT |
| | YCARGGYYGYDEGYPMDYWGQGTLVTVSS | FGQGTKLEIK (SEQ ID NO: 343) |
| | (SEQ ID NO: 334) | |
| Hu22F10G3- | QVQLQESGPGLVKPSQTLSLTCTVSGYSLTSD | DIVMTQSPDSLAVSLGERATINCRASESVDVYGQ |
| 4.2-QS | FAWNWIRQFPGKGLEWMGDIRYSGSTDYNP | SFMHWYQQKPGQPPKLLIYLAFNLKSGVPARFS |
| | SLKGRSTISRDTSKNQFFLKLSSVTAADTAVY | GSGSGTDFTLTISSLQAEDVAVYYCQQNNEDPFT |
| | YCARGGYYGYDEGYPMDYWGQGTLVTVSS | FGQGTKLEIK (SEQ ID NO: 344) |
| | (SEQ ID NO: 334) | |
| Hu22F10G3- | QVQLQESGPGLVKPSQTLSLTCTVSGYSLTSD | DIVMTQSPDSLAVSLGERATISCRASESVDVYGQ |
| 4.3-QS | FAWNWIRQFPGKGLEWMGDIRYSGSTDYNP | SFMHWYQQKPGQPPKLLIYLAFNLKSGVPDRFS |
| | SLKGRSTISRDTSKNQFFLKLSSVTAADTAVY | GSGSGTDFTLTISSLQAEDVAVYYCQQNNEDPFT |
| | YCARGGYYGYDEGYPMDYWGQGTLVTVSS | FGQGTKLEIK (SEQ ID NO: 345) |
| | (SEQ ID NO: 334) | |
| Hu22F10G3- | QVQLQESGPGLVKPSQTLSLTCTVSGYSLTSD | DIVMTQSPDSLAVSLGERATISCRASESVDVYGQ |
| 4.4-QS | FAWNWIRQFPGKGLEWMGDIRYSGSTDYNP | SFMHWYQQKPGQPPKLLIYLAFNLKSGVPARFS |
| | SLKGRSTISRDTSKNQFFLKLSSVTAADTAVY | GSGSGTDFTLTISSLQAEDVATYYCQQNNEDPFT |
| | YCARGGYYGYDEGYPMDYWGQGTLVTVSS | FGQGTKLEIK (SEQ ID NO: 346) |
| | (SEQ ID NO: 334) | |

In certain embodiments, a KLRB1-binding agent comprises a heavy chain variable region comprising heavy chain CDRs 1, 2, and 3, and a light chain variable region comprising light chain CDRs 1, 2, and 3 as shown in Tables 1-18, Table B, or Table C.

In some embodiments, a KLRB1-binding agent (e.g., an antibody) comprises a heavy chain variable region or sequence having at least about 80% (e.g., at least 90%, 95%, 97%, 99%, or 100%) sequence identity to a heavy chain variable region sequence presented herein (e.g., in Tables 1-18, Table B, or Table C), and/or a light chain variable region having at least 80% (e.g., at least 90%, 95%, 97%, 99%, or 100%) sequence identity to a light chain variable region sequence presented herein (e.g., in Tables 1-18, Table B, or Table C). The term “consensus sequence,” as used herein with respect to light chain (VL) and heavy chain (VH) variable regions, refers to a composite or genericized VL or VH sequence defined based on information as to which amino acid residues within the VL or VH chain are amenable to modification without detriment to antigen binding. Thus, in a “consensus sequence” for a VL or VH chain, certain amino acid positions are occupied by one of multiple possible amino acid residues at that position. For example, if an arginine (R) or a serine(S) occur at a particular position, then that particular position within the consensus sequence can be either arginine or serine (R or S). Consensus sequences for VH and VL chain can be defined, for example, by in vitro affinity maturation (e.g., randomizing every amino acid position in a certain CDR using degenerate coding primers), by scanning mutagenesis (e.g., alanine scanning mutagenesis) of amino acid residues within the antibody CDRs, or any other methods known in the art, followed by evaluation of the binding of the mutants to the antigen to determine whether the mutated amino acid position affects antigen binding. In some aspects, mutations are introduced in the CDR regions. In other aspects, mutations are introduced in framework regions. In some other aspects, mutations are introduced in CDR and framework regions. Consensus sequences can be determined by software such as the EMBOSS Cons available at: ebi.ac.uk/Tools/msa/emboss_cons/.

In some embodiments, a KLRB1-binding agent described herein (e.g., an antibody) comprises one or more constant heavy domains (e.g., CH1, CH2 and/or CH3 regions). In some embodiments, the KLRB1-binding agent comprises a constant heavy domain 1 (CH1) having an amino acid sequence set forth herein, e.g., in Tables B or C. In some embodiments, the KLRB1-binding agent comprises a constant heavy domain 2 (CH2) comprising an amino acid sequence set forth herein, e.g., in Tables B or C. In some embodiments, the KLRB1-binding agent comprises a constant heavy domain 3 (CH3) comprising an amino acid sequence set forth herein, e.g., in Tables B or C. In some embodiments, the KLRB1-binding agent comprises a heavy chain constant region comprising an amino acid sequence having at least 80, 85%, 90%, 95%, 97%, or 99% sequence identity to an amino acid sequence set forth herein, e.g., in Tables B or C. In some embodiments, the one or more constant regions of the KLRB1-binding agent has/have been modified. In some embodiments, the antibodies may comprise modifications to one or more of the three heavy chain constant regions (CH1, CH2 or CH3) and/or to the light chain constant region (CL). In some embodiments, the heavy chain constant region of the modified antibodies comprises at least one human constant region. In some embodiments, the heavy chain constant region of the modified antibodies comprises more than one human constant region. In some embodiments, modifications to the constant region comprise additions, deletions, or substitutions of one or more amino acids in one or more regions. In some embodiments, one or more regions are partially or entirely deleted from the constant regions of the modified antibodies. In some embodiments, the entire CH2 domain has been removed from an antibody (ΔCH2 constructs). In some embodiments, a deleted constant region is replaced by a short amino acid spacer that provides some of the molecular flexibility typically imparted by the absent constant region. In some embodiments, a modified antibody comprises a CH3 domain directly fused to the hinge region of the antibody. In some embodiments, a modified antibody comprises a peptide spacer inserted between the hinge region and modified CH2 and/or CH3 domains.

It is known in the art that the constant region(s) of an antibody mediates several effector functions and these effector functions can vary depending on the isotype of the antibody. In addition, the Fc region of an antibody can bind a cell expressing a Fc receptor (FcR). There are a number of Fc receptors which are specific for different classes of antibody, including IgG (gamma receptors), IgE (epsilon receptors), IgA (alpha receptors) and IgM (mu receptors). Binding of antibody to Fc receptors on cell surfaces triggers a number of important and diverse biological responses including engulfment and destruction of antibody-coated particles, clearance of immune complexes, lysis of antibody-coated target cells by killer cells (called antibody-dependent cell cytotoxicity or ADCC), cell-mediated cytotoxicity (ADCC), antibody-dependent cellular phagocytosis (ADCP), complement-dependent cytotoxicity (CDC), release of inflammatory mediators, placental transfer, and control of immunoglobulin production. In some embodiments, an antibody comprises a variant Fc region. The amino acid sequences of the Fc region of human IgG1, IgG2, IgG3, and IgG4 are known to those of ordinary skill in the art (e.g., a representative human IgG1 Fc region is shown in Lobner et al., Immunol Rev. 2016 March; 270 (1): 113-131; see, e.g., Table A, Table C). In some cases, Fc regions with amino acid variations have been identified in native antibodies. In some embodiments, a variant Fc region is engineered with substitutions at specific amino acid positions as compared to a native Fc region. In some embodiments, the Fc region is mutated to alter (reduce) antibody dependent cell-mediated cytotoxicity (ADCC), antibody induced complement dependent cytotoxicity (CDC), and/or antibody dependent cell-mediated phagocytosis (ADCP) (see, e.g., Kang and Jung, Experimental & Molecular Medicine. 2019. 51:1-9; Wang et al., Antibody Therapeutics, Jan. 2021. 4 (1): 45-54; Lobner et al., Immunol Rev. 2016 March; 270 (1): 113-131). In some embodiments, the Fc region is afucosylated (see, e.g., Yamane-Ohnuki and Satoh, MAbs. 2009 May-Jun; 1 (3): 230-236, which describes methods for production of therapeutic antibodies with controlled levels of fucosylation of Fc region N-glycans).

In some embodiments, the modified antibodies (e.g., modified Fc region) provide for altered effector functions that, in turn, affect the biological profile of the antibody. For example, in some embodiments, the deletion or inactivation (through point mutations or other means) of a constant region enhances Fc receptor binding of the modified antibody as it circulates. In some embodiments, the constant region modifications increase the serum half-life of the antibody. In some embodiments, the constant region modifications reduce the serum half-life of the antibody. In some embodiments, the constant region modifications increase or enhance ADCC and/or complement dependent cytotoxicity (CDC) of the antibody. In some embodiments, the constant region is modified to eliminate disulfide linkages or oligosaccharide moieties. In some embodiments, the constant region is modified to add/substitute one or more amino acids to provide one or more cytotoxin, oligosaccharide, or carbohydrate attachment sites.

Modifications to the constant region of antibodies described herein may be made using well known biochemical or molecular engineering techniques. In some embodiments, antibody variants are prepared by introducing appropriate nucleotide changes into the encoding DNA, and/or by synthesis of the desired antibody or polypeptide. Using these antibody variants, it may be possible to enhance the activity or effector function provided by a specific sequence or region while substantially maintaining the structure, binding activity, and other desired characteristics of the modified antibody.

Exemplary complete full Heavy Chain (HC) and Light Chain (LC) sequences are provided in Table C.

TABLE C
Sequences of Exemplary Complete Heavy Chain (HC) and
Light Chain (LC) Regions

| | | |
|---|---|---|
| Hu50A8F2-16- | QVQLVQSGAEVKKPGASVKVSCKASGYTFIDYYINWV | DIQLTQSPSSLSASVGDRVTITCTASSS |
| EN | RQAPGQGLEWIGWIFPGSGHTKYNENFKGKVTLTVDT | VSSTYLHWYQQKPGKSPKLLIYSTSN |
| | STSTVYMELSSLRSEDTAVYFCARGDRSALGYFDYWG | LASGVPSRFSGSGSGTDYTLTISSLQP |
| | QGTTVTVSSASTKGPSVFPLAPSSKSTSGGTAALGCLV | EDAATYYCHQYHRSPLTFGQGTKLEI |
| | KDYFPEPVTVSWNSGALTSGVHTFPAVLQSSGLYSLSS | KRTVAAPSVFIFPPSDEQLKSGTASVV |
| | VVTVPSSSLGTQTYICNVNHKPSNTKVDKKVEPKSCD | CLLNNFYPREAKVQWKVDNALQSGN |
| | KTHTCPPCPAPEAAGAPSVFLFPPKPKDTLMISRTPEVT | SQESVTEQDSKDSTYSLSSTLTLSKAD |
| | CVVVDVSHEDPEVKFNWYVDGVEVHNAKTKPREEQY | YEKHKVYACEVTHQGLSSPVTKSFNR |
| | NSTYRVVSVLTVLHQDWLNGKEYKCKVSNKALPAPIE | GEC (SEQ ID NO: 528) |
| | KTISKAKGQPREPQVYTLPPSRDELTKNQVSLTCLVKG | |
| | FYPSDIAVEWESNGQPENNYKTTPPVLDSDGSFFLYSK | |
| | LTVDKSRWQQGNVFSCSVMHEALHNHYTQKSLSLSP | |
| | GK (SEQ ID NO: 419) | |
| 22F10G3 | DVQLQESGPGQVKPSQSLSLTCTVTGYSLTSDFAWNW | NIVLTQSPASLAVSLGQRATISCRASE |
| | IRQFPGNRLEWMGDIRYSGSTDYNPSLKGRSSITRDTS | SVDVYGNSFMHWYQQKTGQPPKLLI |
| | KNQFFLQLNSVTTEDTATYYCARGGYYGYDEGYPMD | YLAFNLKSGVPARFSGSGSRTDFTLTI |
| | YWGQGTSVTVSSAKTTPPSVYPLAPGSAAQTNSMVTL | DPVEADDAATYYCQQNNEDPFTFGS |
| | GCLVKGYFPEPVTVTWNSGSLSSGVHTFPAVLQSDLY | GTKLEIKRADAAPTVSIFPPSSEQLTSG |
| | TLSSSVTVPSSPRPSETVTCNVAHPASSTKVDKKIVPRD | GASVVCFLNNFYPKDINVKWKIDGSE |
| | CGCKPCICTVPEVSSVFIFPPKPKDVLTITLTPKVTCVV | RQNGVLNSWTDQDSKDSTYSMSSTL |
| | VDISKDDPEVQFSWFVDDVEVHTAQTQPREEQFNSTF | TLTKDEYERHNSYTCEATHKTSTSPIV |
| | RSVSELPIMHQDWLNGKEFKCRVNSAAFPAPIEKTISK | KSFNRNEC (SEQ ID NO: 529) |
| | TKGRPKAPQVYTIPPPKEQMAKDKVSLTCMITDFFPED | |
| | ITVEWQWNGQPAENYKNTQPIMNTNGSYFVYSKLNV | |
| | QKSNWEAGNTFTCSVLHEGLHNHHTEKSLSHSPGK | |
| | (SEQ ID NO: 420) | |
| 22F10G3-NA | DVQLQESGPGQVKPSQSLSLTCTVTGYSLTSDFAWNW | NIVLTQSPASLAVSLGQRATISCRASE |
| | IRQFPGNRLEWMGDIRYSGSTDYNPSLKGRSSITRDTS | SVDVYGNAFMHWYQQKTGQPPKLLI |
| | KNQFFLQLNSVTTEDTATYYCARGGYYGYDEGYPMD | YLAFNLKSGVPARFSGSGSRTDFTLTI |
| | YWGQGTSVTVSSAKTTPPSVYPLAPGSAAQTNSMVTL | DPVEADDAATYYCQQNNEDPFTFGS |
| | GCLVKGYFPEPVTVTWNSGSLSSGVHTFPAVLQSDLY | GTKLEIKRADAAPTVSIFPPSSEQLTSG |
| | TLSSSVTVPSSPRPSETVTCNVAHPASSTKVDKKIVPRD | GASVVCFLNNFYPKDINVKWKIDGSE |
| | CGCKPCICTVPEVSSVFIFPPKPKDVLTITLTPKVTCVV | RQNGVLNSWTDQDSKDSTYSMSSTL |
| | VDISKDDPEVQFSWFVDDVEVHTAQTQPREEQFNSTF | TLTKDEYERHNSYTCEATHKTSTSPIV |
| | RSVSELPIMHQDWLNGKEFKCRVNSAAFPAPIEKTISK | KSFNRNEC (SEQ ID NO: 530) |
| | TKGRPKAPQVYTIPPPKEQMAKDKVSLTCMITDFFPED | |
| | ITVEWQWNGQPAENYKNTQPIMNTNGSYFVYSKLNV | |
| | QKSNWEAGNTFTCSVLHEGLHNHHTEKSLSHSPGK | |
| | (SEQ ID NO: 421) | |
| 22F10G3-QS | DVQLQESGPGQVKPSQSLSLTCTVTGYSLTSDFAWNW | NIVLTQSPASLAVSLGQRATISCRASE |
| | IRQFPGNRLEWMGDIRYSGSTDYNPSLKGRSSITRDTS | SVDVYGQSFMHWYQQKTGQPPKLLI |
| | KNQFFLQLNSVTTEDTATYYCARGGYYGYDEGYPMD | YLAFNLKSGVPARFSGSGSRTDFTLTI |
| | YWGQGTSVTVSSAKTTPPSVYPLAPGSAAQTNSMVTL | DPVEADDAATYYCQQNNEDPFTFGS |
| | GCLVKGYFPEPVTVTWNSGSLSSGVHTFPAVLQSDLY | GTKLEIKRADAAPTVSIFPPSSEQLTSG |
| | TLSSSVTVPSSPRPSETVTCNVAHPASSTKVDKKIVPRD | GASVVCFLNNFYPKDINVKWKIDGSE |
| | CGCKPCICTVPEVSSVFIFPPKPKDVLTITLTPKVTCVV | RQNGVLNSWTDQDSKDSTYSMSSTL |
| | VDISKDDPEVQFSWFVDDVEVHTAQTQPREEQFNSTF | TLTKDEYERHNSYTCEATHKTSTSPIV |
| | RSVSELPIMHQDWLNGKEFKCRVNSAAFPAPIEKTISK | KSFNRNEC (SEQ ID NO: 531) |
| | TKGRPKAPQVYTIPPPKEQMAKDKVSLTCMITDFFPED | |
| | ITVEWQWNGQPAENYKNTQPIMNTNGSYFVYSKLNV | |
| | QKSNWEAGNTFTCSVLHEGLHNHHTEKSLSHSPGK | |
| | (SEQ ID NO: 422) | |
| 22F10G3- | DVQLQESGPGQVKPSQSLSLTCTVTGYSLTSDFAWNW | NIVLTQSPASLAVSLGQRATISCRASE |
| chimeric | IRQFPGNRLEWMGDIRYSGSTDYNPSLKGRSSITRDTS | SVDVYGNSFMHWYQQKTGQPPKLLI |
| | KNQFFLQLNSVTTEDTATYYCARGGYYGYDEGYPMD | YLAFNLKSGVPARFSGSGSRTDFTLTI |
| | YWGQGTSVTVSSASTKGPSVFPLAPSSKSTSGGTAALG | DPVEADDAATYYCQQNNEDPFTFGS |
| | CLVKDYFPEPVTVSWNSGALTSGVHTFPAVLQSSGLY | GTKLEIKRTVAAPSVFIFPPSDEQLKS |
| | SLSSVVTVPSSSLGTQTYICNVNHKPSNTKVDKKVEPK | GTASVVCLLNNFYPREAKVQWKVDN |
| | SCDKTHTCPPCPAPELLGGPSVFLFPPKPKDTLMISRTP | ALQSGNSQESVTEQDSKDSTYSLSSTL |
| | EVTCVVVDVSHEDPEVKFNWYVDGVEVHNAKTKPRE | TLSKADYEKHKVYACEVTHQGLSSP |
| | EQYNSTYRVVSVLTVLHQDWLNGKEYKCKVSNKALP | VTKSFNRGEC (SEQ ID NO: 532) |
| | APIEKTISKAKGQPREPQVYTLPPSRDELTKNQVSLTCL | |
| | VKGFYPSDIAVEWESNGQPENNYKTTPPVLDSDGSFFL | |
| | YSKLTVDKSRWQQGNVFSCSVMHEALHNHYTQKSLS | |
| | LSPGK (SEQ ID NO: 423) | |
| Hu22F10G3- | QVQLQESGPGLVKPSQTLSLTCTVSGYSLTSDFAWNW | DIVMTQSPDSLAVSLGERATINCRASE |
| 1.1 | IRQHPGKGLEWIGDIRYSGSTDYNPSLKGRVTISVDTS | SVDVYGNSFMHWYQQKPGQPPKLLI |
| | KNQFSLKLSSVTAADTAVYYCARGGYYGYDEGYPMD | YLAFNLKSGVPDRFSGSGSGTDFTLTI |
| | YWGQGTLVTVSSASTKGPSVFPLAPSSKSTSGGTAALG | SSLQAEDVAVYYCQQNNEDPFTFGQ |
| | CLVKDYFPEPVTVSWNSGALTSGVHTFPAVLQSSGLY | GTKLEIKRTVAAPSVFIFPPSDEQLKS |
| | SLSSVVTVPSSSLGTQTYICNVNHKPSNTKVDKKVEPK | GTASVVCLLNNFYPREAKVQWKVDN |
| | SCDKTHTCPPCPAPELLGGPSVFLFPPKPKDTLMISRTP | ALQSGNSQESVTEQDSKDSTYSLSSTL |
| | EVTCVVVDVSHEDPEVKFNWYVDGVEVHNAKTKPRE | TLSKADYEKHKVYACEVTHQGLSSP |
| | EQYNSTYRVVSVLTVLHQDWLNGKEYKCKVSNKALP | VTKSFNRGEC (SEQ ID NO: 533) |
| | APIEKTISKAKGQPREPQVYTLPPSRDELTKNQVSLTCL | |
| | VKGFYPSDIAVEWESNGQPENNYKTTPPVLDSDGSFFL | |
| | YSKLTVDKSRWQQGNVFSCSVMHEALHNHYTQKSLS | |
| | LSPGK (SEQ ID NO: 424) | |
| Hu22F10G3- | QVQLQESGPGLVKPSQTLSLTCTVSGYSLTSDFAWNW | DIVMTQSPDSLAVSLGERATINCRASE |
| 1.2 | IRQHPGKGLEWIGDIRYSGSTDYNPSLKGRVTISVDTS | SVDVYGNSFMHWYQQKPGQPPKLLI |
| | KNQFSLKLSSVTAADTAVYYCARGGYYGYDEGYPMD | YLAFNLKSGVPARFSGSGSGTDFTLTI |
| | YWGQGTLVTVSSASTKGPSVFPLAPSSKSTSGGTAALG | SSLQAEDVAVYYCQQNNEDPFTFGQ |
| | CLVKDYFPEPVTVSWNSGALTSGVHTFPAVLQSSGLY | GTKLEIKRTVAAPSVFIFPPSDEQLKS |
| | SLSSVVTVPSSSLGTQTYICNVNHKPSNTKVDKKVEPK | GTASVVCLLNNFYPREAKVQWKVDN |
| | SCDKTHTCPPCPAPELLGGPSVFLFPPKPKDTLMISRTP | ALQSGNSQESVTEQDSKDSTYSLSSTL |
| | EVTCVVVDVSHEDPEVKFNWYVDGVEVHNAKTKPRE | TLSKADYEKHKVYACEVTHQGLSSP |
| | EQYNSTYRVVSVLTVLHQDWLNGKEYKCKVSNKALP | VTKSFNRGEC (SEQ ID NO: 534) |
| | APIEKTISKAKGQPREPQVYTLPPSRDELTKNQVSLTCL | |
| | VKGFYPSDIAVEWESNGQPENNYKTTPPVLDSDGSFFL | |
| | YSKLTVDKSRWQQGNVFSCSVMHEALHNHYTQKSLS | |
| | LSPGK (SEQ ID NO: 425) | |
| Hu22F10G3- | QVQLQESGPGLVKPSQTLSLTCTVSGYSLTSDFAWNW | DIVMTQSPDSLAVSLGERATISCRASE |
| 1.3 | IRQHPGKGLEWIGDIRYSGSTDYNPSLKGRVTISVDTS | SVDVYGNSFMHWYQQKPGQPPKLLI |
| | KNQFSLKLSSVTAADTAVYYCARGGYYGYDEGYPMD | YLAFNLKSGVPDRFSGSGSGTDFTLTI |
| | YWGQGTLVTVSSASTKGPSVFPLAPSSKSTSGGTAALG | SSLQAEDVAVYYCQQNNEDPFTFGQ |
| | CLVKDYFPEPVTVSWNSGALTSGVHTFPAVLQSSGLY | GTKLEIKRTVAAPSVFIFPPSDEQLKS |
| | SLSSVVTVPSSSLGTQTYICNVNHKPSNTKVDKKVEPK | GTASVVCLLNNFYPREAKVQWKVDN |
| | SCDKTHTCPPCPAPELLGGPSVFLFPPKPKDTLMISRTP | ALQSGNSQESVTEQDSKDSTYSLSSTL |
| | EVTCVVVDVSHEDPEVKFNWYVDGVEVHNAKTKPRE | TLSKADYEKHKVYACEVTHQGLSSP |
| | EQYNSTYRVVSVLTVLHQDWLNGKEYKCKVSNKALP | VTKSFNRGEC (SEQ ID NO: 535) |
| | APIEKTISKAKGQPREPQVYTLPPSRDELTKNQVSLTCL | |
| | VKGFYPSDIAVEWESNGQPENNYKTTPPVLDSDGSFFL | |
| | YSKLTVDKSRWQQGNVFSCSVMHEALHNHYTQKSLS | |
| | LSPGK (SEQ ID NO: 426) | |
| Hu22F10G3- | QVQLQESGPGLVKPSQTLSLTCTVSGYSLTSDFAWNW | DIVMTQSPDSLAVSLGERATISCRASE |
| 1.4 | IRQHPGKGLEWIGDIRYSGSTDYNPSLKGRVTISVDTS | SVDVYGNSFMHWYQQKPGQPPKLLI |
| | KNQFSLKLSSVTAADTAVYYCARGGYYGYDEGYPMD | YLAFNLKSGVPARFSGSGSGTDFTLTI |
| | YWGQGTLVTVSSASTKGPSVFPLAPSSKSTSGGTAALG | SSLQAEDVATYYCQQNNEDPFTFGQ |
| | CLVKDYFPEPVTVSWNSGALTSGVHTFPAVLQSSGLY | GTKLEIKRTVAAPSVFIFPPSDEQLKS |
| | SLSSVVTVPSSSLGTQTYICNVNHKPSNTKVDKKVEPK | GTASVVCLLNNFYPREAKVQWKVDN |
| | SCDKTHTCPPCPAPELLGGPSVFLFPPKPKDTLMISRTP | ALQSGNSQESVTEQDSKDSTYSLSSTL |
| | EVTCVVVDVSHEDPEVKFNWYVDGVEVHNAKTKPRE | TLSKADYEKHKVYACEVTHQGLSSP |
| | EQYNSTYRVVSVLTVLHQDWLNGKEYKCKVSNKALP | VTKSFNRGEC (SEQ ID NO: 536) |
| | APIEKTISKAKGQPREPQVYTLPPSRDELTKNQVSLTCL | |
| | VKGFYPSDIAVEWESNGQPENNYKTTPPVLDSDGSFFL | |
| | YSKLTVDKSRWQQGNVFSCSVMHEALHNHYTQKSLS | |
| | LSPGK (SEQ ID NO: 427) | |
| Hu22F10G3- | QVQLQESGPGLVKPSQTLSLTCTVSGYSLTSDFAWNW | DIVMTQSPDSLAVSLGERATINCRASE |
| 2.1 | IRQHPGKGLEWMGDIRYSGSTDYNPSLKGRVTISRDTS | SVDVYGNSFMHWYQQKPGQPPKLLI |
| | KNQFSLKLSSVTAADTAVYYCARGGYYGYDEGYPMD | YLAFNLKSGVPDRFSGSGSGTDFTLTI |
| | YWGQGTLVTVSSASTKGPSVFPLAPSSKSTSGGTAALG | SSLQAEDVAVYYCQQNNEDPFTFGQ |
| | CLVKDYFPEPVTVSWNSGALTSGVHTFPAVLQSSGLY | GTKLEIKRTVAAPSVFIFPPSDEQLKS |
| | SLSSVVTVPSSSLGTQTYICNVNHKPSNTKVDKKVEPK | GTASVVCLLNNFYPREAKVQWKVDN |
| | SCDKTHTCPPCPAPELLGGPSVFLFPPKPKDTLMISRTP | ALQSGNSQESVTEQDSKDSTYSLSSTL |
| | EVTCVVVDVSHEDPEVKFNWYVDGVEVHNAKTKPRE | TLSKADYEKHKVYACEVTHQGLSSP |
| | EQYNSTYRVVSVLTVLHQDWLNGKEYKCKVSNKALP | VTKSFNRGEC (SEQ ID NO: 537) |
| | APIEKTISKAKGQPREPQVYTLPPSRDELTKNQVSLTCL | |
| | VKGFYPSDIAVEWESNGQPENNYKTTPPVLDSDGSFFL | |
| | YSKLTVDKSRWQQGNVFSCSVMHEALHNHYTQKSLS | |
| | LSPGK (SEQ ID NO: 428) | |
| Hu22F10G3- | QVQLQESGPGLVKPSQTLSLTCTVSGYSLTSDFAWNW | DIVMTQSPDSLAVSLGERATINCRASE |
| 2.2 | IRQHPGKGLEWMGDIRYSGSTDYNPSLKGRVTISRDTS | SVDVYGNSFMHWYQQKPGQPPKLLI |
| | KNQFSLKLSSVTAADTAVYYCARGGYYGYDEGYPMD | YLAFNLKSGVPARFSGSGSGTDFTLTI |
| | YWGQGTLVTVSSASTKGPSVFPLAPSSKSTSGGTAALG | SSLQAEDVAVYYCQQNNEDPFTFGQ |
| | CLVKDYFPEPVTVSWNSGALTSGVHTFPAVLQSSGLY | GTKLEIKRTVAAPSVFIFPPSDEQLKS |
| | SLSSVVTVPSSSLGTQTYICNVNHKPSNTKVDKKVEPK | GTASVVCLLNNFYPREAKVQWKVDN |
| | SCDKTHTCPPCPAPELLGGPSVFLFPPKPKDTLMISRTP | ALQSGNSQESVTEQDSKDSTYSLSSTL |
| | EVTCVVVDVSHEDPEVKFNWYVDGVEVHNAKTKPRE | TLSKADYEKHKVYACEVTHQGLSSP |
| | EQYNSTYRVVSVLTVLHQDWLNGKEYKCKVSNKALP | VTKSFNRGEC (SEQ ID NO: 538) |
| | APIEKTISKAKGQPREPQVYTLPPSRDELTKNQVSLTCL | |
| | VKGFYPSDIAVEWESNGQPENNYKTTPPVLDSDGSFFL | |
| | YSKLTVDKSRWQQGNVFSCSVMHEALHNHYTQKSLS | |
| | LSPGK (SEQ ID NO: 429) | |
| Hu22F10G3- | QVQLQESGPGLVKPSQTLSLTCTVSGYSLTSDFAWNW | DIVMTQSPDSLAVSLGERATISCRASE |
| 2.3 | IRQHPGKGLEWMGDIRYSGSTDYNPSLKGRVTISRDTS | SVDVYGNSFMHWYQQKPGQPPKLLI |
| | KNQFSLKLSSVTAADTAVYYCARGGYYGYDEGYPMD | YLAFNLKSGVPDRFSGSGSGTDFTLTI |
| | YWGQGTLVTVSSASTKGPSVFPLAPSSKSTSGGTAALG | SSLQAEDVAVYYCQQNNEDPFTFGQ |
| | CLVKDYFPEPVTVSWNSGALTSGVHTFPAVLQSSGLY | GTKLEIKRTVAAPSVFIFPPSDEQLKS |
| | SLSSVVTVPSSSLGTQTYICNVNHKPSNTKVDKKVEPK | GTASVVCLLNNFYPREAKVQWKVDN |
| | SCDKTHTCPPCPAPELLGGPSVFLFPPKPKDTLMISRTP | ALQSGNSQESVTEQDSKDSTYSLSSTL |
| | EVTCVVVDVSHEDPEVKFNWYVDGVEVHNAKTKPRE | TLSKADYEKHKVYACEVTHQGLSSP |
| | EQYNSTYRVVSVLTVLHQDWLNGKEYKCKVSNKALP | VTKSFNRGEC (SEQ ID NO: 539) |
| | APIEKTISKAKGQPREPQVYTLPPSRDELTKNQVSLTCL | |
| | VKGFYPSDIAVEWESNGQPENNYKTTPPVLDSDGSFFL | |
| | YSKLTVDKSRWQQGNVFSCSVMHEALHNHYTQKSLS | |
| | LSPGK (SEQ ID NO: 430) | |
| Hu22F10G3- | QVQLQESGPGLVKPSQTLSLTCTVSGYSLTSDFAWNW | DIVMTQSPDSLAVSLGERATISCRASE |
| 2.4 | IRQHPGKGLEWMGDIRYSGSTDYNPSLKGRVTISRDTS | SVDVYGNSFMHWYQQKPGQPPKLLI |
| | KNQFSLKLSSVTAADTAVYYCARGGYYGYDEGYPMD | YLAFNLKSGVPARFSGSGSGTDFTLTI |
| | YWGQGTLVTVSSASTKGPSVFPLAPSSKSTSGGTAALG | SSLQAEDVATYYCQQNNEDPFTFGQ |
| | CLVKDYFPEPVTVSWNSGALTSGVHTFPAVLQSSGLY | GTKLEIKRTVAAPSVFIFPPSDEQLKS |
| | SLSSVVTVPSSSLGTQTYICNVNHKPSNTKVDKKVEPK | GTASVVCLLNNFYPREAKVQWKVDN |
| | SCDKTHTCPPCPAPELLGGPSVFLFPPKPKDTLMISRTP | ALQSGNSQESVTEQDSKDSTYSLSSTL |
| | EVTCVVVDVSHEDPEVKFNWYVDGVEVHNAKTKPRE | TLSKADYEKHKVYACEVTHQGLSSP |
| | EQYNSTYRVVSVLTVLHQDWLNGKEYKCKVSNKALP | VTKSFNRGEC (SEQ ID NO: 540) |
| | APIEKTISKAKGQPREPQVYTLPPSRDELTKNQVSLTCL | |
| | VKGFYPSDIAVEWESNGQPENNYKTTPPVLDSDGSFFL | |
| | YSKLTVDKSRWQQGNVFSCSVMHEALHNHYTQKSLS | |
| | LSPGK (SEQ ID NO: 431) | |
| Hu22F10G3- | QVQLQESGPGLVKPSQTLSLTCTVSGYSLTSDFAWNW | DIVMTQSPDSLAVSLGERATINCRASE |
| 3.1 | IRQHPGKGLEWMGDIRYSGSTDYNPSLKGRSTISRDTS | SVDVYGNSFMHWYQQKPGQPPKLLI |
| | KNQFSLKLSSVTAADTAVYYCARGGYYGYDEGYPMD | YLAFNLKSGVPDRFSGSGSGTDFTLTI |
| | YWGQGTLVTVSSASTKGPSVFPLAPSSKSTSGGTAALG | SSLQAEDVAVYYCQQNNEDPFTFGQ |
| | CLVKDYFPEPVTVSWNSGALTSGVHTFPAVLQSSGLY | GTKLEIKRTVAAPSVFIFPPSDEQLKS |
| | SLSSVVTVPSSSLGTQTYICNVNHKPSNTKVDKKVEPK | GTASVVCLLNNFYPREAKVQWKVDN |
| | SCDKTHTCPPCPAPELLGGPSVFLFPPKPKDTLMISRTP | ALQSGNSQESVTEQDSKDSTYSLSSTL |
| | EVTCVVVDVSHEDPEVKFNWYVDGVEVHNAKTKPRE | TLSKADYEKHKVYACEVTHQGLSSP |
| | EQYNSTYRVVSVLTVLHQDWLNGKEYKCKVSNKALP | VTKSFNRGEC (SEQ ID NO: 541) |
| | APIEKTISKAKGQPREPQVYTLPPSRDELTKNQVSLTCL | |
| | VKGFYPSDIAVEWESNGQPENNYKTTPPVLDSDGSFFL | |
| | YSKLTVDKSRWQQGNVFSCSVMHEALHNHYTQKSLS | |
| | LSPGK (SEQ ID NO: 432) | |
| Hu22F10G3- | QVQLQESGPGLVKPSQTLSLTCTVSGYSLTSDFAWNW | DIVMTQSPDSLAVSLGERATINCRASE |
| 3.2 | IRQHPGKGLEWMGDIRYSGSTDYNPSLKGRSTISRDTS | SVDVYGNSFMHWYQQKPGQPPKLLI |
| | KNQFSLKLSSVTAADTAVYYCARGGYYGYDEGYPMD | YLAFNLKSGVPARFSGSGSGTDFTLTI |
| | YWGQGTLVTVSSASTKGPSVFPLAPSSKSTSGGTAALG | SSLQAEDVAVYYCQQNNEDPFTFGQ |
| | CLVKDYFPEPVTVSWNSGALTSGVHTFPAVLQSSGLY | GTKLEIKRTVAAPSVFIFPPSDEQLKS |
| | SLSSVVTVPSSSLGTQTYICNVNHKPSNTKVDKKVEPK | GTASVVCLLNNFYPREAKVQWKVDN |
| | SCDKTHTCPPCPAPELLGGPSVFLFPPKPKDTLMISRTP | ALQSGNSQESVTEQDSKDSTYSLSSTL |
| | EVTCVVVDVSHEDPEVKFNWYVDGVEVHNAKTKPRE | TLSKADYEKHKVYACEVTHQGLSSP |
| | EQYNSTYRVVSVLTVLHQDWLNGKEYKCKVSNKALP | VTKSFNRGEC (SEQ ID NO: 542) |
| | APIEKTISKAKGQPREPQVYTLPPSRDELTKNQVSLTCL | |
| | VKGFYPSDIAVEWESNGQPENNYKTTPPVLDSDGSFFL | |
| | YSKLTVDKSRWQQGNVFSCSVMHEALHNHYTQKSLS | |
| | LSPGK (SEQ ID NO: 433) | |
| Hu22F10G3- | QVQLQESGPGLVKPSQTLSLTCTVSGYSLTSDFAWNW | DIVMTQSPDSLAVSLGERATISCRASE |
| 3.3 | IRQHPGKGLEWMGDIRYSGSTDYNPSLKGRSTISRDTS | SVDVYGNSFMHWYQQKPGQPPKLLI |
| | KNQFSLKLSSVTAADTAVYYCARGGYYGYDEGYPMD | YLAFNLKSGVPDRFSGSGSGTDFTLTI |
| | YWGQGTLVTVSSASTKGPSVFPLAPSSKSTSGGTAALG | SSLQAEDVAVYYCQQNNEDPFTFGQ |
| | CLVKDYFPEPVTVSWNSGALTSGVHTFPAVLQSSGLY | GTKLEIKRTVAAPSVFIFPPSDEQLKS |
| | SLSSVVTVPSSSLGTQTYICNVNHKPSNTKVDKKVEPK | GTASVVCLLNNFYPREAKVQWKVDN |
| | SCDKTHTCPPCPAPELLGGPSVFLFPPKPKDTLMISRTP | ALQSGNSQESVTEQDSKDSTYSLSSTL |
| | EVTCVVVDVSHEDPEVKFNWYVDGVEVHNAKTKPRE | TLSKADYEKHKVYACEVTHQGLSSP |
| | EQYNSTYRVVSVLTVLHQDWLNGKEYKCKVSNKALP | VTKSFNRGEC (SEQ ID NO: 543) |
| | APIEKTISKAKGQPREPQVYTLPPSRDELTKNQVSLTCL | |
| | VKGFYPSDIAVEWESNGQPENNYKTTPPVLDSDGSFFL | |
| | YSKLTVDKSRWQQGNVFSCSVMHEALHNHYTQKSLS | |
| | LSPGK (SEQ ID NO: 434) | |
| Hu22F10G3- | QVQLQESGPGLVKPSQTLSLTCTVSGYSLTSDFAWNW | DIVMTQSPDSLAVSLGERATISCRASE |
| 3.4 | IRQHPGKGLEWMGDIRYSGSTDYNPSLKGRSTISRDTS | SVDVYGNSFMHWYQQKPGQPPKLLI |
| | KNQFSLKLSSVTAADTAVYYCARGGYYGYDEGYPMD | YLAFNLKSGVPARFSGSGSGTDFTLTI |
| | YWGQGTLVTVSSASTKGPSVFPLAPSSKSTSGGTAALG | SSLQAEDVATYYCQQNNEDPFTFGQ |
| | CLVKDYFPEPVTVSWNSGALTSGVHTFPAVLQSSGLY | GTKLEIKRTVAAPSVFIFPPSDEQLKS |
| | SLSSVVTVPSSSLGTQTYICNVNHKPSNTKVDKKVEPK | GTASVVCLLNNFYPREAKVQWKVDN |
| | SCDKTHTCPPCPAPELLGGPSVFLFPPKPKDTLMISRTP | ALQSGNSQESVTEQDSKDSTYSLSSTL |
| | EVTCVVVDVSHEDPEVKFNWYVDGVEVHNAKTKPRE | TLSKADYEKHKVYACEVTHQGLSSP |
| | EQYNSTYRVVSVLTVLHQDWLNGKEYKCKVSNKALP | VTKSFNRGEC (SEQ ID NO: 544) |
| | APIEKTISKAKGQPREPQVYTLPPSRDELTKNQVSLTCL | |
| | VKGFYPSDIAVEWESNGQPENNYKTTPPVLDSDGSFFL | |
| | YSKLTVDKSRWQQGNVFSCSVMHEALHNHYTQKSLS | |
| | LSPGK (SEQ ID NO: 435) | |
| Hu22F10G3- | QVQLQESGPGLVKPSQTLSLTCTVSGYSLTSDFAWNW | DIVMTQSPDSLAVSLGERATINCRASE |
| 4.1 | IRQFPGKGLEWMGDIRYSGSTDYNPSLKGRSTISRDTS | SVDVYGNSFMHWYQQKPGQPPKLLI |
| | KNQFFLKLSSVTAADTAVYYCARGGYYGYDEGYPMD | YLAFNLKSGVPDRFSGSGSGTDFTLTI |
| | YWGQGTLVTVSSASTKGPSVFPLAPSSKSTSGGTAALG | SSLQAEDVAVYYCQQNNEDPFTFGQ |
| | CLVKDYFPEPVTVSWNSGALTSGVHTFPAVLQSSGLY | GTKLEIKRTVAAPSVFIFPPSDEQLKS |
| | SLSSVVTVPSSSLGTQTYICNVNHKPSNTKVDKKVEPK | GTASVVCLLNNFYPREAKVQWKVDN |
| | SCDKTHTCPPCPAPELLGGPSVFLFPPKPKDTLMISRTP | ALQSGNSQESVTEQDSKDSTYSLSSTL |
| | EVTCVVVDVSHEDPEVKFNWYVDGVEVHNAKTKPRE | TLSKADYEKHKVYACEVTHQGLSSP |
| | EQYNSTYRVVSVLTVLHQDWLNGKEYKCKVSNKALP | VTKSFNRGEC (SEQ ID NO: 545) |
| | APIEKTISKAKGQPREPQVYTLPPSRDELTKNQVSLTCL | |
| | VKGFYPSDIAVEWESNGQPENNYKTTPPVLDSDGSFFL | |
| | YSKLTVDKSRWQQGNVFSCSVMHEALHNHYTQKSLS | |
| | LSPGK (SEQ ID NO: 436) | |
| Hu22F10G3- | QVQLQESGPGLVKPSQTLSLTCTVSGYSLTSDFAWNW | DIVMTQSPDSLAVSLGERATINCRASE |
| 4.2 | IRQFPGKGLEWMGDIRYSGSTDYNPSLKGRSTISRDTS | SVDVYGNSFMHWYQQKPGQPPKLLI |
| | KNQFFLKLSSVTAADTAVYYCARGGYYGYDEGYPMD | YLAFNLKSGVPARFSGSGSGTDFTLTI |
| | YWGQGTLVTVSSASTKGPSVFPLAPSSKSTSGGTAALG | SSLQAEDVAVYYCQQNNEDPFTFGQ |
| | CLVKDYFPEPVTVSWNSGALTSGVHTFPAVLQSSGLY | GTKLEIKRTVAAPSVFIFPPSDEQLKS |
| | SLSSVVTVPSSSLGTQTYICNVNHKPSNTKVDKKVEPK | GTASVVCLLNNFYPREAKVQWKVDN |
| | SCDKTHTCPPCPAPELLGGPSVFLFPPKPKDTLMISRTP | ALQSGNSQESVTEQDSKDSTYSLSSTL |
| | EVTCVVVDVSHEDPEVKFNWYVDGVEVHNAKTKPRE | TLSKADYEKHKVYACEVTHQGLSSP |
| | EQYNSTYRVVSVLTVLHQDWLNGKEYKCKVSNKALP | VTKSFNRGEC (SEQ ID NO: 546) |
| | APIEKTISKAKGQPREPQVYTLPPSRDELTKNQVSLTCL | |
| | VKGFYPSDIAVEWESNGQPENNYKTTPPVLDSDGSFFL | |
| | YSKLTVDKSRWQQGNVFSCSVMHEALHNHYTQKSLS | |
| | LSPGK (SEQ ID NO: 437) | |
| Hu22F10G3- | QVQLQESGPGLVKPSQTLSLTCTVSGYSLTSDFAWNW | DIVMTQSPDSLAVSLGERATISCRASE |
| 4.3 | IRQFPGKGLEWMGDIRYSGSTDYNPSLKGRSTISRDTS | SVDVYGNSFMHWYQQKPGQPPKLLI |
| | KNQFFLKLSSVTAADTAVYYCARGGYYGYDEGYPMD | YLAFNLKSGVPDRFSGSGSGTDFTLTI |
| | YWGQGTLVTVSSASTKGPSVFPLAPSSKSTSGGTAALG | SSLQAEDVAVYYCQQNNEDPFTFGQ |
| | CLVKDYFPEPVTVSWNSGALTSGVHTFPAVLQSSGLY | GTKLEIKRTVAAPSVFIFPPSDEQLKS |
| | SLSSVVTVPSSSLGTQTYICNVNHKPSNTKVDKKVEPK | GTASVVCLLNNFYPREAKVQWKVDN |
| | SCDKTHTCPPCPAPELLGGPSVFLFPPKPKDTLMISRTP | ALQSGNSQESVTEQDSKDSTYSLSSTL |
| | EVTCVVVDVSHEDPEVKFNWYVDGVEVHNAKTKPRE | TLSKADYEKHKVYACEVTHQGLSSP |
| | EQYNSTYRVVSVLTVLHQDWLNGKEYKCKVSNKALP | VTKSFNRGEC (SEQ ID NO: 547) |
| | APIEKTISKAKGQPREPQVYTLPPSRDELTKNQVSLTCL | |
| | VKGFYPSDIAVEWESNGQPENNYKTTPPVLDSDGSFFL | |
| | YSKLTVDKSRWQQGNVFSCSVMHEALHNHYTQKSLS | |
| | LSPGK (SEQ ID NO: 438) | |
| Hu22F10G3- | QVQLQESGPGLVKPSQTLSLTCTVSGYSLTSDFAWNW | DIVMTQSPDSLAVSLGERATISCRASE |
| 4.4 | IRQFPGKGLEWMGDIRYSGSTDYNPSLKGRSTISRDTS | SVDVYGNSFMHWYQQKPGQPPKLLI |
| | KNQFFLKLSSVTAADTAVYYCARGGYYGYDEGYPMD | YLAFNLKSGVPARFSGSGSGTDFTLTI |
| | YWGQGTLVTVSSASTKGPSVFPLAPSSKSTSGGTAALG | SSLQAEDVATYYCQQNNEDPFTFGQ |
| | CLVKDYFPEPVTVSWNSGALTSGVHTFPAVLQSSGLY | GTKLEIKRTVAAPSVFIFPPSDEQLKS |
| | SLSSVVTVPSSSLGTQTYICNVNHKPSNTKVDKKVEPK | GTASVVCLLNNFYPREAKVQWKVDN |
| | SCDKTHTCPPCPAPELLGGPSVFLFPPKPKDTLMISRTP | ALQSGNSQESVTEQDSKDSTYSLSSTL |
| | EVTCVVVDVSHEDPEVKFNWYVDGVEVHNAKTKPRE | TLSKADYEKHKVYACEVTHQGLSSP |
| | EQYNSTYRVVSVLTVLHQDWLNGKEYKCKVSNKALP | VTKSFNRGEC (SEQ ID NO: 548) |
| | APIEKTISKAKGQPREPQVYTLPPSRDELTKNQVSLTCL | |
| | VKGFYPSDIAVEWESNGQPENNYKTTPPVLDSDGSFFL | |
| | YSKLTVDKSRWQQGNVFSCSVMHEALHNHYTQKSLS | |
| | LSPGK (SEQ ID NO: 439) | |
| Hu22F10G3- | QVQLQESGPGLVKPSQTLSLTCTVSGYSLTSDFAWNW | DIVMTQSPDSLAVSLGERATINCRASE |
| 1.1-NA | IRQHPGKGLEWIGDIRYSGSTDYNPSLKGRVTISVDTS | SVDVYGNAFMHWYQQKPGQPPKLLI |
| | KNQFSLKLSSVTAADTAVYYCARGGYYGYDEGYPMD | YLAFNLKSGVPDRFSGSGSGTDFTLTI |
| | YWGQGTLVTVSSASTKGPSVFPLAPSSKSTSGGTAALG | SSLQAEDVAVYYCQQNNEDPFTFGQ |
| | CLVKDYFPEPVTVSWNSGALTSGVHTFPAVLQSSGLY | GTKLEIKRTVAAPSVFIFPPSDEQLKS |
| | SLSSVVTVPSSSLGTQTYICNVNHKPSNTKVDKKVEPK | GTASVVCLLNNFYPREAKVQWKVDN |
| | SCDKTHTCPPCPAPELLGGPSVFLFPPKPKDTLMISRTP | ALQSGNSQESVTEQDSKDSTYSLSSTL |
| | EVTCVVVDVSHEDPEVKFNWYVDGVEVHNAKTKPRE | TLSKADYEKHKVYACEVTHQGLSSP |
| | EQYNSTYRVVSVLTVLHQDWLNGKEYKCKVSNKALP | VTKSFNRGEC (SEQ ID NO: 549) |
| | APIEKTISKAKGQPREPQVYTLPPSRDELTKNQVSLTCL | |
| | VKGFYPSDIAVEWESNGQPENNYKTTPPVLDSDGSFFL | |
| | YSKLTVDKSRWQQGNVFSCSVMHEALHNHYTQKSLS | |
| | LSPGK (SEQ ID NO: 440) | |
| Hu22F10G3- | QVQLQESGPGLVKPSQTLSLTCTVSGYSLTSDFAWNW | DIVMTQSPDSLAVSLGERATINCRASE |
| 1.2-NA | IRQHPGKGLEWIGDIRYSGSTDYNPSLKGRVTISVDTS | SVDVYGNAFMHWYQQKPGQPPKLLI |
| | KNQFSLKLSSVTAADTAVYYCARGGYYGYDEGYPMD | YLAFNLKSGVPARFSGSGSGTDFTLTI |
| | YWGQGTLVTVSSASTKGPSVFPLAPSSKSTSGGTAALG | SSLQAEDVAVYYCQQNNEDPFTFGQ |
| | CLVKDYFPEPVTVSWNSGALTSGVHTFPAVLQSSGLY | GTKLEIKRTVAAPSVFIFPPSDEQLKS |
| | SLSSVVTVPSSSLGTQTYICNVNHKPSNTKVDKKVEPK | GTASVVCLLNNFYPREAKVQWKVDN |
| | SCDKTHTCPPCPAPELLGGPSVFLFPPKPKDTLMISRTP | ALQSGNSQESVTEQDSKDSTYSLSSTL |
| | EVTCVVVDVSHEDPEVKFNWYVDGVEVHNAKTKPRE | TLSKADYEKHKVYACEVTHQGLSSP |
| | EQYNSTYRVVSVLTVLHQDWLNGKEYKCKVSNKALP | VTKSFNRGEC (SEQ ID NO: 550) |
| | APIEKTISKAKGQPREPQVYTLPPSRDELTKNQVSLTCL | |
| | VKGFYPSDIAVEWESNGQPENNYKTTPPVLDSDGSFFL | |
| | YSKLTVDKSRWQQGNVFSCSVMHEALHNHYTQKSLS | |
| | LSPGK (SEQ ID NO: 441) | |
| Hu22F10G3- | QVQLQESGPGLVKPSQTLSLTCTVSGYSLTSDFAWNW | DIVMTQSPDSLAVSLGERATISCRASE |
| 1.3-NA | IRQHPGKGLEWIGDIRYSGSTDYNPSLKGRVTISVDTS | SVDVYGNAFMHWYQQKPGQPPKLLI |
| | KNQFSLKLSSVTAADTAVYYCARGGYYGYDEGYPMD | YLAFNLKSGVPDRFSGSGSGTDFTLTI |
| | YWGQGTLVTVSSASTKGPSVFPLAPSSKSTSGGTAALG | SSLQAEDVAVYYCQQNNEDPFTFGQ |
| | CLVKDYFPEPVTVSWNSGALTSGVHTFPAVLQSSGLY | GTKLEIKRTVAAPSVFIFPPSDEQLKS |
| | SLSSVVTVPSSSLGTQTYICNVNHKPSNTKVDKKVEPK | GTASVVCLLNNFYPREAKVQWKVDN |
| | SCDKTHTCPPCPAPELLGGPSVFLFPPKPKDTLMISRTP | ALQSGNSQESVTEQDSKDSTYSLSSTL |
| | EVTCVVVDVSHEDPEVKFNWYVDGVEVHNAKTKPRE | TLSKADYEKHKVYACEVTHQGLSSP |
| | EQYNSTYRVVSVLTVLHQDWLNGKEYKCKVSNKALP | VTKSFNRGEC (SEQ ID NO: 551) |
| | APIEKTISKAKGQPREPQVYTLPPSRDELTKNQVSLTCL | |
| | VKGFYPSDIAVEWESNGQPENNYKTTPPVLDSDGSFFL | |
| | YSKLTVDKSRWQQGNVFSCSVMHEALHNHYTQKSLS | |
| | LSPGK (SEQ ID NO: 442) | |
| Hu22F10G3- | QVQLQESGPGLVKPSQTLSLTCTVSGYSLTSDFAWNW | DIVMTQSPDSLAVSLGERATISCRASE |
| 1.4-NA | IRQHPGKGLEWIGDIRYSGSTDYNPSLKGRVTISVDTS | SVDVYGNAFMHWYQQKPGQPPKLLI |
| | KNQFSLKLSSVTAADTAVYYCARGGYYGYDEGYPMD | YLAFNLKSGVPARFSGSGSGTDFTLTI |
| | YWGQGTLVTVSSASTKGPSVFPLAPSSKSTSGGTAALG | SSLQAEDVATYYCQQNNEDPFTFGQ |
| | CLVKDYFPEPVTVSWNSGALTSGVHTFPAVLQSSGLY | GTKLEIKRTVAAPSVFIFPPSDEQLKS |
| | SLSSVVTVPSSSLGTQTYICNVNHKPSNTKVDKKVEPK | GTASVVCLLNNFYPREAKVQWKVDN |
| | SCDKTHTCPPCPAPELLGGPSVFLFPPKPKDTLMISRTP | ALQSGNSQESVTEQDSKDSTYSLSSTL |
| | EVTCVVVDVSHEDPEVKFNWYVDGVEVHNAKTKPRE | TLSKADYEKHKVYACEVTHQGLSSP |
| | EQYNSTYRVVSVLTVLHQDWLNGKEYKCKVSNKALP | VTKSFNRGEC (SEQ ID NO: 552) |
| | APIEKTISKAKGQPREPQVYTLPPSRDELTKNQVSLTCL | |
| | VKGFYPSDIAVEWESNGQPENNYKTTPPVLDSDGSFFL | |
| | YSKLTVDKSRWQQGNVFSCSVMHEALHNHYTQKSLS | |
| | LSPGK (SEQ ID NO: 443) | |
| Hu22F10G3- | QVQLQESGPGLVKPSQTLSLTCTVSGYSLTSDFAWNW | DIVMTQSPDSLAVSLGERATINCRASE |
| 2.1-NA | IRQHPGKGLEWMGDIRYSGSTDYNPSLKGRVTISRDTS | SVDVYGNAFMHWYQQKPGQPPKLLI |
| | KNQFSLKLSSVTAADTAVYYCARGGYYGYDEGYPMD | YLAFNLKSGVPDRFSGSGSGTDFTLTI |
| | YWGQGTLVTVSSASTKGPSVFPLAPSSKSTSGGTAALG | SSLQAEDVAVYYCQQNNEDPFTFGQ |
| | CLVKDYFPEPVTVSWNSGALTSGVHTFPAVLQSSGLY | GTKLEIKRTVAAPSVFIFPPSDEQLKS |
| | SLSSVVTVPSSSLGTQTYICNVNHKPSNTKVDKKVEPK | GTASVVCLLNNFYPREAKVQWKVDN |
| | SCDKTHTCPPCPAPELLGGPSVFLFPPKPKDTLMISRTP | ALQSGNSQESVTEQDSKDSTYSLSSTL |
| | EVTCVVVDVSHEDPEVKFNWYVDGVEVHNAKTKPRE | TLSKADYEKHKVYACEVTHQGLSSP |
| | EQYNSTYRVVSVLTVLHQDWLNGKEYKCKVSNKALP | VTKSFNRGEC (SEQ ID NO: 553) |
| | APIEKTISKAKGQPREPQVYTLPPSRDELTKNQVSLTCL | |
| | VKGFYPSDIAVEWESNGQPENNYKTTPPVLDSDGSFFL | |
| | YSKLTVDKSRWQQGNVFSCSVMHEALHNHYTQKSLS | |
| | LSPGK (SEQ ID NO: 444) | |
| Hu22F10G3- | QVQLQESGPGLVKPSQTLSLTCTVSGYSLTSDFAWNW | DIVMTQSPDSLAVSLGERATINCRASE |
| 2.2-NA | IRQHPGKGLEWMGDIRYSGSTDYNPSLKGRVTISRDTS | SVDVYGNAFMHWYQQKPGQPPKLLI |
| | KNQFSLKLSSVTAADTAVYYCARGGYYGYDEGYPMD | YLAFNLKSGVPARFSGSGSGTDFTLTI |
| | YWGQGTLVTVSSASTKGPSVFPLAPSSKSTSGGTAALG | SSLQAEDVAVYYCQQNNEDPFTFGQ |
| | CLVKDYFPEPVTVSWNSGALTSGVHTFPAVLQSSGLY | GTKLEIKRTVAAPSVFIFPPSDEQLKS |
| | SLSSVVTVPSSSLGTQTYICNVNHKPSNTKVDKKVEPK | GTASVVCLLNNFYPREAKVQWKVDN |
| | SCDKTHTCPPCPAPELLGGPSVFLFPPKPKDTLMISRTP | ALQSGNSQESVTEQDSKDSTYSLSSTL |
| | EVTCVVVDVSHEDPEVKFNWYVDGVEVHNAKTKPRE | TLSKADYEKHKVYACEVTHQGLSSP |
| | EQYNSTYRVVSVLTVLHQDWLNGKEYKCKVSNKALP | VTKSFNRGEC (SEQ ID NO: 554) |
| | APIEKTISKAKGQPREPQVYTLPPSRDELTKNQVSLTCL | |
| | VKGFYPSDIAVEWESNGQPENNYKTTPPVLDSDGSFFL | |
| | YSKLTVDKSRWQQGNVFSCSVMHEALHNHYTQKSLS | |
| | LSPGK (SEQ ID NO: 445) | |
| Hu22F10G3- | QVQLQESGPGLVKPSQTLSLTCTVSGYSLTSDFAWNW | DIVMTQSPDSLAVSLGERATISCRASE |
| 2.3-NA | IRQHPGKGLEWMGDIRYSGSTDYNPSLKGRVTISRDTS | SVDVYGNAFMHWYQQKPGQPPKLLI |
| | KNQFSLKLSSVTAADTAVYYCARGGYYGYDEGYPMD | YLAFNLKSGVPDRFSGSGSGTDFTLTI |
| | YWGQGTLVTVSSASTKGPSVFPLAPSSKSTSGGTAALG | SSLQAEDVAVYYCQQNNEDPFTFGQ |
| | CLVKDYFPEPVTVSWNSGALTSGVHTFPAVLQSSGLY | GTKLEIKRTVAAPSVFIFPPSDEQLKS |
| | SLSSVVTVPSSSLGTQTYICNVNHKPSNTKVDKKVEPK | GTASVVCLLNNFYPREAKVQWKVDN |
| | SCDKTHTCPPCPAPELLGGPSVFLFPPKPKDTLMISRTP | ALQSGNSQESVTEQDSKDSTYSLSSTL |
| | EVTCVVVDVSHEDPEVKFNWYVDGVEVHNAKTKPRE | TLSKADYEKHKVYACEVTHQGLSSP |
| | EQYNSTYRVVSVLTVLHQDWLNGKEYKCKVSNKALP | VTKSFNRGEC (SEQ ID NO: 555) |
| | APIEKTISKAKGQPREPQVYTLPPSRDELTKNQVSLTCL | |
| | VKGFYPSDIAVEWESNGQPENNYKTTPPVLDSDGSFFL | |
| | YSKLTVDKSRWQQGNVFSCSVMHEALHNHYTQKSLS | |
| | LSPGK (SEQ ID NO: 446) | |
| Hu22F10G3- | QVQLQESGPGLVKPSQTLSLTCTVSGYSLTSDFAWNW | DIVMTQSPDSLAVSLGERATISCRASE |
| 2.4-NA | IRQHPGKGLEWMGDIRYSGSTDYNPSLKGRVTISRDTS | SVDVYGNAFMHWYQQKPGQPPKLLI |
| | KNQFSLKLSSVTAADTAVYYCARGGYYGYDEGYPMD | YLAFNLKSGVPARFSGSGSGTDFTLTI |
| | YWGQGTLVTVSSASTKGPSVFPLAPSSKSTSGGTAALG | SSLQAEDVATYYCQQNNEDPFTFGQ |
| | CLVKDYFPEPVTVSWNSGALTSGVHTFPAVLQSSGLY | GTKLEIKRTVAAPSVFIFPPSDEQLKS |
| | SLSSVVTVPSSSLGTQTYICNVNHKPSNTKVDKKVEPK | GTASVVCLLNNFYPREAKVQWKVDN |
| | SCDKTHTCPPCPAPELLGGPSVFLFPPKPKDTLMISRTP | ALQSGNSQESVTEQDSKDSTYSLSSTL |
| | EVTCVVVDVSHEDPEVKFNWYVDGVEVHNAKTKPRE | TLSKADYEKHKVYACEVTHQGLSSP |
| | EQYNSTYRVVSVLTVLHQDWLNGKEYKCKVSNKALP | VTKSFNRGEC (SEQ ID NO: 556) |
| | APIEKTISKAKGQPREPQVYTLPPSRDELTKNQVSLTCL | |
| | VKGFYPSDIAVEWESNGQPENNYKTTPPVLDSDGSFFL | |
| | YSKLTVDKSRWQQGNVFSCSVMHEALHNHYTQKSLS | |
| | LSPGK (SEQ ID NO: 447) | |
| Hu22F10G3- | QVQLQESGPGLVKPSQTLSLTCTVSGYSLTSDFAWNW | DIVMTQSPDSLAVSLGERATINCRASE |
| 3.1-NA | IRQHPGKGLEWMGDIRYSGSTDYNPSLKGRSTISRDTS | SVDVYGNAFMHWYQQKPGQPPKLLI |
| | KNQFSLKLSSVTAADTAVYYCARGGYYGYDEGYPMD | YLAFNLKSGVPDRFSGSGSGTDFTLTI |
| | YWGQGTLVTVSSASTKGPSVFPLAPSSKSTSGGTAALG | SSLQAEDVAVYYCQQNNEDPFTFGQ |
| | CLVKDYFPEPVTVSWNSGALTSGVHTFPAVLQSSGLY | GTKLEIKRTVAAPSVFIFPPSDEQLKS |
| | SLSSVVTVPSSSLGTQTYICNVNHKPSNTKVDKKVEPK | GTASVVCLLNNFYPREAKVQWKVDN |
| | SCDKTHTCPPCPAPELLGGPSVFLFPPKPKDTLMISRTP | ALQSGNSQESVTEQDSKDSTYSLSSTL |
| | EVTCVVVDVSHEDPEVKFNWYVDGVEVHNAKTKPRE | TLSKADYEKHKVYACEVTHQGLSSP |
| | EQYNSTYRVVSVLTVLHQDWLNGKEYKCKVSNKALP | VTKSFNRGEC (SEQ ID NO: 557) |
| | APIEKTISKAKGQPREPQVYTLPPSRDELTKNQVSLTCL | |
| | VKGFYPSDIAVEWESNGQPENNYKTTPPVLDSDGSFFL | |
| | YSKLTVDKSRWQQGNVFSCSVMHEALHNHYTQKSLS | |
| | LSPGK (SEQ ID NO: 448) | |
| Hu22F10G3- | QVQLQESGPGLVKPSQTLSLTCTVSGYSLTSDFAWNW | DIVMTQSPDSLAVSLGERATINCRASE |
| 3.2-NA | IRQHPGKGLEWMGDIRYSGSTDYNPSLKGRSTISRDTS | SVDVYGNAFMHWYQQKPGQPPKLLI |
| | KNQFSLKLSSVTAADTAVYYCARGGYYGYDEGYPMD | YLAFNLKSGVPARFSGSGSGTDFTLTI |
| | YWGQGTLVTVSSASTKGPSVFPLAPSSKSTSGGTAALG | SSLQAEDVAVYYCQQNNEDPFTFGQ |
| | CLVKDYFPEPVTVSWNSGALTSGVHTFPAVLQSSGLY | GTKLEIKRTVAAPSVFIFPPSDEQLKS |
| | SLSSVVTVPSSSLGTQTYICNVNHKPSNTKVDKKVEPK | GTASVVCLLNNFYPREAKVQWKVDN |
| | SCDKTHTCPPCPAPELLGGPSVFLFPPKPKDTLMISRTP | ALQSGNSQESVTEQDSKDSTYSLSSTL |
| | EVTCVVVDVSHEDPEVKFNWYVDGVEVHNAKTKPRE | TLSKADYEKHKVYACEVTHQGLSSP |
| | EQYNSTYRVVSVLTVLHQDWLNGKEYKCKVSNKALP | VTKSFNRGEC (SEQ ID NO: 558) |
| | APIEKTISKAKGQPREPQVYTLPPSRDELTKNQVSLTCL | |
| | VKGFYPSDIAVEWESNGQPENNYKTTPPVLDSDGSFFL | |
| | YSKLTVDKSRWQQGNVFSCSVMHEALHNHYTQKSLS | |
| | LSPGK (SEQ ID NO: 449) | |
| Hu22F10G3- | QVQLQESGPGLVKPSQTLSLTCTVSGYSLTSDFAWNW | DIVMTQSPDSLAVSLGERATISCRASE |
| 3.3-NA | IRQHPGKGLEWMGDIRYSGSTDYNPSLKGRSTISRDTS | SVDVYGNAFMHWYQQKPGQPPKLLI |
| | KNQFSLKLSSVTAADTAVYYCARGGYYGYDEGYPMD | YLAFNLKSGVPDRFSGSGSGTDFTLTI |
| | YWGQGTLVTVSSASTKGPSVFPLAPSSKSTSGGTAALG | SSLQAEDVAVYYCQQNNEDPFTFGQ |
| | CLVKDYFPEPVTVSWNSGALTSGVHTFPAVLQSSGLY | GTKLEIKRTVAAPSVFIFPPSDEQLKS |
| | SLSSVVTVPSSSLGTQTYICNVNHKPSNTKVDKKVEPK | GTASVVCLLNNFYPREAKVQWKVDN |
| | SCDKTHTCPPCPAPELLGGPSVFLFPPKPKDTLMISRTP | ALQSGNSQESVTEQDSKDSTYSLSSTL |
| | EVTCVVVDVSHEDPEVKFNWYVDGVEVHNAKTKPRE | TLSKADYEKHKVYACEVTHQGLSSP |
| | EQYNSTYRVVSVLTVLHQDWLNGKEYKCKVSNKALP | VTKSFNRGEC (SEQ ID NO: 559) |
| | APIEKTISKAKGQPREPQVYTLPPSRDELTKNQVSLTCL | |
| | VKGFYPSDIAVEWESNGQPENNYKTTPPVLDSDGSFFL | |
| | YSKLTVDKSRWQQGNVFSCSVMHEALHNHYTQKSLS | |
| | LSPGK (SEQ ID NO: 450) | |
| Hu22F10G3- | QVQLQESGPGLVKPSQTLSLTCTVSGYSLTSDFAWNW | DIVMTQSPDSLAVSLGERATISCRASE |
| 3.4-NA | IRQHPGKGLEWMGDIRYSGSTDYNPSLKGRSTISRDTS | SVDVYGNAFMHWYQQKPGQPPKLLI |
| | KNQFSLKLSSVTAADTAVYYCARGGYYGYDEGYPMD | YLAFNLKSGVPARFSGSGSGTDFTLTI |
| | YWGQGTLVTVSSASTKGPSVFPLAPSSKSTSGGTAALG | SSLQAEDVATYYCQQNNEDPFTFGQ |
| | CLVKDYFPEPVTVSWNSGALTSGVHTFPAVLQSSGLY | GTKLEIKRTVAAPSVFIFPPSDEQLKS |
| | SLSSVVTVPSSSLGTQTYICNVNHKPSNTKVDKKVEPK | GTASVVCLLNNFYPREAKVQWKVDN |
| | SCDKTHTCPPCPAPELLGGPSVFLFPPKPKDTLMISRTP | ALQSGNSQESVTEQDSKDSTYSLSSTL |
| | EVTCVVVDVSHEDPEVKFNWYVDGVEVHNAKTKPRE | TLSKADYEKHKVYACEVTHQGLSSP |
| | EQYNSTYRVVSVLTVLHQDWLNGKEYKCKVSNKALP | VTKSFNRGEC (SEQ ID NO: 560) |
| | APIEKTISKAKGQPREPQVYTLPPSRDELTKNQVSLTCL | |
| | VKGFYPSDIAVEWESNGQPENNYKTTPPVLDSDGSFFL | |
| | YSKLTVDKSRWQQGNVFSCSVMHEALHNHYTQKSLS | |
| | LSPGK (SEQ ID NO: 451) | |
| Hu22F10G3- | QVQLQESGPGLVKPSQTLSLTCTVSGYSLTSDFAWNW | DIVMTQSPDSLAVSLGERATINCRASE |
| 4.1-NA | IRQFPGKGLEWMGDIRYSGSTDYNPSLKGRSTISRDTS | SVDVYGNAFMHWYQQKPGQPPKLLI |
| | KNQFFLKLSSVTAADTAVYYCARGGYYGYDEGYPMD | YLAFNLKSGVPDRFSGSGSGTDFTLTI |
| | YWGQGTLVTVSSASTKGPSVFPLAPSSKSTSGGTAALG | SSLQAEDVAVYYCQQNNEDPFTFGQ |
| | CLVKDYFPEPVTVSWNSGALTSGVHTFPAVLQSSGLY | GTKLEIKRTVAAPSVFIFPPSDEQLKS |
| | SLSSVVTVPSSSLGTQTYICNVNHKPSNTKVDKKVEPK | GTASVVCLLNNFYPREAKVQWKVDN |
| | SCDKTHTCPPCPAPELLGGPSVFLFPPKPKDTLMISRTP | ALQSGNSQESVTEQDSKDSTYSLSSTL |
| | EVTCVVVDVSHEDPEVKFNWYVDGVEVHNAKTKPRE | TLSKADYEKHKVYACEVTHQGLSSP |
| | EQYNSTYRVVSVLTVLHQDWLNGKEYKCKVSNKALP | VTKSFNRGEC (SEQ ID NO: 561) |
| | APIEKTISKAKGQPREPQVYTLPPSRDELTKNQVSLTCL | |
| | VKGFYPSDIAVEWESNGQPENNYKTTPPVLDSDGSFFL | |
| | YSKLTVDKSRWQQGNVFSCSVMHEALHNHYTQKSLS | |
| | LSPGK (SEQ ID NO: 452) | |
| Hu22F10G3- | QVQLQESGPGLVKPSQTLSLTCTVSGYSLTSDFAWNW | DIVMTQSPDSLAVSLGERATINCRASE |
| 4.2-NA | IRQFPGKGLEWMGDIRYSGSTDYNPSLKGRSTISRDTS | SVDVYGNAFMHWYQQKPGQPPKLLI |
| | KNQFFLKLSSVTAADTAVYYCARGGYYGYDEGYPMD | YLAFNLKSGVPARFSGSGSGTDFTLTI |
| | YWGQGTLVTVSSASTKGPSVFPLAPSSKSTSGGTAALG | SSLQAEDVAVYYCQQNNEDPFTFGQ |
| | CLVKDYFPEPVTVSWNSGALTSGVHTFPAVLQSSGLY | GTKLEIKRTVAAPSVFIFPPSDEQLKS |
| | SLSSVVTVPSSSLGTQTYICNVNHKPSNTKVDKKVEPK | GTASVVCLLNNFYPREAKVQWKVDN |
| | SCDKTHTCPPCPAPELLGGPSVFLFPPKPKDTLMISRTP | ALQSGNSQESVTEQDSKDSTYSLSSTL |
| | EVTCVVVDVSHEDPEVKFNWYVDGVEVHNAKTKPRE | TLSKADYEKHKVYACEVTHQGLSSP |
| | EQYNSTYRVVSVLTVLHQDWLNGKEYKCKVSNKALP | VTKSFNRGEC (SEQ ID NO: 562) |
| | APIEKTISKAKGQPREPQVYTLPPSRDELTKNQVSLTCL | |
| | VKGFYPSDIAVEWESNGQPENNYKTTPPVLDSDGSFFL | |
| | YSKLTVDKSRWQQGNVFSCSVMHEALHNHYTQKSLS | |
| | LSPGK (SEQ ID NO: 453) | |
| Hu22F10G3- | QVQLQESGPGLVKPSQTLSLTCTVSGYSLTSDFAWNW | DIVMTQSPDSLAVSLGERATISCRASE |
| 4.3-NA | IRQFPGKGLEWMGDIRYSGSTDYNPSLKGRSTISRDTS | SVDVYGNAFMHWYQQKPGQPPKLLI |
| | KNQFFLKLSSVTAADTAVYYCARGGYYGYDEGYPMD | YLAFNLKSGVPDRFSGSGSGTDFTLTI |
| | YWGQGTLVTVSSASTKGPSVFPLAPSSKSTSGGTAALG | SSLQAEDVAVYYCQQNNEDPFTFGQ |
| | CLVKDYFPEPVTVSWNSGALTSGVHTFPAVLQSSGLY | GTKLEIKRTVAAPSVFIFPPSDEQLKS |
| | SLSSVVTVPSSSLGTQTYICNVNHKPSNTKVDKKVEPK | GTASVVCLLNNFYPREAKVQWKVDN |
| | SCDKTHTCPPCPAPELLGGPSVFLFPPKPKDTLMISRTP | ALQSGNSQESVTEQDSKDSTYSLSSTL |
| | EVTCVVVDVSHEDPEVKFNWYVDGVEVHNAKTKPRE | TLSKADYEKHKVYACEVTHQGLSSP |
| | EQYNSTYRVVSVLTVLHQDWLNGKEYKCKVSNKALP | VTKSFNRGEC (SEQ ID NO: 563) |
| | APIEKTISKAKGQPREPQVYTLPPSRDELTKNQVSLTCL | |
| | VKGFYPSDIAVEWESNGQPENNYKTTPPVLDSDGSFFL | |
| | YSKLTVDKSRWQQGNVFSCSVMHEALHNHYTQKSLS | |
| | LSPGK (SEQ ID NO: 454) | |
| Hu22F10G3- | QVQLQESGPGLVKPSQTLSLTCTVSGYSLTSDFAWNW | DIVMTQSPDSLAVSLGERATISCRASE |
| 4.4-NA | IRQFPGKGLEWMGDIRYSGSTDYNPSLKGRSTISRDTS | SVDVYGNAFMHWYQQKPGQPPKLLI |
| | KNQFFLKLSSVTAADTAVYYCARGGYYGYDEGYPMD | YLAFNLKSGVPARFSGSGSGTDFTLTI |
| | YWGQGTLVTVSSASTKGPSVFPLAPSSKSTSGGTAALG | SSLQAEDVATYYCQQNNEDPFTFGQ |
| | CLVKDYFPEPVTVSWNSGALTSGVHTFPAVLQSSGLY | GTKLEIKRTVAAPSVFIFPPSDEQLKS |
| | SLSSVVTVPSSSLGTQTYICNVNHKPSNTKVDKKVEPK | GTASVVCLLNNFYPREAKVQWKVDN |
| | SCDKTHTCPPCPAPELLGGPSVFLFPPKPKDTLMISRTP | ALQSGNSQESVTEQDSKDSTYSLSSTL |
| | EVTCVVVDVSHEDPEVKFNWYVDGVEVHNAKTKPRE | TLSKADYEKHKVYACEVTHQGLSSP |
| | EQYNSTYRVVSVLTVLHQDWLNGKEYKCKVSNKALP | VTKSFNRGEC (SEQ ID NO: 564) |
| | APIEKTISKAKGQPREPQVYTLPPSRDELTKNQVSLTCL | |
| | VKGFYPSDIAVEWESNGQPENNYKTTPPVLDSDGSFFL | |
| | YSKLTVDKSRWQQGNVFSCSVMHEALHNHYTQKSLS | |
| | LSPGK (SEQ ID NO: 455) | |
| Hu22F10G3- | QVQLQESGPGLVKPSQTLSLTCTVSGYSLTSDFAWNW | DIVMTQSPDSLAVSLGERATINCRASE |
| 1.1-QS | IRQHPGKGLEWIGDIRYSGSTDYNPSLKGRVTISVDTS | SVDVYGQSFMHWYQQKPGQPPKLLI |
| | KNQFSLKLSSVTAADTAVYYCARGGYYGYDEGYPMD | YLAFNLKSGVPDRFSGSGSGTDFTLTI |
| | YWGQGTLVTVSSASTKGPSVFPLAPSSKSTSGGTAALG | SSLQAEDVAVYYCQQNNEDPFTFGQ |
| | CLVKDYFPEPVTVSWNSGALTSGVHTFPAVLQSSGLY | GTKLEIKRTVAAPSVFIFPPSDEQLKS |
| | SLSSVVTVPSSSLGTQTYICNVNHKPSNTKVDKKVEPK | GTASVVCLLNNFYPREAKVQWKVDN |
| | SCDKTHTCPPCPAPELLGGPSVFLFPPKPKDTLMISRTP | ALQSGNSQESVTEQDSKDSTYSLSSTL |
| | EVTCVVVDVSHEDPEVKFNWYVDGVEVHNAKTKPRE | TLSKADYEKHKVYACEVTHQGLSSP |
| | EQYNSTYRVVSVLTVLHQDWLNGKEYKCKVSNKALP | VTKSFNRGEC (SEQ ID NO: 565) |
| | APIEKTISKAKGQPREPQVYTLPPSRDELTKNQVSLTCL | |
| | VKGFYPSDIAVEWESNGQPENNYKTTPPVLDSDGSFFL | |
| | YSKLTVDKSRWQQGNVFSCSVMHEALHNHYTQKSLS | |
| | LSPGK (SEQ ID NO: 456) | |
| Hu22F10G3- | QVQLQESGPGLVKPSQTLSLTCTVSGYSLTSDFAWNW | DIVMTQSPDSLAVSLGERATINCRASE |
| 1.2-QS | IRQHPGKGLEWIGDIRYSGSTDYNPSLKGRVTISVDTS | SVDVYGQSFMHWYQQKPGQPPKLLI |
| | KNQFSLKLSSVTAADTAVYYCARGGYYGYDEGYPMD | YLAFNLKSGVPARFSGSGSGTDFTLTI |
| | YWGQGTLVTVSSASTKGPSVFPLAPSSKSTSGGTAALG | SSLQAEDVAVYYCQQNNEDPFTFGQ |
| | CLVKDYFPEPVTVSWNSGALTSGVHTFPAVLQSSGLY | GTKLEIKRTVAAPSVFIFPPSDEQLKS |
| | SLSSVVTVPSSSLGTQTYICNVNHKPSNTKVDKKVEPK | GTASVVCLLNNFYPREAKVQWKVDN |
| | SCDKTHTCPPCPAPELLGGPSVFLFPPKPKDTLMISRTP | ALQSGNSQESVTEQDSKDSTYSLSSTL |
| | EVTCVVVDVSHEDPEVKFNWYVDGVEVHNAKTKPRE | TLSKADYEKHKVYACEVTHQGLSSP |
| | EQYNSTYRVVSVLTVLHQDWLNGKEYKCKVSNKALP | VTKSFNRGEC (SEQ ID NO: 566) |
| | APIEKTISKAKGQPREPQVYTLPPSRDELTKNQVSLTCL | |
| | VKGFYPSDIAVEWESNGQPENNYKTTPPVLDSDGSFFL | |
| | YSKLTVDKSRWQQGNVFSCSVMHEALHNHYTQKSLS | |
| | LSPGK (SEQ ID NO: 457) | |
| Hu22F10G3- | QVQLQESGPGLVKPSQTLSLTCTVSGYSLTSDFAWNW | DIVMTQSPDSLAVSLGERATISCRASE |
| 1.3-QS | IRQHPGKGLEWIGDIRYSGSTDYNPSLKGRVTISVDTS | SVDVYGQSFMHWYQQKPGQPPKLLI |
| | KNQFSLKLSSVTAADTAVYYCARGGYYGYDEGYPMD | YLAFNLKSGVPDRFSGSGSGTDFTLTI |
| | YWGQGTLVTVSSASTKGPSVFPLAPSSKSTSGGTAALG | SSLQAEDVAVYYCQQNNEDPFTFGQ |
| | CLVKDYFPEPVTVSWNSGALTSGVHTFPAVLQSSGLY | GTKLEIKRTVAAPSVFIFPPSDEQLKS |
| | SLSSVVTVPSSSLGTQTYICNVNHKPSNTKVDKKVEPK | GTASVVCLLNNFYPREAKVQWKVDN |
| | SCDKTHTCPPCPAPELLGGPSVFLFPPKPKDTLMISRTP | ALQSGNSQESVTEQDSKDSTYSLSSTL |
| | EVTCVVVDVSHEDPEVKFNWYVDGVEVHNAKTKPRE | TLSKADYEKHKVYACEVTHQGLSSP |
| | EQYNSTYRVVSVLTVLHQDWLNGKEYKCKVSNKALP | VTKSFNRGEC (SEQ ID NO: 567) |
| | APIEKTISKAKGQPREPQVYTLPPSRDELTKNQVSLTCL | |
| | VKGFYPSDIAVEWESNGQPENNYKTTPPVLDSDGSFFL | |
| | YSKLTVDKSRWQQGNVFSCSVMHEALHNHYTQKSLS | |
| | LSPGK (SEQ ID NO: 458) | |
| Hu22F10G3- | QVQLQESGPGLVKPSQTLSLTCTVSGYSLTSDFAWNW | DIVMTQSPDSLAVSLGERATISCRASE |
| 1.4-QS | IRQHPGKGLEWIGDIRYSGSTDYNPSLKGRVTISVDTS | SVDVYGQSFMHWYQQKPGQPPKLLI |
| | KNQFSLKLSSVTAADTAVYYCARGGYYGYDEGYPMD | YLAFNLKSGVPARFSGSGSGTDFTLTI |
| | YWGQGTLVTVSSASTKGPSVFPLAPSSKSTSGGTAALG | SSLQAEDVATYYCQQNNEDPFTFGQ |
| | CLVKDYFPEPVTVSWNSGALTSGVHTFPAVLQSSGLY | GTKLEIKRTVAAPSVFIFPPSDEQLKS |
| | SLSSVVTVPSSSLGTQTYICNVNHKPSNTKVDKKVEPK | GTASVVCLLNNFYPREAKVQWKVDN |
| | SCDKTHTCPPCPAPELLGGPSVFLFPPKPKDTLMISRTP | ALQSGNSQESVTEQDSKDSTYSLSSTL |
| | EVTCVVVDVSHEDPEVKFNWYVDGVEVHNAKTKPRE | TLSKADYEKHKVYACEVTHQGL SSP |
| | EQYNSTYRVVSVLTVLHQDWLNGKEYKCKVSNKALP | VTKSFNRGEC (SEQ ID NO: 568) |
| | APIEKTISKAKGQPREPQVYTLPPSRDELTKNQVSLTCL | |
| | VKGFYPSDIAVEWESNGQPENNYKTTPPVLDSDGSFFL | |
| | YSKLTVDKSRWQQGNVFSCSVMHEALHNHYTQKSLS | |
| | LSPGK (SEQ ID NO: 459) | |
| Hu22F10G3- | QVQLQESGPGLVKPSQTLSLTCTVSGYSLTSDFAWNW | DIVMTQSPDSLAVSLGERATINCRASE |
| 2.1-QS | IRQHPGKGLEWMGDIRYSGSTDYNPSLKGRVTISRDTS | SVDVYGQSFMHWYQQKPGQPPKLLI |
| | KNQFSLKLSSVTAADTAVYYCARGGYYGYDEGYPMD | YLAFNLKSGVPDRFSGSGSGTDFTLTI |
| | YWGQGTLVTVSSASTKGPSVFPLAPSSKSTSGGTAALG | SSLQAEDVAVYYCQQNNEDPFTFGQ |
| | CLVKDYFPEPVTVSWNSGALTSGVHTFPAVLQSSGLY | GTKLEIKRTVAAPSVFIFPPSDEQLKS |
| | SLSSVVTVPSSSLGTQTYICNVNHKPSNTKVDKKVEPK | GTASVVCLLNNFYPREAKVQWKVDN |
| | SCDKTHTCPPCPAPELLGGPSVFLFPPKPKDTLMISRTP | ALQSGNSQESVTEQDSKDSTYSLSSTL |
| | EVTCVVVDVSHEDPEVKFNWYVDGVEVHNAKTKPRE | TLSKADYEKHKVYACEVTHQGLSSP |
| | EQYNSTYRVVSVLTVLHQDWLNGKEYKCKVSNKALP | VTKSFNRGEC (SEQ ID NO: 569) |
| | APIEKTISKAKGQPREPQVYTLPPSRDELTKNQVSLTCL | |
| | VKGFYPSDIAVEWESNGQPENNYKTTPPVLDSDGSFFL | |
| | YSKLTVDKSRWQQGNVFSCSVMHEALHNHYTQKSLS | |
| | LSPGK (SEQ ID NO: 460) | |
| Hu22F10G3- | QVQLQESGPGLVKPSQTLSLTCTVSGYSLTSDFAWNW | DIVMTQSPDSLAVSLGERATINCRASE |
| 2.2-QS | IRQHPGKGLEWMGDIRYSGSTDYNPSLKGRVTISRDTS | SVDVYGQSFMHWYQQKPGQPPKLLI |
| | KNQFSLKLSSVTAADTAVYYCARGGYYGYDEGYPMD | YLAFNLKSGVPARFSGSGSGTDFTLTI |
| | YWGQGTLVTVSSASTKGPSVFPLAPSSKSTSGGTAALG | SSLQAEDVAVYYCQQNNEDPFTFGQ |
| | CLVKDYFPEPVTVSWNSGALTSGVHTFPAVLQSSGLY | GTKLEIKRTVAAPSVFIFPPSDEQLKS |
| | SLSSVVTVPSSSLGTQTYICNVNHKPSNTKVDKKVEPK | GTASVVCLLNNFYPREAKVQWKVDN |
| | SCDKTHTCPPCPAPELLGGPSVFLFPPKPKDTLMISRTP | ALQSGNSQESVTEQDSKDSTYSLSSTL |
| | EVTCVVVDVSHEDPEVKFNWYVDGVEVHNAKTKPRE | TLSKADYEKHKVYACEVTHQGLSSP |
| | EQYNSTYRVVSVLTVLHQDWLNGKEYKCKVSNKALP | VTKSFNRGEC (SEQ ID NO: 570) |
| | APIEKTISKAKGQPREPQVYTLPPSRDELTKNQVSLTCL | |
| | VKGFYPSDIAVEWESNGQPENNYKTTPPVLDSDGSFFL | |
| | YSKLTVDKSRWQQGNVFSCSVMHEALHNHYTQKSLS | |
| | LSPGK (SEQ ID NO: 461) | |
| Hu22F10G3- | QVQLQESGPGLVKPSQTLSLTCTVSGYSLTSDFAWNW | DIVMTQSPDSLAVSLGERATISCRASE |
| 2.3-QS | IRQHPGKGLEWMGDIRYSGSTDYNPSLKGRVTISRDTS | SVDVYGQSFMHWYQQKPGQPPKLLI |
| | KNQFSLKLSSVTAADTAVYYCARGGYYGYDEGYPMD | YLAFNLKSGVPDRFSGSGSGTDFTLTI |
| | YWGQGTLVTVSSASTKGPSVFPLAPSSKSTSGGTAALG | SSLQAEDVAVYYCQQNNEDPFTFGQ |
| | CLVKDYFPEPVTVSWNSGALTSGVHTFPAVLQSSGLY | GTKLEIKRTVAAPSVFIFPPSDEQLKS |
| | SLSSVVTVPSSSLGTQTYICNVNHKPSNTKVDKKVEPK | GTASVVCLLNNFYPREAKVQWKVDN |
| | SCDKTHTCPPCPAPELLGGPSVFLFPPKPKDTLMISRTP | ALQSGNSQESVTEQDSKDSTYSLSSTL |
| | EVTCVVVDVSHEDPEVKFNWYVDGVEVHNAKTKPRE | TLSKADYEKHKVYACEVTHQGLSSP |
| | EQYNSTYRVVSVLTVLHQDWLNGKEYKCKVSNKALP | VTKSFNRGEC (SEQ ID NO: 571) |
| | APIEKTISKAKGQPREPQVYTLPPSRDELTKNQVSLTCL | |
| | VKGFYPSDIAVEWESNGQPENNYKTTPPVLDSDGSFFL | |
| | YSKLTVDKSRWQQGNVFSCSVMHEALHNHYTQKSLS | |
| | LSPGK (SEQ ID NO: 462) | |
| Hu22F10G3- | QVQLQESGPGLVKPSQTLSLTCTVSGYSLTSDFAWNW | DIVMTQSPDSLAVSLGERATISCRASE |
| 2.4-QS | IRQHPGKGLEWMGDIRYSGSTDYNPSLKGRVTISRDTS | SVDVYGQSFMHWYQQKPGQPPKLLI |
| | KNQFSLKLSSVTAADTAVYYCARGGYYGYDEGYPMD | YLAFNLKSGVPARFSGSGSGTDFTLTI |
| | YWGQGTLVTVSSASTKGPSVFPLAPSSKSTSGGTAALG | SSLQAEDVATYYCQQNNEDPFTFGQ |
| | CLVKDYFPEPVTVSWNSGALTSGVHTFPAVLQSSGLY | GTKLEIKRTVAAPSVFIFPPSDEQLKS |
| | SLSSVVTVPSSSLGTQTYICNVNHKPSNTKVDKKVEPK | GTASVVCLLNNFYPREAKVQWKVDN |
| | SCDKTHTCPPCPAPELLGGPSVFLFPPKPKDTLMISRTP | ALQSGNSQESVTEQDSKDSTYSLSSTL |
| | EVTCVVVDVSHEDPEVKFNWYVDGVEVHNAKTKPRE | TLSKADYEKHKVYACEVTHQGLSSP |
| | EQYNSTYRVVSVLTVLHQDWLNGKEYKCKVSNKALP | VTKSFNRGEC (SEQ ID NO: 572) |
| | APIEKTISKAKGQPREPQVYTLPPSRDELTKNQVSLTCL | |
| | VKGFYPSDIAVEWESNGQPENNYKTTPPVLDSDGSFFL | |
| | YSKLTVDKSRWQQGNVFSCSVMHEALHNHYTQKSLS | |
| | LSPGK (SEQ ID NO: 463) | |
| Hu22F10G3- | QVQLQESGPGLVKPSQTLSLTCTVSGYSLTSDFAWNW | DIVMTQSPDSLAVSLGERATINCRASE |
| 3.1-QS | IRQHPGKGLEWMGDIRYSGSTDYNPSLKGRSTISRDTS | SVDVYGQSFMHWYQQKPGQPPKLLI |
| | KNQFSLKLSSVTAADTAVYYCARGGYYGYDEGYPMD | YLAFNLKSGVPDRFSGSGSGTDFTLTI |
| | YWGQGTLVTVSSASTKGPSVFPLAPSSKSTSGGTAALG | SSLQAEDVAVYYCQQNNEDPFTFGQ |
| | CLVKDYFPEPVTVSWNSGALTSGVHTFPAVLQSSGLY | GTKLEIKRTVAAPSVFIFPPSDEQLKS |
| | SLSSVVTVPSSSLGTQTYICNVNHKPSNTKVDKKVEPK | GTASVVCLLNNFYPREAKVQWKVDN |
| | SCDKTHTCPPCPAPELLGGPSVFLFPPKPKDTLMISRTP | ALQSGNSQESVTEQDSKDSTYSLSSTL |
| | EVTCVVVDVSHEDPEVKFNWYVDGVEVHNAKTKPRE | TLSKADYEKHKVYACEVTHQGL SSP |
| | EQYNSTYRVVSVLTVLHQDWLNGKEYKCKVSNKALP | VTKSFNRGEC (SEQ ID NO: 573) |
| | APIEKTISKAKGQPREPQVYTLPPSRDELTKNQVSLTCL | |
| | VKGFYPSDIAVEWESNGQPENNYKTTPPVLDSDGSFFL | |
| | YSKLTVDKSRWQQGNVFSCSVMHEALHNHYTQKSLS | |
| | LSPGK (SEQ ID NO: 464) | |
| Hu22F10G3- | QVQLQESGPGLVKPSQTLSLTCTVSGYSLTSDFAWNW | DIVMTQSPDSLAVSLGERATINCRASE |
| 3.2-QS | IRQHPGKGLEWMGDIRYSGSTDYNPSLKGRSTISRDTS | SVDVYGQSFMHWYQQKPGQPPKLLI |
| | KNQFSLKLSSVTAADTAVYYCARGGYYGYDEGYPMD | YLAFNLKSGVPARFSGSGSGTDFTLTI |
| | YWGQGTLVTVSSASTKGPSVFPLAPSSKSTSGGTAALG | SSLQAEDVAVYYCQQNNEDPFTFGQ |
| | CLVKDYFPEPVTVSWNSGALTSGVHTFPAVLQSSGLY | GTKLEIKRTVAAPSVFIFPPSDEQLKS |
| | SLSSVVTVPSSSLGTQTYICNVNHKPSNTKVDKKVEPK | GTASVVCLLNNFYPREAKVQWKVDN |
| | SCDKTHTCPPCPAPELLGGPSVFLFPPKPKDTLMISRTP | ALQSGNSQESVTEQDSKDSTYSLSSTL |
| | EVTCVVVDVSHEDPEVKFNWYVDGVEVHNAKTKPRE | TLSKADYEKHKVYACEVTHQGLSSP |
| | EQYNSTYRVVSVLTVLHQDWLNGKEYKCKVSNKALP | VTKSFNRGEC (SEQ ID NO: 574) |
| | APIEKTISKAKGQPREPQVYTLPPSRDELTKNQVSLTCL | |
| | VKGFYPSDIAVEWESNGQPENNYKTTPPVLDSDGSFFL | |
| | YSKLTVDKSRWQQGNVFSCSVMHEALHNHYTQKSLS | |
| | LSPGK (SEQ ID NO: 465) | |
| Hu22F10G3- | QVQLQESGPGLVKPSQTLSLTCTVSGYSLTSDFAWNW | DIVMTQSPDSLAVSLGERATISCRASE |
| 3.3-QS | IRQHPGKGLEWMGDIRYSGSTDYNPSLKGRSTISRDTS | SVDVYGQSFMHWYQQKPGQPPKLLI |
| | KNQFSLKLSSVTAADTAVYYCARGGYYGYDEGYPMD | YLAFNLKSGVPDRFSGSGSGTDFTLTI |
| | YWGQGTLVTVSSASTKGPSVFPLAPSSKSTSGGTAALG | SSLQAEDVAVYYCQQNNEDPFTFGQ |
| | CLVKDYFPEPVTVSWNSGALTSGVHTFPAVLQSSGLY | GTKLEIKRTVAAPSVFIFPPSDEQLKS |
| | SLSSVVTVPSSSLGTQTYICNVNHKPSNTKVDKKVEPK | GTASVVCLLNNFYPREAKVQWKVDN |
| | SCDKTHTCPPCPAPELLGGPSVFLFPPKPKDTLMISRTP | ALQSGNSQESVTEQDSKDSTYSLSSTL |
| | EVTCVVVDVSHEDPEVKFNWYVDGVEVHNAKTKPRE | TLSKADYEKHKVYACEVTHQGLSSP |
| | EQYNSTYRVVSVLTVLHQDWLNGKEYKCKVSNKALP | VTKSFNRGEC (SEQ ID NO: 575) |
| | APIEKTISKAKGQPREPQVYTLPPSRDELTKNQVSLTCL | |
| | VKGFYPSDIAVEWESNGQPENNYKTTPPVLDSDGSFFL | |
| | YSKLTVDKSRWQQGNVFSCSVMHEALHNHYTQKSLS | |
| | LSPGK (SEQ ID NO: 466) | |
| Hu22F10G3- | QVQLQESGPGLVKPSQTLSLTCTVSGYSLTSDFAWNW | DIVMTQSPDSLAVSLGERATISCRASE |
| 3.4-QS | IRQHPGKGLEWMGDIRYSGSTDYNPSLKGRSTISRDTS | SVDVYGQSFMHWYQQKPGQPPKLLI |
| | KNQFSLKLSSVTAADTAVYYCARGGYYGYDEGYPMD | YLAFNLKSGVPARFSGSGSGTDFTLTI |
| | YWGQGTLVTVSSASTKGPSVFPLAPSSKSTSGGTAALG | SSLQAEDVATYYCQQNNEDPFTFGQ |
| | CLVKDYFPEPVTVSWNSGALTSGVHTFPAVLQSSGLY | GTKLEIKRTVAAPSVFIFPPSDEQLKS |
| | SLSSVVTVPSSSLGTQTYICNVNHKPSNTKVDKKVEPK | GTASVVCLLNNFYPREAKVQWKVDN |
| | SCDKTHTCPPCPAPELLGGPSVFLFPPKPKDTLMISRTP | ALQSGNSQESVTEQDSKDSTYSLSSTL |
| | EVTCVVVDVSHEDPEVKFNWYVDGVEVHNAKTKPRE | TLSKADYEKHKVYACEVTHQGLSSP |
| | EQYNSTYRVVSVLTVLHQDWLNGKEYKCKVSNKALP | VTKSFNRGEC (SEQ ID NO: 576) |
| | APIEKTISKAKGQPREPQVYTLPPSRDELTKNQVSLTCL | |
| | VKGFYPSDIAVEWESNGQPENNYKTTPPVLDSDGSFFL | |
| | YSKLTVDKSRWQQGNVFSCSVMHEALHNHYTQKSLS | |
| | LSPGK (SEQ ID NO: 467) | |
| Hu22F10G3- | QVQLQESGPGLVKPSQTLSLTCTVSGYSLTSDFAWNW | DIVMTQSPDSLAVSLGERATINCRASE |
| 4.1-QS | IRQFPGKGLEWMGDIRYSGSTDYNPSLKGRSTISRDTS | SVDVYGQSFMHWYQQKPGQPPKLLI |
| | KNQFFLKLSSVTAADTAVYYCARGGYYGYDEGYPMD | YLAFNLKSGVPDRFSGSGSGTDFTLTI |
| | YWGQGTLVTVSSASTKGPSVFPLAPSSKSTSGGTAALG | SSLQAEDVAVYYCQQNNEDPFTFGQ |
| | CLVKDYFPEPVTVSWNSGALTSGVHTFPAVLQSSGLY | GTKLEIKRTVAAPSVFIFPPSDEQLKS |
| | SLSSVVTVPSSSLGTQTYICNVNHKPSNTKVDKKVEPK | GTASVVCLLNNFYPREAKVQWKVDN |
| | SCDKTHTCPPCPAPELLGGPSVFLFPPKPKDTLMISRTP | ALQSGNSQESVTEQDSKDSTYSLSSTL |
| | EVTCVVVDVSHEDPEVKFNWYVDGVEVHNAKTKPRE | TLSKADYEKHKVYACEVTHQGLSSP |
| | EQYNSTYRVVSVLTVLHQDWLNGKEYKCKVSNKALP | VTKSFNRGEC (SEQ ID NO: 577) |
| | APIEKTISKAKGQPREPQVYTLPPSRDELTKNQVSLTCL | |
| | VKGFYPSDIAVEWESNGQPENNYKTTPPVLDSDGSFFL | |
| | YSKLTVDKSRWQQGNVFSCSVMHEALHNHYTQKSLS | |
| | LSPGK (SEQ ID NO: 468) | |
| Hu22F10G3- | QVQLQESGPGLVKPSQTLSLTCTVSGYSLTSDFAWNW | DIVMTQSPDSLAVSLGERATINCRASE |
| 4.2-QS | IRQFPGKGLEWMGDIRYSGSTDYNPSLKGRSTISRDTS | SVDVYGQSFMHWYQQKPGQPPKLLI |
| | KNQFFLKLSSVTAADTAVYYCARGGYYGYDEGYPMD | YLAFNLKSGVPARFSGSGSGTDFTLTI |
| | YWGQGTLVTVSSASTKGPSVFPLAPSSKSTSGGTAALG | SSLQAEDVAVYYCQQNNEDPFTFGQ |
| | CLVKDYFPEPVTVSWNSGALTSGVHTFPAVLQSSGLY | GTKLEIKRTVAAPSVFIFPPSDEQLKS |
| | SLSSVVTVPSSSLGTQTYICNVNHKPSNTKVDKKVEPK | GTASVVCLLNNFYPREAKVQWKVDN |
| | SCDKTHTCPPCPAPELLGGPSVFLFPPKPKDTLMISRTP | ALQSGNSQESVTEQDSKDSTYSLSSTL |
| | EVTCVVVDVSHEDPEVKFNWYVDGVEVHNAKTKPRE | TLSKADYEKHKVYACEVTHQGLSSP |
| | EQYNSTYRVVSVLTVLHQDWLNGKEYKCKVSNKALP | VTKSFNRGEC (SEQ ID NO: 578) |
| | APIEKTISKAKGQPREPQVYTLPPSRDELTKNQVSLTCL | |
| | VKGFYPSDIAVEWESNGQPENNYKTTPPVLDSDGSFFL | |
| | YSKLTVDKSRWQQGNVFSCSVMHEALHNHYTQKSLS | |
| | LSPGK (SEQ ID NO: 469) | |
| Hu22F10G3- | QVQLQESGPGLVKPSQTLSLTCTVSGYSLTSDFAWNW | DIVMTQSPDSLAVSLGERATISCRASE |
| 4.3-QS | IRQFPGKGLEWMGDIRYSGSTDYNPSLKGRSTISRDTS | SVDVYGQSFMHWYQQKPGQPPKLLI |
| | KNQFFLKLSSVTAADTAVYYCARGGYYGYDEGYPMD | YLAFNLKSGVPDRFSGSGSGTDFTLTI |
| | YWGQGTLVTVSSASTKGPSVFPLAPSSKSTSGGTAALG | SSLQAEDVAVYYCQQNNEDPFTFGQ |
| | CLVKDYFPEPVTVSWNSGALTSGVHTFPAVLQSSGLY | GTKLEIKRTVAAPSVFIFPPSDEQLKS |
| | SLSSVVTVPSSSLGTQTYICNVNHKPSNTKVDKKVEPK | GTASVVCLLNNFYPREAKVQWKVDN |
| | SCDKTHTCPPCPAPELLGGPSVFLFPPKPKDTLMISRTP | ALQSGNSQESVTEQDSKDSTYSLSSTL |
| | EVTCVVVDVSHEDPEVKFNWYVDGVEVHNAKTKPRE | TLSKADYEKHKVYACEVTHQGLSSP |
| | EQYNSTYRVVSVLTVLHQDWLNGKEYKCKVSNKALP | VTKSFNRGEC (SEQ ID NO: 579) |
| | APIEKTISKAKGQPREPQVYTLPPSRDELTKNQVSLTCL | |
| | VKGFYPSDIAVEWESNGQPENNYKTTPPVLDSDGSFFL | |
| | YSKLTVDKSRWQQGNVFSCSVMHEALHNHYTQKSLS | |
| | LSPGK (SEQ ID NO: 470) | |
| Hu22F10G3- | QVQLQESGPGLVKPSQTLSLTCTVSGYSLTSDFAWNW | DIVMTQSPDSLAVSLGERATISCRASE |
| 4.4-QS | IRQFPGKGLEWMGDIRYSGSTDYNPSLKGRSTISRDTS | SVDVYGQSFMHWYQQKPGQPPKLLI |
| | KNQFFLKLSSVTAADTAVYYCARGGYYGYDEGYPMD | YLAFNLKSGVPARFSGSGSGTDFTLTI |
| | YWGQGTLVTVSSASTKGPSVFPLAPSSKSTSGGTAALG | SSLQAEDVATYYCQQNNEDPFTFGQ |
| | CLVKDYFPEPVTVSWNSGALTSGVHTFPAVLQSSGLY | GTKLEIKRTVAAPSVFIFPPSDEQLKS |
| | SLSSVVTVPSSSLGTQTYICNVNHKPSNTKVDKKVEPK | GTASVVCLLNNFYPREAKVQWKVDN |
| | SCDKTHTCPPCPAPELLGGPSVFLFPPKPKDTLMISRTP | ALQSGNSQESVTEQDSKDSTYSLSSTL |
| | EVTCVVVDVSHEDPEVKFNWYVDGVEVHNAKTKPRE | TLSKADYEKHKVYACEVTHQGLSSP |
| | EQYNSTYRVVSVLTVLHQDWLNGKEYKCKVSNKALP | VTKSFNRGEC (SEQ ID NO: 580) |
| | APIEKTISKAKGQPREPQVYTLPPSRDELTKNQVSLTCL | |
| | VKGFYPSDIAVEWESNGQPENNYKTTPPVLDSDGSFFL | |
| | YSKLTVDKSRWQQGNVFSCSVMHEALHNHYTQKSLS | |
| | LSPGK (SEQ ID NO: 471) | |

In some embodiments, a KLRB1-binding agent (e.g., an antibody) as described herein comprises a heavy chain region having at least about 80% (e.g., at least 90%, 95%, 97%, 99%, or 100%) sequence identity to a HC sequence set forth in Table C, and/or a light chain region having at least about 80% (e.g., at least 90%, 95%, 97%, 99%, or 100%) sequence identity to a LC sequence set forth in Table C. In some embodiments, the VH comprises or consists of an amino acid sequence having at least 95% sequence identity to the VH amino acid sequence set forth in Table C, and the VL comprises or consists of an amino acid sequence having at least 95% sequence identity to the VL amino acid sequence set forth in Table C.

The present disclosure further embraces additional variants and equivalents that are substantially homologous to the recombinant, monoclonal, chimeric, humanized, and human antibodies, or antibody fragments thereof, described herein. In some embodiments, it is desirable to improve the binding affinity of the antibody. In some embodiments, it is desirable to modulate biological properties of the antibody, including but not limited to, specificity, thermostability, expression level, effector function(s), glycosylation, immunogenicity, or solubility. Those skilled in the art will appreciate that amino acid changes may alter post-translational processes of an antibody, such as changing the number or position of glycosylation sites or altering membrane anchoring characteristics.

Variations may be a substitution, deletion, or insertion of one or more nucleotides encoding the antibody or polypeptide that results in a change in the amino acid sequence as compared with the native antibody or polypeptide sequence. In some embodiments, amino acid substitutions are the result of replacing one amino acid with another amino acid having similar structural and/or chemical properties, such as the replacement of a leucine with a serine, e.g., conservative amino acid replacements. Insertions or deletions may optionally be in the range of about 1 to 5 amino acids. In some embodiments, the substitution, deletion, or insertion includes less than 25 amino acid substitutions, less than 20 amino acid substitutions, less than 15 amino acid substitutions, less than 10 amino acid substitutions, less than 5 amino acid substitutions, less than 4 amino acid substitutions, less than 3 amino acid substitutions, or less than 2 amino acid substitutions relative to the parent molecule. In some embodiments, variations in the amino acid sequence that are biologically useful and/or relevant are determined by systematically making insertions, deletions, or substitutions in the sequence and testing the resulting variant proteins for activity as compared to the parental antibody.

In some embodiments, variants may include addition of amino acid residues at the amino- and/or carboxyl-terminal end of the antibody or polypeptide. The length of additional amino acids residues may range from one residue to a hundred or more residues. In some embodiments, a variant comprises an N-terminal methionyl residue. In some embodiments, the variant comprises an additional polypeptide/protein, i.e., a fusion protein. In some embodiments, a variant is engineered to be detectable and may comprise a detectable label and/or protein (e.g., an enzyme).

In some embodiments, a cysteine residue not involved in maintaining the proper conformation of an antibody may be substituted or deleted to modulate the antibody's characteristics, for example, to improve oxidative stability and/or prevent aberrant disulfide crosslinking. Conversely, in some embodiments, one or more cysteine residues may be added to create disulfide bond(s) to improve stability.

The variant antibodies or polypeptides described herein may be generated using methods known in the art, including but not limited to, site-directed mutagenesis, alanine scanning mutagenesis, and PCR mutagenesis.

In some embodiments, KLRB1-binding agents described herein are chemically modified. In some embodiments, the KLRB1-binding agents are anti-KLRB1 antibodies that have been chemically modified by glycosylation, acetylation, pegylation, phosphorylation, amidation, derivatization by known protecting/blocking groups, proteolytic cleavage, and/or linkage to a cellular ligand or other protein. Any of numerous chemical modifications may be carried out by known techniques.

The present disclosure encompasses KLRB1-binding agents built upon non-immunoglobulin backbones, wherein the agents bind to the same epitope or essentially the same epitope as an anti-KLRB1 antibody disclosed herein. In some embodiments, a non-immunoglobulin-based binding agent is an agent that competes with an anti-KLRB1 antibody described herein in a competitive binding assay. In some embodiments, alternative KLRB1-binding agents comprise a scaffold protein. Generally, scaffold proteins can be assigned to one of three groups based on the architecture of their backbone (1) scaffolds consisting of α-helices; (2) small scaffolds with few secondary structures or an irregular architecture of α-helices and β-sheets; and (3) scaffolds consisting of predominantly β-sheets. Scaffold proteins include, but are not limited to, anticalins, which are based upon the lipocalin scaffold; adnectins, which are based on the 10th domain of human fibronectin type 3; affibodies, which are based on the B-domain in the Ig-binding region of Staphylococcus aureus protein A; darpins, which are based on ankyrin repeat domain proteins; fynomers, which are based on the SH3 domain of the human Fyn protein kinase; affitins, which are based on Sac7d from Sulfolobus acidocaldarius; affilins, which are based on human γ-B-crystallin or human ubiquitin; avimers, which are based on the A-domains of membrane receptor proteins; knottins (cysteine knot miniproteins), which are based upon a stable 30-amino acid anti-parallel β-strand protein fold; and Kunitz domain inhibitor scaffolds, which are based upon a structure that contains three disulfide bonds and three loops. In some embodiments, a KLRB1-binding agent comprises an engineered scaffold protein comprising a heavy chain CDR1, CDR2, and CDR3 and a light chain CDR1, CDR2, and CDR3 shown in any one of Table 1 to 18, e.g., in Table 8, 9, or 18.

In general, antigen-antibody interactions are non-covalent and reversible, formed by a combination of hydrogen bonds, hydrophobic interactions, electrostatic and van der Waals forces. When describing the strength of an antigen-antibody complex, the terms affinity and/or avidity are commonly used mentioned. The binding of an antibody to its antigen is a reversible process, and the affinity of the binding is typically reported as an equilibrium dissociation constant (K_{D}). K_{D} is the ratio of an antibody dissociation rate (k_{off}) (how quickly it dissociates from its antigen) to the antibody association rate (kₒₙ) (how quickly it binds to its antigen). In some embodiments, K_{D} values are determined by measuring the kₒₙ and k_{off} rates of a specific antibody/antigen interaction and then using a ratio of these values to calculate the K_{D} value. K_{D} values may be used to evaluate and rank order the strength of individual antibody/antigen interactions. The lower the K_{D} of an antibody, the higher the affinity of the antibody for its target. In some embodiments, affinity is measured using SPR technology in a Biacore system. Avidity gives a measure of the overall strength of an antibody-antigen complex. It is dependent on three major parameters: (i) affinity of the antibody for the target, (ii) valency of both the antibody and antigen, and (iii) structural arrangement of the parts that interact.

In some embodiments, a KLRB1-binding agent (e.g., an antibody) binds KLRB1 (e.g., human KLRB1) with a dissociation constant (K_{D}) of about 1 μM or less, about 100 nM or less, about 40 nM or less, about 20 nM or less, about 10 nM or less, about 1 nM or less, about 0.1 nM or less, 50 pM or less, 10 pM or less, or 1 pM or less. In some embodiments, a KLRB1-binding agent binds KLRB1 (e.g., human KLRB1) with a K_{D} of about 20 nM or less. In some embodiments, a KLRB1-binding agent binds KLRB1 (e.g., human KLRB1) with a K_{D} of about 10 nM or less. In some embodiments, a KLRB1-binding agent binds KLRB1 (e.g., human KLRB1) with a K_{D} of about 1 nM or less. In some embodiments, a KLRB1-binding agent binds KLRB1 (e.g., human KLRB1) with a K_{D} of about 0.5 nM or less. In some embodiments, a KLRB1-binding agent binds KLRB1 (e.g., human KLRB1) with a K_{D} of about 0.1 nM or less. In some embodiments, a KLRB1-binding agent binds KLRB1 (e.g., human KLRB1) with a K_{D} of about 50 pM or less. In some embodiments, a KLRB1-binding agent binds KLRB1 (e.g., human KLRB1) with a K_{D} of about 25 pM or less. In some embodiments, a KLRB1-binding agent binds KLRB1 (e.g., human KLRB1) with a K_{D} of about 10 pM or less. In some embodiments, a KLRB1-binding agent binds KLRB1 (e.g., human KLRB1) with a K_{D} of about 1 pM or less. In some embodiments, the dissociation constant of the binding agent (e.g., an antibody) for KLRB1 is the dissociation constant determined using a KLRB1 protein immobilized on a Biacore chip and the binding agent flowed over the chip. In some embodiments, the dissociation constant of the binding agent (e.g., an antibody) for KLRB1 is the dissociation constant determined using the binding agent captured by an anti-human IgG antibody on a Biacore chip and soluble KLRB1 flowed over the chip.

In some embodiments, a KLRB1-binding agent (e.g., an antibody) binds KLRB1 (e.g., human KLRB1) with a half maximal effective concentration (EC50) of about 1 μM or less, about 100 nM or less, about 40 nM or less, about 20 nM or less, about 10 nM or less, about 1 nM or less, or about 0.1 nM or less. In some embodiments, a KLRB1-binding agent binds to human KLRB1 with an EC50 of about 1 μM or less, about 100 nM or less, about 40 nM or less, about 20 nM or less, about 10 nM or less, about 1 nM or less, or about 0.1 nM or less. In some embodiments, a KLRB1-binding agent binds cyno KLRB1 and/or human KLRB1 with an EC50 of about 40 nM or less, about 20 nM or less, about 10 nM or less, about 1 nM or less or about 0.1 nM or less.

The KLRB1-binding agents (e.g., antibodies) described herein can be produced by any suitable method known in the art. Such methods range from direct protein synthesis methods to constructing a DNA sequence encoding polypeptide sequences and expressing those sequences in a suitable host. In some embodiments, a DNA sequence is constructed using recombinant technology by isolating or synthesizing a DNA sequence encoding a wild-type protein of interest. Optionally, the sequence can be mutagenized by site-specific mutagenesis to provide functional variants thereof. In some embodiments, a DNA sequence encoding a polypeptide of interest is constructed by chemical synthesis using an oligonucleotide synthesizer. Oligonucleotides can be designed based on the amino acid sequence of the desired polypeptide and selecting those codons that are favored in the host cell in which the recombinant polypeptide of interest will be produced. Standard methods can be applied to synthesize a polynucleotide sequence encoding an isolated polypeptide of interest. For example, a complete amino acid sequence can be used to construct a back-translated gene. Further, a DNA oligomer containing a nucleotide sequence coding for the particular isolated polypeptide can be synthesized. For example, several small oligonucleotides coding for portions of the desired polypeptide can be synthesized and then ligated. The individual oligonucleotides typically contain 5′ or 3′ overhangs for complementary assembly.

Once assembled (by synthesis, site-directed mutagenesis, or another method), the polynucleotide sequences encoding a particular polypeptide of interest can be inserted into an expression vector and operatively linked to an expression control sequence appropriate for expression of the protein in a desired host. Proper assembly can be confirmed by nucleotide sequencing, restriction enzyme mapping, and/or expression of a biologically active polypeptide in a suitable host. As is well-known in the art, in order to obtain high expression levels of a transfected gene in a host, the gene must be operatively linked to transcriptional and translational expression control sequences that are functional in the chosen expression host.

In some embodiments, recombinant expression vectors are used to amplify and express DNA encoding antibodies, or fragments thereof, against human KLRB1. For example, recombinant expression vectors can be replicable DNA constructs which have synthetic or cDNA-derived DNA fragments encoding a polypeptide chain of a KLRB1-binding agent, such as an anti-KLRB1 antibody, or antigen-binding fragment thereof, operatively linked to suitable transcriptional and/or translational regulatory elements derived from mammalian, microbial, viral or insect genes. A transcriptional unit generally comprises an assembly of (1) a genetic element or elements having a regulatory role in gene expression, for example, transcriptional promoters or enhancers, (2) a structural or coding sequence which is transcribed into mRNA and translated into protein, and (3) appropriate transcription and translation initiation and termination sequences. Regulatory elements can include an operator sequence to control transcription. The ability to replicate in a host, usually conferred by an origin of replication, and a selection gene to facilitate recognition of transformants can additionally be incorporated. DNA regions are “operatively linked” when they are functionally related to each other. For example, DNA for a signal peptide (secretory leader) is operatively linked to DNA for a polypeptide if it is expressed as a precursor which participates in the secretion of the polypeptide; a promoter is operatively linked to a coding sequence if it controls the transcription of the sequence; or a ribosome binding site is operatively linked to a coding sequence if it is positioned so as to permit translation. In some embodiments, structural elements intended for use in yeast expression systems include a leader sequence enabling extracellular secretion of translated protein by a host cell. In some embodiments, in situations where recombinant protein is expressed without a leader or transport sequence, a polypeptide may include an N-terminal methionine residue. This residue can optionally be subsequently cleaved from the expressed recombinant protein to provide a final product.

The choice of an expression control sequence and an expression vector generally depends upon the choice of host. A wide variety of expression host/vector combinations can be employed. Useful expression vectors for eukaryotic hosts include, for example, vectors comprising expression control sequences from SV40, bovine papilloma virus, adenovirus, and cytomegalovirus. Useful expression vectors for bacterial hosts include known bacterial plasmids, such as plasmids from E. coli, including pCR1, pBR322, pMB9 and their derivatives, and wider host range plasmids, such as M13 and other filamentous single-stranded DNA phages.

The KLRB1-binding agents (e.g., antibodies) of the present disclosure can be expressed from one or more vectors. For example, in some embodiments, a heavy chain polypeptide is expressed by one vector and a light chain polypeptide is expressed by a second vector. In some embodiments, a heavy chain polypeptide and a light chain polypeptide are expressed by one vector.

Suitable host cells for expression of a KLRB1-binding agent (e.g., an antibody) or a KLRB1 protein or fragment thereof to use as an antigen or immunogen include prokaryotes, yeast cells, insect cells, or higher eukaryotic cells under the control of appropriate promoters. Prokaryotes include gram-negative or gram-positive organisms, for example E. coli or Bacillus. Higher eukaryotic cells include established cell lines of mammalian origin as described herein. Cell-free translation systems may also be employed. Appropriate cloning and expression vectors for use with bacterial, fungal, yeast, and mammalian cellular hosts, as well as methods of protein production, including antibody production are well known in the art.

Various mammalian culture systems may be used to express recombinant polypeptides. Expression of recombinant proteins in mammalian cells may be desirable because these proteins are generally correctly folded, appropriately modified, and biologically functional. Examples of suitable mammalian host cell lines include, but are not limited to, COS-7 (monkey kidney-derived), L-929 (murine fibroblast-derived), C127 (murine mammary tumor-derived), 3T3 (murine fibroblast-derived), CHO (Chinese hamster ovary-derived), HeLa (human cervical cancer-derived), BHK (hamster kidney fibroblast-derived), HEK-293 (human embryonic kidney-derived) cell lines and variants thereof. Mammalian expression vectors can comprise non-transcribed elements such as an origin of replication, a suitable promoter and enhancer linked to the gene to be expressed, and other 5′ or 3′ flanking non-transcribed sequences, and 5′ or 3′ non-translated sequences, such as necessary ribosome binding sites, a polyadenylation site, splice donor and acceptor sites, and transcriptional termination sequences.

Expression of recombinant proteins in insect cell culture systems (e.g., baculovirus) also offers a robust method for producing correctly folded and biologically functional proteins. Baculovirus systems for production of heterologous proteins in insect cells are well-known to those of skill in the art. Thus, the present disclosure provides cells comprising the KLRB1-binding agents described herein. In some embodiments, the cells produce the KLRB1-binding agents described herein. In some embodiments, the cells produce an antibody. In some embodiments, the cells produce an antibody that binds human KLRB1. In some embodiments, the cells produce an antibody that binds cyno KLRB1. In some embodiments, the cells produce an antibody that binds human KLRB1 and cyno KLRB1. In some embodiments, the cells produce an antibody designated 18G11F11, 9C10G11, 57E2E4, 75B10F12, 7B6C7, 52G9B12B5, 5B1B11, 50A8F2, 47A5H2, 66G9C11, 33F11A6, 37G6A7, 39C5D6, 62H1D6, 92E12F2, 13H2D11, 5A11D10, 22F10G3, or variants thereof. In some embodiments, the cells produce a scFv version of antibody 18G11F11, 9C10G11, 57E2E4, 75B10F12, 7B6C7, 52G9B12B5, 5B1B11, 50A8F2, 47A5H2, 66G9C11, 33F11A6, 37G6A7, 39C5D6, 62H1D6, 92E12F2, 13H2D11, 5A11D10, or 22F10G3. In some embodiments, the cell is a prokaryotic cell (e.g., E. coli). In some embodiments, the cell is an eukaryotic cell. In some embodiments, the cell is a mammalian cell. In some embodiments, the cell is a hybridoma cell. Proteins produced by a host cell can be purified according to any suitable method. Standard methods include chromatography (e.g., ion exchange, affinity, and sizing column chromatography), centrifugation, differential solubility, or by any other standard technique for protein purification. Affinity tags such as hexa-histidine, maltose binding domain, influenza coat sequence, and glutathione-S-transferase can be attached to the protein to allow easy purification by passage over an appropriate affinity column. Affinity chromatography used for purifying immunoglobulins can include Protein A, Protein G, and Protein L chromatography. Isolated proteins can be physically characterized using such techniques as proteolysis, size exclusion chromatography (SEC), mass spectrometry (MS), nuclear magnetic resonance (NMR), isoelectric focusing (IEF), high performance liquid chromatography (HPLC), and x-ray crystallography. The purity of isolated proteins can be determined using techniques known to those of skill in the art, including but not limited to, SDS-PAGE, SEC, capillary gel electrophoresis, IEF, and capillary isoelectric focusing (cIEF). In some embodiments, supernatants from expression systems which secrete recombinant protein into culture media are first concentrated using a commercially available protein concentration filter, for example, an Amicon® or Millipore Pellicon® ultrafiltration unit. Following the concentration step, the concentrate can be applied to a suitable purification matrix. In some embodiments, an anion exchange resin is employed, for example, a matrix or substrate having pendant diethylaminoethyl (DEAE) groups. The matrices can be acrylamide, agarose, dextran, cellulose, or other types commonly employed in protein purification. In some embodiments, a cation exchange step is employed. Suitable cation exchangers include various insoluble matrices comprising sulfopropyl or carboxymethyl groups. In some embodiments, a hydroxyapatite media is employed, including but not limited to, ceramic hydroxyapatite (CHT). In some embodiments, one or more reverse-phase HPLC steps employing hydrophobic RP-HPLC media, e.g., silica gel having pendant methyl or other aliphatic groups, are employed to further purify a recombinant protein. In some embodiments, hydrophobic interaction chromatography (HIC) is used to separate recombinant proteins based on their hydrophobicity. HIC is a useful separation technique for purifying proteins while maintaining biological activity due to the use of conditions and matrices that operate under less denaturing conditions than some other techniques. Some or all of the foregoing purification steps, in various combinations, can be employed to provide a homogeneous recombinant protein.

Anti-KLRB1 antibodies of the present disclosure may be analyzed for their physical/chemical properties and/or biological activities by various assays known in the art. In some embodiments, an anti-KLRB1 antibody is tested for its ability to bind KLRB1 (e.g., human KLRB1 and/or cyno KLRB1). Binding assays include, but are not limited to, SPR (e.g., Biacore), ELISA, and FACS. In some embodiments, an anti-KLRB1 antibody is tested for its ability to induce ADCC, ADCP, and/or CDC, as well as the ability of the antibody to kill KLRB1 target cells (cell depletion). Assays include, but are not limited to, ADCC cell lysis assays, e.g., that use LDH release and detection of formazan salt. In addition, antibodies may be evaluated for solubility, stability, thermostability, viscosity, expression levels, expression quality, and/or purification efficiency.

In some embodiments, purified anti-KLRB1 antibodies are characterized by assays including, but not limited to, N-terminal sequencing, amino acid analysis, high pressure liquid chromatography (HPLC), mass spectrometry, ion exchange chromatography, and papain digestion.

### Antibody Conjugates

The present disclosure also provides conjugates comprising an anti-KLRB1 antibody or antigen-binding fragment thereof described herein. In some embodiments, the antibody or antigen-binding fragment thereof is attached to a second molecule. In some embodiments, the antibody or antigen-binding fragment thereof is conjugated to a cytotoxic agent or moiety. In some embodiments, the antibody or antigen-binding fragment thereof is conjugated to a cytotoxic agent to form an ADC (antibody-drug conjugate). In some embodiments, the cytotoxic agent is a chemotherapeutic agent including, but not limited to, methotrexate, adriamycin/doxorubicin, melphalan, mitomycin C, chlorambucil, duocarmycin, daunorubicin, pyrrolobenzodiazepines (PBDs), or other intercalating agents. In some embodiments, the cytotoxic agent is a microtubule inhibitor including, but not limited to, auristatins, maytansinoids (e.g., DM1 and DM4), and tubulysins. In some embodiments, the cytotoxic agent is an enzymatically active toxin of bacterial, fungal, plant, or animal origin, or fragments thereof, including, but not limited to, diphtheria A chain, non-binding active fragments of diphtheria toxin, exotoxin A chain, ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes. In some embodiments, an antibody is conjugated to one or more small molecule toxins, such as calicheamicins, maytansinoids, trichothenes, and CC1065. A derivative of any one of these toxins may be used as long as the derivative retains the cytotoxic activity of the parent molecule.

Conjugates comprising an anti-KLRB1 antibody or antigen-binding fragment thereof described herein can be made using any suitable method known in the art. In some embodiments, conjugates are made using a variety of bifunctional protein-coupling agents such as N-succinimidyl-3-(2-pyridyidithiol) propionate (SPDP), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis(p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene).

In some embodiments, an anti-KLRB1 antibody or antigen-binding fragment thereof described herein is conjugated to a detectable substance or molecule that allows the antibody to be used for diagnosis and/or detection. A detectable substance can include but is not limited to, enzymes, such as horseradish peroxidase, alkaline phosphatase, beta-galactosidase, and acetylcholinesterase; prosthetic groups, such as biotin and flavine(s); fluorescent materials, such as, umbelliferone, fluorescein, fluorescein isothiocyanate (FITC), rhodamine, tetramethylrhodamine isothiocyanate (TRITC), dichlorotriazinylamine fluorescein, dansyl chloride, cyanine (Cy3), and phycoerythrin; bioluminescent materials, such as luciferase; radioactive materials, such as ²¹²Bi, ¹⁴C, ⁵⁷Co, ⁵¹Cr, ⁶⁷Cu, ¹⁸F, ⁶⁸Ga, ⁶⁷Ga, ¹⁵³Gd, ¹⁵⁹Gd, ⁶⁸Ge, ³H, ¹⁶⁶Ho, ¹³¹I, ¹²⁵I, ¹²³I, ¹²¹I, ¹¹⁵In, ¹¹³In, ¹¹²In, ¹¹¹In, ¹⁴⁰La, ¹⁷⁷Lu, ⁵⁴Mn, ⁹⁹Mo, ³²P, ¹⁰³Pd, ¹⁴⁹Pm, ¹⁴²Pr, ¹⁸⁶Re, ¹⁸⁸Re, ¹⁰⁵Rh, ⁹⁷Ru, ³⁵S, ⁴⁷Sc, ⁷⁵Se, ¹⁵³Sm, ¹¹³Sn, ¹¹⁷Sn, ⁸⁵Sr, ^{99m}Tc, ²⁰¹Ti, ¹³³Xe, ⁹⁰Y, ⁶⁹Yb, ¹⁷⁵Yb, ⁶⁵Zn; positron emitting metals; and magnetic metal ions.

An anti-KLRB1 antibody or antigen-binding fragment thereof described herein can also be conjugated to a second antibody to form an antibody heteroconjugate.

An anti-KLRB1 antibody or antigen-binding fragment thereof as described herein may be attached to a solid support. Such solid supports include, but are not limited to, glass, cellulose, polyacrylamide, nylon, polystyrene, polyvinyl chloride, or polypropylene. In some embodiments, immobilized anti-KLRB1 antibodies are used in immunoassays. In some embodiments, immobilized anti-KLRB1 antibodies are used in purification of the target antigen.

### Polynucleotides/Methods of Making Binding Agents

Also provided herein are nucleic acids encoding a polypeptide described herein and vectors, preferably expression vectors, containing the nucleic acid encoding a polypeptide described herein. As used herein, the term “vector” refers to a nucleic acid molecule capable of transporting another nucleic acid to which it has been linked and can include a plasmid, cosmid or viral vector. The vector can be capable of autonomous replication or it can integrate into a host DNA. Viral vectors include, e.g., replication defective retroviruses, adenoviruses and adeno-associated viruses.

A vector can include a nucleic acid in a form suitable for expression of the nucleic acid in a host cell. Preferably the recombinant expression vector includes one or more regulatory sequences operatively linked to the nucleic acid sequence to be expressed. The term “regulatory sequence” includes promoters, enhancers and other expression control elements (e.g., polyadenylation signals). Regulatory sequences include those which direct constitutive expression of a nucleotide sequence, as well as tissue-specific regulatory and/or inducible sequences. The design of the expression vector can depend on such factors as the choice of the host cell to be transformed, the level of expression of protein desired, and the like. The expression vectors of the invention can be introduced into host cells to thereby produce proteins or polypeptides, including fusion proteins or polypeptides, encoded by nucleic acids as described herein that encode a KLRB1 binding agent as described herein.

The recombinant expression vectors of the invention can be designed for expression of KLRB1 binding agent proteins in prokaryotic cells. Preferably, the KLRB1 binding agents can be expressed in mammalian cells, preferably human cells. See, e.g., Frenzel et al., Front Immunol. 2013; 4:217. When used in mammalian cells, the expression vector's control functions are often provided by viral regulatory elements. For example, commonly used promoters are derived from polyoma, Adenovirus 2, cytomegalovirus and Simian Virus 40.

Vector DNA can be introduced into host cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation.

A host cell can be used to produce (i.e., express) a KLRB1 binding agent protein. Accordingly, the invention further provides methods for producing a KLRB1 binding agent protein using the host cells of the invention. In one embodiment, the method includes culturing the host cell of the invention (into which a recombinant expression vector encoding a KLRB1 binding agent protein has been introduced) in a suitable medium such that a KLRB1 binding agent protein is produced. In another embodiment, the method further includes isolating a KLRB1 binding agent protein from the medium or the host cell. In some embodiments, the Fc region is afucosylated (see, e.g., Yamane-Ohnuki and Satoh, MAbs. 2009 May-Jun; 1 (3): 230-236, which describes methods for production of therapeutic antibodies with controlled levels of fucosylation of Fc region N-glycans).

### Pharmaceutical Compositions

Also provided herein are pharmaceutical compositions comprising a KLRB1-binding agent as described herein as an active ingredient. In various embodiments, the KLRB1 binding agent is prepared as a pharmaceutical composition, for example as a pharmaceutical composition for use as a medicament. In various embodiments, the pharmaceutical composition is for use as a medicament for treating a disease as described herein, optionally an autoimmune disease, an allergic disease, a transplant rejection, or a hematologic malignancy, in a subject in need thereof. In some embodiments, the autoimmune disease is rheumatoid arthritis, Sjögren's syndrome, inclusion body myositis (IBM), discoid lupus, psoriasis, idiopathic pulmonary fibrosis, diabetes, alopecia universalis, primary biliary cholangitis, multiple sclerosis, lymphocytic colitis, palmoplantar pustulosis, hidradenitis suppurativa, Crohn's disease, ulcerative colitis, or celiac disease. In some embodiments, the allergic disease is asthma, allergic eosinophilic asthma, allergy, atopic dermatitis, nasal polyposis, eosinophilic gastrointestinal disorder, or hypereosinophilic syndrome. In some embodiments, the transplant rejection can be a rejection of a kidney, lung, heart, liver, limb, skin, or multi-organ transplant. In some embodiments, the hematological malignancy is a leukemia, e.g., T cell leukemia, NK cell leukemia, T cell lymphoma, or large granular lymphocytic leukemia (LGLL). In some embodiments, the lymphoma is hepatosplenic T cell lymphoma (HSTCL), NK/T cell lymphomas (NKTCL), extranodal NK/T cell lymphomas (ENKL), aggressive NK cell leukemia (ANKL), mycosis fungoides, Sezary syndrome, peripheral T cell lymphoma, angioimmunoblastic T cell lymphoma (AITL), and peripheral T cell lymphoma not otherwise specified (PTCL-NOS). In some embodiments, the leukemia is aggressive NK cell leukemia or T cell prolymphocytic leukemia.

One skilled in the art can formulate the KLRB1-binding agent as a pharmaceutical composition according to known methods.

Pharmaceutical compositions can include a carrier. “Carriers” as used herein can include pharmaceutically acceptable carriers, excipients, or stabilizers that are nontoxic (or relatively non-toxic) to the cell or subject being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™.

In various embodiments, the KLRB1 binding agent is comprised in an injectable formulation, for example, a subcutaneous, intravenous, intramuscular, intrathecal or intraperitoneal injection formulation. Injectable formulations can be aqueous solutions, for example in physiologically compatible buffers such as Hanks' solution, Ringer's solution, or physiological saline buffer. The injectable formulation can contain formulatory agents such as suspending, stabilizing and/or dispersing agents. Alternatively, the KLRB1 binding agent can be in a dried or powder form for constitution with a suitable vehicle, e.g., sterile pyrogen-free water, before use.

The binding agents of the present disclosure can be formulated in any suitable form for delivery to a target cell/tissue. In some embodiments, a KLRB1-binding agent can be formulated as a liposome, microparticle, microcapsule, albumin microsphere, microemulsion, nano-particle, nanocapsule, or macroemulsion. In some embodiments, the pharmaceutical formulation includes an agent of the present disclosure complexed with liposomes. Methods to produce liposomes are known to those of skill in the art. For example, some liposomes can be generated by reverse phase evaporation with a lipid composition comprising phosphatidylcholine, cholesterol, and PEG-derivatized phosphatidylethanolamine (PEG-PE).

In some embodiments, a KLRB1-binding agent is formulated as a sustained-release preparation. Suitable examples of sustained-release preparations include semi-permeable matrices of solid hydrophobic polymers containing an agent, where the matrices are in the form of shaped articles (e.g., films or microcapsules). Sustained-release matrices include but are not limited to polyesters, hydrogels such as poly(2-hydroxyethyl-methacrylate) or poly(vinyl alcohol), polylactides, copolymers of L-glutamic acid and 7 ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), sucrose acetate isobutyrate, and poly-D-(−)-3-hydroxybutyric acid.

### Treatment and Administration

The disclosure provides methods comprising administering a KLRB1 binding agent as described herein, or a pharmaceutical composition comprising a KLRB1 binding agent as described herein, to a subject in need thereof. In some embodiments, the subject is a human. In some embodiments, the methods are carried out in vivo (e.g., as opposed to ex vivo). As used herein, “treatment” refers to therapeutic treatment (treating a subject who has a disease); the methods can also be used for prophylactic or preventative measures, wherein the object is to prevent or slow down (lessen) the targeted pathologic condition or disorder, in a subject who does not have the disease. Those in need of treatment can include those already with the disorder, those prone to have the disorder, or those in whom the disorder is to be prevented (as used herein, “prevent” means to reduce the risk of developing).

In various aspects and embodiments, the disclosure provides methods for treating an autoimmune disease, an allergic disease, a transplant rejection, or a hematologic malignancy, in a subject in need thereof. As described herein, KLRB1 expressing cells are implicated in the pathogenesis of these diseases. In accordance with the present disclosure, depletion of such KLRB1 expressing cells provides a therapeutic benefit. In some embodiments, the autoimmune disease is rheumatoid arthritis, Sjögren's syndrome, inclusion body myositis (IBM), discoid lupus, psoriasis, idiopathic pulmonary fibrosis, diabetes, alopecia universalis, primary biliary cholangitis, multiple sclerosis, lymphocytic colitis, palmoplantar pustulosis, hidradenitis suppurativa, Crohn's disease, ulcerative colitis, or celiac disease. In some embodiments, the allergic disease is asthma, allergic eosinophilic asthma, allergy, atopic dermatitis, nasal polyposis, eosinophilic gastrointestinal disorder, or hypereosinophilic syndrome. In some embodiments, the transplant rejection can be a rejection of a kidney, lung, heart, liver, limb, skin, or multi-organ transplant. In some embodiments, the hematological malignancy is a leukemia, e.g., T cell leukemia, NK cell leukemia, T cell lymphoma, T cell prolymphocytic leukemia (T-PLL), or large granular lymphocytic leukemia (LGLL). In some embodiments, the lymphoma is hepatosplenic T cell lymphoma (HSTCL), NK/T cell lymphomas (NKTCL), extranodal NK/T cell lymphomas (ENKL), aggressive NK cell leukemia (ANKL), mycosis fungoides, Sezary syndrome, peripheral T cell lymphoma, angioimmunoblastic T cell lymphoma (AITL), and peripheral T cell lymphoma not otherwise specified (PTCL-NOS). In some embodiments, the leukemia is aggressive NK cell leukemia or T cell prolymphocytic leukemia.

In various aspects and embodiments, the disclosure provides methods for treating or preventing transplant rejection. The transplant rejection can be, for example, kidney rejection. “Administration” and “treatment,” as it applies to an animal, human, experimental subject, cell, tissue, organ, or biological fluid, can include contacting an exogenous pharmaceutical, therapeutic agent, diagnostic agent, or composition to the animal, human, subject, cell, tissue, organ, or biological fluid. “Administration” and “treatment” include in vivo, as well as in some embodiments in vitro or ex vivo treatments.

Typically, the agent is administered in an amount effective to alleviate one or more disease symptoms in the treated subject or population, whether by inducing the regression of or inhibiting the progression of such symptom(s) by any clinically measurable degree. The amount of a therapeutic agent that is effective to alleviate any particular disease symptom can vary according to factors such as the disease state, age, and weight of the patient, and the ability of the drug to elicit a desired response in the subject. Whether a disease symptom has been alleviated can be assessed by any clinical measurement typically used by physicians or other skilled healthcare providers to assess the severity or progression status of that symptom.

As such, in various embodiments, the term “effective amount” or therapeutically effective amount” is a concentration or amount of the KLRB1 binding agent that results in achieving a particular stated purpose, e.g., reduction in one or more symptoms of a disease described herein. An “effective amount” of a KLRB1 binding agent can be determined empirically. Furthermore, a “therapeutically effective amount” is a concentration or amount of a KLRB1 binding agent which is effective for achieving a stated therapeutic effect. This amount can also be determined empirically.

In some embodiments, treatment with the KLRB1-binding agents of the disclosure can kill at least about 20%, e.g., at least about 30%, 40%, 50%, 60%, 70%, or 80% of the KLRB1 expressing cells that are implicated in the pathogenesis of the diseases disclosed herein.

The term “subject” refers to any animal (e.g., a mammal), including, but not limited to, humans and non-human veterinary subjects including non-human primates.

As used herein, reference to “about” or “approximately” a value or parameter includes (and describes) embodiments that are directed to that value or parameter. For example, a description referring to “about X” includes description of “X”. “About” as used herein means plus or minus 10 percent.

As used in the present disclosure and claims, the singular forms “a”, “an” and “the” include plural forms unless the context clearly dictates otherwise.

It is understood that wherever embodiments are described herein with the term “comprising” otherwise analogous embodiments described in terms of “consisting of” and/or “consisting essentially of” are also provided. It is also understood that wherever embodiments are described herein with the phrase “consisting essentially of” otherwise analogous embodiments described in terms of “consisting of” are also provided.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

In various embodiments, the KLRB1 binding agent can be administered by providing an mRNA encoding the binding agent to the subject.

The following examples are illustrative and not restrictive. Many variations of the technology will become apparent to those of skill in the art upon review of this disclosure. The scope of the technology should, therefore, be determined not with reference to the examples, but instead should be determined with reference to the appended claims along with their full scope of equivalents.

### EXAMPLES

### Example 1: Gene Expression of KLRB1 Marks a Unique Collection of Immune Cells, and is Limited to Immune Cells

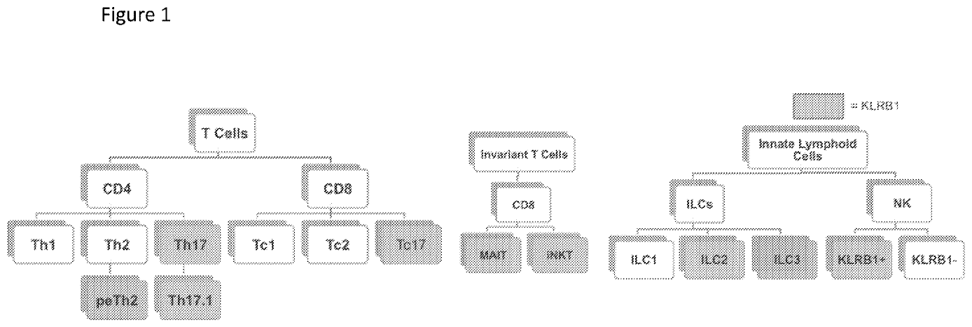

KLRB1 expression cuts across traditional lymphocyte classification and marks Th17, Th17.1, ex-Th17, Tc17, iNKTs, ILC2, ILC3, peTh2, and a subset of NK cells (FIG. 1).

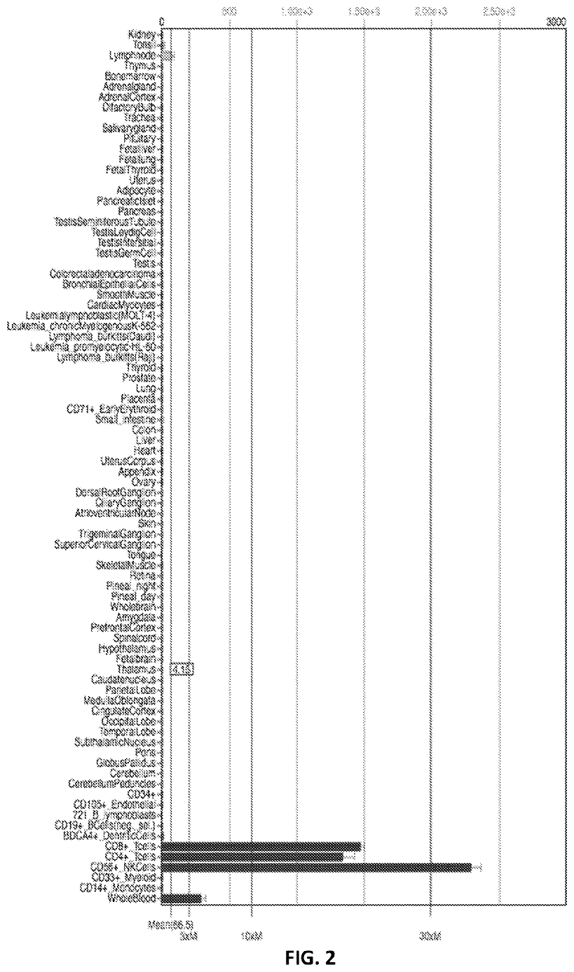

Body-wide KLRB1 expression profiling data indicates KLRB1 has no significant expression on any cell type other than immune cells (FIG. 2).

### Example 2: KLRB1 Expression is Enhanced in Various Immune Conditions

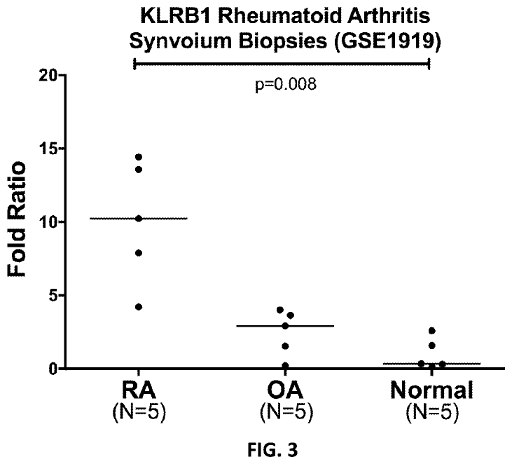

Rheumatoid arthritis: Analysis of expression data (GSE1919) from synovium biopsies from patients with rheumatoid arthritis compared to normal patients shows increased expression of KLRB1 (10 fold ratio) (FIG. 3). Accordingly, rheumatoid arthritis is a particularly attractive target for therapies according to the present disclosure.

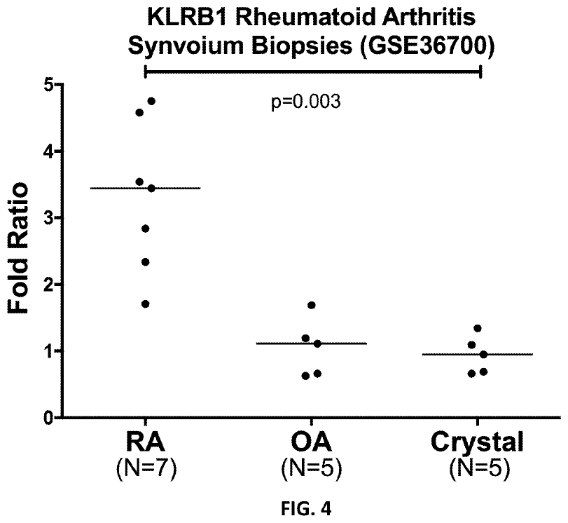

Analysis of expression data (GSE36700) from synovium biopsies from patients with rheumatoid arthritis compared to crystal-induced arthritis patients shows increased expression of KLRB1 (3.3 fold ratio) (FIG. 4). Accordingly, rheumatoid arthritis is a particularly attractive target for therapies according to the present disclosure.

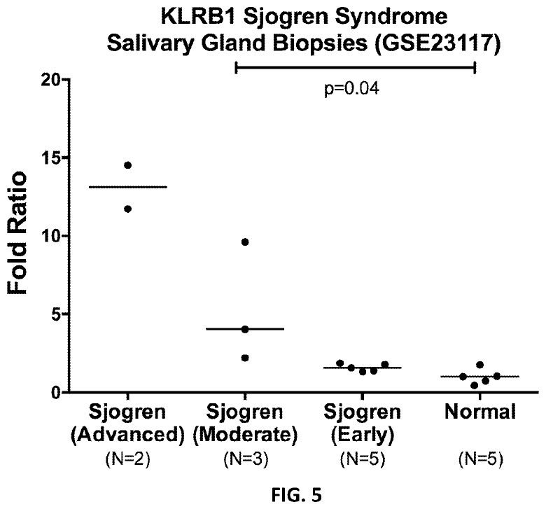

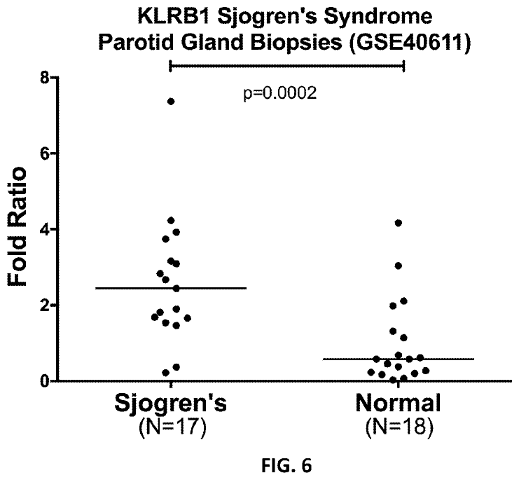

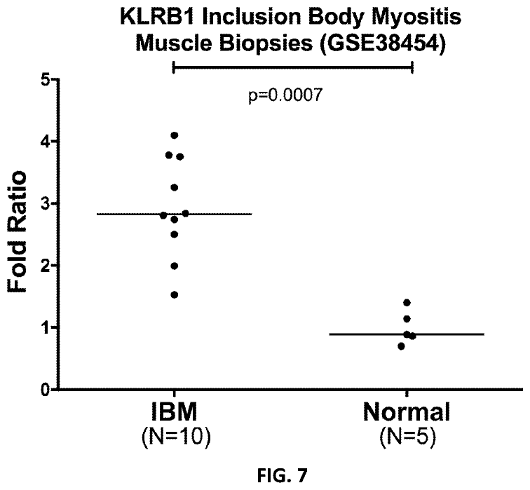

Sjögren's syndrome: Analysis of expression data (GSE23117) from salivary gland biopsies from patients with Sjogren's syndrome compared to normal shows increased expression of KLRB1 in advanced stage (13.1 fold ratio), moderate stage (5.3 fold ratio), and early stage (1.6 fold ratio) (FIG. 5). Accordingly, Sjogren's syndrome is a particularly attractive target for therapies according to the present disclosure.

Analysis of expression data (GSE40611) from parotid gland biopsies from patients with Sjogren's syndrome compared to normal shows increased expression of KLRB1 (2.6 fold ratio) (FIG. 6). Accordingly, Sjogren's syndrome is a particularly attractive target for therapies according to the present disclosure.

Inclusion body myositis: Analysis of expression data (GSE38454) from muscle biopsies from patients with inclusion body myositis compared to normal shows increased expression of KLRB1 (2.9 fold ratio) (FIG. 7). Accordingly, inclusion body myositis is a particularly attractive target for therapies according to the present disclosure.

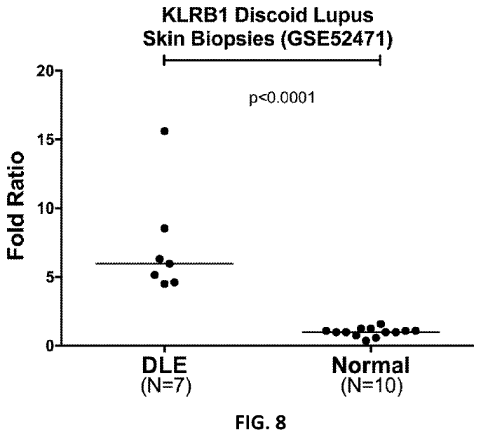

Discoid lupus: Analysis of expression data (GSE52471) from skin biopsies from patients with discoid lupus compared to normal shows increased expression of KLRB1 (7.2 fold ratio) (FIG. 8). Accordingly, discoid lupus is a particularly attractive target for therapies according to the present disclosure.

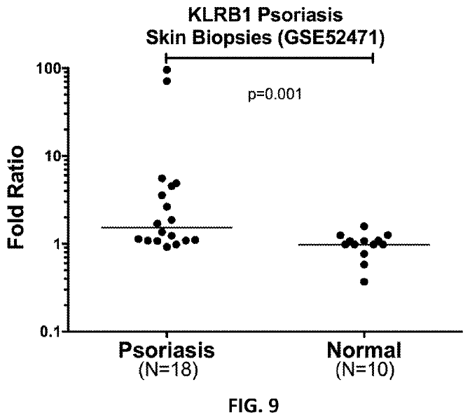

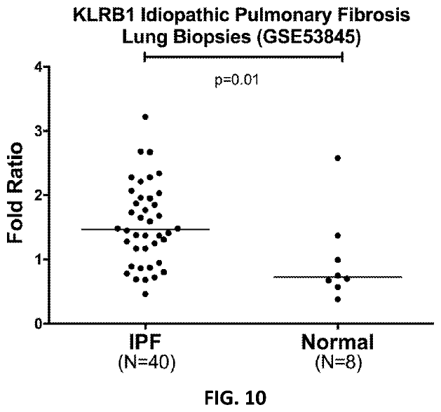

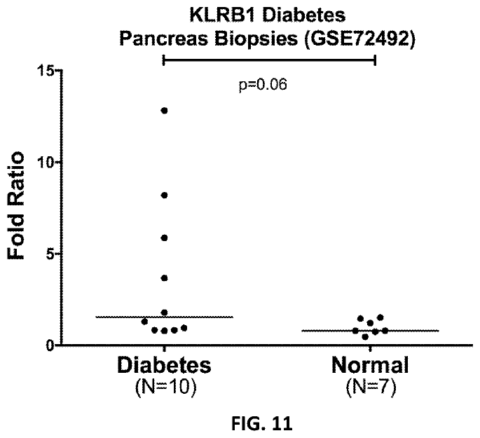

Psoriasis: Analysis of expression data (GSE52471) from skin biopsies from patients with psoriasis compared to normal shows increased expression of KLRB1 (11.2 fold ratio) (FIG. 9). Accordingly, discoid lupus is a particularly attractive target for therapies according to the present disclosure.

Idiopathic pulmonary fibrosis: Analysis of expression data (GSE53845) from lung biopsies from patients with idiopathic pulmonary fibrosis compared to normal shows increased expression of KLRB1 (1.5 fold ratio) (FIG. 10). Accordingly, idiopathic pulmonary fibrosis is a particularly attractive target for therapies according to the present disclosure.

Diabetes: Analysis of expression data (GSE72492) from pancreas biopsies from patients with diabetes compared to normal shows increased expression of KLRB1 (3.7 fold ratio) (FIG. 11). Accordingly, diabetes is a particularly attractive target for therapies according to the present disclosure.

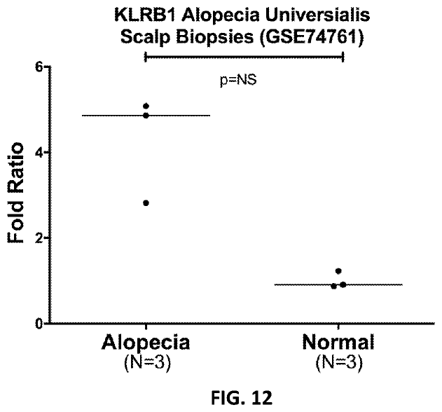

Alopecia universalis: Analysis of expression data (GSE74761) from scalp biopsies from patients with idiopathic pulmonary fibrosis compared to normal shows increased expression of KLRB1 (4.3 fold ratio) (FIG. 12). Accordingly, alopecia universalis is a particularly attractive target for therapies according to the present disclosure.

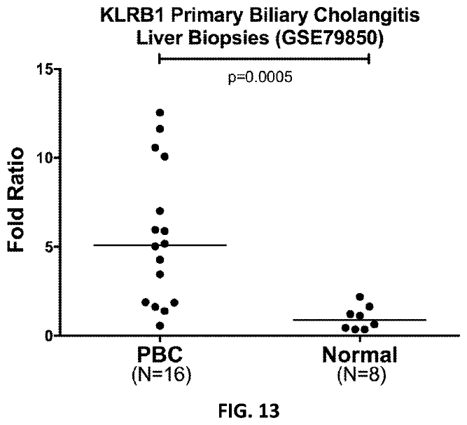

Primary biliary cholangitis: Analysis of expression data (GSE79850) from liver biopsies from patients with primary biliary cholangitis eventually requiring liver transplantation compared to normal shows increased expression of KLRB1 (5.6 fold ratio) (FIG. 13). Accordingly, primary biliary cholangitis is a particularly attractive target for therapies according to the present disclosure.

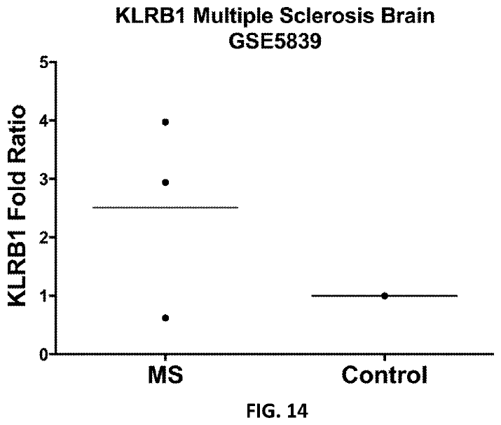

Multiple Sclerosis: Analysis of expression data (GSE5839) from brain biopsies from patients with multiple sclerosis show elevated expression of KLRB1 compared to control brain (2.5-fold) (FIG. 14). Accordingly, multiple sclerosis is a particularly attractive target for therapies according to the present disclosure.

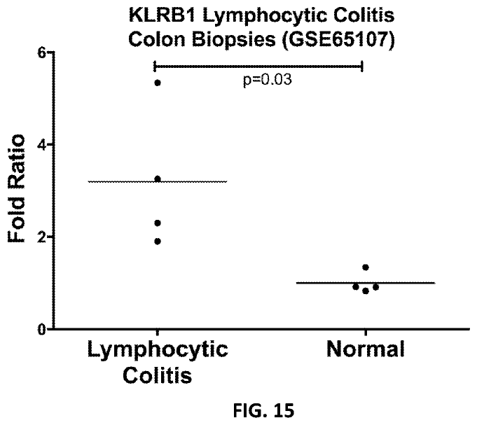

Lymphocytic colitis: Analysis of expression data (GSE65107) from colon biopsies from 4 patients with lymphocytic colitis compared to 4 healthy persons shows increased expression of KLRB1 (3.2 fold ratio) (FIG. 15). Accordingly, lymphocytic colitis is a particularly attractive target for therapies according to the present disclosure.

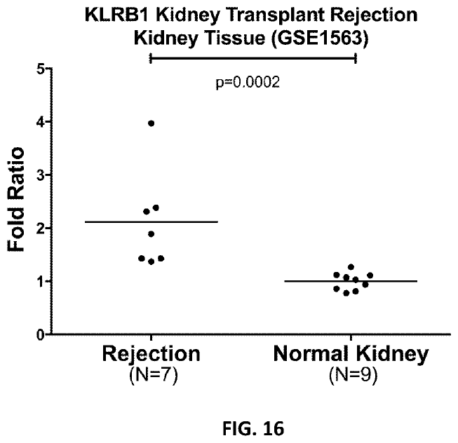

Kidney transplant rejection: Analysis of expression data (GSE1563) from kidney biopsies from 7 patients with acute kidney rejection compared to 9 healthy persons shows increased expression of KLRB1 (2.1 fold ratio) (FIG. 16). Accordingly, kidney transplant rejection is a particularly attractive target for therapies according to the present disclosure.

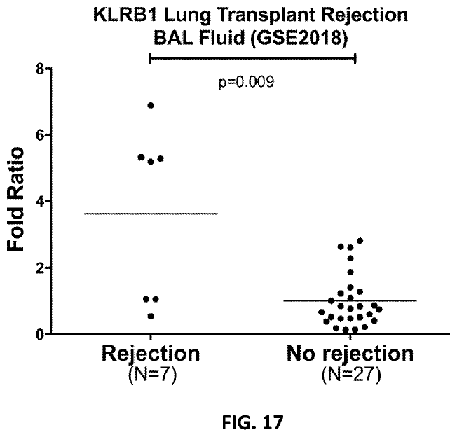

Lung transplant: Analysis of expression data (GSE65107) from lung broncheoalveolar lavage (BAL) fluid from 7 patients with lung transplant rejection compared to 27 patients with lung transplants not undergoing rejection shows increased expression of KLRB1 (3.6 fold ratio) (FIG. 17). Accordingly, lung transplant rejection is a particularly attractive target for therapies according to the present disclosure.

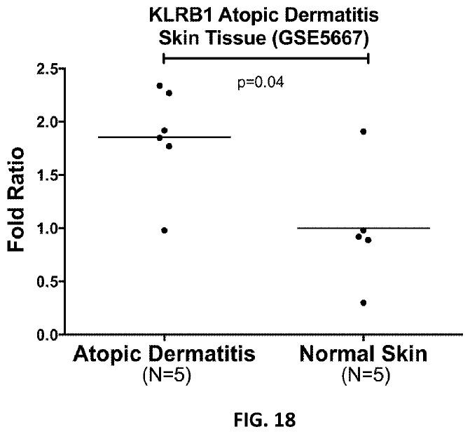

Atopic dermatitis: Analysis of expression data (GSE65107) from skin biopsies from 5 patients with atopic dermatitis compared to 5 healthy persons shows increased expression of KLRB1 (1.9 fold ratio) (FIG. 18). Accordingly, atopic dermatitis is a particularly attractive target for therapies according to the present disclosure.

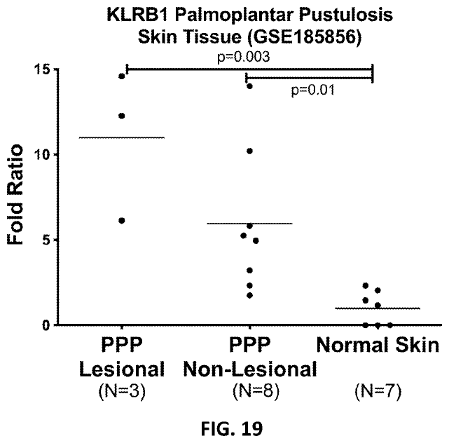

Palmoplantar pustulosis: Analysis of expression data (GSE185856) from skin biopsies from 3 patients with palmoplantar pustulosis lesional skin and 8 patients palmoplantar pustulosis non-lesional skin compared to 7 healthy persons shows increased expression of KLRB1 (11.0 fold ratio lesional to healthy; 5.9 fold ratio non-lesional to healthy) (FIG. 19). Accordingly, palmoplantar pustulosis is a particularly attractive target for therapies according to the present disclosure.

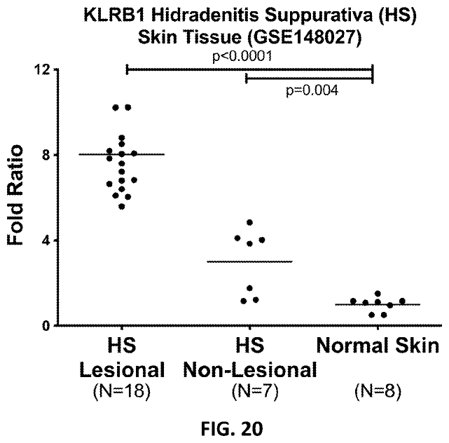

Hidradenitis suppurativa: Analysis of expression data (GSE148027) from skin biopsies from 18 patients with hidradenitis suppurativa lesional skin and 7 patients hidradenitis suppurativa non-lesional skin compared to 8 healthy persons shows increased expression of KLRB1 (8.0 fold ratio lesional to healthy; 3.0 fold ratio non-lesional to healthy) (FIG. 20). Accordingly, hidradenitis suppurativa is a particularly attractive target for therapies according to the present disclosure.

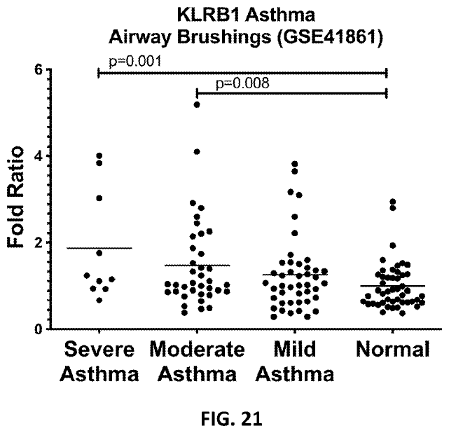

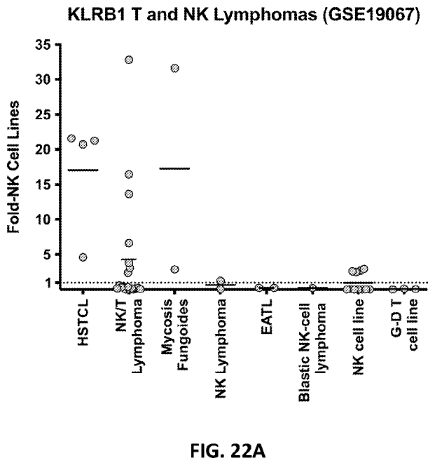

Asthma: Analysis of expression data (GSE41861) from lung airway brushings from 10 patients with severe asthma, 37 patients with moderate asthma, and 44 patients with mild asthma compared to 47 healthy persons shows increased expression of KLRB1 (1.9 fold ratio severe asthma to healthy; 1.5 fold ratio moderate asthma to healthy; and 1.3 fold ratio mild asthma to healthy) (FIG. 21). Accordingly, atopic dermatitis is a particularly attractive target for therapies according to the present disclosure.

### Example 3: KLRB1 Expression is Enhanced in Various T and NK Cell Lymphomas and Leukemias

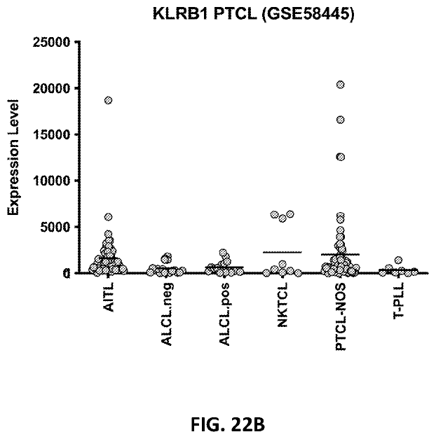

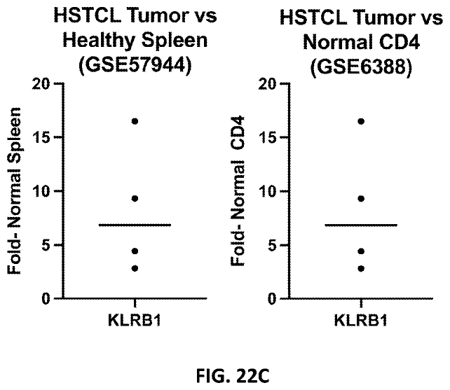

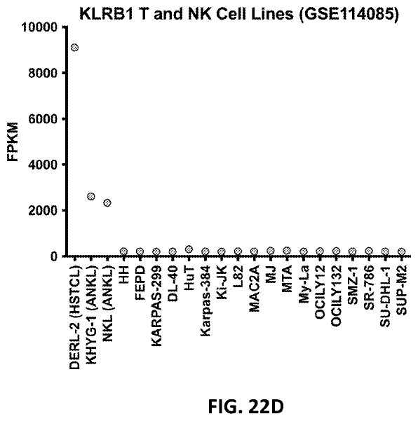

Analysis of expression data (GSE19067) from tumor cells from patients with various T and NK cell lymphomas and leukemias compared to normal NK cell expression shows increased expression of KLRB1 (e.g., hepatosplenic T cell lymphoma has 17 fold ratio compared to normal NK cell line) (FIG. 22). Accordingly, various T and NK cell lymphomas are particularly attractive targets for therapies according to the present disclosure.

In particular, some patients with hepatosplenic T cell lymphoma (HSTCL), NK/T cell lymphomas (NKTCL), aggressive NK cell leukemia (ANKL), mycosis fungoides, Sezary syndrome, peripheral T cell lymphoma not otherwise specified (PTCL-NOS), T cell prolymphocytic leukemia (T-PLL), and peripheral T cell lymphoma (PTCL) have increased expression of KLRB1 or similar expression of KLRB1 to CD4+ T cells (FIG. 22). Accordingly, hepatosplenic T cell lymphoma (HSTCL), NK/T cell lymphomas (NKTCL), aggressive NK cell leukemia (ANKL), mycosis fungoides, Sezary syndrome, peripheral T cell lymphoma not otherwise specified (PTCL-NOS), T cell prolymphocytic leukemia (T-PLL), and peripheral T cell lymphoma (PTCL) are particularly attractive targets for therapies according to the present disclosure.

### Example 4: Monoclonal Murine Anti-KLRB1 Antibodies 9C10G11, 57E2E4, 75B10F12, 7B6C7, 52G9B12B5, 5B1B11, 50A8F2, 47A5H2, 66G9C11, 33F11A6, 37G6A7, 39C5D6, 62H1D6, 92E12F2, 13H2D11, 5A11D10, 18G11F11, and 22F10G3 and Various Derived Chimeric Antibodies Bind to Human KLRB1

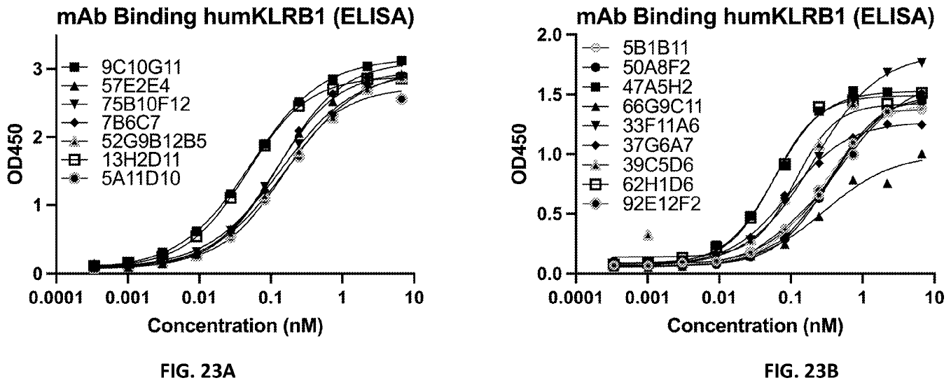

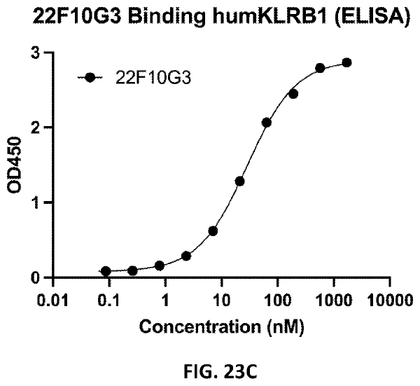

Monoclonal antibodies were generated against human KLRB1 by immunizing mice (5 balb/c and 5 SJL) with human KLRB1 protein extracellular domain (ECD) (Uniprot ID Q12918, amino acids 67-225) to create mouse hybridomas. Parental hybridoma clones were subsequently screened for binding by ELISA against human KLRB1 ECD and by FACS against stable CHO-K1 cell lines expressing human KLRB1 (SEQ ID NO:1) and separately expressing cynomolgus KLRB1 (SEQ ID NO:2). Twenty hybridomas were selected for subcloning based on binding to both human and cynomolgus CHO-K1 expressing KLRB1 cells, resulting in 91 subclones. These 91 subclones underwent repeat FACS binding studies to CHO-K1 human-KLRB1 and CHO-K1 cynomolgus KLRB1. A number of these mAbs, designated 9C10G11, 57E2E4, 75B10F12, 7B6C7, 52G9B12B5, 13H2D11, 5A11D10, 5B1B11, 50A8F2, 47A5H2, 66G9C11, 33F11A6, 37G6A7, 39C5D6, 62H1D6, 92E12F2, and 22F10G3 were tested for binding ability in an ELISA assay. Human KLRB1 ECD was coated on ELISA plates and the plates were incubated with these antibodies. Secondary goat anti-mouse peroxidase labelled IgG was used to detect antibodies bound to the ELISA plate (see FIGS. 23A-C).

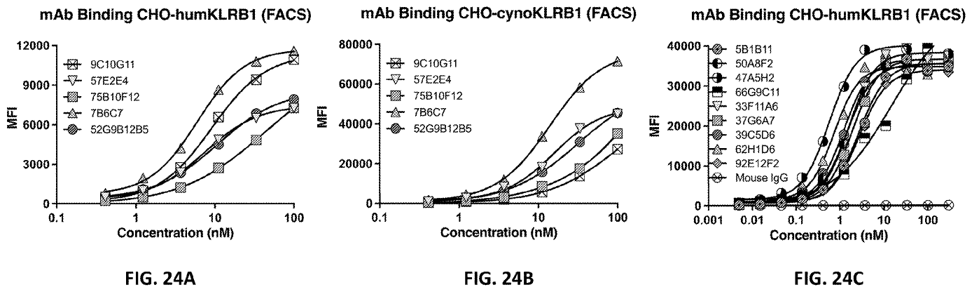

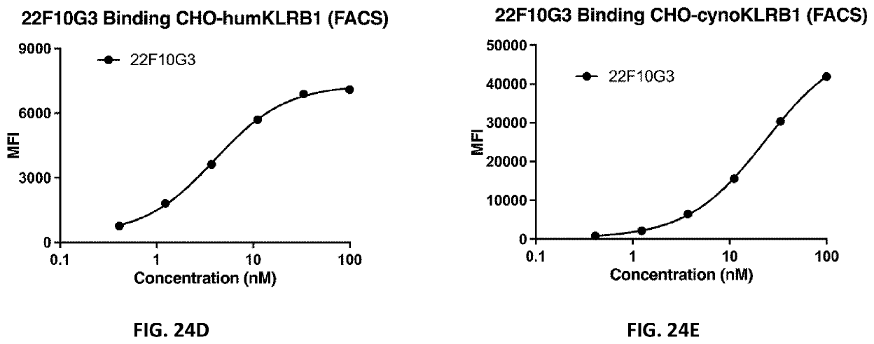

Antibodies 9C10G11, 57E2E4, 75B10F12, 7B6C7, 52G9B12B5, 5B1B11, 50A8F2, 47A5H2, 66G9C11, 33F11A6, 37G6A7, 39C5D6, 62H1D6, 92E12F2, and 22F10G3 were also tested in dose-dependent FACS binding assays against CHO-K1 cells expressing human KLRB1 (CHO-hum-KLRB1). Secondary goat anti-mouse peroxidase labelled IgG was used to detect antibodies bound to CHO-hum-KLRB1 (see FIG. 24A, FIG. 24C, and FIG. 24D).

Antibodies 9C10G11, 57E2E4, 75B10F12, 7B6C7, 52G9B12B5, and 22F10G3 were also tested in dose-dependent FACS binding assays against CHO-K1 cells expressing cynomolgus KLRB1 (CHO-cyno-KLRB1). Secondary goat anti-mouse peroxidase labelled IgG was used to detect antibodies bound to CHO-cyno-KLRB1 (see FIG. 24B and FIG. 24E).

Antibodies 9C10G11, 57E2E4, 75B10F12, 7B6C7, 52G9B12B5, 5B1B11, 50A8F2, 47A5H2, 66G9C11, 33F11A6, 37G6A7, 39C5D6, 62H1D6, 92E12F2, 13H2D11, 5A11D10, 18G11F11, and 22F10G3 were also tested in single-concentration FACS binding assays against both CHO-hum-KLRB1 and CHO-cyno-KLRB1, with untransfected CHO-K1 cells as control. Secondary goat anti-mouse peroxidase labelled IgG was used to detect antibodies bound to CHO-hum-KLRB1 or CHO-cyno-KLRB1. All antibodies bound to CHO-hum-KLRB1, and all except for 50A8F2 bound to CHO-cyno-KLRB1. The results are shown in Table 19.

Various murine and mouse/human chimeric antibodies with human IgG1-kappa were assayed for binding against human KLRB1 extracellular domain (ECD) via surface plasma resonance (SPR). The results are shown in Tables 20 and 21A-B.

TABLE 19
Single concentration FACS binding for murine antibodies

| | CHO- | CHO-K1- | CHO-K1- |
|---|---|---|---|
| | K1 | humKLRB1 | cynoKLRB1 |
| Clone | MFI | MFI | MFI |
| 9C10G11 | 823 | 110415 | 255874 |
| 57E2E4 | 824 | 107889 | 416000 |
| 75B10F12 | 719 | 78369 | 183829 |
| 7B6C7 | 851 | 53698 | 364738 |
| 52G9B12B5 | 125 | 16435 | 50090 |
| 5B1B11 | 695 | 98591 | 410000 |
| 50A8F2 | 631 | 117066 | 820 |
| 47A5H2 | 782 | 128296 | 366429 |
| 66G9C11 | 668 | 123959 | 348930 |
| 33F11A6 | 690 | 115928 | 427000 |
| 37G6A7 | 820 | 102625 | 257329 |
| 39C5D6 | 868 | 112330 | 447000 |
| 62H1D6 | 979 | 88456 | 303625 |
| 92E12F2 | 825 | 87494 | 437000 |
| 13H2D11 | 726 | 171616 | 217381 |
| 5A11D10 | 688 | 160716 | 434000 |
| 18G11F11 | 857 | 178673 | 498000 |
| 22F10G3 | 749 | 132551 | 442000 |

TABLE 20
Binding kinetics by SPR for murine and mouse/human
chimeric antibodies

| | | | | Rmax |
|---|---|---|---|---|
| Antibody | ka (1/Ms) | kd(1/s) | KD (M) | (RU) |
| Hybridoma 9C10G11 | 3.12E+05 | 4.63E−03 | 1.48E−08 | 28.4 |
| Chimeric 57E2E4 | 1.23E+05 | 7.10E−05 | 5.75E−10 | 49.2 |
| Chimeric 7B6C7 | 1.14E+06 | 1.68E−05 | 1.47E−11 | 52 |
| Chimeric 52G9B12B5 | 2.77E+05 | 1.22E−03 | 4.40E−09 | 19.3 |
| Chimeric 5B1B11 | 7.77E+05 | 1.98E−04 | 2.55E−10 | 46.5 |
| Chimeric 50A8F2 | 1.85E+06 | 9.64E−05 | 5.21E−11 | 47.5 |
| Chimeric 47A5H2 | 1.90E+06 | 5.81E−04 | 3.06E−10 | 58.6 |
| Hybridoma 66G9C11 | 1.16E+05 | 7.41E−04 | 6.38E−09 | 24.3 |
| Hybridoma 33F11A6 | NA | NA | NA | NA |
| Hybridoma 37G6A7 | 3.58E+05 | 8.33E−03 | 2.33E−08 | 30.9 |
| Hybridoma 39C5D6 | 5.19E+05 | 6.28E−04 | 1.21E−09 | 39.1 |
| Hybridoma 62H1D6 | 1.16E+06 | 8.12E−03 | 7.03E−09 | 39.3 |
| Hybridoma 92E12F2 | 2.67E+05 | 8.88E−04 | 3.33E−09 | 20.2 |
| Hybridoma 13H2D11 | 8.06E+05 | 1.38E−02 | 1.71E−08 | 29.4 |
| Chimeric 5A11D10 | 4.18E+06 | 2.47E−04 | 5.91E−11 | 66.5 |

TABLE 21A
Binding kinetics by SPR for 22F10G3 and 22F10G3
humanized variants

| | | | | Rmax | Chi² |
|---|---|---|---|---|---|
| Antibody | ka (1/Ms) | kd(1/s) | KD (M) | (RU) | (RU²) |
| Chimeric | 5.14E+06 | 2.21E−04 | 4.29E−11 | 39.2 | |
| 22F10G3 | | | | | |
| Hu22F10G3 | 5.92E+06 | 3.44E−04 | 5.82E−11 | 59 | 9.24E−02 |
| Hu22F10G3-1.1 | 4.74E+06 | 6.33E−04 | 1.34E−10 | 54.3 | 1.08E−01 |
| Hu22F10G3-2.3 | 5.09E+06 | 5.23E−04 | 1.03E−10 | 62.3 | 1.58E−01 |
| Hu22F10G3-3.4 | 4.81E+06 | 6.23E−04 | 1.29E−10 | 50.3 | 1.68E−01 |

TABLE 21B
Binding kinetics by SPR for 22F10G3 and 22F10G3-2.3
humanized variants

| | | | | Rmax | Chi² |
|---|---|---|---|---|---|
| Antibody | ka (1/Ms) | kd (1/s) | KD (M) | (RU) | (RU²) |
| Hu22F10G3-2.3 | 9.60E+06 | 6.60E−04 | 6.88E−11 | 48.2 | 3.56E−02 |
| Hu22F10G3- | 8.83E+06 | 1.24E−03 | 1.40E−10 | 42.6 | 2.48E−02 |
| 2.3-NA | | | | | |
| Hu22F10G3- | 7.79E+06 | 5.08E−03 | 6.53E−10 | 36.3 | 2.16E−02 |
| 2.3-QS | | | | | |

### Example 5: Monoclonal Anti-KLRB1 Antibodies can Elicit Antibody-Dependent Cell-Mediated Cytotoxicity

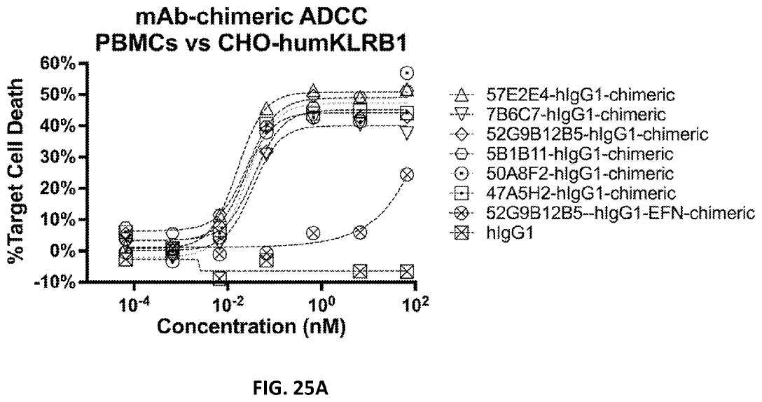

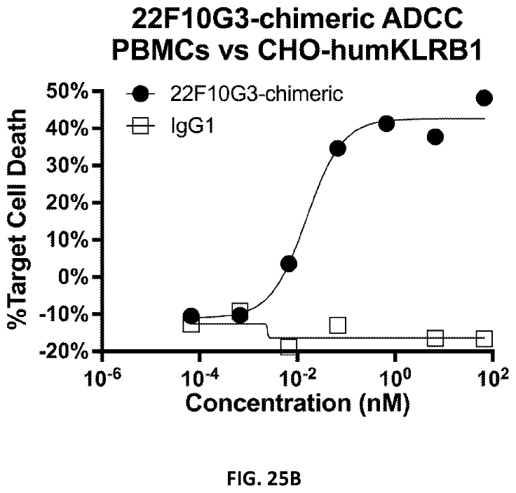

To determine whether the anti-KLRB1 antibodies will be effective in killing KLRB1 positive cells, the ability of anti-KLRB1 antibodies to elicit antibody-dependent cell-mediated cytotoxicity (ADCC) was tested. ADCC elicited by was evaluated using a cell lysis assay involving LDH release and detection of formazan salt (Genscript, SC1544). CHO-hum-KLRB1 were incubated with human peripheral blood mononuclear cells (PBMCs) and various antibodies. Antibodies tested were mouse/human IgG1-kappa (wild-type) chimeric antibodies 57E2E4-Chimeric, 7B6C7-Chimeric, 52G9B12B5-Chimeric, 5B1B11-Chimeric, 50A8F2-Chimeric, 47A5H2-Chimeric; 52G9B12B5-Chimeric-EN (a mouse/human chimeric KLRB1-binding antibody with 52G9B12B5 variable regions and human IgG1-kappa mutant L234A/L235A/G237A mutation; used as a negative control); and human IgG1 (negative control) (FIG. 25A), or 22F10G3-Chimeric and human IgG1 (negative control) (FIG. 25B).

### Example 6: Monoclonal Anti-KLRB1 Antibodies can Block Binding of CLEC2D to KLRB1

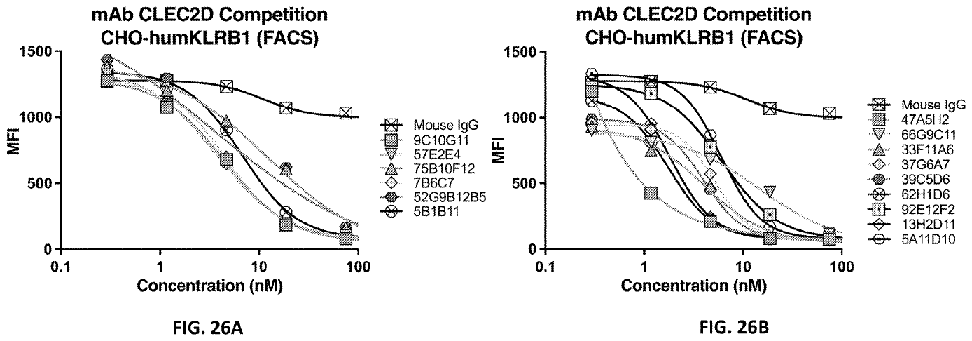

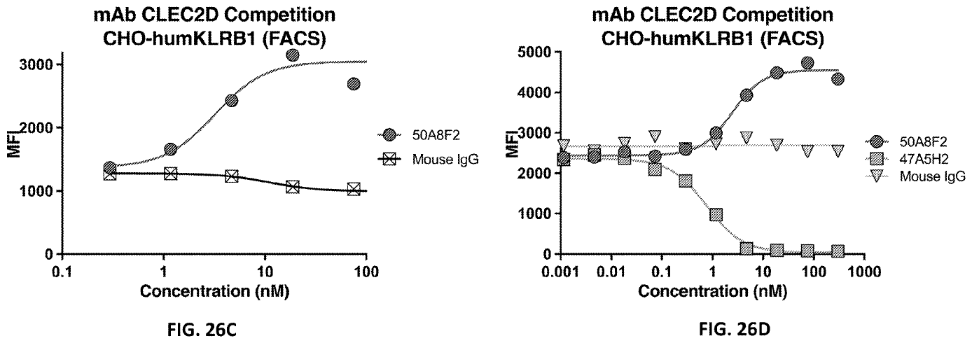

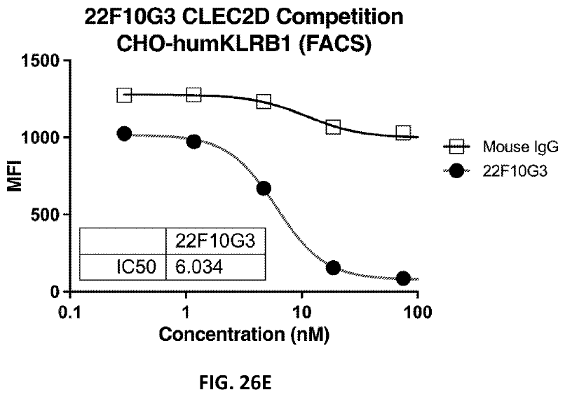

CLEC2D is the natural ligand for KLRB1. A fusion protein of CLEC2D-Fc-biotin was produced through transfection of CHO cells with an expression construct. The ability of monoclonal antibodies 9C10G11, 57E2E4, 75B10F12, 7B6C7, 52G9B12B5, 5B1B11, 50A8F2, 47A5H2, 66G9C11, 33F11A6, 37G6A7, 39C5D6, 62H1D6, 92E12F2, 13H2D11, 5A11D10, and 22F10G3 to compete with CLEC2D-Fc-biotin against CHO-K1 cells expressing human KLRB1 (CHO-hum-KLRB1) was tested using streptavidin-AF488 to detect presence or absence of CLEC2D bound to these cells. All antibodies except 50A8F2 showed ability to block CLEC2D binding (FIG. 26A, FIG. 26B, and FIG. 26E).

### Example 7: Monoclonal Anti-KLRB1 Antibody 50A8F2 Enhances CLEC2D Binding to KLRB1

CLEC2D is the natural ligand for KLRB1. A fusion protein of CLEC2D-Fc-biotin was produced through transfection of CHO cells with an expression construct. Monoclonal antibody 50A8F2 ability to compete with CLEC2D-Fc-biotin against CHO-K1 cells expressing human KLRB1 (CHO-hum-KLRB1) was tested using streptavidin-AF488 to detect presence or absence of CLEC2D bound to these cells. 50A8F2 enhanced CLEC2D binding in two independent experiments (FIG. 26C and FIG. 26D).

### Example 8. Humanization of 47A5H2

Humanized sequence variants of 47A5H2 were produced for both heavy and light chains by CDR grafting (Almagro et al. 2008) of the murine antibody 47A5H2 onto human frameworks IGHV1-46*01/JH1, IGKV4-1*01/JK2, and IGKV3-11*01/JK2. These are shown in Table 22.

The sequence variants were combined and expressed as human IgG1 kappa antibodies with IgG1 L234A/L235A/G237A mutations, to produce 16 antibodies as shown in Table 23.

TABLE 22
47A5H2 humanized sequence variants for individual heavy and light chains

| Antibody Variable | |
|---|---|
| Chain | Sequence |
| 47A5H2-VH_g0-VH | QVQLVQSGAEVKKPGASVKVSCKASGYSFTGYWMHWVRQAPGQGLEWMGMIHPSDS |
| | ETRLNQKFKDRVTMTRDTSTSTVYMELSSLRSEDTAVYYCASEGLRRGSFAYWGQGTL |
| | VTVSS (SEQ ID NO: 347) |
| 47A5H2-VH_g1-VH | QVQLVQSGAEVKKPGASVKVSCKASGYSFTGYWMHWVRQAPGQGLEWMGMIHPSDS |
| | ETRLNQKFKDRVTMTVDTSTSTVYMELSSLRSEDTAVYYCASEGLRRGSFAYWGQGTL |
| | VTVSS (SEQ ID NO: 348) |
| 47A5H2-VH_g2-VH | QVQLVQSGAEVKKPGASVKVSCKASGYSFTGYWMHWVRQAPGQGLEWMGMIHPSDS |
| | ETRLNQKFKDRVTMTVDKSTSTVYMELSSLRSEDTAVYYCASEGLRRGSFAYWGQGTL |
| | VTVSS (SEQ ID NO: 349) |
| 47A5H2-VH_g3-VH | QVQLVQSGAEVKKPGASVKVSCKGSGYSFTGYWMHWVRQAPGQGLEWMGMIHPSDS |
| | ETRLNQKFKDKVTMTVDKSTSTVYMELSSLRSEDTAVYYCASEGLRRGSFAYWGQGTL |
| | VTVSS (SEQ ID NO: 350) |
| 47A5H2-Vκ1_g0-VL | DIVMTQSPDSLAVSLGERATINCRASESVDNYGMTFMSWYQQKPGQPPKLLIYAASNQG |
| | SGVPDRFSGSGSGTDFTLTISSLQAEDVAVYYCQQSKEVPYTFGQGTKLEIK |
| | (SEQ ID NO: 351) |
| 47A5H2-Vκ1_g1-VL | DIVLTQSPDSLAVSLGERATINCRASESVDNYGMTFMSWYQQKPGQPPKLLIYAASNQG |
| | SGVPDRFSGSGSGTDFTLTISSLQAEDVAVYYCQQSKEVPYTFGQGTKLEIK |
| | (SEQ ID NO: 352) |
| 47A5H2-Vκ1_g2-VL | DIVLTQSPDSLAVSLGERATINCRASESVDNYGMTFMSWFQQKPGQPPKLLIYAASNQG |
| | SGVPDRFSGSGSGTDFTLTISSLQAEDVAVYYCQQSKEVPYTFGQGTKLEIK |
| | (SEQ ID NO: 353) |
| 47A5H2-Vκ2_g0-VL | EIVLTQSPATLSLSPGERATLSCRASESVDNYGMTFMSWYQQKPGQAPRLLIYAASNQG |
| | SGIPARFSGSGSGTDFTLTISSLEPEDFAVYYCQQSKEVPYTFGQGTKLEIK |
| | (SEQ ID NO: 354) |

TABLE 23
Combinations of 47A5H2 humanized sequence variants individual variable heavy
and light chains produced as human IgG1 kappa antibodies containing the effector null
IgG1 Fc variant L234A/L235A/G237A.

| | | | | | SEC |
|---|---|---|---|---|---|
| | | | Back | Yield | purity |
| Antibody | Heavy Chain | Light Chain | Mutation | (mg) | (%) |
| HU47A5H2-01 | 47A5H2-VH_g0 (SEQ | 47A5H2-Vκ1_g0 (SEQ | 0 | 2.9 | 94.9 |
| | ID NO: 347) | ID NO: 351) | | | |
| HU47A5H2-02 | 47A5H2-VH_g0 (SEQ | 47A5H2-Vκ1_g1 (SEQ | 1 | 2.8 | 94.5 |
| | ID NO: 347) | ID NO: 352) | | | |
| HU47A5H2-03 | 47A5H2-VH_g0 (SEQ | 47A5H2-Vκ1_g2 (SEQ | 2 | 2.9 | 98.4 |
| | ID NO: 347) | ID NO: 353) | | | |
| HU47A5H2-04 | 47A5H2-VH_g0 (SEQ | 47A5H2-Vκ2_g0 (SEQ | 0 | 3.0 | 94.8 |
| | ID NO: 347) | ID NO: 354) | | | |
| HU47A5H2-05 | 47A5H2-VH_g1 (SEQ | 47A5H2-Vκ1_g0 (SEQ | 2 | 6.0 | 96.8 |
| | ID NO: 348) | ID NO: 351) | | | |
| HU47A5H2-06 | 47A5H2-VH_g1 (SEQ | 47A5H2-Vκ1_g1 (SEQ | 3 | 5.6 | 92.7 |
| | ID NO: 348) | ID NO: 352) | | | |
| HU47A5H2-07 | 47A5H2-VH_g1 (SEQ | 47A5H2-Vκ1_g2 (SEQ | 4 | 5.9 | 100.0 |
| | ID NO: 348) | ID NO: 353) | | | |
| HU47A5H2-08 | 47A5H2-VH_g1 (SEQ | 47A5H2-Vκ2_g0 (SEQ | 2 | 5.2 | 92.9 |
| | ID NO: 348) | ID NO: 354) | | | |
| HU47A5H2-09 | 47A5H2-VH_g2 (SEQ | 47A5H2-Vκ1_g0 (SEQ | 3 | 2.8 | 95.8 |
| | ID NO: 349) | ID NO: 351) | | | |
| HU47A5H2-10 | 47A5H2-VH_g2 (SEQ | 47A5H2-Vκ1_g1 (SEQ | 4 | 2.9 | 94.6 |
| | ID NO: 349) | ID NO: 352) | | | |
| HU47A5H2-11 | 47A5H2-VH_g2 (SEQ | 47A5H2-Vκ1_g2 (SEQ | 5 | 2.6 | 100.0 |
| | ID NO: 349) | ID NO: 353) | | | |
| HU47A5H2-12 | 47A5H2-VH_g2 (SEQ | 47A5H2-Vκ2_g0 (SEQ | 3 | 2.4 | 96.4 |
| | ID NO: 349) | ID NO: 354) | | | |
| HU47A5H2-13 | 47A5H2-VH_g3 (SEQ | 47A5H2-Vκ1_g0 (SEQ | 5 | 3.0 | 91.7 |
| | ID NO: 350) | ID NO: 351) | | | |
| HU47A5H2-14 | 47A5H2-VH_g3 (SEQ | 47A5H2-Vκ1_g1 (SEQ | 6 | 2.5 | 90.7 |
| | ID NO: 350) | ID NO: 352) | | | |
| HU47A5H2-15 | 47A5H2-VH_g3 (SEQ | 47A5H2-Vκ1_g2 (SEQ | 7 | 2.5 | 100.0 |
| | ID NO: 350) | ID NO: 353) | | | |
| HU47A5H2-16 | 47A5H2-VH_g3 (SEQ | 47A5H2-Vκ2_g0 (SEQ | 5 | 2.5 | 93.7 |
| | ID NO: 350) | ID NO: 354) | | | |
| HU47A5H2-chimeric | 47A5H2-VH (SEQ ID | 47A5H2-Vκ (SEQ ID | 0 | 1.1 | 95.3 |
| | NO: 192) | NO: 193) | | | |

### Example 9. Humanization of 50A8F2

Humanized sequence variants of 50A8F2 were produced for both heavy and light chains by CDR grafting (Almagro et al. 2008) of the murine antibody 50A8F2 onto human frameworks IGHV1-46*01/JH6 and IGKV1-39*01/JK2. These are shown in Table 24.

These sequence variants were combined and expressed as human IgG1 kappa antibodies with IgG1 L234A/L235A/G237A mutations, to produce 16 antibodies as shown in Table 25.

TABLE 24
50A8F2 humanized sequence variants for individual heavy and light chains

| Antibody Variable | |
|---|---|
| Chain | Sequence |
| 50A8F2-VH_g0-VH | QVQLVQSGAEVKKPGASVKVSCKASGYTFIDYYINWVRQAPGQGLEWMGWIFPGSGHTKY |
| | NENFKGRVTMTRDTSTSTVYMELSSLRSEDTAVYYCARGDRSALGYFDYWGQGTTVTVSS |
| | (SEQ ID NO: 355) |
| 50A8F2-VH_g1-VH | QVQLVQSGAEVKKPGASVKVSCKASGYTFIDYYINWVRQAPGQGLEWMGWIFPGSGHTKY |
| | NENFKGKVTMTVDTSTSTVYMELSSLRSEDTAVYYCARGDRSALGYFDYWGQGTTVTVSS |
| | (SEQ ID NO: 356) |
| 50A8F2-VH_g2-VH | QVQLVQSGAEVKKPGASVKVSCKASGYTFIDYYINWVRQAPGQGLEWMGWIFPGSGHTKY |
| | NENFKGKVTLTVDTSTSTVYMELSSLRSEDTAVYYCARGDRSALGYFDYWGQGTTVTVSS |
| | (SEQ ID NO: 357) |
| 50A8F2-VH_g3-VH | QVQLVQSGAEVKKPGASVKVSCKASGYTFIDYYINWVRQAPGQGLEWIGWIFPGSGHTKY |
| | NENFKGKVTLTVDTSTSTVYMELSSLRSEDTAVYFCARGDRSALGYFDYWGQGTTVTVSS |
| | (SEQ ID NO: 358) |
| 50A8F2-Vκ_g0-VL | DIQMTQSPSSLSASVGDRVTITCTASSSVSSTYLHWYQQKPGKAPKLLIYSTSNLASGVP |
| | SRFSGSGSGTDFTLTISSLQPEDFATYYCHQYHRSPLTFGQGTKLEIK |
| | (SEQ ID NO: 359) |
| 50A8F2-Vκ_g1-VL | DIQMTQSPSSLSASVGDRVTITCTASSSVSSTYLHWYQQKPGKAPKLLIYSTSNLASGVP |
| | SRFSGSGSGTDYTLTISSLQPEDFATYYCHQYHRSPLTFGQGTKLEIK |
| | (SEQ ID NO: 360) |
| 50A8F2-Vκ_g2-VL | DIQLTQSPSSLSASVGDRVTITCTASSSVSSTYLHWYQQKPGKSPKLLIYSTSNLASGVP |
| | SRFSGSGSGTDYTLTISSLQPEDFATYYCHQYHRSPLTFGQGTKLEIK |
| | (SEQ ID NO: 361) |
| 50A8F2-Vκ_g3-VL | DIQLTQSPSSLSASVGDRVTITCTASSSVSSTYLHWYQQKPGKSPKLLIYSTSNLASGVP |
| | SRFSGSGSGTDYTLTISSLQPEDAATYYCHQYHRSPLTFGQGTKLEIK |
| | (SEQ ID NO: 362) |

TABLE 25
Combinations of 50A8F2 humanized sequence variants individual variable heavy
and light chains produced as human IgG1 kappa antibodies containing the effector null
IgG1 Fc variant L234A/L235A/G237A.

| | | | | | SEC |
|---|---|---|---|---|---|
| | | | Back | Yield | purity |
| Antibody | Heavy Chain | Light Chain | Mutation | (mg) | (%) |
| HU50A8F2-01 | 50A8F2-VH_g0 (SEQ | 50A8F2-Vκ_g0 (SEQ ID | 0 | 0.903 | 99.95 |
| | ID NO: 355) | NO: 359) | | | |
| HU50A8F2-02 | 50A8F2-VH_g0 (SEQ | 50A8F2-Vκ_g1 (SEQ ID | 1 | 0.939 | 100 |
| | ID NO: 355) | NO: 360) | | | |
| HU50A8F2-03 | 50A8F2-VH_g0 (SEQ | 50A8F2-Vκ_g2 (SEQ ID | 3 | 1.158 | 100 |
| | ID NO: 355) | NO: 361) | | | |
| HU50A8F2-04 | 50A8F2-VH_g0 (SEQ | 50A8F2-Vκ_g3 (SEQ ID | 4 | 1.461 | 100 |
| | ID NO: 355) | NO: 362) | | | |
| HU50A8F2-05 | 50A8F2-VH_g1 (SEQ | 50A8F2-Vκ_g0 (SEQ ID | 2 | 1.269 | 99.46 |
| | ID NO: 356) | NO: 359) | | | |
| HU50A8F2-06 | 50A8F2-VH_g1 (SEQ | 50A8F2-Vκ_g1 (SEQ ID | 3 | 1.86 | 99.02 |
| | ID NO: 356) | NO: 360) | | | |
| HU50A8F2-07 | 50A8F2-VH_g1 (SEQ | 50A8F2-Vκ_g2 (SEQ ID | 5 | 1.455 | 99.31 |
| | ID NO: 356) | NO: 361) | | | |
| HU50A8F2-08 | 50A8F2-VH_g1 (SEQ | 50A8F2-Vκ_g3 (SEQ ID | 6 | 2.073 | 98.89 |
| | ID NO: 356) | NO: 362) | | | |
| HU50A8F2-09 | 50A8F2-VH_g2 (SEQ | 50A8F2-Vκ_g0 (SEQ ID | 3 | 1.464 | 99.28 |
| | ID NO: 357) | NO: 359) | | | |
| HU50A8F2-10 | 50A8F2-VH_g2 (SEQ | 50A8F2-Vκ_g1 (SEQ ID | 4 | 1.614 | 99.39 |
| | ID NO: 357) | NO: 360) | | | |
| HU50A8F2-11 | 50A8F2-VH_g2 (SEQ | 50A8F2-Vκ_g2 (SEQ ID | 6 | 1.989 | 99.04 |
| | ID NO: 357) | NO: 361) | | | |
| HU50A8F2-12 | 50A8F2-VH_g2 (SEQ | 50A8F2-Vκ_g3 (SEQ ID | 7 | 1.749 | 99.04 |
| | ID NO: 357) | NO: 362) | | | |
| HU50A8F2-13 | 50A8F2-VH_g3 (SEQ | 50A8F2-Vκ_g0 (SEQ ID | 5 | 1.644 | 99.13 |
| | ID NO: 358) | NO: 359) | | | |
| HU50A8F2-14 | 50A8F2-VH_g3 (SEQ | 50A8F2-Vκ_g1 (SEQ ID | 6 | 1.8575 | 98.98 |
| | ID NO: 358) | NO: 360) | | | |
| HU50A8F2-15 | 50A8F2-VH_g3 (SEQ | 50A8F2-Vκ_g2 (SEQ ID | 8 | 1.851 | 99.24 |
| | ID NO: 358) | NO: 361) | | | |
| HU50A8F2-16 | 50A8F2-VH_g3 (SEQ | 50A8F2-Vκ_g3 (SEQ ID | 9 | 2.409 | 98.87 |
| | ID NO: 358) | NO: 362) | | | |
| HU50A8F2-chimeric | 50A8F2-VH (SEQ ID | 50A8F2-Vκ (SEQ ID | 0 | 0.732 | 97.86 |
| | NO: 169) | NO: 170) | | | |

### Example 10: Humanization of 22F10G3

Humanized sequence variants of 22F10G3 were produced for both heavy and light chains by CDR grafting (Almagro et al. 2008) of the murine antibody 22F10G3 onto human frameworks IGHV4-31*02/J4*01 and IGKV4-1*01/J2*01. These are shown in Table 26A.

These sequence variants were combined and expressed as human IgG1 kappa antibodies to produce 16 antibodies as shown in Table 26B.

TABLE 26A
22F10G3 humanized sequence variants for individual heavy and light chains

| Antibody | |
|---|---|
| Variable Chain | Sequence |
| 22F10G3-VH1 | QVQLQESGPGLVKPSQTLSLTCTVSGYSLTSDFAWNWIRQHPGKGLEWIGDIRYSGSTDYNPSL |
| | KGRVTISVDTSKNQFSLKLSSVTAADTAVYYCARGGYYGYDEGYPMDYWGQGTLVTVSS |
| | (SEQ ID NO: 331) |
| 22F10G3-VH2 | QVQLQESGPGLVKPSQTLSLTCTVSGYSLTSDFAWNWIRQHPGKGLEWMGDIRYSGSTDYNPSL |
| | KGRVTISRDTSKNQFSLKLSSVTAADTAVYYCARGGYYGYDEGYPMDYWGQGTLVTVSS |
| | (SEQ ID NO: 332) |
| 22F10G3-VH3 | QVQLQESGPGLVKPSQTLSLTCTVSGYSLTSDFAWNWIRQHPGKGLEWMGDIRYSGSTDYNPSL |
| | KGRSTISRDTSKNQFSLKLSSVTAADTAVYYCARGGYYGYDEGYPMDYWGQGTLVTVSS |
| | (SEQ ID NO: 333) |
| 22F10G3-VH4 | QVQLQESGPGLVKPSQTLSLTCTVSGYSLTSDFAWNWIRQFPGKGLEWMGDIRYSGSTDYNPSL |
| | KGRSTISRDTSKNQFFLKLSSVTAADTAVYYCARGGYYGYDEGYPMDYWGQGTLVTVSS |
| | (SEQ ID NO: 334) |
| 22F10G3-VL1 | DIVMTQSPDSLAVSLGERATINCRASESVDVYGNSFMHWYQQKPGQPPKLLIYLAFNLKSGVPDR |
| | FSGSGSGTDFTLTISSLQAEDVAVYYCQQNNEDPFTFGQGTKLEIK (SEQ ID NO: 335) |
| 22F10G3-VL2 | DIVMTQSPDSLAVSLGERATINCRASESVDVYGNSFMHWYQQKPGQPPKLLIYLAFNLKSGVPAR |
| | FSGSGSGTDFTLTISSLQAEDVAVYYCQQNNEDPFTFGQGTKLEIK (SEQ ID NO: 336) |
| 22F10G3-VL3 | DIVMTQSPDSLAVSLGERATISCRASESVDVYGNSFMHWYQQKPGQPPKLLIYLAFNLKSGVPDR |
| | FSGSGSGTDFTLTISSLQAEDVAVYYCQQNNEDPFTFGQGTKLEIK (SEQ ID NO: 337) |
| 22F10G3-VL4 | DIVMTQSPDSLAVSLGERATISCRASESVDVYGNSFMHWYQQKPGQPPKLLIYLAFNLKSGVPAR |
| | FSGSGSGTDFTLTISSLQAEDVATYYCQQNNEDPFTFGQGTKLEIK (SEQ ID NO: 338) |
| 22F10G3-VL3-NA | DIVMTQSPDSLAVSLGERATISCRASESVDVYGNAFMHWYQQKPGQPPKLLIYLAFNLKSGVPDR |
| | FSGSGSGTDFTLTISSLQAEDVAVYYCQQNNEDPFTFGQGTKLEIK (SEQ ID NO: 341) |
| 22F10G3-VL3-QS | DIVMTQSPDSLAVSLGERATISCRASESVDVYGQSFMHWYQQKPGQPPKLLIYLAFNLKSGVPDR |
| | FSGSGSGTDFTLTISSLQAEDVAVYYCQQNNEDPFTFGQGTKLEIK (SEQ ID NO: 345) |

TABLE 26B
Combinations of 22F10G3 humanized sequence variants individual
variable heavy and light chains produced as human
IgG1 kappa antibodies.

| Antibody | Heavy Chain | Light Chain |
|---|---|---|
| Hu22F10G3- | 22F10G3-VH | 22F10G3-VL |
| chimeric | (SEQ ID NO: 321) | (SEQ ID NO: 322) |
| Hu22F10G3-1.1 | 22F10G3-VH1 | 22F10G3-VL1 |
| | (SEQ ID NO: 331) | (SEQ ID NO: 335) |
| Hu22F10G3-1.2 | 22F10G3-VH1 | 22F10G3-VL2 |
| | (SEQ ID NO: 331) | (SEQ ID NO: 336) |
| Hu22F10G3-1.3 | 22F10G3-VH1 | 22F10G3-VL3 |
| | (SEQ ID NO: 331) | (SEQ ID NO: 337) |
| Hu22F10G3-1.4 | 22F10G3-VH1 | 22F10G3-VL4 |
| | (SEQ ID NO: 331) | (SEQ ID NO: 338) |
| Hu22F10G3-2.1 | 22F10G3-VH2 | 22F10G3-VL1 |
| | (SEQ ID NO: 332) | (SEQ ID NO: 335) |
| Hu22F10G3-2.2 | 22F10G3-VH2 | 22F10G3-VL2 |
| | (SEQ ID NO: 332) | (SEQ ID NO: 336) |
| Hu22F10G3-2.3 | 22F10G3-VH2 | 22F10G3-VL3 |
| | (SEQ ID NO: 332) | (SEQ ID NO: 337) |
| Hu22F10G3-2.4 | 22F10G3-VH2 | 22F10G3-VL4 |
| | (SEQ ID NO: 332) | (SEQ ID NO: 338) |
| Hu22F10G3-3.1 | 22F10G3-VH3 | 22F10G3-VL1 |
| | (SEQ ID NO: 332) | (SEQ ID NO: 335) |
| Hu22F10G3-3.2 | 22F10G3-VH3 | 22F10G3-VL2 |
| | (SEQ ID NO: 333) | (SEQ ID NO: 336) |
| Hu22F10G3-3.3 | 22F10G3-VH3 | 22F10G3-VL3 |
| | (SEQ ID NO: 333) | (SEQ ID NO: 337) |
| Hu22F10G3-3.4 | 22F10G3-VH3 | 22F10G3-VL4 |
| | (SEQ ID NO: 333) | (SEQ ID NO: 338) |
| Hu22F10G3-4.1 | 22F10G3-VH4 | 22F10G3-VL1 |
| | (SEQ ID NO: 334) | (SEQ ID NO: 335) |
| Hu22F10G3-4.2 | 22F10G3-VH4 | 22F10G3-VL2 |
| | (SEQ ID NO: 334) | (SEQ ID NO: 336) |
| Hu22F10G3-4.3 | 22F10G3-VH4 | 22F10G3-VL3 |
| | (SEQ ID NO: 334) | (SEQ ID NO: 337) |
| Hu22F10G3-4.4 | 22F10G3-VH4 | 22F10G3-VL4 |
| | (SEQ ID NO: 334) | (SEQ ID NO: 338) |
| Hu22F10G3- | 22F10G3-VH2 | 22F10G3-VL3-NA |
| 2.3-NA | (SEQ ID NO: 332) | (SEQ ID NO: 341) |
| Hu22F10G3- | 22F10G3-VH2 | 22F10G3-VL3-QS |
| 2.3-QS | (SEQ ID NO: 332) | (SEQ ID NO: 345) |

### Example 11: Monoclonal Anti-KLRB1 47A5H2 Chimeric and Humanized Antibodies Bind to Human KLRB1 and Cynomolgus KLRB1 by ELISA

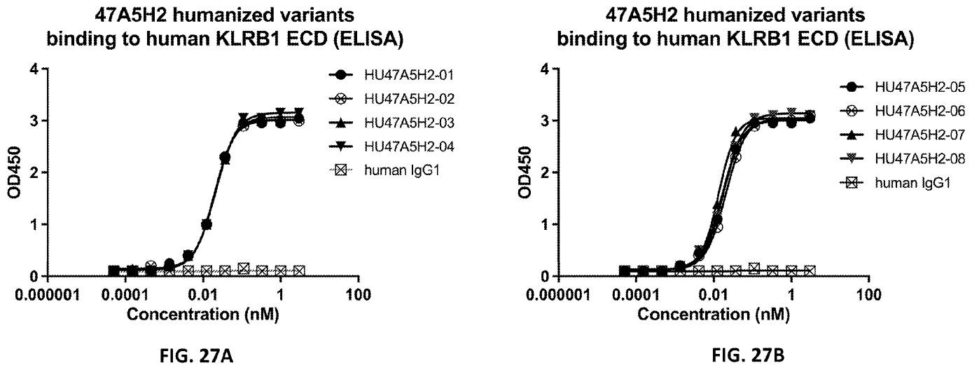

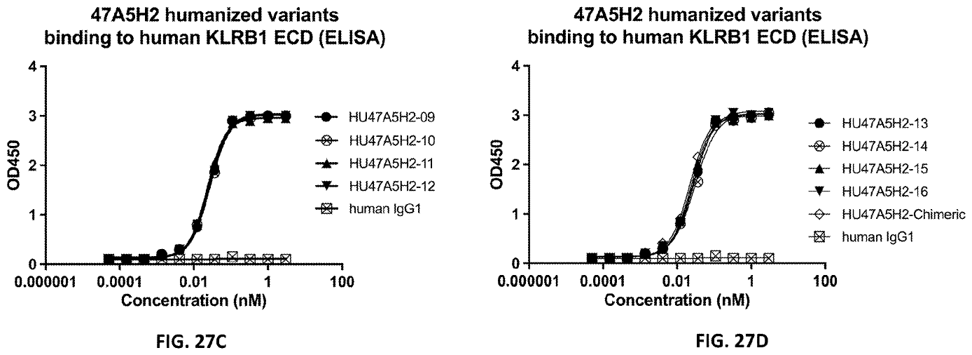

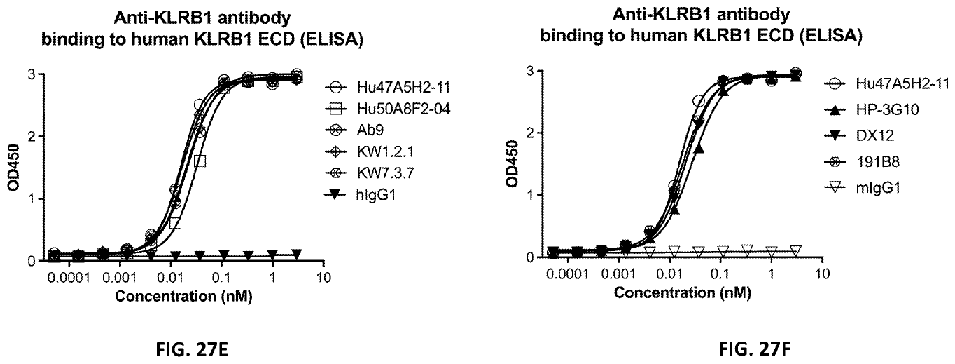

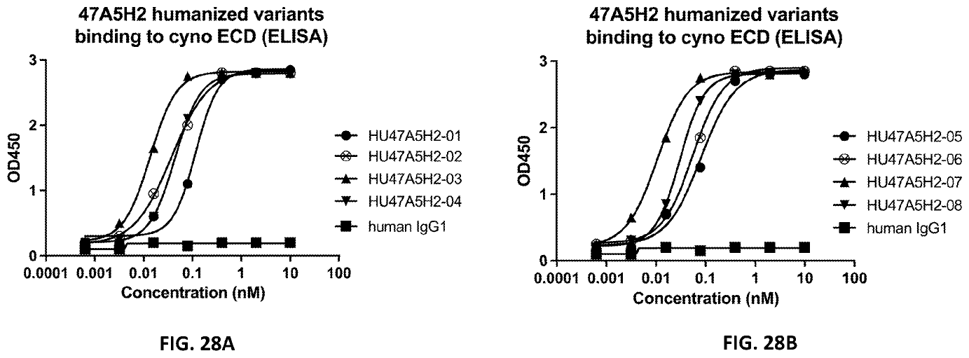

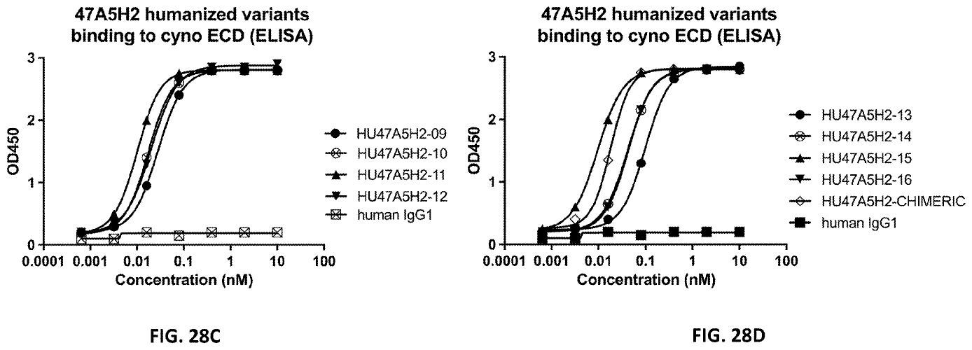

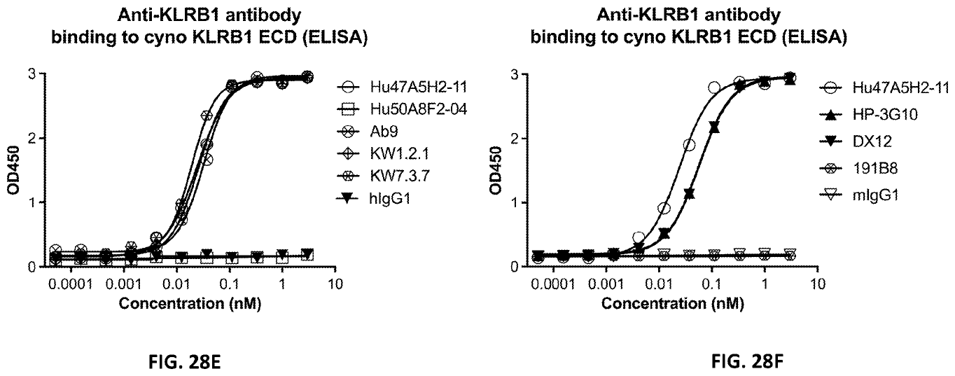

Humanized variants of 47A5H2 created by CDR grafting (Almagro et al. 2008) were assayed for binding against human and cynomolgus KLRB1 protein extracellular domain (ECD) via ELISA. Human KLRB1 extracellular domain was amino acids 67-225 from human KLRB1 protein (SEQ ID NO:1). Cynomolgus extracellular domain was amino acids 67-227 from cynomolgus KLRB1 protein (SEQ ID NO:3). ELISA plates were coated with KLRB1 ECD at 1 μg/ml, 4 °C, overnight prior to adding primary antibody (for human, 3 nM starting concentration, and 3-fold dilution; for cynomolgus, 10 nM starting concentration and 5-fold dilution) at 37 °C for 1 hour and then secondary anti-human IgG antibody 1:5000 at 37 °C for 45 minutes. The results are shown in FIGS. 27A-D and 28A-D, and Table 27.

Comparison experiments of Hu47A5H2-11 and Hu50A8F2-04 were performed with other antibodies Ab9, KW1.2.1, and KW7.3.7 comprising N297A effector null mutations, and HP-3G10, DX12, and 191B8. Ab9 is described in PCT publication WO2023028501A1 and KW1.2.1 and KW7.3.7 are described in US patent application publication US20210122826A1. HP-3G10, DX12 and 191B8 are commercial antibody reagents.

These experiments showed that Hu47A5H2-11 demonstrated a lower EC50 for human KLRB1 binding, and that Hu50A8F2-04 and 191B8 did not bind to cynomolgus KLRB1 protein. The results are shown in FIGS. 27E-F and 28E-F and summarized in Tables 28 and 29.

TABLE 27
EC50 and maximum optical density 450 (OD450) of binding
of 47A5H2 humanized variants to human and cynomolgus
KLRB1 extracellular domain protein

| | ELISA binding with | | ELISA binding with | |
|---|---|---|---|---|
| | human KLRB1 | | cyno KLRB1 | |
| | EC50 | Top | EC50 | Top |
| Antibody | (nM) | OD450 | (nM) | OD450 |
| HU47A5H2-01 | 0.01949 | 3.01 | 0.1162 | 2.86 |
| HU47A5H2-02 | 0.01978 | 3.02 | 0.03723 | 2.85 |
| HU47A5H2-03 | 0.02048 | 3.07 | 0.01372 | 2.82 |
| HU47A5H2-04 | 0.0212 | 3.16 | 0.04411 | 2.79 |
| HU47A5H2-05 | 0.01719 | 3.01 | 0.08239 | 2.87 |
| HU47A5H2-06 | 0.02038 | 3.05 | 0.05621 | 2.9 |
| HU47A5H2-07 | 0.0134 | 3.05 | 0.01081 | 2.84 |
| HU47A5H2-08 | 0.01739 | 3.14 | 0.03064 | 2.81 |
| HU47A5H2-09 | 0.02726 | 3.04 | 0.02826 | 2.81 |
| HU47A5H2-10 | 0.02724 | 3.03 | 0.01744 | 2.8 |
| HU47A5H2-11 | 0.02377 | 2.96 | 0.009999 | 2.81 |
| HU47A5H2-12 | 0.02626 | 3.03 | 0.02005 | 2.88 |
| HU47A5H2-13 | 0.0266 | 3.04 | 0.09837 | 2.84 |
| HU47A5H2-14 | 0.03089 | 3.03 | 0.04141 | 2.8 |
| HU47A5H2-15 | 0.02406 | 2.99 | 0.009731 | 2.81 |
| HU47A5H2-16 | 0.02872 | 3.08 | 0.04289 | 2.8 |
| HU47A5H2-Chimeric | 0.02211 | 3.02 | 0.01822 | 2.81 |

TABLE 28
Comparative studies of Hu47A5H2-11 with other antibodies for binding and blocking.
Antibodies Hu50A8F2-04 and 191B8 do not bind to cynomolgus KLRB1.

| | Binding | | | | | | | | Blocking | |
|---|---|---|---|---|---|---|---|---|---|---|
| | ELISA to Human | | ELISA to Cyno | | FACS CHO-hum | | FACS CHO-cyno | | % Inhibition CHO-hum | |
| | EC50 | Top | EC50 | Top | EC50 | Max | EC50 | Max | IC50 | Max |
| Antibody | (nM) | OD450 | (nM) | OD450 | (nM) | MFI | (nM) | MFI | (nM) | Inhibition |
| Hu47A5H2-11 | 0.016 | 2.90 | 0.024 | 2.94 | 3.01 | 73,935 | 14.88 | 100,972 | 3.61 | 98.07 |
| Hu50A8F2-04 | 0.033 | 2.96 | N/A | 0.19 | 6.81 | 78,805 | N/A | 31 | N/A | N/A |
| Ab9 | 0.022 | 3.00 | 0.033 | 2.96 | 3.12 | 78,634 | 14.63 | 98,114 | 4.32 | 98.84 |
| KW1.2.1 | 0.021 | 2.93 | 0.026 | 2.92 | 4.66 | 67,927 | 22.01 | 105,843 | 7.42 | 95.49 |
| KW7.3.7 | 0.017 | 2.92 | 0.019 | 2.90 | 2.49 | 63,838 | 11.85 | 81,428 | 5.48 | 96.61 |
| HP-3G10 | 0.028 | 2.93 | 0.057 | 2.97 | 4.61 | 10,079 | 18.73 | 12,426 | 9.75 | 98.40 |
| DX12 | 0.021 | 2.93 | 0.057 | 2.95 | 5.57 | 9,887 | 10.73 | 7,498 | 10.87 | 98.00 |
| 191B8 | 0.018 | 2.92 | N/A | 0.17 | 1.92 | 10,793 | N/A | 79 | 5.24 | 98.23 |

N/A = not applicable

TABLE 29
Fold-ratio of Hu47A5H2-11 to other antibodies. Table 28 values for Hu47A5H2-11 were normalized to 1.00.
For EC50 and IC50, fold-ratio of >1 for a comparator antibody indicates better performance of Hu47A4H2-11
than the comparator antibody (e.g., improved potency). For maximum effect, fold-ratio of <1 indicates
better performance of Hu47A4H2 than the comparator antibody (e.g., improved maximum binding or blocking).

| | Binding | | | | | | | | Blocking | |
|---|---|---|---|---|---|---|---|---|---|---|
| | ELISA to Human | | ELISA to Cyno | | FACS CHO-hum | | FACS CHO-cyno | | % Inhibition CHO-hum | |
| | | Top | | Top | | Max | | Max | | Max |
| Antibody | EC50 | OD450 | EC50 | OD450 | EC50 | MFI | EC50 | MFI | IC50 | Inhibition |
| Hu47A5H2-11 | 1.00 | 1.00 | 1.00 | 1.00 | 1.00 | 1.00 | 1.00 | 1.00 | 1.00 | 1.00 |
| Hu50A8F2-04 | 2.10 | 1.02 | N/A | N/A | 2.27 | 1.07 | N/A | N/A | N/A | N/A |
| Ab9 | 1.42 | 1.03 | 1.35 | 1.01 | 1.04 | 1.06 | 0.98 | 0.97 | 1.19 | 1.01 |
| KW1.2.1 | 1.32 | 1.01 | 1.07 | 0.99 | 1.55 | 0.92 | 1.48 | 1.05 | 2.05 | 0.97 |
| KW7.3.7 | 1.07 | 1.01 | 0.79 | 0.99 | 0.83 | 0.86 | 0.80 | 0.81 | 1.52 | 0.99 |
| HP-3G10 | 1.77 | 1.01 | 2.34 | 1.01 | 1.53 | 0.14 | 1.26 | 0.12 | 2.70 | 1.00 |
| DX12 | 1.32 | 1.01 | 2.36 | 1.00 | 1.85 | 0.13 | 0.72 | 0.07 | 3.01 | 1.00 |
| 191B8 | 1.17 | 1.01 | N/A | N/A | 0.64 | 0.15 | N/A | N/A | 1.45 | 1.00 |

N/A = not applicable.

### Example 12: Monoclonal Anti-KLRB1 47A5H2 Chimeric and Humanized Antibodies Bind to Human KLRB1 and Cynomolgus KLRB1 Expressed on CHO-K1 Cells

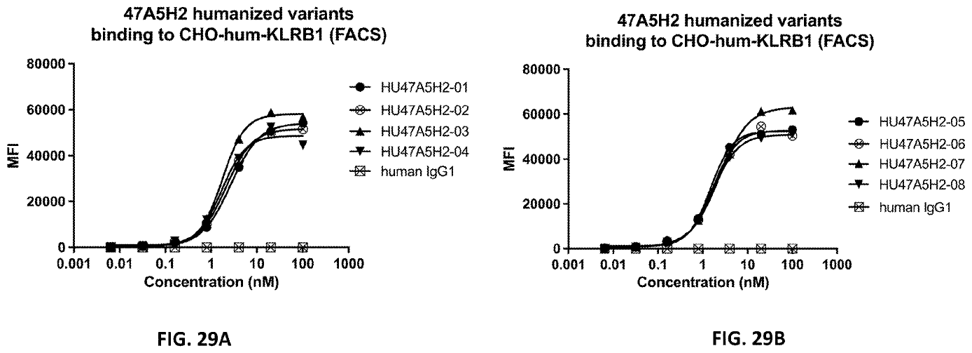

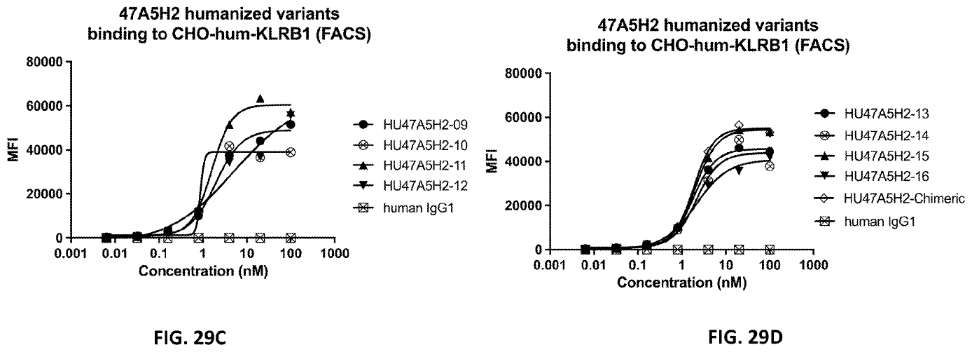

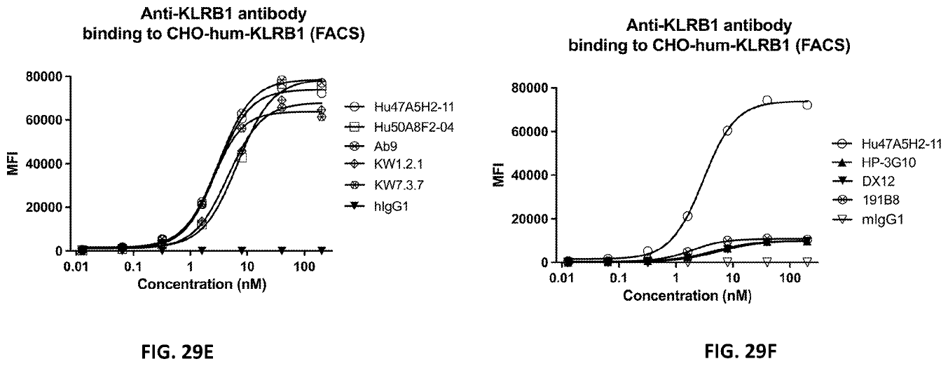

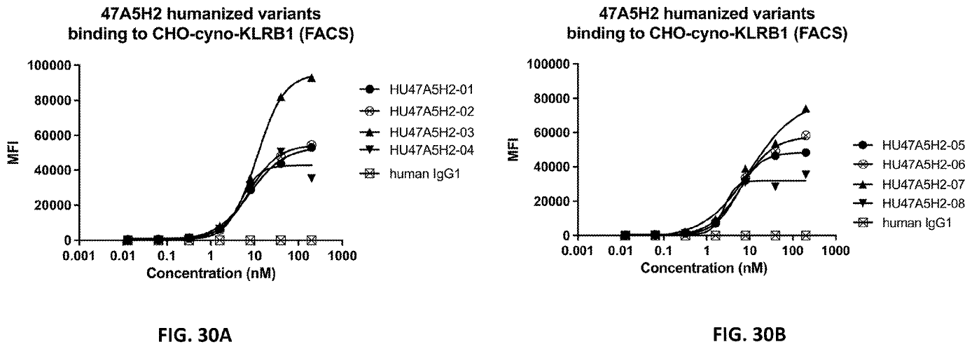

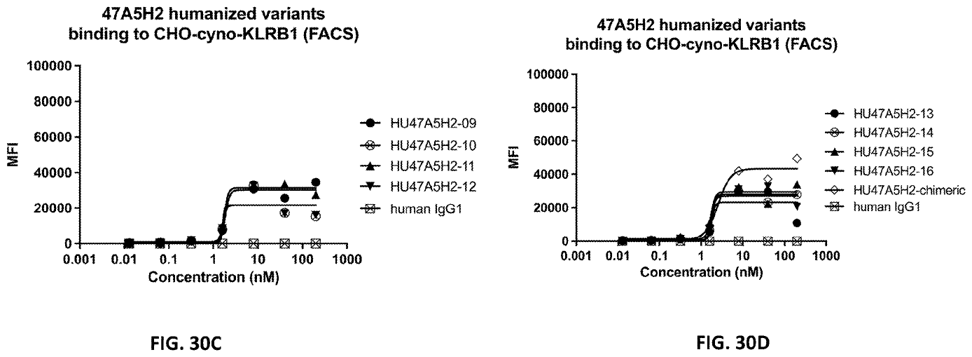

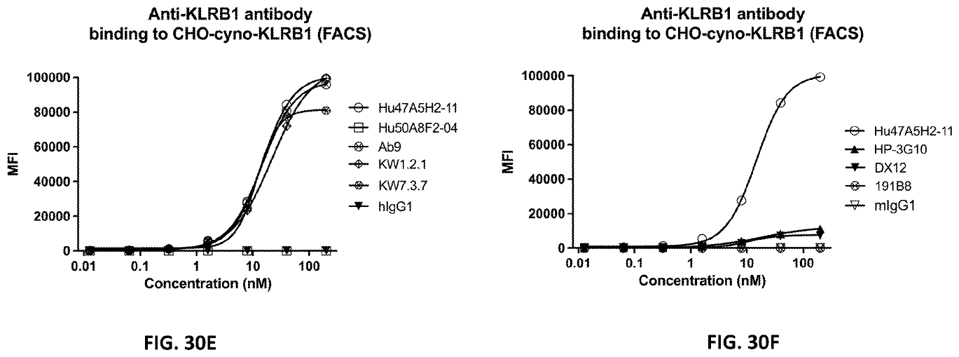

47A5H2 chimeric and humanized antibodies were assayed by FACS for binding to CHO-K1 cells expressing human KLRB1 (CHO-hum-KLRB1) and CHO-K1 cells expressing cynomolgus KLRB1 (CHO-cyno-KLRB1) (SEQ ID NO:2). Antibodies starting at 100 nM for human assay and 200 nM for cynomolgus assay were 5-fold serial diluted into wells with 2.0E+05 CHO-hum-KLRB1 or CHO-cyno-KLRB1 cells per well. Secondary Alexa 488 anti-human Fc antibody (Jackson, CAT #109-545-098, 1:500) and LIVE/DEAD dye (Invitrogen, CAT #L34964A, 1:1000) were used for FACS detection. Binding is shown in FIGS. 29A-D and 30A-D, and Table 30.

Comparison experiments of Hu47A5H2-11 with Hu50A8F2-04 and other antibodies showed that Hu50A8F2-04 and 191B8 did not bind to cynomolgus KLRB1 protein. We believe 47A5H2 chimeric and humanized antibodies bind to a different epitope than 191B8. The results are shown in FIGS. 29E-F and 30E-F and summarized in Tables 28 and 29 (above).

TABLE 30
EC50 and maximum mean fluorescent intensity (MFI) for
binding of 47A5H2 humanized variants to CHO-hum-KLRB1
and CHO-cyno-KLRB1 cells by FACS

| | FACS binding | | FACS binding | |
|---|---|---|---|---|
| | with CHOK1- | | with CHOK1- | |
| | human KLRB1 | | cyno KLRB1 | |
| Antibody | EC50 (nM) | Max MFI | EC50 (nM) | Max MFI |
| HU47A5H2-01 | 2.65 | 54258 | 7.76 | 53416 |
| HU47A5H2-02 | 2.08 | 51705 | 7.51 | 54696 |
| HU47A5H2-03 | 1.73 | 58146 | 11.55 | 95213 |
| HU47A5H2-04 | 1.67 | 48664 | 4.709 | 42864 |
| HU47A5H2-05 | 1.49 | 52432 | 5.216 | 48561 |
| HU47A5H2-06 | 1.67 | 52755 | 6.62 | 57804 |
| HU47A5H2-07 | 2.35 | 63129 | 10.79 | 79050 |
| HU47A5H2-08 | 1.68 | 50855 | 2.554 | 31909 |
| HU47A5H2-09 | 1.96 | 49003 | ~1.860 | 30344 |
| HU47A5H2-10 | 0.88 | 39103 | ~1.650 | 21809 |
| HU47A5H2-11 | 1.6 | 60464 | ~1.771 | 31488 |
| HU47A5H2-12 | 5.16 | 63101 | ~1.685 | 21800 |
| HU47A5H2-13 | 1.79 | 45626 | ~1.808 | 23225 |
| HU47A5H2-14 | 2.12 | 43801 | ~1.820 | 27003 |
| HU47A5H2-15 | 1.98 | 54478 | ~1.736 | 29507 |
| HU47A5H2-16 | 2.11 | 40821 | ~1.800 | 28017 |
| HU47A5H2-Chimeric | 1.84 | 54995 | 2.586 | 43287 |

### Example 13. Monoclonal Anti-KLRB1 47A5H2 Chimeric and Humanized Antibodies Inhibit Binding of CLEC2D to Human KLRB1 Expressed on CHO-K1 Cells

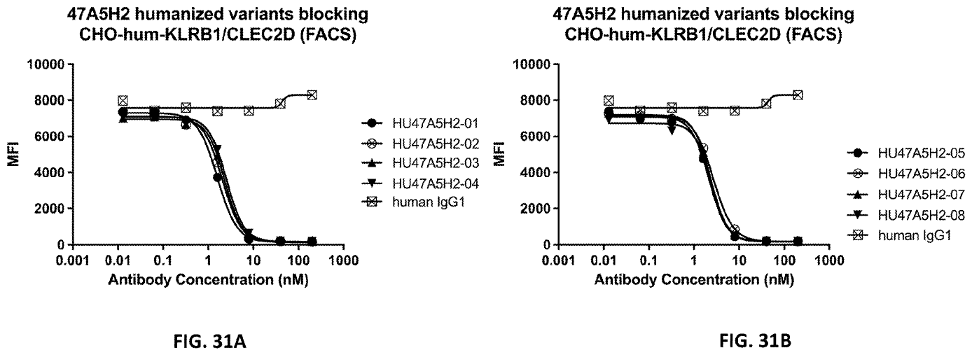

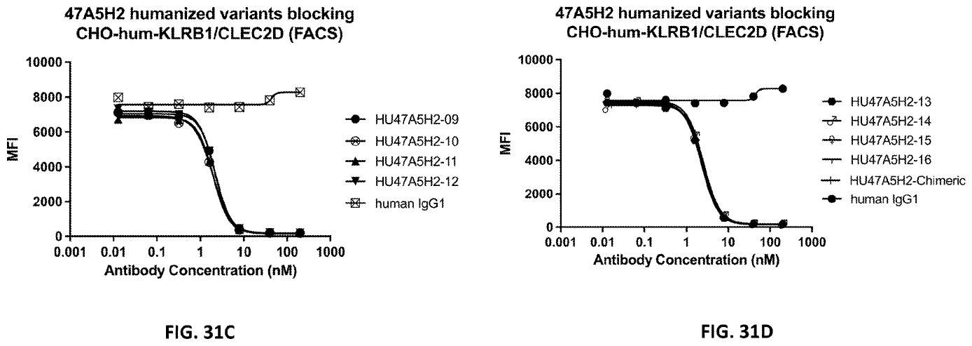

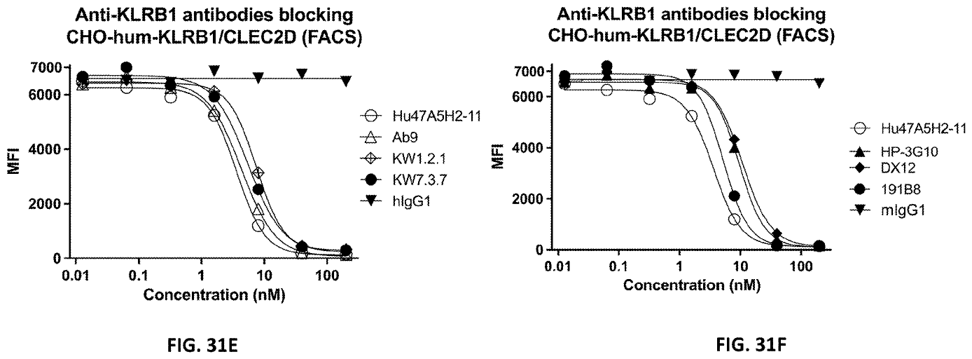

Competition assays of humanized variants of 47A5H2 with a biotin labeled CLEC2D fusion protein were performed by FACS. Antibodies starting at 200 nM were 5-fold serial diluted into wells along with 50 ul biotin-CLEC2D (final concentration 60 nM) with 2.0E+05 CHO-hum-KLRB1 or CHO-cyno-KLRB1 (SEQ ID NO:2) cells per well, at 4 degrees Celsius for 1 hour. Secondary streptavidin-Alexa 467 (Thermo, CAT #S21374, 1:1000) and LIVE/DEAD dye (Invitrogen, CAT #L34964A, 1:1000) were used for FACS detection. Blocking of CLEC2D binding by these humanized antibodies is shown in FIG. 31A-D and Table 31.

Comparison experiments of Hu47A5H2-11 with Hu50A8F2-04 and other antibodies showed that Hu47A5H2-11 demonstrated a lower IC50 than other antibodies tested. The results are shown in FIGS. 31E-F and summarized in Tables 28 and 29 (above).

TABLE 31
IC50 and maximum inhibition of blocking of CLEC2D
binding to CHOK1-human KLRB1 cells by 47A5H2
chimeric and humanized variants.

| | Inhibition of CLEC2D | |
|---|---|---|
| | Binding to CHO-hum-KLRB1 | |
| | IC50 | Maximum Inhibition |
| Antibody | (nM) | (%) |
| HU47A5H2-01 | 1.59 | 98.14 |
| HU47A5H2-02 | 2.05 | 98.28 |
| HU47A5H2-03 | 2.31 | 97.66 |
| HU47A5H2-04 | 2.53 | 97.73 |
| HU47A5H2-05 | 2.16 | 97.85 |
| HU47A5H2-06 | 2.66 | 97.72 |
| HU47A5H2-07 | 2.32 | 97.57 |
| HU47A5H2-08 | 2.42 | 97.83 |
| HU47A5H2-09 | 2.22 | 97.59 |
| HU47A5H2-10 | 1.96 | 98.21 |
| HU47A5H2-11 | 1.97 | 97.51 |
| HU47A5H2-12 | 2.17 | 97.51 |
| HU47A5H2-13 | 2.31 | 97.88 |
| HU47A5H2-14 | 2.54 | 97.59 |
| HU47A5H2-15 | 2.47 | 97.69 |
| HU47A5H2-16 | 2.41 | 97.77 |
| HU47A5H2-Chimeric | 2.54 | 97.62 |

### Example 14: Monoclonal Anti-KLRB1 47A5H2 and 22F10G3 Chimeric and Humanized Antibodies Binding Kinetics Against Human KLRB1

47A5H2 chimeric and humanized antibodies and other anti-KLRB1 antibodies were assayed for binding against human KLRB1 extracellular domain protein (ECD) via surface plasma resonance (SPR). Human KLRB1 extracellular domain was amino acids 67-225 from human KLRB1 protein (SEQ ID NO:1). A Biacore 8K instrument was used with series S sensor CM5 chips, multiple cycle kinetics and affinity capture in 96-well microplates, and full range antibody concentrations of 0.2 nM, 0.39 nM, 0.78 nM, 1.56 nM, 3.13 nM, 6.25 nM. 12.5 nM, and 25 nM. Antibody was fixed to the chip with anti-human-Fc antibody and analyte of human KLRB1 extracellular domain (ECD) was flowed over the surface at 30 μl/min, with contact time 180 seconds and dissociation time 400 seconds. Comparison experiments were done with other antibodies: Ab9, KW1.2.1, KW7.3.7, HP-3G10, DX12, and 191B8. The results are shown in Table 32A.

TABLE 32A
Binding kinetics of 47A5H2 chimeric and humanized
antibodies and other anti-KLRB1 antibodies against human
KLRB1 extracellular domain (ECD).

| Antibody | Kinetics Chi² (RU²) | ka (1/Ms) | kd (1/s) | KD (M) |
|---|---|---|---|---|
| 47A5H2-chimeric | 1.45E−01 | 8.50E+05 | 6.05E−04 | 7.12E−10 |
| Hu47A5H2-07 | 2.68E−01 | 9.05E+05 | 9.39E−04 | 1.04E−09 |
| Hu47A5H2-11 | 9.85E−02 | 1.04E+06 | 6.65E−04 | 6.39E−10 |
| Ab9 | 2.99E−02 | 1.78E+06 | 6.63E−04 | 3.72E−10 |
| KW1.2.1 | 8.46E−01 | 5.40E+05 | 4.12E−02 | 7.64E−08 |
| KW7.3.7 | 5.15E−01 | 3.62E+06 | 5.19E−01 | 1.43E−07 |
| HP-3G10 | 1.42E−01 | 5.22E+04 | 1.45E−04 | 2.77E−09 |
| DX12 | 7.44E−02 | 1.78E+05 | 5.50E−03 | 3.10E−08 |
| 191B8 | 9.74E−02 | 6.40E+05 | 1.02E−04 | 1.60E−10 |

In addition, 22F10G3 chimeric and humanized antibodies were assayed for binding against human KLRB1 extracellular domain protein (ECD) via surface plasma resonance (SPR). Human KLRB1 extracellular domain was amino acids 67-225 from human KLRB1 protein (SEQ ID NO: 1). A Biacore 8K instrument was used with multiple cycle kinetics and affinity capture in 96-well microplates, and single antibody concentration of 20 nM. Antibody was fixed to the chip and analyte of human KLRB1 extracellular domain (ECD) was flowed over the surface at 30 ul/min, with contact time 120 seconds and dissociation time 360 seconds. The results are shown in Table 32B.

TABLE 32B
Binding kinetics of 22F10G3 chimeric and humanized antibodies against
human KLRB1 extracellular domain (ECD).

| | | | | | Rmax |
|---|---|---|---|---|---|
| Antibody | Chi² (RU²) | ka (1/Ms) | kd (1/s) | KD (M) | (RU) |
| 22F10G3-chimeric | 3.63E+00 | 6.25E+06 | 4.16E−04 | 6.65E−11 | 498.2 |
| Hu22F10G3-1.1 | 8.73E−02 | 5.77E+06 | 7.18E−04 | 1.24E−10 | 51.1 |
| Hu22F10G3-1.2 | 1.38E−01 | 5.52E+06 | 8.42E−04 | 1.52E−10 | 55 |
| Hu22F10G3-1.3 | 1.66E−01 | 6.97E+06 | 1.24E−03 | 1.77E−10 | 45.2 |
| Hu22F10G3-1.4 | 9.92E−02 | 4.89E+06 | 7.48E−04 | 1.53E−10 | 73.4 |
| Hu22F10G3-2.1 | 4.04E−01 | 6.20E+06 | 6.42E−04 | 1.04E−10 | 140.5 |
| Hu22F10G3-2.2 | 2.94E−01 | 5.37E+06 | 7.24E−04 | 1.35E−10 | 127.7 |
| Hu22F10G3-2.3 | 1.93E−01 | 6.10E+06 | 5.89E−04 | 9.66E−11 | 100.7 |
| Hu22F10G3-2.4 | 7.15E−01 | 5.87E+06 | 6.81E−04 | 1.16E−10 | 205.9 |
| Hu22F10G3-3.1 | 8.44E−02 | 8.23E+06 | 1.00E−03 | 1.22E−10 | 19.5 |
| Hu22F10G3-3.2 | 3.05E−02 | 3.66E+06 | 6.93E−04 | 1.89E−10 | 14.4 |
| Hu22F10G3-3.3 | 3.34E−03 | 7.01E+06 | 1.26E−03 | 1.80E−10 | 12.9 |
| Hu22F10G3-3.4 | 5.48E−02 | 4.27E+06 | 5.13E−04 | 1.20E−10 | 36.7 |
| Hu22F10G3-4.1 | 3.82E−01 | 6.07E+06 | 6.37E−04 | 1.05E−10 | 137.2 |
| Hu22F10G3-4.2 | 1.89E−01 | 5.74E+06 | 7.44E−04 | 1.30E−10 | 103.1 |
| Hu22F10G3-4.3 | 2.87E−01 | 7.00E+06 | 6.81E−04 | 9.72E−11 | 117.4 |
| Hu22F10G3-4.4 | 4.85E−01 | 5.20E+06 | 6.31E−04 | 1.21E−10 | 212.7 |

### Example 15: Monoclonal Anti-KLRB1 47A5H2 Chimeric and Humanized Antibodies Binding Kinetics Against Cynomolgus KLRB1

47A5H2 chimeric and humanized antibodies and other anti-KLRB1 antibodies were assayed for binding against cynomolgus KLRB1 extracellular domain protein (ECD) via surface plasma resonance (SPR). Cynomolgus extracellular domain was amino acids 67-227 from cynomolgus KLRB1 protein (SEQ ID NO:3). A Biacore 8K instrument was used with series S sensor CM5 chips, multiple cycle kinetics and affinity capture in 96-well microplates, and full range antibody concentrations of 0.2 nM, 0.39 nM, 0.78 nM, 1.56 nM, 3.13 nM, 6.25 nM. 12.5 nM, and 25 nM. Antibody was fixed to the chip with anti-human-Fc antibody and analyte of cynomolgus KLRB1 extracellular domain (ECD) was flowed over the surface at 30 μl/min, with contact time 180 seconds and dissociation time 400 seconds. Comparison experiments were done with other antibodies: Ab9, KW7.3.7, HP-3G10, DX12, and 191B8. The results are shown in Table 33.

TABLE 33
Binding kinetics of 47A5H2 chimeric and humanized antibodies
and other anti-KLRB1 antibodies against cynomolgus
KLRB1 extracellular domain (ECD).

| Antibody | Kinetics Chi² (RU²) | ka (1/Ms) | kd (1/s) | KD (M) |
|---|---|---|---|---|
| 47A5H2-chimeric | 6.72E−01 | 9.28E+05 | 6.13E−03 | 6.61E−09 |
| Hu47A5H2-07 | 1.93E−01 | 5.40E+05 | 1.75E−03 | 3.25E−09 |
| Hu47A5H2-11 | 1.18E−01 | 5.71E+05 | 1.11E−03 | 1.94E−09 |
| Ab9 | 3.61E−02 | 3.39E+05 | 4.70E−04 | 1.39E−09 |
| KW7.3.7 | 7.28E−02 | 2.77E+05 | 3.03E−04 | 1.09E−09 |
| HP-3G10 | 4.62E−01 | 3.23E+05 | 2.60E−03 | 8.05E−09 |
| DX12 | 2.34E−01 | 8.16E+04 | 1.38E−03 | 1.69E−08 |
| 191B8 | N/A | N/A | N/A | N/A |

N/A = not applicable

### Example 16: Monoclonal Anti-KLRB1 50A8F2 Chimeric and Humanized Antibodies Bind to Human KLRB1 by ELISA

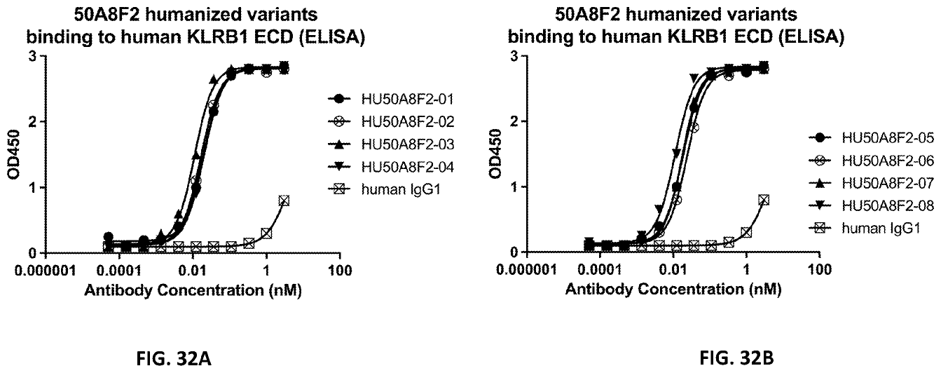

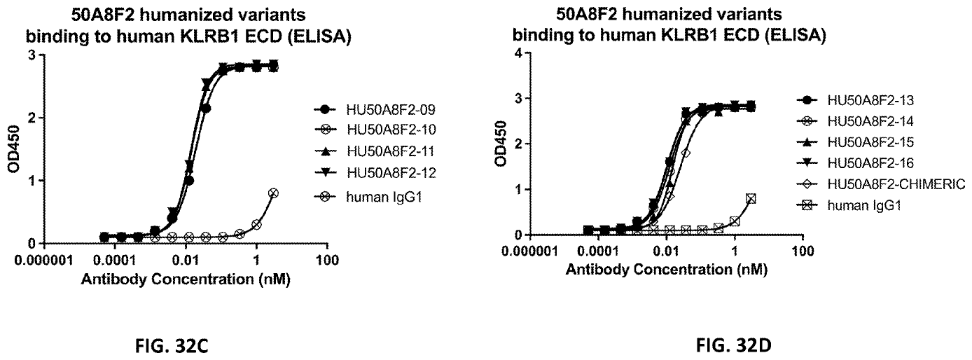

Humanized variants of 50A8F2 created by CDR grafting (Almagro et al. 2008) were assayed for binding against human KLRB1 protein extracellular domain (ECD) via ELISA. ELISA plates were coated with KLRB1 ECD at 1 μg/ml, 4 °C, overnight prior to adding primary antibody (3 nM starting concentration, and 3-fold dilution) at 37 °C for 1 hour and then secondary anti-human IgG antibody 1:5000 at 37 °C for 45 minutes. The results are shown in FIGS. 32A-D and Table 34.

TABLE 34
EC50 and maximum optical density 450 (OD450) of binding
of 50A8F2 humanized variants to human KLRB1 protein

| | ELISA binding with | |
|---|---|---|
| | human KLRB1 | |
| | EC50 | Top |
| Antibody | (nM) | OD450 |
| HU50A8F2-01 | 0.0197 | 2.83 |
| HU50A8F2-02 | 0.01671 | 2.81 |
| HU50A8F2-03 | 0.01127 | 2.83 |
| HU50A8F2-04 | 0.02009 | 2.83 |
| HU50A8F2-05 | 0.01831 | 2.82 |
| HU50A8F2-06 | 0.02355 | 2.79 |
| HU50A8F2-07 | 0.01744 | 2.79 |
| HU50A8F2-08 | 0.01114 | 2.84 |
| HU50A8F2-09 | 0.01893 | 2.84 |
| HU50A8F2-10 | 0.01875 | 2.83 |
| HU50A8F2-11 | 0.0144 | 2.82 |
| HU50A8F2-12 | 0.01403 | 2.85 |
| HU50A8F2-13 | 0.01042 | 2.83 |
| HU50A8F2-14 | 0.01215 | 2.82 |
| HU50A8F2-15 | 0.01484 | 2.77 |
| HU50A8F2-16 | 0.01027 | 2.86 |
| HU50A8F2-Chimeric | 0.02521 | 2.86 |

### Example 17: Monoclonal Anti-KLRB1 50A8F2 Chimeric and Humanized Antibodies Bind to Human KLRB1 Expressed on CHO-K1 Cells but do not Bind to Cynomolgus KLRB1 Expressed on CHO-K1 Cells

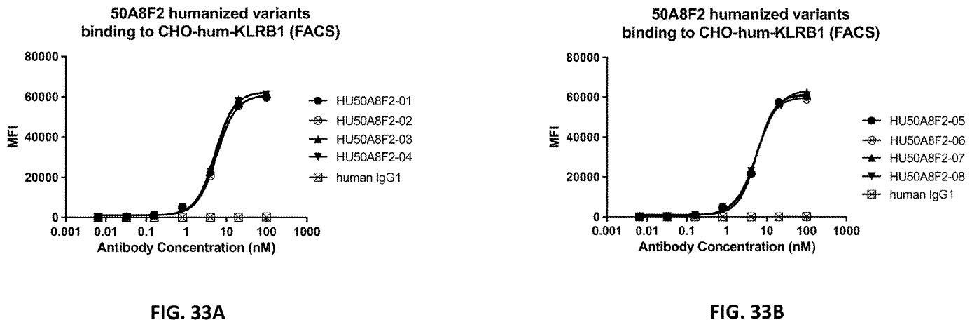

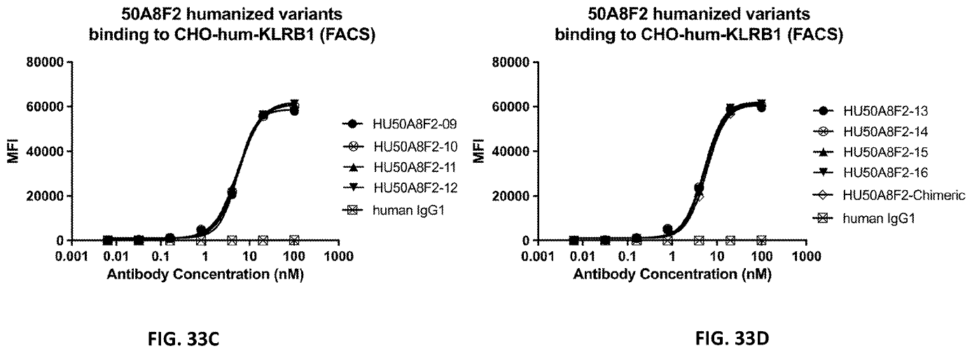

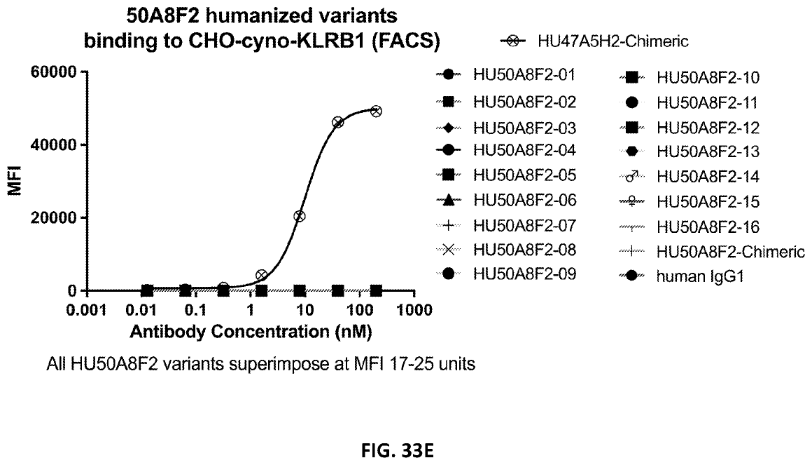

50A8F2 chimeric and humanized antibodies were assayed by FACS for binding to CHO-K1 cells expressing human KLRB1 (CHO-hum-KLRB1) (SEQ ID NO:1) and CHO-K1 cells expressing cynomolgus KLRB1 (CHO-cyno-KLRB1) (SEQ ID NO:2). Antibodies starting at 100 nM for human assay and 200 nM for cynomolgus assay were 5-fold serial diluted into wells with 2.0E+05 CHO-hum-KLRB1 or CHO-cyno-KLRB1 cells per well. Secondary Alexa 488 anti-human Fc antibody (Jackson, CAT #109-545-098, 1:500) and LIVE/DEAD dye (Invitrogen, CAT #L34964A, 1:1000) were used for FACS detection. Binding of 50A8F2 chimeric and humanized antibodies to CHO-hum-KLRB1 is shown in FIGS. 33A-D and Table 35. Absence of binding of 50A8F2 chimeric and humanized antibodies to CHO-cyno-KLRB1, along with presence of binding of 47A5H2 chimeric (also called HU47A5H2 chimeric) as a positive control, is shown in FIG. 33E.

TABLE 35
EC50 and maximum mean fluorescent intensity (MFI) for binding
of 50A8F2 humanized variants to CHO-hum-KLRB1 by FACS

| | FACS binding with CHOK1- | |
|---|---|---|
| | human KLRB1 | |
| Antibody | EC50 (nM) | Max MFI |
| HU50A8F2-01 | 5.54 | 61094 |
| HU50A8F2-02 | 5.81 | 60718 |
| HU50A8F2-03 | 5.17 | 62479 |
| HU50A8F2-04 | 5.45 | 62370 |
| HU50A8F2-05 | 5.51 | 60925 |
| HU50A8F2-06 | 5.44 | 59762 |
| HU50A8F2-07 | 5.72 | 63559 |
| HU50A8F2-08 | 5.44 | 61772 |
| HU50A8F2-09 | 5.4 | 58668 |
| HU50A8F2-10 | 5.67 | 61172 |
| HU50A8F2-11 | 5.43 | 62228 |
| HU50A8F2-12 | 5.77 | 61981 |
| HU50A8F2-13 | 5.06 | 60566 |
| HU50A8F2-14 | 5.12 | 61539 |
| HU50A8F2-15 | 5.39 | 61524 |
| HU50A8F2-16 | 5.27 | 62169 |
| HU50A8F2-Chimeric | 5.87 | 61007 |

### Example 18. Monoclonal Anti-KLRB1 50A8F2 Chimeric and Humanized Antibodies Augment Binding of CLEC2D to Human KLRB1 Expressed on CHO-K1 Cells

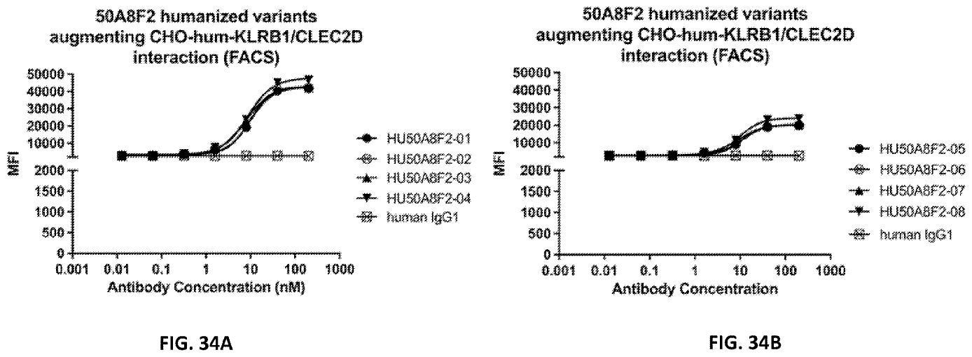

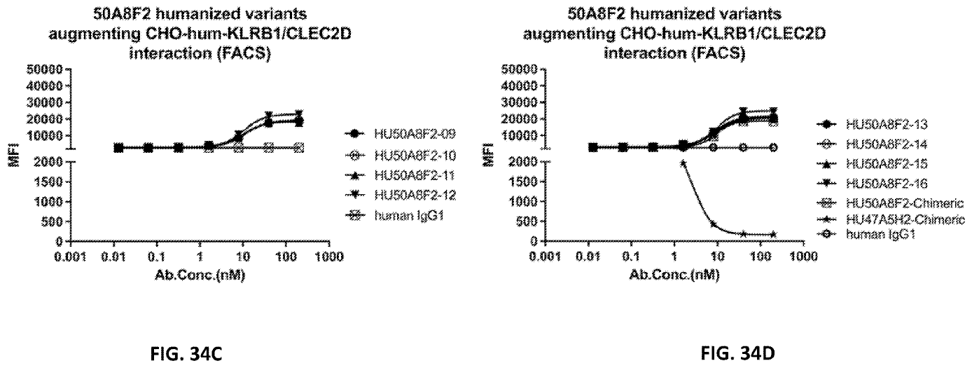

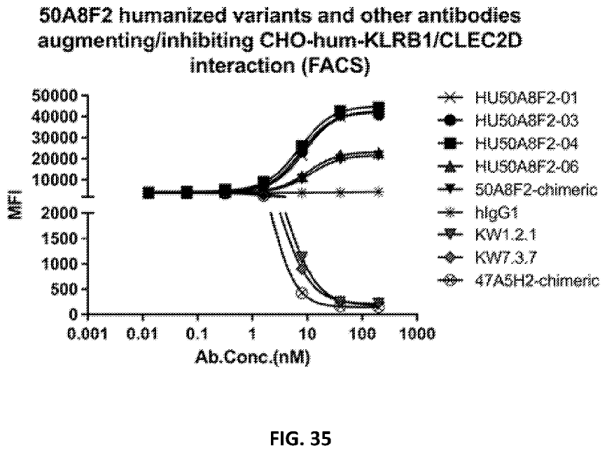

Competition assays of 50A8F2 chimeric and humanized antibodies with biotin labeled CLEC2D were performed by FACS. Antibodies starting at 200 nM were 5-fold serial diluted into wells along with 50 μl biotin-CLEC2D (final concentration 60 nM) with 2.0E+05 CHO-hum-KLRB1 cells per well, at 4 degrees Celsius for 1 hour. Secondary streptavidin-Alexa 467 (Thermo, CAT #S21374, 1:1000) and LIVE/DEAD dye (Invitrogen, CAT #L34964A, 1:1000) were used for FACS detection. Augmentation of (increased) CLEC2D binding by these humanized antibodies is shown in FIGS. 34A-D and Table 36. In a separate comparison experiment, 50A8F2 humanized antibodies demonstrated augmentation of CLEC2D binding but antibodies KW1.2.1 and KW7.3.7 did not, rather these later antibodies inhibited CLEC2D binding (FIG. 35 and Table 37), as previously shown in FIG. 31E.

TABLE 36
EC50 and maximum mean fluorescent intensity (MFI) of
augmentation of CLEC2D binding to CHO-hum-KLRB1 cells by
50A8F2 chimeric and humanized antibodies.

| | Cell based RBA with | |
|---|---|---|
| | CHOK1-human KLRB1 | |
| Antibody | EC50 (nM) | Max MFI |
| HU50A8F2-01 | 9.73 | 42455 |
| HU50A8F2-02 | 9.64 | 42885 |
| HU50A8F2-03 | 7.87 | 43117 |
| HU50A8F2-04 | 8.6 | 47847 |
| HU50A8F2-05 | 11.18 | 20183 |
| HU50A8F2-06 | 10.9 | 20284 |
| HU50A8F2-07 | 9.02 | 20607 |
| HU50A8F2-08 | 9.65 | 24104 |
| HU50A8F2-09 | 10.98 | 18965 |
| HU50A8F2-10 | 11.09 | 19473 |
| HU50A8F2-11 | 9.83 | 18366 |
| HU50A8F2-12 | 10.32 | 23078 |
| HU50A8F2-13 | 8.47 | 21650 |
| HU50A8F2-14 | 8.99 | 20559 |
| HU50A8F2-15 | 8.49 | 20795 |
| HU50A8F2-16 | 9.31 | 24932 |
| HU50A8F2-Chimeric | 9.37 | 18895 |

TABLE 37
EC50 and maximum augmentation of CLEC2D binding to
CHOK1-human KLRB1 cells by 50A8F2 chimeric and humanized
antibodies compared with antibodies KW1.2.1 and KW7.3.7.

| | Augmentation of CLEC2D | |
|---|---|---|
| | Binding to CHO-hum-KLRB1 | |
| | | Max |
| | | Concentration |
| Antibody | EC50 (nM) | MFI |
| HU50A8F2-chimeric | 11.31 | 21770 |
| HU50A8F2-01 | 8.87 | 42732 |
| HU50A8F2-03 | 7.62 | 42023 |
| HU50A8F2-04 | 7.17 | 45175 |
| HU50A8F2-06 | 10.06 | 23214 |
| KW1.2.1 | Inhibits, N/A | 213 |
| KW7.3.7 | Inhibits, N/A | 231 |

### Example 19: Monoclonal Anti-KLRB1 50A8F2 Chimeric and Humanized Antibodies Binding Kinetics Against Human KLRB1

Humanized variants of 50A8F2 and other antibodies were assayed for binding against human KLRB1 extracellular domain protein (ECD) via surface plasma resonance (SPR). A Biacore 8K instrument was used with series S sensor CM5 chips, multiple cycle kinetics and affinity capture in 96-well microplates, and full range antibody concentrations of 0.2 nM, 0.39 nM, 0.78 nM, 1.56 nM, 3.13 nM, 6.25 nM. 12.5 nM, and 25 nM. Antibody was fixed to the chip with anti-human-Fc antibody and analyte of human KLRB1 extracellular domain (ECD) was flowed over the surface at 30 μl/min, with contact time 180 seconds and dissociation time 400 seconds. The results are shown in Table 38.

TABLE 38
Binding kinetics of 50A8F2 chimeric and humanized and
other antibodies to human KLRB1.

| | Kinetics Chi² | | | |
|---|---|---|---|---|
| Antibody | (RU²) | ka (1/Ms) | kd (1/s) | KD (M) |
| 50A8F2-chimeric | 1.02E−01 | 5.71E+05 | 1.31E−04 | 2.29E−10 |
| Hu50A8F2-01 | 3.52E−01 | 3.63E+05 | 5.61E−04 | 1.55E−09 |
| Hu50A8F2-03 | 4.24E−02 | 4.61E+05 | 3.06E−04 | 6.65E−10 |
| Hu50A8F2-04 | 3.09E−01 | 5.68E+05 | 3.94E−04 | 6.93E−10 |
| Hu50A8F2-06 | 6.25E−01 | 4.86E+05 | 4.02E−04 | 8.28E−10 |
| Ab9 | 2.99E−02 | 1.78E+06 | 6.63E−04 | 3.72E−10 |
| KW1.2.1 | 8.46E−01 | 5.40E+05 | 4.12E−02 | 7.64E−08 |
| KW7.3.7 | 5.15E−01 | 3.62E+06 | 5.19E−01 | 1.43E−07 |
| HP-3G10 | 1.42E−01 | 5.22E+04 | 1.45E−04 | 2.77E−09 |
| DX12 | 7.44E−02 | 1.78E+05 | 5.50E−03 | 3.10E−08 |
| 191B8 | 9.74E−02 | 6.40E+05 | 1.02E−04 | 1.60E−10 |

### Example 20. Anti-KLRB1 47A5H2 Chimeric and Humanized Antibodies Increase Primary NK Cell Activation

An NK cell activation assay was developed as follows:

K562 cells were stably transfected with human CLEC2D to create pools and single clones of K562-CLEC2D target cells, with clone 1D1 selected for subsequent single clone experiments.

Primary NK cells were isolated from human PBMCs with the human NK Cell Enrichment Kit (Stemcell, Cat #19055). Primary NK cells 1.0E+06 were cultured in RPMI 1640 plus 10% FBS with 100 ng/mL rhIL-2 (Peprotech, Cat #200-02) overnight. On Day 2, NK cells were washed and resuspended with assay buffer (RPMI 1640 plus 10% FBS). 50 μl of NK cells (1.0E+05 cells/well) were incubated with various antibodies (50 μl/well) at 37 °C for 0.5h. K562-CLEC2D target cells were confirmed by FACS to express human CLEC2D using goat anti-human CLEC2D polyclonal antibody (Invitrogen, Cat #PA5-47496) and Alexa 488 anti-goat IgG (Invitrogen, Cat #A11055). K562 or K562-CLEC2D target cells, either pools or single clones, at 100 μl of cells (5.0E+05 per well), with various controls, were incubated with RPMI 1640 plus 10% FBS and 100 ng/mL rhIL-2 at 37 °C for 4 hours. Anti-CD107a-APC (BioLegend, Cat #328620) was added at the start of the incubation. Bredeldin A (BioLegend, Cat #420601) and monensin (BioLegend, Cat #420701) were added after 1 hour of incubation. Cells were then washed twice and blocked with human Fc (BD Cat #564220) for 10 minutes at room temperature. Flow detection antibodies (Live/dead dye, Invitrogen, Cat #L34964; anti-CD3-PE-Cy7, BioLegend, Cat #317334; anti-CD56-FITC, BioLegend, Cat #304604) were added and incubated at 4 °C for 30 minutes in the dark. Cells were then washed twice, suspended in 100 μl of Cytofix/Cytoperm solution (BD, Cat #554722) for 20 minutes at 4 °C, washed again and stained with IFN-gamma-PE (BioLegend, Cat #502509) at 4 °C for 30 minutes in the dark, washed again, and analyzed by FACS.

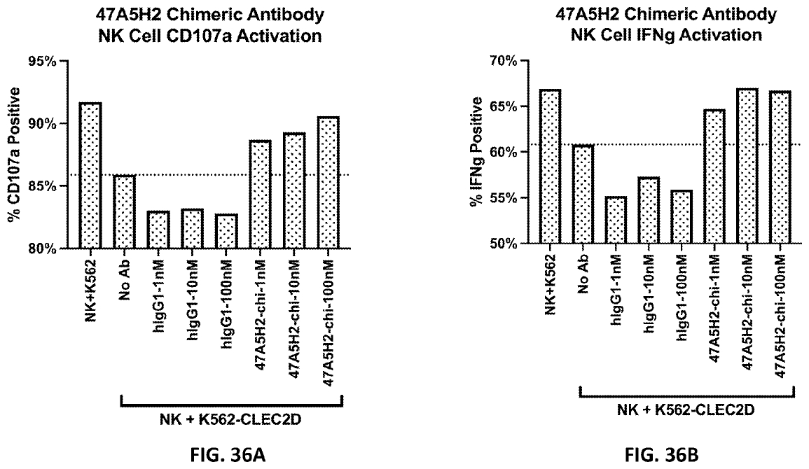

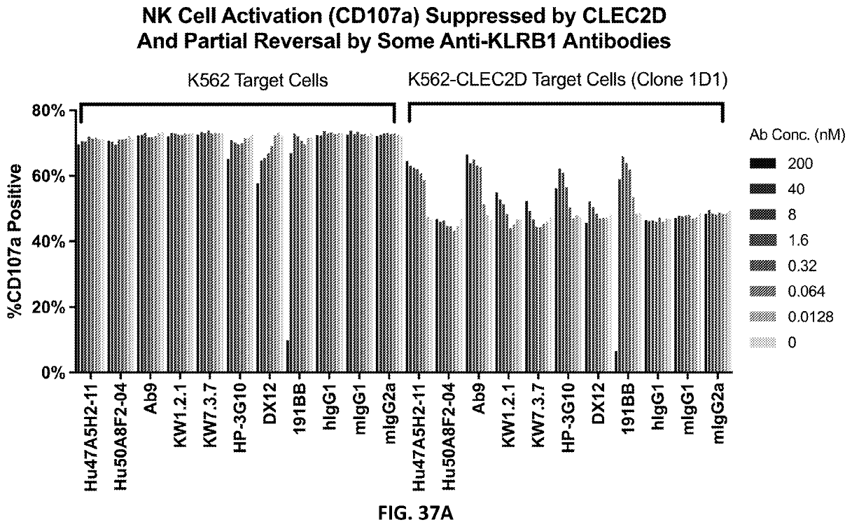

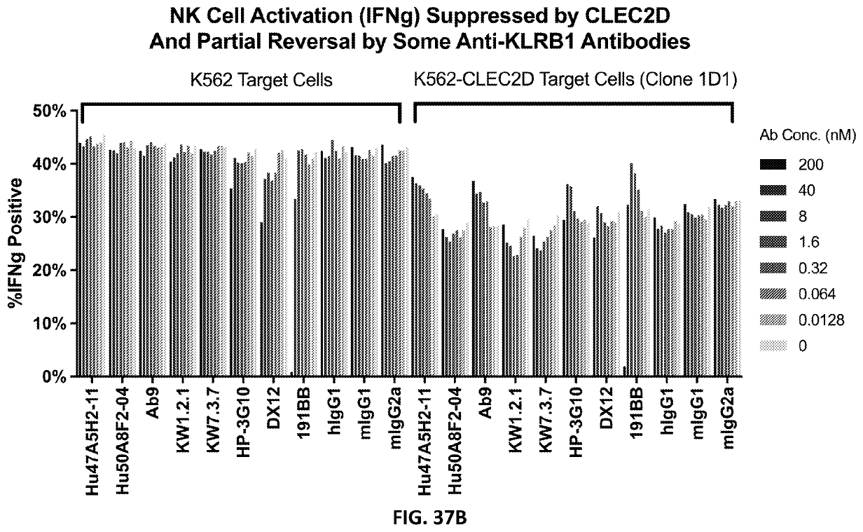

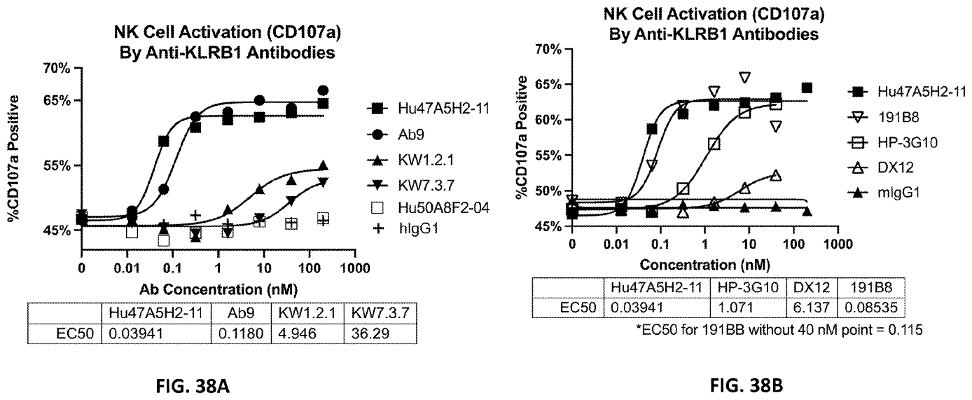

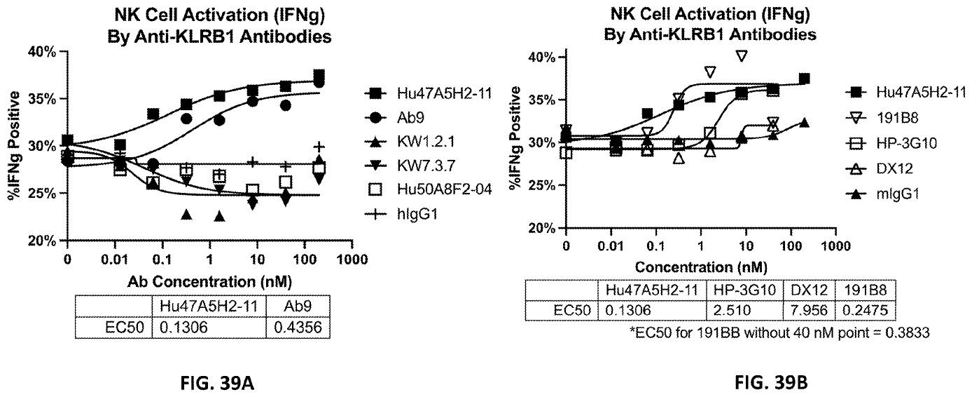

Results are shown in FIGS. 36-39 and summarized in Tables 39 and 40 below, and demonstrated that 47A5H2 chimeric and humanized antibodies increased NK cell activation using primary NK cells. These further demonstrated that NK cell activation is suppressed by CLEC2D (K562-CLEC2D cells cause less NK activation than K562 cells, FIGS. 36 and 37) and that Hu47A5H2-11 antibody increased NK cell activation with target cells K562-CLEC2D single clone 1D1. The EC50s for Hu47A5H2-11 NK cell activation for both CD107a expression and IFNg expression were superior to that of antibodies Ab9, KW1.2.1, and KW7.3.7 (FIGS. 38A, 39A). Antibodies KW1.2.1, and KW7.3.7 did not activate NK cell IFNg expression (FIG. 39A). Antibody 191B8 at 40 nM and at 200 nM caused NK cell toxicity and had a decreased effect on NK cell activation (FIGS. 37A, 37B, 38B, and 39B). Antibody Hu50A8F2-04 did not activate NK cell CD107a or IFNg expression (FIGS. 38A, 39A).

### Example 21. Anti-KLRB1 47A5H2 Humanized Variants Increase Primary NK Cell Killing

An NK cell killing assay was developed as follows:

Donors with high expression of KLRB1 (CD161) on NK cells, detected by FACS using PE-anti-CD161 antibody (BioLegend, Cat #339903), were selected for further studies. Primary NK cells were isolated from human PBMCs with the human NK cell enrichment Kit (Stemcell, Cat #19055). Primary NK cells 1.0E+06 were cultured in RPMI 1640 plus 10% FBS with 100 ng/mL rhIL-2 (Peprotech, Cat #200-02) overnight. On Day 2, NK cells were washed and resuspended with assay buffer (RPMI 1640 plus 10% FBS). 100 ul of NK cells (4.0E+04 cells/well) were incubated with various antibodies (50 ul/well) at various concentrations at 37 °C for 0.5 h. Raji target cells were confirmed by FACS to express human CLEC2D using goat anti-human CLEC2D polyclonal antibody (Invitrogen, Cat #PA5-47496) and Alexa 488 anti-goat IgG (Invitrogen, Cat #A11055, 1:1000). Raji target cells were stained with CellTrace Violet (CTV, Invitrogen, Cat #C34557). The staining was stopped with the same volume FBS for 5 minutes at RT, washed with sterile FACS buffer, and 50 ul of cells (4.0E+04 per well) were co-incubated with the primary NK cell/antibody mixtures at 37 °C for 4 hours. Cells were then washed twice with FACS buffer and stained with 100 ul/well Live/Dead far red fluorescent reactive dye (Invitrogen, Cat #L10120) at 4 °C for 0.5 hours. Cells were then washed twice with FACS buffer and analyzed by FACS.

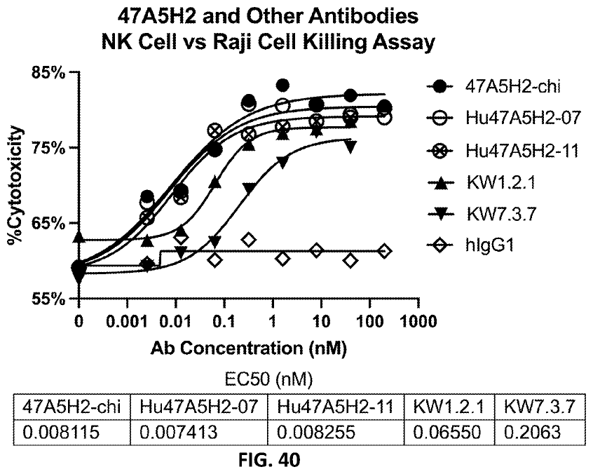

Results of one experiment (Experiment 21.1) are shown in FIG. 40, and demonstrated that 47A5H2-chimeric, Hu47A5H2-07, and Hu47A5H2-11 antibodies increased NK cell killing. In comparison to anti-KLRB1 antibodies KW1.2.1 and KW7.3.7, 47A5H2 chimeric and humanized antibodies had superior potency (smaller EC50s).

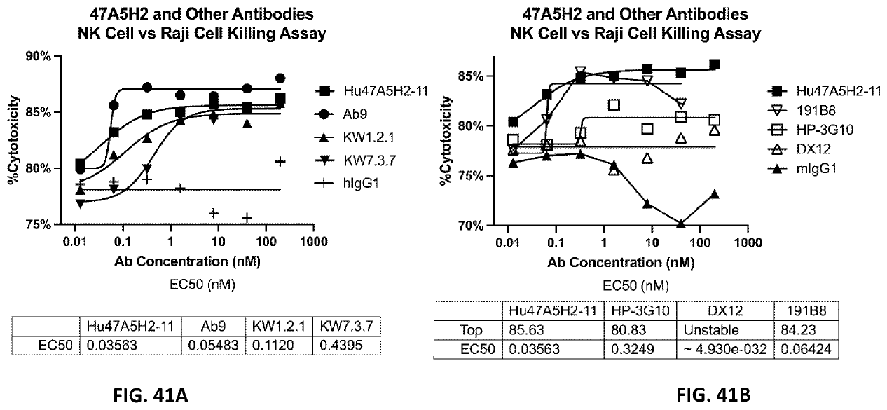

In another experiment (Experiment 21.2) shown in FIG. 41, Hu47A5H2-11 had superior potency (smaller EC50) compared with Ab9, KW1.2.1, KW7.3.7, HP-3G10, DX12, and 191B8. The results are summarized in Tables 39 and 40, below.

### Example 22. Anti-KLRB1 47A5H2 and Humanized Variants Activate Jurkat T Cells

A T cell activation assay was developed as follows:

Jurkat-NFAT cells were stably transfected with 1G4-TCR and human CD161 (KLRB1) to create pools and single clones of Jurkat-NFAT-1G4TCR-CD161 effector cells. Expression was confirmed by FACS using anti-HA tag on TCR alpha (BioLegend, Cat #682404), anti-PC tag on TCR beta (Genscript, Cat #A01774-100), and anti-CD161 Hu47A5H2-7 antibody.

K562 cells were stably transfected with human CLEC2D and HLA A*02:01 and β2m to create pools and single clones of K562-CLEC2D-HLA target cells.

Jurkat-NFAT-1G4TCR-CD161 cells were cultured with K562-CLEC2D-HLA cells pulsed with NY-ESO-1 peptide (SLLMWITQC) under a variety of conditions. In a typical experiment, various concentrations of NY-ESO-1 peptide were added to assay medium (RPMI 1640 plus 10% FBS), and 25 ul of assay medium and 25 ul target K562-CLEC2D-HLA cells were incubated at 37 °C for 120 minutes. Jurkat-NFAT-1G4TCR-CD161 effector cells 25 μl with 25 μl of various antibodies were added and incubated at 37 °C for 5 hours. ONE-Glo (Promega, Cat #E6120) 100 μl was added and incubated at room temperature for 3 minutes. Well luminescence was then measured with a Varioskan Lux plate reader.

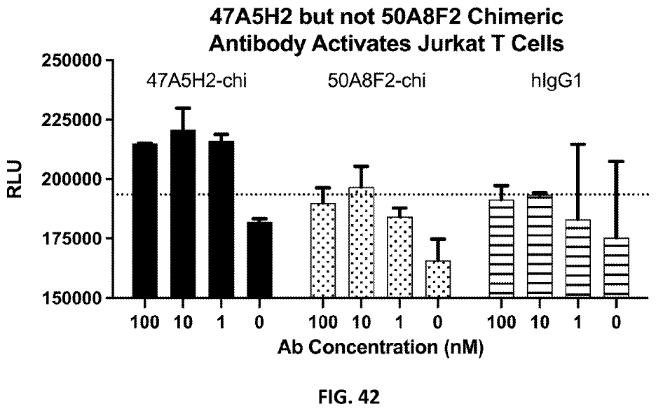

The results of one experiment (Experiment 22.1) using Jurkat cell pools and K562-CLEC2D-HLA cell pools are shown in FIG. 42, and demonstrated that 47A5H2 chimeric antibody but not 50A8F2 chimeric antibody, increased T cell activation.

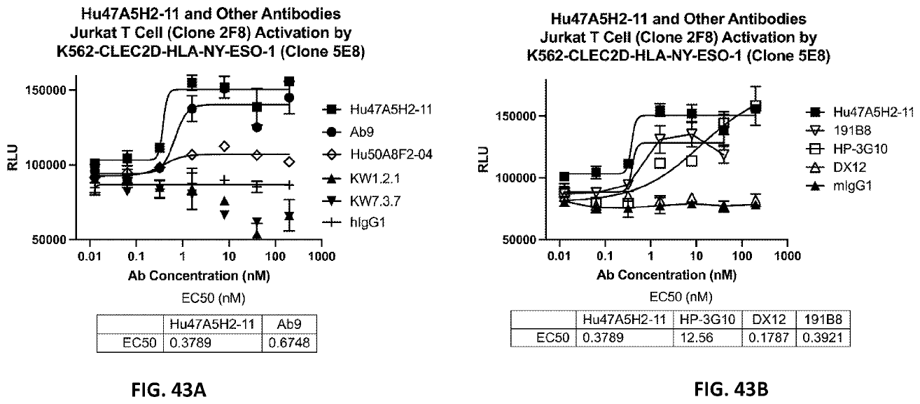

In another experiment (Experiment 22.2) using single clones of Jurkat cells (clone 2F8) and single clones of K562-CLEC2D-HLA cells (clone 5E8) shown in FIG. 43, Hu47A5H2-11 had superior potency (smaller EC50) compared with Ab9, KW1.2.1, KW7.3.7, HP-3G10, DX12, and 191B8. The results are summarized in Tables 39 and 40, below.

TABLE 39
Comparative studies of Hu47A5H2-11 with other antibodies in functional
assays. Functional assays summarized are described in Examples 20-22.
Functional Activation

| | NK Activation | | NK Activation | | NK Killing | | NK Killing | | T Cell Activation | |
|---|---|---|---|---|---|---|---|---|---|---|
| | CD107a | | IFNg | | (Experiment 21.1) | | (Experiment 21.2) | | (Experiment 22.2) | |
| | | Max | | Max | | Max | | Max | | Max |
| | | Activation | | Activation | | Cyto- | | Cyto- | | Activation |
| | EC50 | (% | EC50 | (% | EC50 | toxicity | EC50 | toxicity | EC50 | (% |
| Antibody | (nM) | Positive) | (nM) | Positive) | (nM) | (%) | (nM) | (%) | (nM) | Positive) |
| Hu47A5H2-11 | 0.039 | 62.62 | 0.131 | 36.94 | 0.008 | 79.18 | 0.036 | 85.63 | 0.379 | 150400 |
| Hu50A8F2-04 | N/A | 44.90 | N/A | 28.90 | 0.070 | 72.72 | N/P | N/P | 0.374 | 107153 |
| Ab9 | 0.118 | 64.72 | 0.436 | 35.70 | N/P | N/P | 0.055 | 87.04 | 0.675 | 140371 |
| KW1.2.1 | 4.946 | 54.48 | None | 29.44 | 0.066 | 77.70 | 0.112 | 84.88 | N/A | 99485 |
| KW7.3.7 | 36.29 | 52.92 | None | 30.62 | 0.206 | 76.36 | 0.440 | 85.31 | N/A | 90075 |
| HP-3G10 | 1.071 | 62.28 | 2.510 | 36.18 | N/P | N/P | 0.325 | 80.83 | 12.560 | 172217 |
| DX12 | 6.137 | 52.27 | 7.956 | 32.00 | N/P | N/P | N/A | N/A | N/A | 85140 |
| 191B8 | 0.085 | 62.89 | 0.248 | 36.87 | N/P | N/P | 0.064 | 84.23 | 0.392 | 128350 |
| hIgG1 | N/A | 46.72 | N/A | 28.69 | N/A | 61.29 | N/A | 78.85 | N/A | 86861 |
| mIgG1 | N/A | 48.70 | N/A | 32.89 | N/P | N/P | N/A | 77.40 | N/A | 85055 |
| mIgG2a | N/A | 49.60 | N/A | 33.00 | N/P | N/P | N/A | 78.60 | N/A | 79288 |

N/A = not applicable.
N/P = not performed.
None = no effect.

TABLE 40
Fold-Ratio of Hu47A5H2-11 to Other Antibodies. Table 39 values for Hu47A5H2-11 were normalized
to 1.00. For EC50, fold-ratio of >1 for a comparator antibody indicates better performance of
Hu47A4H2-11 than the comparator antibody (e.g., improved potency). For maximum effect, fold-ratio
of <1 for a comparator antibody indicates better performance of Hu47A4H2-11 than the comparator
antibody (e.g., improved maximum activation).
Functional Activation

| | NK Activation | | NK Activation | | NK Killing | | NK Killing | | T Cell Activation | |
|---|---|---|---|---|---|---|---|---|---|---|
| | CD107a | | IFNg | | (Experiment 21.1) | | (Experiment 21.2) | | (Experiment 22.2) | |
| | | Max | | Max | | Max | | Max | | Max |
| | | Activation | | Activation | | Cyto- | | Cyto- | | Activation |
| | | (% | | (% | | toxicity | | toxicity | | (% |
| Antibody | EC50 | Positive) | EC50 | Positive) | EC50 | (%) | EC50 | (%) | EC50 | Positive) |
| Hu47A5H2-11 | 1.00 | 1.00 | 1.00 | 1.00 | 1.00 | 1.00 | 1.00 | 1.00 | 1.00 | 1.00 |
| Hu50A8F2-04 | N/A | 0.72 | N/A | 0.78 | 8.48 | 1.09 | N/P | N/P | 0.99 | 0.71 |
| Ab9 | 2.99 | 1.03 | 3.34 | 0.97 | N/P | N/P | 1.53 | 1.02 | 1.78 | 0.93 |
| KW1.2.1 | 125.50 | 0.87 | None | 0.80 | 7.93 | 0.98 | 3.11 | 0.99 | N/A | 0.66 |
| KW7.3.7 | 920.83 | 0.85 | None | 0.83 | 24.99 | 0.96 | 12.22 | 1.00 | N/A | 0.60 |
| HP-3G10 | 27.18 | 0.99 | 19.22 | 0.98 | N/P | N/P | 9.03 | 0.94 | 33.15 | 1.15 |
| DX12 | 155.72 | 0.83 | 60.92 | 0.87 | N/P | N/P | N/A | N/A | N/A | 0.57 |
| 191B8 | 2.17 | 1.00 | 1.90 | 1.00 | N/P | N/P | 1.72 | 0.98 | 1.03 | 0.85 |

N/A = not applicable.
N/P = not performed.
None = no effect.

### REFERENCES

Almagro, J. C., et al. (2008). “Humanization of antibodies.” Front Biosci 13:1619-1633. Aldemir, H., et al. (2005). “Cutting edge: lectin-like transcript 1 is a ligand for the CD161 receptor.” J Immunol 175 (12): 7791-7795.
Basdeo, S. A., et al. (2017). “Ex-Th17 (Nonclassical Th1) Cells Are Functionally Distinct from Classical Th1 and Th17 Cells and Are Not Constrained by Regulatory T Cells.” J Immunol 198(6): 2249-2259.
Fergusson, J. R., et al. (2014). “CD161 defines a transcriptional and functional phenotype across distinct human T cell lineages.” Cell Rep 9 (3): 1075-1088.
Maggi, L., et al. (2010). “CD161 is a marker of all human IL-17-producing T-cell subsets and is induced by RORC.” Eur J Immunol 40 (8): 2174-81.
Mathewson, N. D., et al. (2021). “Inhibitory CD161 receptor identified in glioma-infiltrating T cells by single-cell analysis.” Cell 184 (5): 1281-1298.
Satoh, M., et al. (2008). “Non-fucosylated therapeutic antibodies as next-generation therapeutic antibodies.” Expert Opin Biol Ther 6 (11): 1161-1173.
Yang, J., et al. (2014). “Targeting Th17 cells in autoimmune diseases.” Trends Pharmacol Sci 35(10): 493-500.

What is claimed is:

1. An antibody or antigen binding portion thereof that specifically binds to killer cell lectin-like receptor subfamily B, member 1, (KLRB1; optionally SEQ ID NO:1, SEQ ID NO: 2, or SEQ ID NO:3), wherein the antibody or antigen binding portion thereof comprises at least one of
(a) a heavy chain variable region (VH) comprising a VH complementarity determining region (CDR) 1 comprising a sequence that is at least 95% identical to a VH CDR1 amino acid sequence set forth in one of Table 1 to 18, preferably Table 9, 8, or 18; a VH CDR2 comprising a sequence that is at least 95% identical to a VH CDR2 amino acid sequence set forth in one of Table 1 to 18, preferably Table 9, 8, or 18; and a VH CDR3 comprising a sequence that is at least 95% identical to a VH CDR3 amino acid sequence set forth in one of Table 1 to 18, preferably Table 9, 8, or 18; and
(b) a light chain variable region (VL) comprising a VL CDR1 comprising a sequence that is at least 95% identical to a VL CDR1 amino acid sequence set forth in one of Table 1 to 18, preferably Table 9, 8, or 18, a VL CDR2 comprising a sequence that is at least 95% identical to a VL CDR2 amino acid sequence set forth in one of Table 1 to 18, preferably Table 9, 8, or 18, and a VL CDR3 comprising a sequence that is at least 95% identical to a VL CDR3 amino acid sequence set forth in one of Table 1 to 18, preferably Table 9, 8, or 18.

2. The antibody or antigen binding portion thereof of claim 1, wherein the VH comprises or consists of an VH amino acid sequence set forth in Table 9, 8, or 18.

3. The antibody or antigen binding portion thereof of claim 1, wherein the VL comprises or consists of an VL amino acid sequence set forth in Table 9, 8, or 18.

4. The antibody or antigen binding portion thereof of claim 1, wherein the VH comprises or consists of the VH amino acid sequence set forth in Table 9, and the VL comprises or consists of the VH amino acid sequence set forth in Table 9.

5. The antibody or antigen binding portion thereof of claim 1, wherein the VH comprises or consists of the VH amino acid sequence set forth in Table 8, and the VL comprises or consists of the VH amino acid sequence set forth in Table 8.

6. The antibody or antigen binding portion thereof of claim 1, wherein the VH comprises or consists of the VH amino acid sequence set forth in Table 18, and the VL comprises or consists of the VH amino acid sequence set forth in Table 18.

7. The antibody or antigen binding portion thereof of claim 1, wherein the VH comprises or consists of the VH amino acid sequence set forth in Table 3 or 5, and the VL comprises or consists of the VH amino acid sequence set forth in Table 3 or 5.

8. The antibody or antigen binding portion thereof of claim 1, wherein the VH comprises or consists of an amino acid sequence having at least 95% sequence identity to the VH amino acid sequence set forth in Table 9, 8, or 18, and the VL comprises or consists of an amino acid sequence having at least 95% sequence identity to the VL amino acid sequence set forth in (the same) Table 9, 8, or 18.

9. The antibody or antigen binding portion thereof of claim 1, wherein the VH comprises or consists of an amino acid sequence having at least 95% sequence identity to the VH amino acid sequence set forth in Table C, and the VL comprises or consists of an amino acid sequence having at least 95% sequence identity to the amino acid sequence set forth in Table C, preferably wherein the VH and VL are from the same row in the Table.

10. The antibody or antigen binding portion thereof of claims 1-9, which comprises a constant region heavy chain and light chain, wherein the constant region heavy chain and/or light chain comprises or consists of an amino acid sequence set forth in Table A.

11. An antibody or antigen binding portion thereof that specifically binds to human killer cell lectin-like receptor subfamily B, member 1, (KLRB1; SEQ ID NO:1), comprising or consisting of a variable region heavy chain consisting of the VH amino acid sequence set forth in Table 9 and a variable region light chain consisting of the VL amino acid sequence set forth in Table 9, and optionally a constant region, optionally wherein the antibody or antigen binding portion thereof comprises a sequence that is at least 95% identical to a sequence set forth in Table C.

12. An antibody or antigen binding portion thereof that specifically binds to human killer cell lectin-like receptor subfamily B, member 1, (KLRB1; SEQ ID NOs: 1), comprising or consisting of a variable region heavy chain consisting of the VH amino acid sequence set forth in Table 8 and a variable region light chain consisting of the VL amino acid sequence set forth in Table 8, and optionally a constant region, optionally wherein the antibody or antigen binding portion thereof comprises a sequence that is at least 95% identical to a sequence set forth in Table C.

13. An antibody or antigen binding portion thereof that specifically binds to human killer cell lectin-like receptor subfamily B, member 1, (KLRB1; SEQ ID NO:1), comprising or consisting of a variable region heavy chain consisting of the HC amino acid sequence set forth in Table 18, and a variable region light chain consisting of the LC amino acid sequence set forth in Table 18, and optionally a constant region, optionally wherein the antibody or antigen binding portion thereof comprises a sequence that is at least 95% identical to a sequence set forth in Table C.

14. An antibody or antigen binding portion thereof that specifically binds to human killer cell lectin-like receptor subfamily B, member 1, (KLRB1; SEQ ID NO:1), comprising or consisting of a variable region heavy chain consisting of the VH amino acid sequence set forth in Table 3 or 5, and a variable region light chain consisting of the VL amino acid sequence set forth in Table 3 or 5, and optionally a constant region, optionally wherein the antibody or antigen binding portion thereof comprises a sequence that is at least 95% identical to a sequence set forth in Table C.

15. The antibody or antigen binding portion thereof of claims 1 to 14, further comprising a heavy chain constant region hinge region and Fc domain.

16. The antibody or antigen binding portion thereof of claim 1, which is an antibody that comprises a heavy chain constant region comprising an amino acid sequence having at least 80%, 90%, 95%, or 97% sequence identity to a heavy chain constant region amino acid sequence set forth in Table A.

17. The antibody or antigen binding portion thereof of claim 1, wherein the antibody is a monoclonal antibody.

18. The antibody or antigen binding portion thereof of claim 1, wherein the antibody is a chimeric, humanized, or human antibody, and/or comprises one or more mutations (optionally in a CDR) that remove Asn (N)-glycosylation sites or remove Cys, Asp, Met, Trp or Lys.

19. The antibody or antigen binding portion thereof of claim 1, wherein the antibody is an immunoglobulin G (IgG) subtype IgG1 antibody, an IgG2 antibody, or an IgG4 antibody.

20. The antibody or antigen binding portion thereof of claim 1, which is an antibody that comprises an Fc region, preferably human IgG1, that binds to Fc gamma receptors (FcγRs) and induces antibody dependent cell-mediated cytotoxicity (ADCC) to deplete cells expressing KLRB1, or that binds to C1q and induce complement dependent cytotoxicity (CDC).

21. The antibody or antigen binding portion thereof of claim 1, which is conjugated to a cytotoxic agent.

22. The antibody or antigen binding portion thereof of claim 1, which comprises an Fc region that is afucosylated

23. A polynucleotide comprising a nucleic acid sequence encoding an antibody or antigen binding portion thereof of claims 1 to 22.

24. The polynucleotide of claim 23, wherein the nucleic acid sequence is operably linked to a promoter.

25. A vector comprising the polynucleotide of any one of claims 23-24.

26. A host cell comprising the polynucleotide of any one of claims 23-24, or the vector of claim 25, and optionally expressing the antibody or antigen binding portion thereof.

27. A pharmaceutical composition that comprises the antibody or antigen binding portion thereof of any one of the preceding claims, and a pharmaceutically acceptable carrier or diluent.

28. A method of making the antibody or antigen binding portion thereof of any one of the preceding claims, comprising culturing the host cell of claim 26 under conditions sufficient to express the antibody or antigen binding portion thereof and isolating the antibody or antigen binding portion thereof.

29. The method of claim 28, further comprising formulating the antibody as a pharmaceutical composition.

30. A method of treating one or more of an autoimmune disease, an allergic disease, a transplant rejection, and a hematologic malignancy in a subject in need thereof, the method comprising administering to the subject an antibody that binds KLRB1, preferably the antibody of any one of claims 1-22, the polynucleotide of any one of claims 23-24, the vector of claim 25, the pharmaceutical composition of claim 27, or the host cell of claim 26 that expresses the antibody or antigen binding portion thereof.

31. The method of claim 30, wherein the autoimmune disease is rheumatoid arthritis, Sjogren's syndrome, inclusion body myositis (IBM), discoid lupus, psoriasis, idiopathic pulmonary fibrosis, diabetes, alopecia universalis, primary biliary cholangitis, multiple sclerosis, lymphocytic colitis, palmoplantar pustulosis, or hidradenitis suppurativa.

32. The method of claim 30, wherein the allergic disease is asthma, allergic eosinophilic asthma, allergy, atopic dermatitis, nasal polyposis, eosinophilic gastrointestinal disorder, or hypereosinophilic syndrome.

33. The method of claim 30, wherein the transplant rejection can be a rejection of a kidney, lung, heart, liver, limb, skin, or multi-organ transplant.

34. The method of claim 30, wherein the hematological malignancy is a lymphoma or leukemia.

35. The method of claim 34, wherein the lymphoma NK/T cell lymphoma, mycosis fungoides, Sezary syndrome, peripheral T cell lymphoma, angioimmunoblastic T cell lymphoma (AITL), or peripheral T cell lymphoma not otherwise specified (PTCL-NOS) or mycosis fungoides.

36. The method of claim 34, wherein the leukemia is T cell leukemia, aggressive NK cell leukemia, T cell prolymphocytic leukemia (T-PLL), or large granular lymphocytic leukemia (LGLL).

37. The antibody of any one of claims 1-22, the polynucleotide of any one of claims 23-24, the vector of claim 25, or the method of claims 30-36, wherein the antibody binds to and depletes Th17, Th17.1, ex-Th17, Tc17, mucosal-associated invariant T cells (MAIT), invariant NK-T cells (INKT), innate lymphoid cells types 2 and 3 (ILC2, and ILC3), pathogenic effector Th2 (peTh2) cells, NK cells, and/or neoplastic T or NK cells expressing KLRB1.

38. The antibody of any one of claims 1-22, which is not clone B199.2 (Invitrogen), HP-3G10 (Invitrogen), OTI1D8 (OriGene), 14F1F11 (OriGene), 702228 (R&D Systems), B-D51 (Cell Sciences), 2F3 (Novus Biologics), EP7169 (Abcam), DX1, DX12, 191B8, Ab9, KW1.2.1, KW7.3.7, or JNH25G2G22 (Creative Diagnostics).

39. The antibody of any one of claims 1-22, the polynucleotide of any one of claims 23-24, or the vector of claim 25, for use in a method of treating one or more of an autoimmune disease, an allergic disease, a transplant rejection, and a hematologic malignancy in a subject in need thereof.

40. The antibody, polynucleotide, or vector for the use of claim 39, wherein the autoimmune disease is rheumatoid arthritis, Sjogren's syndrome, inclusion body myositis (IBM), discoid lupus, psoriasis, idiopathic pulmonary fibrosis, diabetes, alopecia universalis, primary biliary cholangitis, multiple sclerosis, lymphocytic colitis, palmoplantar pustulosis, or hidradenitis suppurativa.

41. The antibody, polynucleotide, or vector for the use of claim 39, wherein the allergic disease is asthma, allergic eosinophilic asthma, allergy, atopic dermatitis, nasal polyposis, eosinophilic gastrointestinal disorder, or hypereosinophilic syndrome.

42. The antibody, polynucleotide, or vector for the use of claim 39, wherein the transplant rejection can be a rejection of a kidney, lung, heart, liver, limb, skin, or multi-organ transplant.

43. The antibody, polynucleotide, or vector for the use of claim 39, wherein the hematological malignancy is a lymphoma or leukemia.

44. The antibody, polynucleotide, or vector for the use of claim 43, wherein the lymphoma is NK/T cell lymphoma, mycosis fungoides, Sezary syndrome, peripheral T cell lymphoma, angioimmunoblastic T cell lymphoma (AITL), or peripheral T cell lymphoma not otherwise specified (PTCL-NOS).

45. The antibody, polynucleotide, or vector for the use of claim 43, wherein the leukemia is T cell leukemia, NK cell leukemia, T cell prolymphocytic leukemia (T-PLL), or large granular lymphocytic leukemia (LGLL).


SEQUENCE LISTING (586 sequences)

SEQ ID NO: 1 - protein, 225 aa, Homo sapiens
  source 1..225: note = HumanKLRB1-Hum-KLRB1
MDQQAIYAELNLPTDSGPESSSPSSLPRDVCQGSPWHQFALKLSCAGIILLVLVVTGLSVSVTSLIQKSSIEKCSVDIQQSRNKTTERPGLLNCPIYWQQLREKCLLFSHTVNPWNNSLADCSTKESSLLLIRDKDELIHTQNLIRDKAILFWIGLNFSLSEKNWKWINGSFLNSNDLEIRGDAKENSCISISQTSVYSEYCSTEIRWICQKELTPVRNKVYPDS

SEQ ID NO: 2 - protein, 227 aa, Macaca fascicularis
  source 1..227: note = CynoKLRB1UniParc_UPI0003ABB264-Cyno,retired
MDQQMMYAELTLPKDSGPESSSPSSLPRDVCQGSPWHQFALKLSCAGIILLVLVVTGLSLSVASLLQKPSIGKCSVDIQQNRTKTTERPDLLNCPIYWQQVQEKCLLFSHTVNPWNNSLADCSTKESSLLLIQDKDELTRTQNLIHDKAISFWIGLNFSLSEKNWKWINGSFLSSNDLKITGDAKENSCVYISQTSVYSEYCSTEMKWICQKELTLVRNKVSPDSWL

SEQ ID NO: 3 - protein, 227 aa, Macaca fascicularis
  source 1..227: note = CynoKLRB1UniprotA0A7N9D796-Cyno,current
MDQQMMYAELTLPKDSGPESSSPSSLPRDVCQGSPWHQFALKLSCAGIILLVLVVTGLSLSVASLLQKPSIGKCSVDIQQNRTKTTERPDLLNCPIYWKQVQEKCLLFSHTVNPWNNSLADCSTKESSLLLIQDKDELTRTQNLIHDKAISFWIGLNFSLSEKNWKWINGSFLSSNDLKITGDAKENSCVYISQTSVYSEYCSTEMKWICQKELTLVRNKVSPDSWL

SEQ ID NO: 4 - protein, 330 aa, Homo sapiens [sequence omitted]

SEQ ID NO: 5 - protein, 330 aa, Homo sapiens
  source 1..330: note = Human IgG1 Constant (IGHG1) LALAGA / G1m(z) allele with L234A/L235A/G237A-CH
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 213 aa, Homo sapiens
  source 1..213: note = Human IgG1 Fc region / WT-Fc
LGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 107 aa, Homo sapiens [sequence omitted]

SEQ ID NO: 8 - protein, 324 aa, Mus musculus
  source 1..324: note = Mouse IgG1 Constant (IGHG1 Mouse) / Uniprot P01868-CH
AKTTPPSVYPLAPGSAAQTNSMVTLGCLVKGYFPEPVTVTWNSGSLSSGVHTFPAVLQSDLYTLSSSVTVPSSPRPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGK

SEQ ID NO: 9 - protein, 330 aa, Mus musculus
  source 1..330: note = Mouse IgG2a Constant (IGHG Mouse) / Uniprot P01863-CH
AKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 10 - protein, 404 aa, Mus musculus
  source 1..404: note = Mouse IgG2b Constant (IGG2B Mouse) / Uniprot P01867-CH
KTTPPSVYPLAPGCGDTTGSSVTLGCLVKGYFPESVTVTWNSGSLSSSVHTFPALLQSGLYTMSSSVTVPSSTWPSQTVTCSVAHPASSTTVDKKLEPSGPISTINPCPPCKECHKCPAPNLEGGPSVFIFPPNIKDVLMISLTPKVTCVVVDVSEDDPDVQISWFVNNVEVHTAQTQTHREDYNSTIRVVSTLPIQHQDWMSGKEFKCKVNNKDLPSPIERTISKIKGLVRAPQVYILPPPAEQLSRKDVSLTCLVVGFNPGDISVEWTSNGHTEENYKDTAPVLDSDGSYFIYSKLNMKTSKWEKTDSFSCNVRHEGLKNYYLKKTISRSPGLDLDDICAEAKDGELDGLWTTITIFISLFLLSVCYSASVTLFKVKWIFSSVVELKQKISPDYRNMIGQGA

SEQ ID NO: 11 - protein, 107 aa, Mus musculus
  source 1..107: note = Mouse IgK Constant (IGKC Mouse) / Uniprot P01837-CL
RADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 12 - protein, 5 aa, synthetic construct
  source 1..5: note = 18G11F11-Kabat-CDRH1
DYGVN

SEQ ID NO: 13 - protein, 16 aa, synthetic construct
  source 1..16: note = 18G11F11-Kabat-CDRH2
MIWGDGSTDYNSALKS

SEQ ID NO: 14 - protein, 12 aa, synthetic construct
  source 1..12: note = 18G11F11-Kabat-CDRH3
TYNSYHYWYFDV

SEQ ID NO: 15 - protein, 11 aa, synthetic construct
  source 1..11: note = 18G11F11-Kabat-CDRL1
RASGNIHNYLA

SEQ ID NO: 16 - protein, 7 aa, synthetic construct
  source 1..7: note = 18G11F11-Kabat-CDRL2
NAETLAD

SEQ ID NO: 17 - protein, 9 aa, synthetic construct
  source 1..9: note = 18G11F11-Kabat-CDRL3
QHFWSTPYT

SEQ ID NO: 18 - protein, 7 aa, synthetic construct
  source 1..7: note = 18G11F11-Chothia-CDRH1
GFSLTDY

SEQ ID NO: 19 - protein, 5 aa, synthetic construct
  source 1..5: note = 18G11F11-Chothia-CDRH2
WGDGS

SEQ ID NO: 20 - protein, 10 aa, synthetic construct
  source 1..10: note = 18G11F11-AbM-CDRH1
GFSLTDYGVN

SEQ ID NO: 21 - protein, 9 aa, synthetic construct
  source 1..9: note = 18G11F11-AbM-CDRH2
MIWGDGSTD

SEQ ID NO: 22 - protein, 6 aa, synthetic construct
  source 1..6: note = 18G11F11-Contact-CDRH1
TDYGVN

SEQ ID NO: 23 - protein, 12 aa, synthetic construct
  source 1..12: note = 18G11F11-Contact-CDRH2
WLGMIWGDGSTD

SEQ ID NO: 24 - protein, 13 aa, synthetic construct
  source 1..13: note = 18G11F11-Contact-CDRH3
ARTYNSYHYWYFD

SEQ ID NO: 25 - protein, 7 aa, synthetic construct
  source 1..7: note = 18G11F11-Contact-CDRL1
HNYLAWY

SEQ ID NO: 26 - protein, 10 aa, synthetic construct
  source 1..10: note = 18G11F11-Contact-CDRL2
LLVYNAETLA

SEQ ID NO: 27 - protein, 8 aa, synthetic construct
  source 1..8: note = 18G11F11-Contact-CDRL3
QHFWSTPY

SEQ ID NO: 28 - protein, 8 aa, synthetic construct
  source 1..8: note = 18G11F11-IMGT-CDRH1
GFSLTDYG

SEQ ID NO: 29 - protein, 7 aa, synthetic construct
  source 1..7: note = 18G11F11-IMGT-CDRH2
IWGDGST

SEQ ID NO: 30 - protein, 14 aa, synthetic construct
  source 1..14: note = 18G11F11-IMGT-CDRH3
ARTYNSYHYWYFDV

SEQ ID NO: 31 - protein, 6 aa, synthetic construct
  source 1..6: note = 18G11F11-IMGT-CDRL1
GNIHNY

SEQ ID NO: 32 - skipped

SEQ ID NO: 33 - protein, 120 aa, synthetic construct
  source 1..120: note = 18G11F11-VH
QVQLKESGPGLVAPSQSLSITCTVSGFSLTDYGVNWVRQPPGKGLEWLGMIWGDGSTDYNSALKSRLTINKDNSKSQVFLNMNSLHTDDTASYYCARTYNSYHYWYFDVWGAGTAVTVSS

SEQ ID NO: 34 - protein, 107 aa, synthetic construct
  source 1..107: note = 18G11F11-VL
DIQMTQSPASLSASVGETVTITCRASGNIHNYLAWYQQKQGKSPQLLVYNAETLADGVPSRFSGSGSGTQYSLKINSLQPEDFGSYYCQHFWSTPYTFGGGTKLEIK

SEQ ID NO: 35 - protein, 5 aa, synthetic construct
  source 1..5: note = 9C10G11-Kabat-CDRH1
GYGVN

SEQ ID NO: 36 - protein, 12 aa, synthetic construct
  source 1..12: note = 9C10G11-Kabat-CDRH3
TYNNYHYWYFDV

SEQ ID NO: 37 - protein, 7 aa, synthetic construct
  source 1..7: note = 9C10G11-Kabat-CDRL2
NAKTLAD

SEQ ID NO: 38 - protein, 7 aa, synthetic construct
  source 1..7: note = 9C10G11-Chothia-CDRH1
GFSLTGY

SEQ ID NO: 39 - protein, 10 aa, synthetic construct
  source 1..10: note = 9C10G11-AbM-CDRH1
GFSLTGYGVN

SEQ ID NO: 40 - protein, 6 aa, synthetic construct
  source 1..6: note = 9C10G11-Contact-CDRH1
TGYGVN

SEQ ID NO: 41 - protein, 13 aa, synthetic construct
  source 1..13: note = 9C10G11-Contact-CDRH3
ARTYNNYHYWYFD

SEQ ID NO: 42 - protein, 10 aa, synthetic construct
  source 1..10: note = 9C10G11-Contact-CDRL2
LLVYNAKTLA

SEQ ID NO: 43 - protein, 8 aa, synthetic construct
  source 1..8: note = 9C10G11-IMGT-CDRH1
GFSLTGYG

SEQ ID NO: 44 - protein, 14 aa, synthetic construct
  source 1..14: note = 9C10G11-IMGT-CDRH3
ARTYNNYHYWYFDV

SEQ ID NO: 45 - protein, 120 aa, synthetic construct
  source 1..120: note = 9C10G11-VH
QVQLKESGPGLVAPSQSLSITCTVSGFSLTGYGVNWVRQPPGKGLEWLGMIWGDGSTDYNSALKSRLSISKDNSKSQVFLKMNSLQTDDTARYYCARTYNNYHYWYFDVWGAGTTVTVSS

SEQ ID NO: 46 - protein, 107 aa, synthetic construct
  source 1..107: note = 9C10G11-VL
DIQMTQSPASLSASVGETVTITCRASGNIHNYLAWYQQKQGKSPQLLVYNAKTLADGVPSRFSGSGSGTQYSLKINSLQPEDFGSYYCQHFWSTPYTFGGGTKLEIK

SEQ ID NO: 47 - protein, 6 aa, synthetic construct
  source 1..6: note = 57E2E4-Kabat-CDRH1
SGYYWN

SEQ ID NO: 48 - protein, 16 aa, synthetic construct
  source 1..16: note = 57E2E4-Kabat-CDRH2
YISYDGSNNYNPSLKN

SEQ ID NO: 49 - protein, 8 aa, synthetic construct
  source 1..8: note = 57E2E4-Kabat-CDRH3
GDFYGYVY

SEQ ID NO: 50 - protein, 15 aa, synthetic construct
  source 1..15: note = 57E2E4-Kabat-CDRL1
RASKRVSTSDYSYMH

SEQ ID NO: 51 - protein, 7 aa, synthetic construct
  source 1..7: note = 57E2E4-Kabat-CDRL2
LASNLES

SEQ ID NO: 52 - protein, 9 aa, synthetic construct
  source 1..9: note = 57E2E4-Kabat-CDRL3
QHSREFPRT

SEQ ID NO: 53 - protein, 8 aa, synthetic construct
  source 1..8: note = 57E2E4-Chothia-CDRH1
GYSISSGY

SEQ ID NO: 54 - protein, 5 aa, synthetic construct
  source 1..5: note = 57E2E4-Chothia-CDRH2
SYDGS

SEQ ID NO: 55 - protein, 11 aa, synthetic construct
  source 1..11: note = 57E2E4-AbM-CDRH1
GYSISSGYYWN

SEQ ID NO: 56 - protein, 9 aa, synthetic construct
  source 1..9: note = 57E2E4-AbM-CDRH2
YISYDGSNN

SEQ ID NO: 57 - protein, 7 aa, synthetic construct
  source 1..7: note = 57E2E4-Contact-CDRH1
SSGYYWN

SEQ ID NO: 58 - protein, 12 aa, synthetic construct
  source 1..12: note = 57E2E4-Contact-CDRH2
WMAYISYDGSNN

SEQ ID NO: 59 - protein, 9 aa, synthetic construct
  source 1..9: note = 57E2E4-Contact-CDRH3
TRGDFYGYV

SEQ ID NO: 60 - protein, 11 aa, synthetic construct
  source 1..11: note = 57E2E4-Contact-CDRL1
STSDYSYMHWY

SEQ ID NO: 61 - protein, 10 aa, synthetic construct
  source 1..10: note = 57E2E4-Contact-CDRL2
LLIYLASNLE

SEQ ID NO: 62 - protein, 8 aa, synthetic construct
  source 1..8: note = 57E2E4-Contact-CDRL3
QHSREFPR

SEQ ID NO: 63 - protein, 9 aa, synthetic construct
  source 1..9: note = 57E2E4-IMGT-CDRH1
GYSISSGYY

SEQ ID NO: 64 - protein, 7 aa, synthetic construct
  source 1..7: note = 57E2E4-IMGT-CDRH2
ISYDGSN

SEQ ID NO: 65 - protein, 10 aa, synthetic construct
  source 1..10: note = 57E2E4-IMGT-CDRH3
TRGDFYGYVY

SEQ ID NO: 66 - protein, 10 aa, synthetic construct
  source 1..10: note = 57E2E4-IMGT-CDRL1
KRVSTSDYSY

SEQ ID NO: 67 - skipped

SEQ ID NO: 68 - protein, 117 aa, synthetic construct
  source 1..117: note = 57E2E4-VH
DVQLQESGPGLVKPSQSLSLTCSVTGYSISSGYYWNWIRQFPGNKLEWMAYISYDGSNNYNPSLKNRVSITRDTSKNQFFLRLNSVTAEDTATYYCTRGDFYGYVYWGQGTTLTVSL

SEQ ID NO: 69 - protein, 111 aa, synthetic construct
  source 1..111: note = 57E2E4-VL
DIVLTQSPASLAVSLGQRATISCRASKRVSTSDYSYMHWYQQKPGQPPKLLIYLASNLESGVPARFSGSGSGTDFTLNIHPVEEEDAATYYCQHSREFPRTFGGGTKLEIK

SEQ ID NO: 70 - protein, 5 aa, synthetic construct
  source 1..5: note = 75B10F12-Kabat-CDRH1
SFWIE

SEQ ID NO: 71 - protein, 17 aa, synthetic construct
  source 1..17: note = 75B10F12-Kabat-CDRH2
EIFPGSGRTNFNERFKD

SEQ ID NO: 72 - protein, 9 aa, synthetic construct
  source 1..9: note = 75B10F12-Kabat-CDRH3
FNYDVRFDF

SEQ ID NO: 73 - protein, 11 aa, synthetic construct
  source 1..11: note = 75B10F12-Kabat-CDRL1
RASENVYNNLA

SEQ ID NO: 74 - protein, 7 aa, synthetic construct
  source 1..7: note = 75B10F12-Kabat-CDRL2
GATNLAD

SEQ ID NO: 75 - protein, 9 aa, synthetic construct
  source 1..9: note = 75B10F12-Kabat-CDRL3
QHFWGTPWT

SEQ ID NO: 76 - protein, 7 aa, synthetic construct
  source 1..7: note = 75B10F12-Chothia-CDRH1
GYTFSSF

SEQ ID NO: 77 - protein, 6 aa, synthetic construct
  source 1..6: note = 75B10F12-Chothia-CDRH2
FPGSGR

SEQ ID NO: 78 - protein, 10 aa, synthetic construct
  source 1..10: note = 75B10F12-AbM-CDRH1
GYTFSSFWIE

SEQ ID NO: 79 - protein, 10 aa, synthetic construct
  source 1..10: note = 75B10F12-AbM-CDRH2
EIFPGSGRTN

SEQ ID NO: 80 - protein, 6 aa, synthetic construct
  source 1..6: note = 75B10F12-Contact-CDRH1
SSFWIE

SEQ ID NO: 81 - protein, 13 aa, synthetic construct
  source 1..13: note = 75B10F12-Contact-CDRH2
WIGEIFPGSGRTN

SEQ ID NO: 82 - protein, 10 aa, synthetic construct
  source 1..10: note = 75B10F12-Contact-CDRH3
ARFNYDVRFD

SEQ ID NO: 83 - protein, 7 aa, synthetic construct
  source 1..7: note = 75B10F12-Contact-CDRL1
YNNLAWY

SEQ ID NO: 84 - protein, 10 aa, synthetic construct
  source 1..10: note = 75B10F12-Contact-CDRL2
LLIYGATNLA

SEQ ID NO: 85 - protein, 8 aa, synthetic construct
  source 1..8: note = 75B10F12-Contact-CDRL3
QHFWGTPW

SEQ ID NO: 86 - protein, 8 aa, synthetic construct
  source 1..8: note = 75B10F12-IMGT-CDRH1
GYTFSSFW

SEQ ID NO: 87 - protein, 8 aa, synthetic construct
  source 1..8: note = 75B10F12-IMGT-CDRH2
IFPGSGRT

SEQ ID NO: 88 - protein, 11 aa, synthetic construct
  source 1..11: note = 75B10F12-IMGT-CDRH3
ARFNYDVRFDF

SEQ ID NO: 89 - protein, 6 aa, synthetic construct
  source 1..6: note = 75B10F12-IMGT-CDRL1
ENVYNN

SEQ ID NO: 90 - skipped

SEQ ID NO: 91 - protein, 118 aa, synthetic construct
  source 1..118: note = 75B10F12-VH
QVQLQQSGAELMKPGASVKISCKATGYTFSSFWIEWLKQRPGHGLEWIGEIFPGSGRTNFNERFKDKATFAADTSSNTAYMQLRSLTSEDSAVYYCARFNYDVRFDFWGQGTTLTVSS

SEQ ID NO: 92 - protein, 107 aa, synthetic construct
  source 1..107: note = 75B10F12-VL
DIQMTQSPVSLSISVGETVTITCRASENVYNNLAWYQQKQGKSPQLLIYGATNLADGVPSRFSGSGSGTQFSLKINSLQSEDFGTYYCQHFWGTPWTFGGGTKLEIK

SEQ ID NO: 93 - protein, 12 aa, synthetic construct
  source 1..12: note = 7B6C7-Kabat-CDRH3
TYNYYHYWYFDV

SEQ ID NO: 94 - protein, 11 aa, synthetic construct
  source 1..11: note = 7B6C7-Kabat-CDRL1
RASGNIHNYLS

SEQ ID NO: 95 - protein, 7 aa, synthetic construct
  source 1..7: note = 7B6C7-Chothia-CDRH1
GFSLNDY

SEQ ID NO: 96 - protein, 10 aa, synthetic construct
  source 1..10: note = 7B6C7-AbM-CDRH1
GFSLNDYGVN

SEQ ID NO: 97 - protein, 6 aa, synthetic construct
  source 1..6: note = 7B6C7-Contact-CDRH1
NDYGVN

SEQ ID NO: 98 - protein, 13 aa, synthetic construct
  source 1..13: note = 7B6C7-Contact-CDRH3
ARTYNYYHYWYFD

SEQ ID NO: 99 - protein, 7 aa, synthetic construct
  source 1..7: note = 7B6C7-Contact-CDRL1
HNYLSWY

SEQ ID NO: 100 - protein, 8 aa, synthetic construct
  source 1..8: note = 7B6C7-IMGT-CDRH1
GFSLNDYG

SEQ ID NO: 101 - protein, 14 aa, synthetic construct
  source 1..14: note = 7B6C7-IMGT-CDRH3
ARTYNYYHYWYFDV

SEQ ID NO: 102 - protein, 120 aa, synthetic construct
  source 1..120: note = 7B6C7-VH
QVQLKESGPGLVAPSQSLSITCTVSGFSLNDYGVNWVRQPPGKALEWLGMIWGDGSTDYNSALKSRLTITKDNSKSQVFLKMNSLHTDDTARYFCARTYNYYHYWYFDVWGAGTTVTVSS

SEQ ID NO: 103 - protein, 107 aa, synthetic construct
  source 1..107: note = 7B6C7-VL
DIQMTQSPASLSASVGETVTITCRASGNIHNYLSWYQQKQGKSPQLLVYNAETLADGVPSRFSGSGSGTQYSLKINNLQPEDFGSYYCQHFWSTPYTFGGGTKLEIK

SEQ ID NO: 104 - protein, 5 aa, synthetic construct
  source 1..5: note = 52G9B12B5-Kabat-CDRH1
SFWME

SEQ ID NO: 105 - protein, 17 aa, synthetic construct
  source 1..17: note = 52G9B12B5-Kabat-CDRH2
EIFPGSGSTNYNEKFKG

SEQ ID NO: 106 - protein, 8 aa, synthetic construct
  source 1..8: note = 52G9B12B5-Kabat-CDRH3
LITTGLTY

SEQ ID NO: 107 - protein, 11 aa, synthetic construct
  source 1..11: note = 52G9B12B5-Kabat-CDRL1
KASQDVNTAVA

SEQ ID NO: 108 - protein, 7 aa, synthetic construct
  source 1..7: note = 52G9B12B5-Kabat-CDRL2
WASTRHT

SEQ ID NO: 109 - protein, 9 aa, synthetic construct
  source 1..9: note = 52G9B12B5-Kabat-CDRL3
QQHYRIPFT

SEQ ID NO: 110 - protein, 6 aa, synthetic construct
  source 1..6: note = 52G9B12B5-Chothia-CDRH2
FPGSGS

SEQ ID NO: 111 - protein, 10 aa, synthetic construct
  source 1..10: note = 52G9B12B5-AbM-CDRH1
GYTFSSFWME

SEQ ID NO: 112 - protein, 10 aa, synthetic construct
  source 1..10: note = 52G9B12B5-AbM-CDRH2
EIFPGSGSTN

SEQ ID NO: 113 - protein, 6 aa, synthetic construct
  source 1..6: note = 52G9B12B5-Contact-CDRH1
SSFWME

SEQ ID NO: 114 - protein, 13 aa, synthetic construct
  source 1..13: note = 52G9B12B5-Contact-CDRH2
WIGEIFPGSGSTN

SEQ ID NO: 115 - protein, 9 aa, synthetic construct
  source 1..9: note = 52G9B12B5-Contact-CDRH3
ATLITTGLT

SEQ ID NO: 116 - protein, 7 aa, synthetic construct
  source 1..7: note = 52G9B12B5-Contact-CDRL1
NTAVAWY

SEQ ID NO: 117 - protein, 10 aa, synthetic construct
  source 1..10: note = 52G9B12B5-Contact-CDRL2
LLIYWASTRH

SEQ ID NO: 118 - protein, 8 aa, synthetic construct
  source 1..8: note = 52G9B12B5-Contact-CDRL3
QQHYRIPF

SEQ ID NO: 119 - protein, 8 aa, synthetic construct
  source 1..8: note = 52G9B12B5-IMGT-CDRH2
IFPGSGST

SEQ ID NO: 120 - protein, 10 aa, synthetic construct
  source 1..10: note = 52G9B12B5-IMGT-CDRH3
ATLITTGLTY

SEQ ID NO: 121 - protein, 6 aa, synthetic construct
  source 1..6: note = 52G9B12B5-IMGT-CDRL1
QDVNTA

SEQ ID NO: 122 - skipped

SEQ ID NO: 123 - protein, 117 aa, synthetic construct
  source 1..117: note = 52G9B12B5-VH
QVQLQQSGAELMQPGTSVKIPCKATGYTFSSFWMEWVKQRPGHGLEWIGEIFPGSGSTNYNEKFKGKATFTADTSSNTAYMQLSSLTSEDSAVYYCATLITTGLTYWGQGTLVTVSA

SEQ ID NO: 124 - protein, 107 aa, synthetic construct
  source 1..107: note = 52G9B12B5-VL
NIVMTQSHKFMSTSVGDRVSITCKASQDVNTAVAWYQQIPGRSPKLLIYWASTRHTGVPDRFTGSGSGTDYTLTISSVQAEDLAFYYCQQHYRIPFTFGAGTKLELK

SEQ ID NO: 125 - protein, 5 aa, synthetic construct
  source 1..5: note = 5B1B11-Kabat-CDRH1
NYWMH

SEQ ID NO: 126 - protein, 17 aa, synthetic construct
  source 1..17: note = 5B1B11-Kabat-CDRH2
EINPSNGRTNFNEKFKS

SEQ ID NO: 127 - protein, 11 aa, synthetic construct
  source 1..11: note = 5B1B11-Kabat-CDRH3
SYDSYYAAVDY

SEQ ID NO: 128 - protein, 10 aa, synthetic construct
  source 1..10: note = 5B1B11-Kabat-CDRL1
SASSSVSYMH

SEQ ID NO: 129 - protein, 7 aa, synthetic construct
  source 1..7: note = 5B1B11-Kabat-CDRL2
TTSNLPS

SEQ ID NO: 130 - protein, 9 aa, synthetic construct
  source 1..9: note = 5B1B11-Kabat-CDRL3
HQWSTYPYT

SEQ ID NO: 131 - protein, 7 aa, synthetic construct
  source 1..7: note = 5B1B11-Chothia-CDRH1
GYTFTNY

SEQ ID NO: 132 - protein, 6 aa, synthetic construct
  source 1..6: note = 5B1B11-Chothia-CDRH2
NPSNGR

SEQ ID NO: 133 - protein, 10 aa, synthetic construct
  source 1..10: note = 5B1B11-AbM-CDRH1
GYTFTNYWMH

SEQ ID NO: 134 - protein, 10 aa, synthetic construct
  source 1..10: note = 5B1B11-AbM-CDRH2
EINPSNGRTN

SEQ ID NO: 135 - protein, 6 aa, synthetic construct
  source 1..6: note = 5B1B11-Contact-CDRH1
TNYWMH

SEQ ID NO: 136 - protein, 13 aa, synthetic construct
  source 1..13: note = 5B1B11-Contact-CDRH2
WIGEINPSNGRTN

SEQ ID NO: 137 - protein, 12 aa, synthetic construct
  source 1..12: note = 5B1B11-Contact-CDRH3
ARSYDSYYAAVD

SEQ ID NO: 138 - protein, 6 aa, synthetic construct
  source 1..6: note = 5B1B11-Contact-CDRL1
SYMHWY

SEQ ID NO: 139 - protein, 10 aa, synthetic construct
  source 1..10: note = 5B1B11-Contact-CDRL2
LLIYTTSNLP

SEQ ID NO: 140 - protein, 8 aa, synthetic construct
  source 1..8: note = 5B1B11-Contact-CDRL3
HQWSTYPY

SEQ ID NO: 141 - protein, 8 aa, synthetic construct
  source 1..8: note = 5B1B11-IMGT-CDRH1
GYTFTNYW

SEQ ID NO: 142 - protein, 8 aa, synthetic construct
  source 1..8: note = 5B1B11-IMGT-CDRH2
INPSNGRT

SEQ ID NO: 143 - protein, 13 aa, synthetic construct
  source 1..13: note = 5B1B11-IMGT-CDRH3
ARSYDSYYAAVDY

SEQ ID NO: 144 - protein, 5 aa, synthetic construct
  source 1..5: note = 5B1B11-IMGT-CDRL1
SSVSY

SEQ ID NO: 145 - skipped

SEQ ID NO: 146 - protein, 120 aa, synthetic construct
  source 1..120: note = 5B1B11-VH
QVQLQQPGAELVKPGASVKLSCKASGYTFTNYWMHWVKQRPGQGLEWIGEINPSNGRTNFNEKFKSKATLTVDKSSSTAYMQLSSLTSEDSAVYYCARSYDSYYAAVDYWGQGTSVTVSS

SEQ ID NO: 147 - protein, 106 aa, synthetic construct
  source 1..106: note = 5B1B11-VL
QIVLTQSPAIMSASLGEEITLTCSASSSVSYMHWYQQKSGTSPKLLIYTTSNLPSGVPSRFSGSGSGTFYSLTISSVEAEDAADYYCHQWSTYPYTFGGGTKLEIK

SEQ ID NO: 148 - protein, 5 aa, synthetic construct
  source 1..5: note = 50A8F2-Kabat-CDRH1
DYYIN

SEQ ID NO: 149 - protein, 17 aa, synthetic construct
  source 1..17: note = 50A8F2-Kabat-CDRH2
WIFPGSGHTKYNENFKG

SEQ ID NO: 150 - protein, 11 aa, synthetic construct
  source 1..11: note = 50A8F2-Kabat-CDRH3
GDRSALGYFDY

SEQ ID NO: 151 - protein, 12 aa, synthetic construct
  source 1..12: note = 50A8F2-Kabat-CDRL1
TASSSVSSTYLH

SEQ ID NO: 152 - protein, 7 aa, synthetic construct
  source 1..7: note = 50A8F2-Kabat-CDRL2
STSNLAS

SEQ ID NO: 153 - protein, 9 aa, synthetic construct
  source 1..9: note = 50A8F2-Kabat-CDRL3
HQYHRSPLT

SEQ ID NO: 154 - protein, 7 aa, synthetic construct
  source 1..7: note = 50A8F2-Chothia-CDRH1
GYTFIDY

SEQ ID NO: 155 - protein, 6 aa, synthetic construct
  source 1..6: note = 50A8F2-Chothia-CDRH2
FPGSGH

SEQ ID NO: 156 - protein, 10 aa, synthetic construct
  source 1..10: note = 50A8F2-AbM-CDRH1
GYTFIDYYIN

SEQ ID NO: 157 - protein, 10 aa, synthetic construct
  source 1..10: note = 50A8F2-AbM-CDRH2
WIFPGSGHTK

SEQ ID NO: 158 - protein, 6 aa, synthetic construct
  source 1..6: note = 50A8F2-Contact-CDRH1
IDYYIN

SEQ ID NO: 159 - protein, 13 aa, synthetic construct
  source 1..13: note = 50A8F2-Contact-CDRH2
WIGWIFPGSGHTK

SEQ ID NO: 160 - protein, 12 aa, synthetic construct
  source 1..12: note = 50A8F2-Contact-CDRH3
ARGDRSALGYFD

SEQ ID NO: 161 - protein, 8 aa, synthetic construct
  source 1..8: note = 50A8F2-Contact-CDRL1
SSTYLHWY

SEQ ID NO: 162 - protein, 10 aa, synthetic construct
  source 1..10: note = 50A8F2-Contact-CDRL2
LWIYSTSNLA

SEQ ID NO: 163 - protein, 8 aa, synthetic construct
  source 1..8: note = 50A8F2-Contact-CDRL3
HQYHRSPL

SEQ ID NO: 164 - protein, 8 aa, synthetic construct
  source 1..8: note = 50A8F2-IMGT-CDRH1
GYTFIDYY

SEQ ID NO: 165 - protein, 8 aa, synthetic construct
  source 1..8: note = 50A8F2-IMGT-CDRH2
IFPGSGHT

SEQ ID NO: 166 - protein, 13 aa, synthetic construct
  source 1..13: note = 50A8F2-IMGT-CDRH3
ARGDRSALGYFDY

SEQ ID NO: 167 - protein, 7 aa, synthetic construct
  source 1..7: note = 50A8F2-IMGT-CDRL1
SSVSSTY

SEQ ID NO: 168 - skipped

SEQ ID NO: 169 - protein, 120 aa, synthetic construct
  source 1..120: note = 50A8F2-VH
QIQLQQSGPELVKPGASVKISCKASGYTFIDYYINWVKQKPGQGLEWIGWIFPGSGHTKYNENFKGKATLTVDTSSSTAYMHLSSLTSEDTAVYFCARGDRSALGYFDYWGQGSTLTVSS

SEQ ID NO: 170 - protein, 108 aa, synthetic construct
  source 1..108: note = 50A8F2-VL
QIVLTQSPAIMSASLGERVTMTCTASSSVSSTYLHWYQQKPGSSPKLWIYSTSNLASGVPARFSGSGSGTSYSLTFSSMEAEDAATYYCHQYHRSPLTFGAGTKLELK

SEQ ID NO: 171 - protein, 5 aa, synthetic construct
  source 1..5: note = 47A5H2-Kabat-CDRH1
GYWMH

SEQ ID NO: 172 - protein, 17 aa, synthetic construct
  source 1..17: note = 47A5H2-Kabat-CDRH2
MIHPSDSETRLNQKFKD

SEQ ID NO: 173 - protein, 10 aa, synthetic construct
  source 1..10: note = 47A5H2-Kabat-CDRH3
EGLRRGSFAY

SEQ ID NO: 174 - protein, 15 aa, synthetic construct
  source 1..15: note = 47A5H2-Kabat-CDRL1
RASESVDNYGMTFMS

SEQ ID NO: 175 - protein, 7 aa, synthetic construct
  source 1..7: note = 47A5H2-Kabat-CDRL2
AASNQGS

SEQ ID NO: 176 - protein, 9 aa, synthetic construct
  source 1..9: note = 47A5H2-Kabat-CDRL3
QQSKEVPYT

SEQ ID NO: 177 - protein, 7 aa, synthetic construct
  source 1..7: note = 47A5H2-Chothia-CDRH1
GYSFTGY

SEQ ID NO: 178 - protein, 6 aa, synthetic construct
  source 1..6: note = 47A5H2-Chothia-CDRH2
HPSDSE

SEQ ID NO: 179 - protein, 10 aa, synthetic construct
  source 1..10: note = 47A5H2-AbM-CDRH1
GYSFTGYWMH

SEQ ID NO: 180 - protein, 10 aa, synthetic construct
  source 1..10: note = 47A5H2-AbM-CDRH2
MIHPSDSETR

SEQ ID NO: 181 - protein, 6 aa, synthetic construct
  source 1..6: note = 47A5H2-Contact-CDRH1
TGYWMH

SEQ ID NO: 182 - protein, 13 aa, synthetic construct
  source 1..13: note = 47A5H2-Contact-CDRH2
WIGMIHPSDSETR

SEQ ID NO: 183 - protein, 11 aa, synthetic construct
  source 1..11: note = 47A5H2-Contact-CDRH3
ASEGLRRGSFA

SEQ ID NO: 184 - protein, 11 aa, synthetic construct
  source 1..11: note = 47A5H2-Contact-CDRL1
DNYGMTFMSWF

SEQ ID NO: 185 - protein, 10 aa, synthetic construct
  source 1..10: note = 47A5H2-Contact-CDRL2
LLIYAASNQG

SEQ ID NO: 186 - protein, 8 aa, synthetic construct
  source 1..8: note = 47A5H2-Contact-CDRL3
QQSKEVPY

SEQ ID NO: 187 - protein, 8 aa, synthetic construct
  source 1..8: note = 47A5H2-IMGT-CDRH1
GYSFTGYW

SEQ ID NO: 188 - protein, 8 aa, synthetic construct
  source 1..8: note = 47A5H2-IMGT-CDRH2
IHPSDSET

SEQ ID NO: 189 - protein, 12 aa, synthetic construct
  source 1..12: note = 47A5H2-IMGT-CDRH3
ASEGLRRGSFAY

SEQ ID NO: 190 - protein, 10 aa, synthetic construct
  source 1..10: note = 47A5H2-IMGT-CDRL1
ESVDNYGMTF

SEQ ID NO: 191 - skipped

SEQ ID NO: 192 - protein, 119 aa, synthetic construct
  source 1..119: note = 47A5H2-VH
QVQLQQPGAELVRPGASVKLSCKGSGYSFTGYWMHWVKQRPGQGLEWIGMIHPSDSETRLNQKFKDKATLTVDKSSSTTYMQLSSPTSEDSAVYYCASEGLRRGSFAYWGQGTLVTVSA

SEQ ID NO: 193 - protein, 111 aa, synthetic construct
  source 1..111: note = 47A5H2-VL
DIVLTQSPASLAVSLGQRATISCRASESVDNYGMTFMSWFQQKPGQPPKLLIYAASNQGSGVPARFSGSGSGTDFSLNIHPMEEDDTAMYFCQQSKEVPYTFGGGTKLEIK

SEQ ID NO: 194 - protein, 5 aa, synthetic construct
  source 1..5: note = 66G9C11-Kabat-CDRH1
TFWMN

SEQ ID NO: 195 - protein, 17 aa, synthetic construct
  source 1..17: note = 66G9C11-Kabat-CDRH2
QIFPASGSTNYNEMFEG

SEQ ID NO: 196 - protein, 8 aa, synthetic construct
  source 1..8: note = 66G9C11-Kabat-CDRH3
LERAWFAY

SEQ ID NO: 197 - protein, 11 aa, synthetic construct
  source 1..11: note = 66G9C11-Kabat-CDRL1
RASQSINNYLH

SEQ ID NO: 198 - protein, 7 aa, synthetic construct
  source 1..7: note = 66G9C11-Kabat-CDRL2
YASQSIS

SEQ ID NO: 199 - protein, 9 aa, synthetic construct
  source 1..9: note = 66G9C11-Kabat-CDRL3
QQSNSWPPT

SEQ ID NO: 200 - protein, 7 aa, synthetic construct
  source 1..7: note = 66G9C11-Chothia-CDRH1
GYIFFTF

SEQ ID NO: 201 - protein, 6 aa, synthetic construct
  source 1..6: note = 66G9C11-Chothia-CDRH2
FPASGS

SEQ ID NO: 202 - protein, 10 aa, synthetic construct
  source 1..10: note = 66G9C11-AbM-CDRH1
GYIFFTFWMN

SEQ ID NO: 203 - protein, 10 aa, synthetic construct
  source 1..10: note = 66G9C11-AbM-CDRH2
QIFPASGSTN

SEQ ID NO: 204 - protein, 6 aa, synthetic construct
  source 1..6: note = 66G9C11-Contact-CDRH1
FTFWMN

SEQ ID NO: 205 - protein, 13 aa, synthetic construct
  source 1..13: note = 66G9C11-Contact-CDRH2
WIGQIFPASGSTN

SEQ ID NO: 206 - protein, 9 aa, synthetic construct
  source 1..9: note = 66G9C11-Contact-CDRH3
ASLERAWFA

SEQ ID NO: 207 - protein, 7 aa, synthetic construct
  source 1..7: note = 66G9C11-Contact-CDRL1
NNYLHWY

SEQ ID NO: 208 - protein, 10 aa, synthetic construct
  source 1..10: note = 66G9C11-Contact-CDRL2
LLIKYASQSI

SEQ ID NO: 209 - protein, 8 aa, synthetic construct
  source 1..8: note = 66G9C11-Contact-CDRL3
QQSNSWPP

SEQ ID NO: 210 - protein, 8 aa, synthetic construct
  source 1..8: note = 66G9C11-IMGT-CDRH1
GYIFFTFW

SEQ ID NO: 211 - protein, 8 aa, synthetic construct
  source 1..8: note = 66G9C11-IMGT-CDRH2
IFPASGST

SEQ ID NO: 212 - protein, 10 aa, synthetic construct
  source 1..10: note = 66G9C11-IMGT-CDRH3
ASLERAWFAY

SEQ ID NO: 213 - protein, 6 aa, synthetic construct
  source 1..6: note = 66G9C11-IMGT-CDRL1
QSINNY

SEQ ID NO: 214 - skipped

SEQ ID NO: 215 - protein, 117 aa, synthetic construct
  source 1..117: note = 66G9C11-VH
HVQLQQSGPELVRPGASVKLSCKASGYIFFTFWMNWVKQRPGQGLEWIGQIFPASGSTNYNEMFEGKATLTVDTSSSTAYMQLSSLTSEDSAVYYCASLERAWFAYWGQGTLVTVSA

SEQ ID NO: 216 - protein, 107 aa, synthetic construct
  source 1..107: note = 66G9C11-VL
DIVLTQSPATLSVTPGDRVSLSCRASQSINNYLHWYQQKSHESPRLLIKYASQSISGIPSRFSGSGSGADFTLSINSVETEDFGMYFCQQSNSWPPTFGGGTKLEIK

SEQ ID NO: 217 - protein, 5 aa, synthetic construct
  source 1..5: note = 33F11A6-Kabat-CDRH1
DYAVN

SEQ ID NO: 218 - protein, 16 aa, synthetic construct
  source 1..16: note = 33F11A6-Kabat-CDRH2
MIWGDGSTDFNSPLKS

SEQ ID NO: 219 - protein, 12 aa, synthetic construct
  source 1..12: note = 33F11A6-Kabat-CDRH3, 33F11A6-Chothia-CDRH3
TYNDYHYWYFDV

SEQ ID NO: 220 - protein, 10 aa, synthetic construct
  source 1..10: note = 33F11A6-AbM-CDRH1
GFSLTDYAVN

SEQ ID NO: 221 - protein, 6 aa, synthetic construct
  source 1..6: note = 33F11A6-Contact-CDRH1
TDYAVN

SEQ ID NO: 222 - protein, 13 aa, synthetic construct
  source 1..13: note = 33F11A6-Contact-CDRH3
ARTYNDYHYWYFD

SEQ ID NO: 223 - protein, 10 aa, synthetic construct
  source 1..10: note = 33F11A6-Contact-CDRL2
LLIYNAKTLA

SEQ ID NO: 224 - protein, 8 aa, synthetic construct
  source 1..8: note = 33F11A6-IMGT-CDRH1
GFSLTDYA

SEQ ID NO: 225 - protein, 14 aa, synthetic construct
  source 1..14: note = 33F11A6-IMGT-CDRH3
ARTYNDYHYWYFDV

SEQ ID NO: 226 - protein, 120 aa, synthetic construct
  source 1..120: note = 33F11A6-VH
QVQLKESGPGLVAPSQSLSITCTVSGFSLTDYAVNWVRQPPGKGLEWLGMIWGDGSTDFNSPLKSRLSISKDNSKSQVFLKMNSLQTDDTARYFCARTYNDYHYWYFDVWGAGTTVTVSS

SEQ ID NO: 227 - protein, 107 aa, synthetic construct
  source 1..107: note = 33F11A6-VL
DIQMTQSPASLSASVGETVTITCRASGNIHNYLAWYQQKQGKSPQLLIYNAKTLADGVPSRFSGSGSGTQYFLKINSLQPEDFGSYCCQHFWSTPYTFGGGTKLEIK

SEQ ID NO: 228 - protein, 12 aa, synthetic construct
  source 1..12: note = 37G6A7-Kabat-CDRH3
TYGTYHYWYFDV

SEQ ID NO: 229 - protein, 7 aa, synthetic construct
  source 1..7: note = 37G6A7-Kabat-CDRL2, 37G6A7-Chothia-CDRL2, 37G6A7-AbM-CDRL2
IAKTLAD

SEQ ID NO: 230 - protein, 13 aa, synthetic construct
  source 1..13: note = 37G6A7-Contact-CDRH3
ARTYGTYHYWYFD

SEQ ID NO: 231 - protein, 10 aa, synthetic construct
  source 1..10: note = 37G6A7-Contact-CDRL2
LLVYIAKTLA

SEQ ID NO: 232 - protein, 14 aa, synthetic construct
  source 1..14: note = 37G6A7-IMGT-CDRH3
ARTYGTYHYWYFDV

SEQ ID NO: 233 - skipped

SEQ ID NO: 234 - protein, 120 aa, synthetic construct
  source 1..120: note = 37G6A7-VH
QVQLKESGPGLVAPSQSLSITCTVSGFSLTDYGVNWVRQPPGKGLEWLGMIWGDGSTDYNSALKSRLTISKDNSKSQVFLKMNSLQTDDTARYYCARTYGTYHYWYFDVWGAGTTVTVSS

SEQ ID NO: 235 - protein, 107 aa, synthetic construct
  source 1..107: note = 37G6A7-VL
DIQMTQSPASLSASVGETVTITCRASGNIHNYLAWYQQKQGKSPQLLVYIAKTLADGVPSRFSGSGSGTQYSLKINSLQPEDFGTYYCQHFWSTPYTFGGGTKLEIK

SEQ ID NO: 236 - protein, 16 aa, synthetic construct
  source 1..16: note = 39C5D6-Kabat-CDRH2
MIWGDGSTDYNLPLKS

SEQ ID NO: 237 - protein, 12 aa, synthetic construct
  source 1..12: note = 39C5D6-Kabat-CDRH3
TYANYHYWYFDV

SEQ ID NO: 238 - protein, 7 aa, synthetic construct
  source 1..7: note = 39C5D6-Kabat-CDRL2, 39C5D6-Chothia-CDRL2, 39C5D6-AbM-CDRL2
NAKTLED

SEQ ID NO: 239 - protein, 13 aa, synthetic construct
  source 1..13: note = 39C5D6-Contact-CDRH3
ARTYANYHYWYFD

SEQ ID NO: 240 - protein, 10 aa, synthetic construct
  source 1..10: note = 39C5D6-Contact-CDRL2
LLVYNAKTLE

SEQ ID NO: 241 - protein, 14 aa, synthetic construct
  source 1..14: note = 39C5D6-IMGT-CDRH3
ARTYANYHYWYFDV

SEQ ID NO: 242 - protein, 120 aa, synthetic construct
  source 1..120: note = 39C5D6-VH
QVQLKESGPGLVAPSQSLSITCTVSGFSLTDYGVNWVRQTPGKGLEWLGMIWGDGSTDYNLPLKSRLSITKDNSKSQVFLKMNSLQTDDTARYYCARTYANYHYWYFDVWGAGTTVTVSS

SEQ ID NO: 243 - protein, 107 aa, synthetic construct
  source 1..107: note = 39C5D6-VL
DIQMTQSPASLSASVGETVTITCRASGNIHNYLAWYQQKQGKSPQLLVYNAKTLEDGVPSRFSGSGSGTQYSLKINSLQPDDFGNYFCQHFWSTPYTFGGGTKLEIK

SEQ ID NO: 244 - protein, 5 aa, synthetic construct
  source 1..5: note = 62H1D6-Kabat-CDRH1
DYAMH

SEQ ID NO: 245 - protein, 17 aa, synthetic construct
  source 1..17: note = 62H1D6-Kabat-CDRH2
VISTYYGNTNYNQKFKG

SEQ ID NO: 246 - protein, 13 aa, synthetic construct
  source 1..13: note = 62H1D6-Kabat-CDRH3
EYGNYPYYYAMDY

SEQ ID NO: 247 - protein, 17 aa, synthetic construct
  source 1..17: note = 62H1D6-Kabat-CDRL1
KSSQSLLNSSNQKNYLA

SEQ ID NO: 248 - protein, 7 aa, synthetic construct
  source 1..7: note = 62H1D6-Kabat-CDRL2
FASTRES

SEQ ID NO: 249 - protein, 9 aa, synthetic construct
  source 1..9: note = 62H1D6-Kabat-CDRL3
QQHYNTPFT

SEQ ID NO: 250 - protein, 7 aa, synthetic construct
  source 1..7: note = 62H1D6-Chothia-CDRH1
GYTFTDY

SEQ ID NO: 251 - protein, 6 aa, synthetic construct
  source 1..6: note = 62H1D6-Chothia-CDRH2
STYYGN

SEQ ID NO: 252 - protein, 10 aa, synthetic construct
  source 1..10: note = 62H1D6-AbM-CDRH1
GYTFTDYAMH

SEQ ID NO: 253 - protein, 10 aa, synthetic construct
  source 1..10: note = 62H1D6-AbM-CDRH2
VISTYYGNTN

SEQ ID NO: 254 - protein, 6 aa, synthetic construct
  source 1..6: note = 62H1D6-Contact-CDRH1
TDYAMH

SEQ ID NO: 255 - protein, 13 aa, synthetic construct
  source 1..13: note = 62H1D6-Contact-CDRH2
WIGVISTYYGNTN

SEQ ID NO: 256 - protein, 14 aa, synthetic construct
  source 1..14: note = 62H1D6-Contact-CDRH3
AREYGNYPYYYAMD

SEQ ID NO: 257 - protein, 13 aa, synthetic construct
  source 1..13: note = 62H1D6-Contact-CDRL1
LNSSNQKNYLAWY

SEQ ID NO: 258 - protein, 10 aa, synthetic construct
  source 1..10: note = 62H1D6-Contact-CDRL2
LLVYFASTRE

SEQ ID NO: 259 - protein, 8 aa, synthetic construct
  source 1..8: note = 62H1D6-Contact-CDRL3
QQHYNTPF

SEQ ID NO: 260 - protein, 8 aa, synthetic construct
  source 1..8: note = 62H1D6-IMGT-CDRH1
GYTFTDYA

SEQ ID NO: 261 - protein, 8 aa, synthetic construct
  source 1..8: note = 62H1D6-IMGT-CDRH2
ISTYYGNT

SEQ ID NO: 262 - protein, 15 aa, synthetic construct
  source 1..15: note = 62H1D6-IMGT-CDRH3
AREYGNYPYYYAMDY

SEQ ID NO: 263 - protein, 12 aa, synthetic construct
  source 1..12: note = 62H1D6-IMGT-CDRL1
QSLLNSSNQKNY

SEQ ID NO: 264 - skipped

SEQ ID NO: 265 - protein, 122 aa, synthetic construct
  source 1..122: note = 62H1D6-VH
QVQLQQSGPELVRPGVSVKISCKGSGYTFTDYAMHWVKQSHAKSLEWIGVISTYYGNTNYNQKFKGRATMTVDKSSSTAYMELARLTSEDSAIYYCAREYGNYPYYYAMDYWGQGTSVTVSS

SEQ ID NO: 266 - protein, 113 aa, synthetic construct
  source 1..113: note = 62H1D6-VL
DIVMTQSPSSLAMSVGQKVTMSCKSSQSLLNSSNQKNYLAWYQQKPGQSPKLLVYFASTRESGVPDRFIGSGSGTDFTLTISSVQAEDLADYFCQQHYNTPFTFGSGTKLEIK

SEQ ID NO: 267 - protein, 16 aa, synthetic construct
  source 1..16: note = 92E12F2-Kabat-CDRH2
YISYDGSNNDNPSLTN

SEQ ID NO: 268 - protein, 15 aa, synthetic construct
  source 1..15: note = 92E12F2-Kabat-CDRL1
RASKSVSSSGYTYLH

SEQ ID NO: 269 - protein, 7 aa, synthetic construct
  source 1..7: note = 92E12F2-Kabat-CDRL2
LTSNLDS

SEQ ID NO: 270 - protein, 9 aa, synthetic construct
  source 1..9: note = 92E12F2-Kabat-CDRL3
QHSRELPRT

SEQ ID NO: 271 - protein, 8 aa, synthetic construct
  source 1..8: note = 92E12F2-Chothia-CDRH1
GYSITSGY

SEQ ID NO: 272 - protein, 11 aa, synthetic construct
  source 1..11: note = 92E12F2-AbM-CDRH1
GYSITSGYYWN

SEQ ID NO: 273 - protein, 7 aa, synthetic construct
  source 1..7: note = 92E12F2-Contact-CDRH1
TSGYYWN

SEQ ID NO: 274 - protein, 12 aa, synthetic construct
  source 1..12: note = 92E12F2-Contact-CDRH2
WLGYISYDGSNN

SEQ ID NO: 275 - protein, 9 aa, synthetic construct
  source 1..9: note = 92E12F2-Contact-CDRH3
ARGDFYGYV

SEQ ID NO: 276 - protein, 11 aa, synthetic construct
  source 1..11: note = 92E12F2-Contact-CDRL1
SSSGYTYLHWY

SEQ ID NO: 277 - protein, 10 aa, synthetic construct
  source 1..10: note = 92E12F2-Contact-CDRL2
LLIYLTSNLD

SEQ ID NO: 278 - protein, 8 aa, synthetic construct
  source 1..8: note = 92E12F2-Contact-CDRL3
QHSRELPR

SEQ ID NO: 279 - protein, 9 aa, synthetic construct
  source 1..9: note = 92E12F2-IMGT-CDRH1
GYSITSGYY

SEQ ID NO: 280 - protein, 10 aa, synthetic construct
  source 1..10: note = 92E12F2-IMGT-CDRH3
ARGDFYGYVY

SEQ ID NO: 281 - protein, 10 aa, synthetic construct
  source 1..10: note = 92E12F2-IMGT-CDRL1
KSVSSSGYTY

SEQ ID NO: 282 - skipped

SEQ ID NO: 283 - protein, 117 aa, synthetic construct
  source 1..117: note = 92E12F2-VH
DVQLQESGPGLVKPSQSLSLTCSVTGYSITSGYYWNWIRQFPGNKLEWLGYISYDGSNNDNPSLTNRISISRDTSRNQFFLKFNSVTTDDSATYYCARGDFYGYVYWGQGTTLTVSS

SEQ ID NO: 284 - protein, 111 aa, synthetic construct
  source 1..111: note = 92E12F2-VL
DIVLTQSPASLAVSLGQRATISCRASKSVSSSGYTYLHWYQQKPGQPPKLLIYLTSNLDSGVPARFSGSGSGTDFTLNIHPVEEEDAATYYCQHSRELPRTFGGGTKLEIK

SEQ ID NO: 285 - protein, 5 aa, synthetic construct
  source 1..5: note = 13H2D11-Kabat-CDRH1
SYWMN

SEQ ID NO: 286 - protein, 10 aa, synthetic construct
  source 1..10: note = 13H2D11-Kabat-CDRH3
EGFRRGSFAY

SEQ ID NO: 287 - protein, 15 aa, synthetic construct
  source 1..15: note = 13H2D11-Kabat-CDRL1
RASESVDNYGISFMN

SEQ ID NO: 288 - protein, 7 aa, synthetic construct
  source 1..7: note = 13H2D11-Chothia-CDRH1
GYSFTSY

SEQ ID NO: 289 - protein, 10 aa, synthetic construct
  source 1..10: note = 13H2D11-AbM-CDRH1
GYSFTSYWMN

SEQ ID NO: 290 - protein, 6 aa, synthetic construct
  source 1..6: note = 13H2D11-Contact-CDRH1
TSYWMN

SEQ ID NO: 291 - protein, 11 aa, synthetic construct
  source 1..11: note = 13H2D11-Contact-CDRH3
ASEGFRRGSFA

SEQ ID NO: 292 - protein, 11 aa, synthetic construct
  source 1..11: note = 13H2D11-Contact-CDRL1
DNYGISFMNWF

SEQ ID NO: 293 - protein, 8 aa, synthetic construct
  source 1..8: note = 13H2D11-IMGT-CDRH1
GYSFTSYW

SEQ ID NO: 294 - protein, 12 aa, synthetic construct
  source 1..12: note = 13H2D11-IMGT-CDRH3
ASEGFRRGSFAY

SEQ ID NO: 295 - protein, 10 aa, synthetic construct
  source 1..10: note = 13H2D11-IMGT-CDRL1
ESVDNYGISF

SEQ ID NO: 296 - protein, 119 aa, synthetic construct
  source 1..119: note = 13H2D11-VH
QVQLQQPGAELVRPGASVKLSCKASGYSFTSYWMNWVKQRPGQGLEWIGMIHPSDSETRLNQKFKDKATLTVDKSSSTAYMQLSSPTSEDSAVYYCASEGFRRGSFAYWGQGTLVTVSA

SEQ ID NO: 297 - protein, 111 aa, synthetic construct
  source 1..111: note = 13H2D11-VL
DIVLTQSPASLAVSLGQRATISCRASESVDNYGISFMNWFQQKPGQPPKLLIYAASNQGSGVPARFSGSGSGTDFSLNIHPMEEDDTAMYFCQQSKEVPYTFGGGTKLEIK

SEQ ID NO: 298 - protein, 6 aa, synthetic construct
  source 1..6: note = 5A11D10-Kabat-CDRH1
SDFAWN

SEQ ID NO: 299 - protein, 16 aa, synthetic construct
  source 1..16: note = 5A11D10-Kabat-CDRH2
DIRYSGSTDYNPSLKT

SEQ ID NO: 300 - protein, 14 aa, synthetic construct
  source 1..14: note = 5A11D10-Kabat-CDRH3
GGYYGYDEGYPMDY

SEQ ID NO: 301 - protein, 15 aa, synthetic construct
  source 1..15: note = 5A11D10-Kabat-CDRL1
RASESVDVYGNSFMH

SEQ ID NO: 302 - protein, 7 aa, synthetic construct
  source 1..7: note = 5A11D10-Kabat-CDRL2
LAFNLKS

SEQ ID NO: 303 - protein, 9 aa, synthetic construct
  source 1..9: note = 5A11D10-Kabat-CDRL3
QQNNEDPFT

SEQ ID NO: 304 - protein, 8 aa, synthetic construct
  source 1..8: note = 5A11D10-Chothia-CDRH1
GYSLTSDF

SEQ ID NO: 305 - protein, 5 aa, synthetic construct
  source 1..5: note = 5A11D10-Chothia-CDRH2
RYSGS

SEQ ID NO: 306 - protein, 11 aa, synthetic construct
  source 1..11: note = 5A11D10-AbM-CDRH1
GYSLTSDFAWN

SEQ ID NO: 307 - protein, 9 aa, synthetic construct
  source 1..9: note = 5A11D10-AbM-CDRH2
DIRYSGSTD

SEQ ID NO: 308 - protein, 7 aa, synthetic construct
  source 1..7: note = 5A11D10-Contact-CDRH1
TSDFAWN

SEQ ID NO: 309 - protein, 12 aa, synthetic construct
  source 1..12: note = 5A11D10-Contact-CDRH2
WMGDIRYSGSTD

SEQ ID NO: 310 - protein, 15 aa, synthetic construct
  source 1..15: note = 5A11D10-Contact-CDRH3
ARGGYYGYDEGYPMD

SEQ ID NO: 311 - protein, 11 aa, synthetic construct
  source 1..11: note = 5A11D10-Contact-CDRL1
DVYGNSFMHWY

SEQ ID NO: 312 - protein, 10 aa, synthetic construct
  source 1..10: note = 5A11D10-Contact-CDRL2
LLIYLAFNLK

SEQ ID NO: 313 - protein, 8 aa, synthetic construct
  source 1..8: note = 5A11D10-Contact-CDRL3
QQNNEDPF

SEQ ID NO: 314 - protein, 9 aa, synthetic construct
  source 1..9: note = 5A11D10-IMGT-CDRH1
GYSLTSDFA

SEQ ID NO: 315 - protein, 7 aa, synthetic construct
  source 1..7: note = 5A11D10-IMGT-CDRH2
IRYSGST

SEQ ID NO: 316 - protein, 16 aa, synthetic construct
  source 1..16: note = 5A11D10-IMGT-CDRH3
ARGGYYGYDEGYPMDY

SEQ ID NO: 317 - protein, 10 aa, synthetic construct
  source 1..10: note = 5A11D10-IMGT-CDRL1
ESVDVYGNSF

SEQ ID NO: 318 - protein, 123 aa, synthetic construct
  source 1..123: note = 5A11D10-VH
DVQLQESGPGQVKPSQSLSLTCTVTGYSLTSDFAWNWIRQFPGNKLEWMGDIRYSGSTDYNPSLKTRSSITRDTSKNQFFLHLNSVTTEDTATYYCARGGYYGYDEGYPMDYWGQGTSVTVSS

SEQ ID NO: 319 - protein, 111 aa, synthetic construct
  source 1..111: note = 5A11D10-VL
NIVLTQSPASLAVSLGQRATISCRASESVDVYGNSFMHWYQQKAGQPPKLLIYLAFNLKSGVPARFSGSGSRTDFTLTIDPVEADDAATYYCQQNNEDPFTFGSGTKLEIK

SEQ ID NO: 320 - protein, 16 aa, synthetic construct
  source 1..16: note = 22F10G3-Kabat-CDRH2
DIRYSGSTDYNPSLKG

SEQ ID NO: 321 - protein, 123 aa, synthetic construct
  source 1..123: note = 22F10G3-VH
DVQLQESGPGQVKPSQSLSLTCTVTGYSLTSDFAWNWIRQFPGNRLEWMGDIRYSGSTDYNPSLKGRSSITRDTSKNQFFLQLNSVTTEDTATYYCARGGYYGYDEGYPMDYWGQGTSVTVSS

SEQ ID NO: 322 - protein, 111 aa, synthetic construct
  source 1..111: note = 22F10G3-VL
NIVLTQSPASLAVSLGQRATISCRASESVDVYGNSFMHWYQQKTGQPPKLLIYLAFNLKSGVPARFSGSGSRTDFTLTIDPVEADDAATYYCQQNNEDPFTFGSGTKLEIK

SEQ ID NO: 323 - protein, 15 aa, synthetic construct
  source 1..15: note = 22F10G3-NA-Kabat-CDRL1, 22F10G3-NA-Chothia-CDRL1, 22F10G3-NA-AbM-CDRL1
RASESVDVYGNAFMH

SEQ ID NO: 324 - protein, 11 aa, synthetic construct
  source 1..11: note = 22F10G3-NA-Contact-CDRL1
DVYGNAFMHWY

SEQ ID NO: 325 - protein, 10 aa, synthetic construct
  source 1..10: note = 22F10G3-NA-IMGT-CDRL1
ESVDVYGNAF

SEQ ID NO: 326 - protein, 111 aa, synthetic construct
  source 1..111: note = 22F10G3-NA-VL
NIVLTQSPASLAVSLGQRATISCRASESVDVYGNAFMHWYQQKTGQPPKLLIYLAFNLKSGVPARFSGSGSRTDFTLTIDPVEADDAATYYCQQNNEDPFTFGSGTKLEIK

SEQ ID NO: 327 - protein, 15 aa, synthetic construct
  source 1..15: note = 22F10G3-QS-Kabat-CDRL1, 22F10G3-QS-Chothia-CDRL1, 22F10G3-QS-AbM-CDRL1
RASESVDVYGQSFMH

SEQ ID NO: 328 - protein, 11 aa, synthetic construct
  source 1..11: note = 22F10G3-QS-Contact-CDRL1
DVYGQSFMHWY

SEQ ID NO: 329 - protein, 10 aa, synthetic construct
  source 1..10: note = 22F10G3-QS-IMGT-CDRL1
ESVDVYGQSF

SEQ ID NO: 330 - protein, 111 aa, synthetic construct
  source 1..111: note = 22F10G3-QS-VL
NIVLTQSPASLAVSLGQRATISCRASESVDVYGQSFMHWYQQKTGQPPKLLIYLAFNLKSGVPARFSGSGSRTDFTLTIDPVEADDAATYYCQQNNEDPFTFGSGTKLEIK

SEQ ID NO: 331 - protein, 123 aa, synthetic construct
  source 1..123: note = 22F10G3-VH1-VH
QVQLQESGPGLVKPSQTLSLTCTVSGYSLTSDFAWNWIRQHPGKGLEWIGDIRYSGSTDYNPSLKGRVTISVDTSKNQFSLKLSSVTAADTAVYYCARGGYYGYDEGYPMDYWGQGTLVTVSS

SEQ ID NO: 332 - protein, 123 aa, synthetic construct
  source 1..123: note = 22F10G3-VH2-VH
QVQLQESGPGLVKPSQTLSLTCTVSGYSLTSDFAWNWIRQHPGKGLEWMGDIRYSGSTDYNPSLKGRVTISRDTSKNQFSLKLSSVTAADTAVYYCARGGYYGYDEGYPMDYWGQGTLVTVSS

SEQ ID NO: 333 - protein, 123 aa, synthetic construct
  source 1..123: note = 22F10G3-VH3-VH
QVQLQESGPGLVKPSQTLSLTCTVSGYSLTSDFAWNWIRQHPGKGLEWMGDIRYSGSTDYNPSLKGRSTISRDTSKNQFSLKLSSVTAADTAVYYCARGGYYGYDEGYPMDYWGQGTLVTVSS

SEQ ID NO: 334 - protein, 123 aa, synthetic construct
  source 1..123: note = 22F10G3-VH4-VH
QVQLQESGPGLVKPSQTLSLTCTVSGYSLTSDFAWNWIRQFPGKGLEWMGDIRYSGSTDYNPSLKGRSTISRDTSKNQFFLKLSSVTAADTAVYYCARGGYYGYDEGYPMDYWGQGTLVTVSS

SEQ ID NO: 335 - protein, 111 aa, synthetic construct
  source 1..111: note = 22F10G3-VL1-VL
DIVMTQSPDSLAVSLGERATINCRASESVDVYGNSFMHWYQQKPGQPPKLLIYLAFNLKSGVPDRFSGSGSGTDFTLTISSLQAEDVAVYYCQQNNEDPFTFGQGTKLEIK

SEQ ID NO: 336 - protein, 111 aa, synthetic construct
  source 1..111: note = 22F10G3-VL2-VL
DIVMTQSPDSLAVSLGERATINCRASESVDVYGNSFMHWYQQKPGQPPKLLIYLAFNLKSGVPARFSGSGSGTDFTLTISSLQAEDVAVYYCQQNNEDPFTFGQGTKLEIK

SEQ ID NO: 337 - protein, 111 aa, synthetic construct
  source 1..111: note = 22F10G3-VL3-VL
DIVMTQSPDSLAVSLGERATISCRASESVDVYGNSFMHWYQQKPGQPPKLLIYLAFNLKSGVPDRFSGSGSGTDFTLTISSLQAEDVAVYYCQQNNEDPFTFGQGTKLEIK

SEQ ID NO: 338 - protein, 111 aa, synthetic construct
  source 1..111: note = 22F10G3-VL4-VL
DIVMTQSPDSLAVSLGERATISCRASESVDVYGNSFMHWYQQKPGQPPKLLIYLAFNLKSGVPARFSGSGSGTDFTLTISSLQAEDVATYYCQQNNEDPFTFGQGTKLEIK

SEQ ID NO: 339 - protein, 111 aa, synthetic construct
  source 1..111: note = 22F10G3-VL1-NA-VL
DIVMTQSPDSLAVSLGERATINCRASESVDVYGNAFMHWYQQKPGQPPKLLIYLAFNLKSGVPDRFSGSGSGTDFTLTISSLQAEDVAVYYCQQNNEDPFTFGQGTKLEIK

SEQ ID NO: 340 - protein, 111 aa, synthetic construct
  source 1..111: note = 22F10G3-VL2-NA-VL
DIVMTQSPDSLAVSLGERATINCRASESVDVYGNAFMHWYQQKPGQPPKLLIYLAFNLKSGVPARFSGSGSGTDFTLTISSLQAEDVAVYYCQQNNEDPFTFGQGTKLEIK

SEQ ID NO: 341 - protein, 111 aa, synthetic construct
  source 1..111: note = 22F10G3-VL3-NA-VL
DIVMTQSPDSLAVSLGERATISCRASESVDVYGNAFMHWYQQKPGQPPKLLIYLAFNLKSGVPDRFSGSGSGTDFTLTISSLQAEDVAVYYCQQNNEDPFTFGQGTKLEIK

SEQ ID NO: 342 - protein, 111 aa, synthetic construct
  source 1..111: note = 22F10G3-VL4-NA-VL
DIVMTQSPDSLAVSLGERATISCRASESVDVYGNAFMHWYQQKPGQPPKLLIYLAFNLKSGVPARFSGSGSGTDFTLTISSLQAEDVATYYCQQNNEDPFTFGQGTKLEIK

SEQ ID NO: 343 - protein, 111 aa, synthetic construct
  source 1..111: note = 22F10G3-VL1-QS-VL
DIVMTQSPDSLAVSLGERATINCRASESVDVYGQSFMHWYQQKPGQPPKLLIYLAFNLKSGVPDRFSGSGSGTDFTLTISSLQAEDVAVYYCQQNNEDPFTFGQGTKLEIK

SEQ ID NO: 344 - protein, 111 aa, synthetic construct
  source 1..111: note = 22F10G3-VL2-QS-VL
DIVMTQSPDSLAVSLGERATINCRASESVDVYGQSFMHWYQQKPGQPPKLLIYLAFNLKSGVPARFSGSGSGTDFTLTISSLQAEDVAVYYCQQNNEDPFTFGQGTKLEIK

SEQ ID NO: 345 - protein, 111 aa, synthetic construct
  source 1..111: note = 22F10G3-VL3-QS-VL
DIVMTQSPDSLAVSLGERATISCRASESVDVYGQSFMHWYQQKPGQPPKLLIYLAFNLKSGVPDRFSGSGSGTDFTLTISSLQAEDVAVYYCQQNNEDPFTFGQGTKLEIK

SEQ ID NO: 346 - protein, 111 aa, synthetic construct
  source 1..111: note = 22F10G3-VL4-QS-VL
DIVMTQSPDSLAVSLGERATISCRASESVDVYGQSFMHWYQQKPGQPPKLLIYLAFNLKSGVPARFSGSGSGTDFTLTISSLQAEDVATYYCQQNNEDPFTFGQGTKLEIK

SEQ ID NO: 347 - protein, 119 aa, synthetic construct
  source 1..119: note = 47A5H2-VH_g0-VH
QVQLVQSGAEVKKPGASVKVSCKASGYSFTGYWMHWVRQAPGQGLEWMGMIHPSDSETRLNQKFKDRVTMTRDTSTSTVYMELSSLRSEDTAVYYCASEGLRRGSFAYWGQGTLVTVSS

SEQ ID NO: 348 - protein, 119 aa, synthetic construct
  source 1..119: note = 47A5H2-VH_g1-VH
QVQLVQSGAEVKKPGASVKVSCKASGYSFTGYWMHWVRQAPGQGLEWMGMIHPSDSETRLNQKFKDRVTMTVDTSTSTVYMELSSLRSEDTAVYYCASEGLRRGSFAYWGQGTLVTVSS

SEQ ID NO: 349 - protein, 119 aa, synthetic construct
  source 1..119: note = 47A5H2-VH_g2-VH
QVQLVQSGAEVKKPGASVKVSCKASGYSFTGYWMHWVRQAPGQGLEWMGMIHPSDSETRLNQKFKDRVTMTVDKSTSTVYMELSSLRSEDTAVYYCASEGLRRGSFAYWGQGTLVTVSS

SEQ ID NO: 350 - protein, 119 aa, synthetic construct
  source 1..119: note = 47A5H2-VH_g3-VH
QVQLVQSGAEVKKPGASVKVSCKGSGYSFTGYWMHWVRQAPGQGLEWMGMIHPSDSETRLNQKFKDKVTMTVDKSTSTVYMELSSLRSEDTAVYYCASEGLRRGSFAYWGQGTLVTVSS

SEQ ID NO: 351 - protein, 111 aa, synthetic construct
  source 1..111: note = 47A5H2-Vk1_g0-VL
DIVMTQSPDSLAVSLGERATINCRASESVDNYGMTFMSWYQQKPGQPPKLLIYAASNQGSGVPDRFSGSGSGTDFTLTISSLQAEDVAVYYCQQSKEVPYTFGQGTKLEIK

SEQ ID NO: 352 - protein, 111 aa, synthetic construct
  source 1..111: note = 47A5H2-Vk1_g1-VL
DIVLTQSPDSLAVSLGERATINCRASESVDNYGMTFMSWYQQKPGQPPKLLIYAASNQGSGVPDRFSGSGSGTDFTLTISSLQAEDVAVYYCQQSKEVPYTFGQGTKLEIK

SEQ ID NO: 353 - protein, 111 aa, synthetic construct
  source 1..111: note = 47A5H2-Vk1_g2-VL
DIVLTQSPDSLAVSLGERATINCRASESVDNYGMTFMSWFQQKPGQPPKLLIYAASNQGSGVPDRFSGSGSGTDFTLTISSLQAEDVAVYYCQQSKEVPYTFGQGTKLEIK

SEQ ID NO: 354 - protein, 111 aa, synthetic construct
  source 1..111: note = 47A5H2-Vk2_g0-VL
EIVLTQSPATLSLSPGERATLSCRASESVDNYGMTFMSWYQQKPGQAPRLLIYAASNQGSGIPARFSGSGSGTDFTLTISSLEPEDFAVYYCQQSKEVPYTFGQGTKLEIK

SEQ ID NO: 355 - protein, 120 aa, synthetic construct
  source 1..120: note = 50A8F2-VH_g0-VH
QVQLVQSGAEVKKPGASVKVSCKASGYTFIDYYINWVRQAPGQGLEWMGWIFPGSGHTKYNENFKGRVTMTRDTSTSTVYMELSSLRSEDTAVYYCARGDRSALGYFDYWGQGTTVTVSS

SEQ ID NO: 356 - protein, 120 aa, synthetic construct
  source 1..120: note = 50A8F2-VH_g1-VH
QVQLVQSGAEVKKPGASVKVSCKASGYTFIDYYINWVRQAPGQGLEWMGWIFPGSGHTKYNENFKGKVTMTVDTSTSTVYMELSSLRSEDTAVYYCARGDRSALGYFDYWGQGTTVTVSS

SEQ ID NO: 357 - protein, 120 aa, synthetic construct
  source 1..120: note = 50A8F2-VH_g2-VH
QVQLVQSGAEVKKPGASVKVSCKASGYTFIDYYINWVRQAPGQGLEWMGWIFPGSGHTKYNENFKGKVTLTVDTSTSTVYMELSSLRSEDTAVYYCARGDRSALGYFDYWGQGTTVTVSS

SEQ ID NO: 358 - protein, 120 aa, synthetic construct
  source 1..120: note = 50A8F2-VH_g3-VH
QVQLVQSGAEVKKPGASVKVSCKASGYTFIDYYINWVRQAPGQGLEWIGWIFPGSGHTKYNENFKGKVTLTVDTSTSTVYMELSSLRSEDTAVYFCARGDRSALGYFDYWGQGTTVTVSS

SEQ ID NO: 359 - protein, 108 aa, synthetic construct
  source 1..108: note = 50A8F2-Vk_g0-VL
DIQMTQSPSSLSASVGDRVTITCTASSSVSSTYLHWYQQKPGKAPKLLIYSTSNLASGVPSRFSGSGSGTDFTLTISSLQPEDFATYYCHQYHRSPLTFGQGTKLEIK

SEQ ID NO: 360 - protein, 108 aa, synthetic construct
  source 1..108: note = 50A8F2-Vk_g1-VL
DIQMTQSPSSLSASVGDRVTITCTASSSVSSTYLHWYQQKPGKAPKLLIYSTSNLASGVPSRFSGSGSGTDYTLTISSLQPEDFATYYCHQYHRSPLTFGQGTKLEIK

SEQ ID NO: 361 - protein, 108 aa, synthetic construct
  source 1..108: note = 50A8F2-Vk_g2-VL
DIQLTQSPSSLSASVGDRVTITCTASSSVSSTYLHWYQQKPGKSPKLLIYSTSNLASGVPSRFSGSGSGTDYTLTISSLQPEDFATYYCHQYHRSPLTFGQGTKLEIK

SEQ ID NO: 362 - protein, 108 aa, synthetic construct
  source 1..108: note = 50A8F2-Vk_g3-VL
DIQLTQSPSSLSASVGDRVTITCTASSSVSSTYLHWYQQKPGKSPKLLIYSTSNLASGVPSRFSGSGSGTDYTLTISSLQPEDAATYYCHQYHRSPLTFGQGTKLEIK

SEQ ID NO: 363-418 - skipped (56 entries)

SEQ ID NO: 419 - protein, 450 aa, synthetic construct
  source 1..450: note = Hu50A8F2-16-EN-HC
QVQLVQSGAEVKKPGASVKVSCKASGYTFIDYYINWVRQAPGQGLEWIGWIFPGSGHTKYNENFKGKVTLTVDTSTSTVYMELSSLRSEDTAVYFCARGDRSALGYFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 420 - protein, 447 aa, synthetic construct
  source 1..447: note = 22F10G3-HC
DVQLQESGPGQVKPSQSLSLTCTVTGYSLTSDFAWNWIRQFPGNRLEWMGDIRYSGSTDYNPSLKGRSSITRDTSKNQFFLQLNSVTTEDTATYYCARGGYYGYDEGYPMDYWGQGTSVTVSSAKTTPPSVYPLAPGSAAQTNSMVTLGCLVKGYFPEPVTVTWNSGSLSSGVHTFPAVLQSDLYTLSSSVTVPSSPRPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSPGK

SEQ ID NO: 421 - protein, 447 aa, synthetic construct; identical to SEQ ID NO: 420
  source 1..447: note = 22F10G3-NA-HC

SEQ ID NO: 422 - protein, 447 aa, synthetic construct; identical to SEQ ID NO: 420
  source 1..447: note = 22F10G3-QS-HC

SEQ ID NO: 423 - protein, 453 aa, synthetic construct
  source 1..453: note = 22F10G3-chimeric-HC
DVQLQESGPGQVKPSQSLSLTCTVTGYSLTSDFAWNWIRQFPGNRLEWMGDIRYSGSTDYNPSLKGRSSITRDTSKNQFFLQLNSVTTEDTATYYCARGGYYGYDEGYPMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 424 - protein, 453 aa, synthetic construct
  source 1..453: note = Hu22F10G3-1.1-HC
QVQLQESGPGLVKPSQTLSLTCTVSGYSLTSDFAWNWIRQHPGKGLEWIGDIRYSGSTDYNPSLKGRVTISVDTSKNQFSLKLSSVTAADTAVYYCARGGYYGYDEGYPMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 425 - protein, 453 aa, synthetic construct; identical to SEQ ID NO: 424
  source 1..453: note = Hu22F10G3-1.2-HC

SEQ ID NO: 426 - protein, 453 aa, synthetic construct; identical to SEQ ID NO: 424
  source 1..453: note = Hu22F10G3-1.3-HC

SEQ ID NO: 427 - protein, 453 aa, synthetic construct; identical to SEQ ID NO: 424
  source 1..453: note = Hu22F10G3-1.4-HC

SEQ ID NO: 428 - protein, 453 aa, synthetic construct
  source 1..453: note = Hu22F10G3-2.1-HC
QVQLQESGPGLVKPSQTLSLTCTVSGYSLTSDFAWNWIRQHPGKGLEWMGDIRYSGSTDYNPSLKGRVTISRDTSKNQFSLKLSSVTAADTAVYYCARGGYYGYDEGYPMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 429 - protein, 453 aa, synthetic construct; identical to SEQ ID NO: 428
  source 1..453: note = Hu22F10G3-2.2-HC

SEQ ID NO: 430 - protein, 453 aa, synthetic construct; identical to SEQ ID NO: 428
  source 1..453: note = Hu22F10G3-2.3-HC

SEQ ID NO: 431 - protein, 453 aa, synthetic construct; identical to SEQ ID NO: 428
  source 1..453: note = Hu22F10G3-2.4-HC

SEQ ID NO: 432 - protein, 453 aa, synthetic construct
  source 1..453: note = Hu22F10G3-3.1-HC
QVQLQESGPGLVKPSQTLSLTCTVSGYSLTSDFAWNWIRQHPGKGLEWMGDIRYSGSTDYNPSLKGRSTISRDTSKNQFSLKLSSVTAADTAVYYCARGGYYGYDEGYPMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 433 - protein, 453 aa, synthetic construct; identical to SEQ ID NO: 432
  source 1..453: note = Hu22F10G3-3.2-HC

SEQ ID NO: 434 - protein, 453 aa, synthetic construct; identical to SEQ ID NO: 432
  source 1..453: note = Hu22F10G3-3.3-HC

SEQ ID NO: 435 - protein, 453 aa, synthetic construct; identical to SEQ ID NO: 432
  source 1..453: note = Hu22F10G3-3.4-HC

SEQ ID NO: 436 - protein, 453 aa, synthetic construct
  source 1..453: note = Hu22F10G3-4.1-HC
QVQLQESGPGLVKPSQTLSLTCTVSGYSLTSDFAWNWIRQFPGKGLEWMGDIRYSGSTDYNPSLKGRSTISRDTSKNQFFLKLSSVTAADTAVYYCARGGYYGYDEGYPMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 437 - protein, 453 aa, synthetic construct; identical to SEQ ID NO: 436
  source 1..453: note = Hu22F10G3-4.2-HC

SEQ ID NO: 438 - protein, 453 aa, synthetic construct; identical to SEQ ID NO: 436
  source 1..453: note = Hu22F10G3-4.3-HC

SEQ ID NO: 439 - protein, 453 aa, synthetic construct; identical to SEQ ID NO: 436
  source 1..453: note = Hu22F10G3-4.4-HC

SEQ ID NO: 440 - protein, 453 aa, synthetic construct; identical to SEQ ID NO: 424
  source 1..453: note = Hu22F10G3-1.1-NA-HC

SEQ ID NO: 441 - protein, 453 aa, synthetic construct; identical to SEQ ID NO: 424
  source 1..453: note = Hu22F10G3-1.2-NA-HC

SEQ ID NO: 442 - protein, 453 aa, synthetic construct; identical to SEQ ID NO: 424
  source 1..453: note = Hu22F10G3-1.3-NA-HC

SEQ ID NO: 443 - protein, 453 aa, synthetic construct; identical to SEQ ID NO: 424
  source 1..453: note = Hu22F10G3-1.4-NA-HC

SEQ ID NO: 444 - protein, 453 aa, synthetic construct; identical to SEQ ID NO: 428
  source 1..453: note = Hu22F10G3-2.1-NA-HC

SEQ ID NO: 445 - protein, 453 aa, synthetic construct; identical to SEQ ID NO: 428
  source 1..453: note = Hu22F10G3-2.2-NA-HC

SEQ ID NO: 446 - protein, 453 aa, synthetic construct; identical to SEQ ID NO: 428
  source 1..453: note = Hu22F10G3-2.3-NA-HC

SEQ ID NO: 447 - protein, 453 aa, synthetic construct; identical to SEQ ID NO: 428
  source 1..453: note = Hu22F10G3-2.4-NA-HC

SEQ ID NO: 448 - protein, 453 aa, synthetic construct; identical to SEQ ID NO: 432
  source 1..453: note = Hu22F10G3-3.1-NA-HC

SEQ ID NO: 449 - protein, 453 aa, synthetic construct; identical to SEQ ID NO: 432
  source 1..453: note = Hu22F10G3-3.2-NA-HC

SEQ ID NO: 450 - protein, 453 aa, synthetic construct; identical to SEQ ID NO: 432
  source 1..453: note = Hu22F10G3-3.3-NA-HC

SEQ ID NO: 451 - protein, 453 aa, synthetic construct; identical to SEQ ID NO: 432
  source 1..453: note = Hu22F10G3-3.4-NA-HC

SEQ ID NO: 452 - protein, 453 aa, synthetic construct; identical to SEQ ID NO: 436
  source 1..453: note = Hu22F10G3-4.1-NA-HC

SEQ ID NO: 453 - protein, 453 aa, synthetic construct; identical to SEQ ID NO: 436
  source 1..453: note = Hu22F10G3-4.2-NA-HC

SEQ ID NO: 454 - protein, 453 aa, synthetic construct; identical to SEQ ID NO: 436
  source 1..453: note = Hu22F10G3-4.3-NA-HC

SEQ ID NO: 455 - protein, 453 aa, synthetic construct; identical to SEQ ID NO: 436
  source 1..453: note = Hu22F10G3-4.4-NA-HC

SEQ ID NO: 456 - protein, 453 aa, synthetic construct; identical to SEQ ID NO: 424
  source 1..453: note = Hu22F10G3-1.1-QS-HC

SEQ ID NO: 457 - protein, 453 aa, synthetic construct; identical to SEQ ID NO: 424
  source 1..453: note = Hu22F10G3-1.2-QS-HC

SEQ ID NO: 458 - protein, 453 aa, synthetic construct; identical to SEQ ID NO: 424
  source 1..453: note = Hu22F10G3-1.3-QS-HC

SEQ ID NO: 459 - protein, 453 aa, synthetic construct; identical to SEQ ID NO: 424
  source 1..453: note = Hu22F10G3-1.4-QS-HC

SEQ ID NO: 460 - protein, 453 aa, synthetic construct; identical to SEQ ID NO: 428
  source 1..453: note = Hu22F10G3-2.1-QS-HC

SEQ ID NO: 461 - protein, 453 aa, synthetic construct; identical to SEQ ID NO: 428
  source 1..453: note = Hu22F10G3-2.2-QS-HC

SEQ ID NO: 462 - protein, 453 aa, synthetic construct; identical to SEQ ID NO: 428
  source 1..453: note = Hu22F10G3-2.3-QS-HC

SEQ ID NO: 463 - protein, 453 aa, synthetic construct; identical to SEQ ID NO: 428
  source 1..453: note = Hu22F10G3-2.4-QS-HC

SEQ ID NO: 464 - protein, 453 aa, synthetic construct; identical to SEQ ID NO: 432
  source 1..453: note = Hu22F10G3-3.1-QS-HC

SEQ ID NO: 465 - protein, 453 aa, synthetic construct; identical to SEQ ID NO: 432
  source 1..453: note = Hu22F10G3-3.2-QS-HC

SEQ ID NO: 466 - protein, 453 aa, synthetic construct; identical to SEQ ID NO: 432
  source 1..453: note = Hu22F10G3-3.3-QS-HC

SEQ ID NO: 467 - protein, 453 aa, synthetic construct; identical to SEQ ID NO: 432
  source 1..453: note = Hu22F10G3-3.4-QS-HC

SEQ ID NO: 468 - protein, 453 aa, synthetic construct; identical to SEQ ID NO: 436
  source 1..453: note = Hu22F10G3-4.1-QS-HC

SEQ ID NO: 469 - protein, 453 aa, synthetic construct; identical to SEQ ID NO: 436
  source 1..453: note = Hu22F10G3-4.2-QS-HC

SEQ ID NO: 470 - protein, 453 aa, synthetic construct; identical to SEQ ID NO: 436
  source 1..453: note = Hu22F10G3-4.3-QS-HC

SEQ ID NO: 471 - protein, 453 aa, synthetic construct; identical to SEQ ID NO: 436
  source 1..453: note = Hu22F10G3-4.4-QS-HC

SEQ ID NO: 472 - protein, 214 aa, synthetic construct
  source 1..214: note = 9C10G11-LC
DIQMTQSPASLSASVGETVTITCRASGNIHNYLAWYQQKQGKSPQLLVYNAKTLADGVPSRFSGSGSGTQYSLKINSLQPEDFGSYYCQHFWSTPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 473 - protein, 218 aa, synthetic construct
  source 1..218: note = 57E2E4-LC
DIVLTQSPASLAVSLGQRATISCRASKRVSTSDYSYMHWYQQKPGQPPKLLIYLASNLESGVPARFSGSGSGTDFTLNIHPVEEEDAATYYCQHSREFPRTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 474 - protein, 214 aa, synthetic construct
  source 1..214: note = 75B10F12-LC
DIQMTQSPVSLSISVGETVTITCRASENVYNNLAWYQQKQGKSPQLLIYGATNLADGVPSRFSGSGSGTQFSLKINSLQSEDFGTYYCQHFWGTPWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 475 - protein, 214 aa, synthetic construct
  source 1..214: note = 7B6C7-LC
DIQMTQSPASLSASVGETVTITCRASGNIHNYLSWYQQKQGKSPQLLVYNAETLADGVPSRFSGSGSGTQYSLKINNLQPEDFGSYYCQHFWSTPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 476 - protein, 214 aa, synthetic construct
  source 1..214: note = 52G9B12B5-LC
NIVMTQSHKFMSTSVGDRVSITCKASQDVNTAVAWYQQIPGRSPKLLIYWASTRHTGVPDRFTGSGSGTDYTLTISSVQAEDLAFYYCQQHYRIPFTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 477 - protein, 218 aa, synthetic construct
  source 1..218: note = 13H2D11-LC
DIVLTQSPASLAVSLGQRATISCRASESVDNYGISFMNWFQQKPGQPPKLLIYAASNQGSGVPARFSGSGSGTDFSLNIHPMEEDDTAMYFCQQSKEVPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 478 - protein, 218 aa, synthetic construct
  source 1..218: note = 5A11D10-LC
NIVLTQSPASLAVSLGQRATISCRASESVDVYGNSFMHWYQQKAGQPPKLLIYLAFNLKSGVPARFSGSGSRTDFTLTIDPVEADDAATYYCQQNNEDPFTFGSGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 479 - protein, 213 aa, synthetic construct
  source 1..213: note = 5B1B11-LC
QIVLTQSPAIMSASLGEEITLTCSASSSVSYMHWYQQKSGTSPKLLIYTTSNLPSGVPSRFSGSGSGTFYSLTISSVEAEDAADYYCHQWSTYPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 480 - protein, 215 aa, synthetic construct
  source 1..215: note = 50A8F2-LC
QIVLTQSPAIMSASLGERVTMTCTASSSVSSTYLHWYQQKPGSSPKLWIYSTSNLASGVPARFSGSGSGTSYSLTFSSMEAEDAATYYCHQYHRSPLTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 481 - protein, 218 aa, synthetic construct
  source 1..218: note = 47A5H2-LC
DIVLTQSPASLAVSLGQRATISCRASESVDNYGMTFMSWFQQKPGQPPKLLIYAASNQGSGVPARFSGSGSGTDFSLNIHPMEEDDTAMYFCQQSKEVPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 482 - protein, 214 aa, synthetic construct
  source 1..214: note = 66G9C11-LC
DIVLTQSPATLSVTPGDRVSLSCRASQSINNYLHWYQQKSHESPRLLIKYASQSISGIPSRFSGSGSGADFTLSINSVETEDFGMYFCQQSNSWPPTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 483 - protein, 214 aa, synthetic construct
  source 1..214: note = 33F11A6-LC
DIQMTQSPASLSASVGETVTITCRASGNIHNYLAWYQQKQGKSPQLLIYNAKTLADGVPSRFSGSGSGTQYFLKINSLQPEDFGSYCCQHFWSTPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 484 - protein, 214 aa, synthetic construct
  source 1..214: note = 37G6A7-LC
DIQMTQSPASLSASVGETVTITCRASGNIHNYLAWYQQKQGKSPQLLVYIAKTLADGVPSRFSGSGSGTQYSLKINSLQPEDFGTYYCQHFWSTPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 485 - protein, 214 aa, synthetic construct
  source 1..214: note = 39C5D6-LC
DIQMTQSPASLSASVGETVTITCRASGNIHNYLAWYQQKQGKSPQLLVYNAKTLEDGVPSRFSGSGSGTQYSLKINSLQPDDFGNYFCQHFWSTPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 486 - protein, 220 aa, synthetic construct
  source 1..220: note = 62H1D6-LC
DIVMTQSPSSLAMSVGQKVTMSCKSSQSLLNSSNQKNYLAWYQQKPGQSPKLLVYFASTRESGVPDRFIGSGSGTDFTLTISSVQAEDLADYFCQQHYNTPFTFGSGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 487 - protein, 218 aa, synthetic construct
  source 1..218: note = 92E12F2-LC
DIVLTQSPASLAVSLGQRATISCRASKSVSSSGYTYLHWYQQKPGQPPKLLIYLTSNLDSGVPARFSGSGSGTDFTLNIHPVEEEDAATYYCQHSRELPRTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 488 - protein, 214 aa, synthetic construct
  source 1..214: note = 18G11F11-LC
DIQMTQSPASLSASVGETVTITCRASGNIHNYLAWYQQKQGKSPQLLVYNAETLADGVPSRFSGSGSGTQYSLKINSLQPEDFGSYYCQHFWSTPYTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 489 - protein, 218 aa, synthetic construct
  source 1..218: note = 57E2E4-Chimeric-LC
DIVLTQSPASLAVSLGQRATISCRASKRVSTSDYSYMHWYQQKPGQPPKLLIYLASNLESGVPARFSGSGSGTDFTLNIHPVEEEDAATYYCQHSREFPRTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 490 - protein, 214 aa, synthetic construct
  source 1..214: note = 7B6C7-Chimeric-LC
DIQMTQSPASLSASVGETVTITCRASGNIHNYLSWYQQKQGKSPQLLVYNAETLADGVPSRFSGSGSGTQYSLKINNLQPEDFGSYYCQHFWSTPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 491 - protein, 214 aa, synthetic construct
  source 1..214: note = 52G9B12B5-Chimeric-LC
NIVMTQSHKFMSTSVGDRVSITCKASQDVNTAVAWYQQIPGRSPKLLIYWASTRHTGVPDRFTGSGSGTDYTLTISSVQAEDLAFYYCQQHYRIPFTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 492 - protein, 213 aa, synthetic construct
  source 1..213: note = 5B1B11-Chimeric-LC
QIVLTQSPAIMSASLGEEITLTCSASSSVSYMHWYQQKSGTSPKLLIYTTSNLPSGVPSRFSGSGSGTFYSLTISSVEAEDAADYYCHQWSTYPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 493 - protein, 215 aa, synthetic construct
  source 1..215: note = 47A5H2-Chimeric-LC
QIVLTQSPAIMSASLGERVTMTCTASSSVSSTYLHWYQQKPGSSPKLWIYSTSNLASGVPARFSGSGSGTSYSLTFSSMEAEDAATYYCHQYHRSPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 494 - protein, 218 aa, synthetic construct
  source 1..218: note = 50A8F2-Chimeric-LC
DIVLTQSPASLAVSLGQRATISCRASESVDNYGMTFMSWFQQKPGQPPKLLIYAASNQGSGVPARFSGSGSGTDFSLNIHPMEEDDTAMYFCQQSKEVPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 495 - protein, 214 aa, synthetic construct; identical to SEQ ID NO: 491
  source 1..214: note = 52G9B12B5-Chimeric-EN-LC

SEQ ID NO: 496 - protein, 218 aa, synthetic construct
  source 1..218: note = 22F10G3-Chimeric-LC
NIVLTQSPASLAVSLGQRATISCRASESVDVYGNSFMHWYQQKTGQPPKLLIYLAFNLKSGVPARFSGSGSRTDFTLTIDPVEADDAATYYCQQNNEDPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 497 - protein, 218 aa, synthetic construct
  source 1..218: note = Hu47A5H2-01-EN-LC
DIVMTQSPDSLAVSLGERATINCRASESVDNYGMTFMSWYQQKPGQPPKLLIYAASNQGSGVPDRFSGSGSGTDFTLTISSLQAEDVAVYYCQQSKEVPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 498 - protein, 218 aa, synthetic construct
  source 1..218: note = Hu47A5H2-02-EN-LC
DIVLTQSPDSLAVSLGERATINCRASESVDNYGMTFMSWYQQKPGQPPKLLIYAASNQGSGVPDRFSGSGSGTDFTLTISSLQAEDVAVYYCQQSKEVPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 499 - protein, 218 aa, synthetic construct
  source 1..218: note = Hu47A5H2-03-EN-LC
DIVLTQSPDSLAVSLGERATINCRASESVDNYGMTFMSWFQQKPGQPPKLLIYAASNQGSGVPDRFSGSGSGTDFTLTISSLQAEDVAVYYCQQSKEVPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 500 - protein, 218 aa, synthetic construct
  source 1..218: note = Hu47A5H2-04-EN-LC
EIVLTQSPATLSLSPGERATLSCRASESVDNYGMTFMSWYQQKPGQAPRLLIYAASNQGSGIPARFSGSGSGTDFTLTISSLEPEDFAVYYCQQSKEVPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 501 - protein, 218 aa, synthetic construct; identical to SEQ ID NO: 497
  source 1..218: note = Hu47A5H2-05-EN-LC

SEQ ID NO: 502 - protein, 218 aa, synthetic construct; identical to SEQ ID NO: 498
  source 1..218: note = Hu47A5H2-06-EN-LC

SEQ ID NO: 503 - protein, 218 aa, synthetic construct; identical to SEQ ID NO: 499
  source 1..218: note = Hu47A5H2-07-EN-LC

SEQ ID NO: 504 - protein, 218 aa, synthetic construct; identical to SEQ ID NO: 500
  source 1..218: note = Hu47A5H2-08-EN-LC

SEQ ID NO: 505 - protein, 218 aa, synthetic construct; identical to SEQ ID NO: 497
  source 1..218: note = Hu47A5H2-09-EN-LC

SEQ ID NO: 506 - protein, 218 aa, synthetic construct; identical to SEQ ID NO: 498
  source 1..218: note = Hu47A5H2-10-EN-LC

SEQ ID NO: 507 - protein, 218 aa, synthetic construct; identical to SEQ ID NO: 499
  source 1..218: note = Hu47A5H2-11-EN-LC

SEQ ID NO: 508 - protein, 218 aa, synthetic construct; identical to SEQ ID NO: 500
  source 1..218: note = Hu47A5H2-12-EN-LC

SEQ ID NO: 509 - protein, 218 aa, synthetic construct; identical to SEQ ID NO: 497
  source 1..218: note = Hu47A5H2-13-EN-LC

SEQ ID NO: 510 - protein, 218 aa, synthetic construct; identical to SEQ ID NO: 498
  source 1..218: note = Hu47A5H2-14-EN-LC

SEQ ID NO: 511 - protein, 218 aa, synthetic construct; identical to SEQ ID NO: 499
  source 1..218: note = Hu47A5H2-15-EN-LC

SEQ ID NO: 512 - protein, 218 aa, synthetic construct; identical to SEQ ID NO: 500
  source 1..218: note = Hu47A5H2-16-EN-LC

SEQ ID NO: 513 - protein, 215 aa, synthetic construct
  source 1..215: note = Hu50A8F2-01-EN-LC
DIQMTQSPSSLSASVGDRVTITCTASSSVSSTYLHWYQQKPGKAPKLLIYSTSNLASGVPSRFSGSGSGTDFTLTISSLQPEDFATYYCHQYHRSPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 514 - protein, 215 aa, synthetic construct
  source 1..215: note = Hu50A8F2-02-EN-LC
DIQMTQSPSSLSASVGDRVTITCTASSSVSSTYLHWYQQKPGKAPKLLIYSTSNLASGVPSRFSGSGSGTDYTLTISSLQPEDFATYYCHQYHRSPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 515 - protein, 215 aa, synthetic construct
  source 1..215: note = Hu50A8F2-03-EN-LC
DIQLTQSPSSLSASVGDRVTITCTASSSVSSTYLHWYQQKPGKSPKLLIYSTSNLASGVPSRFSGSGSGTDYTLTISSLQPEDFATYYCHQYHRSPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 516 - protein, 215 aa, synthetic construct
  source 1..215: note = Hu50A8F2-04-EN-LC
DIQLTQSPSSLSASVGDRVTITCTASSSVSSTYLHWYQQKPGKSPKLLIYSTSNLASGVPSRFSGSGSGTDYTLTISSLQPEDAATYYCHQYHRSPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 517 - protein, 215 aa, synthetic construct; identical to SEQ ID NO: 513
  source 1..215: note = Hu50A8F2-05-EN-LC

SEQ ID NO: 518 - protein, 215 aa, synthetic construct; identical to SEQ ID NO: 514
  source 1..215: note = Hu50A8F2-06-EN-LC

SEQ ID NO: 519 - protein, 215 aa, synthetic construct; identical to SEQ ID NO: 515
  source 1..215: note = Hu50A8F2-07-EN-LC

SEQ ID NO: 520 - protein, 215 aa, synthetic construct; identical to SEQ ID NO: 516
  source 1..215: note = Hu50A8F2-08-EN-LC

SEQ ID NO: 521 - protein, 215 aa, synthetic construct; identical to SEQ ID NO: 513
  source 1..215: note = Hu50A8F2-09-EN-LC

SEQ ID NO: 522 - protein, 215 aa, synthetic construct; identical to SEQ ID NO: 514
  source 1..215: note = Hu50A8F2-10-EN-LC

SEQ ID NO: 523 - protein, 215 aa, synthetic construct; identical to SEQ ID NO: 515
  source 1..215: note = Hu50A8F2-11-EN-LC

SEQ ID NO: 524 - protein, 215 aa, synthetic construct; identical to SEQ ID NO: 516
  source 1..215: note = Hu50A8F2-12-EN-LC

SEQ ID NO: 525 - protein, 215 aa, synthetic construct; identical to SEQ ID NO: 513
  source 1..215: note = Hu50A8F2-13-EN-LC

SEQ ID NO: 526 - protein, 215 aa, synthetic construct; identical to SEQ ID NO: 514
  source 1..215: note = Hu50A8F2-14-EN-LC

SEQ ID NO: 527 - protein, 215 aa, synthetic construct; identical to SEQ ID NO: 515
  source 1..215: note = Hu50A8F2-15-EN-LC

SEQ ID NO: 528 - protein, 215 aa, synthetic construct; identical to SEQ ID NO: 516
  source 1..215: note = Hu50A8F2-16-EN-LC

SEQ ID NO: 529 - protein, 218 aa, synthetic construct
  source 1..218: note = 22F10G3-LC
NIVLTQSPASLAVSLGQRATISCRASESVDVYGNSFMHWYQQKTGQPPKLLIYLAFNLKSGVPARFSGSGSRTDFTLTIDPVEADDAATYYCQQNNEDPFTFGSGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 530 - protein, 218 aa, synthetic construct
  source 1..218: note = 22F10G3-NA-LC
NIVLTQSPASLAVSLGQRATISCRASESVDVYGNAFMHWYQQKTGQPPKLLIYLAFNLKSGVPARFSGSGSRTDFTLTIDPVEADDAATYYCQQNNEDPFTFGSGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 531 - protein, 218 aa, synthetic construct
  source 1..218: note = 22F10G3-QS-LC
NIVLTQSPASLAVSLGQRATISCRASESVDVYGQSFMHWYQQKTGQPPKLLIYLAFNLKSGVPARFSGSGSRTDFTLTIDPVEADDAATYYCQQNNEDPFTFGSGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 532 - protein, 218 aa, synthetic construct; identical to SEQ ID NO: 496
  source 1..218: note = 22F10G3-chimeric-LC

SEQ ID NO: 533 - protein, 218 aa, synthetic construct
  source 1..218: note = Hu22F10G3-1.1-LC
DIVMTQSPDSLAVSLGERATINCRASESVDVYGNSFMHWYQQKPGQPPKLLIYLAFNLKSGVPDRFSGSGSGTDFTLTISSLQAEDVAVYYCQQNNEDPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 534 - protein, 218 aa, synthetic construct
  source 1..218: note = Hu22F10G3-1.2-LC
DIVMTQSPDSLAVSLGERATINCRASESVDVYGNSFMHWYQQKPGQPPKLLIYLAFNLKSGVPARFSGSGSGTDFTLTISSLQAEDVAVYYCQQNNEDPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 535 - protein, 218 aa, synthetic construct
  source 1..218: note = Hu22F10G3-1.3-LC
DIVMTQSPDSLAVSLGERATISCRASESVDVYGNSFMHWYQQKPGQPPKLLIYLAFNLKSGVPDRFSGSGSGTDFTLTISSLQAEDVAVYYCQQNNEDPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 536 - protein, 218 aa, synthetic construct
  source 1..218: note = Hu22F10G3-1.4-LC
DIVMTQSPDSLAVSLGERATISCRASESVDVYGNSFMHWYQQKPGQPPKLLIYLAFNLKSGVPARFSGSGSGTDFTLTISSLQAEDVATYYCQQNNEDPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 537 - protein, 218 aa, synthetic construct; identical to SEQ ID NO: 533
  source 1..218: note = Hu22F10G3-2.1-LC

SEQ ID NO: 538 - protein, 218 aa, synthetic construct; identical to SEQ ID NO: 534
  source 1..218: note = Hu22F10G3-2.2-LC

SEQ ID NO: 539 - protein, 218 aa, synthetic construct; identical to SEQ ID NO: 535
  source 1..218: note = Hu22F10G3-2.3-LC

SEQ ID NO: 540 - protein, 218 aa, synthetic construct; identical to SEQ ID NO: 536
  source 1..218: note = Hu22F10G3-2.4-LC

SEQ ID NO: 541 - protein, 218 aa, synthetic construct; identical to SEQ ID NO: 533
  source 1..218: note = Hu22F10G3-3.1-LC

SEQ ID NO: 542 - protein, 218 aa, synthetic construct; identical to SEQ ID NO: 534
  source 1..218: note = Hu22F10G3-3.2-LC

SEQ ID NO: 543 - protein, 218 aa, synthetic construct; identical to SEQ ID NO: 535
  source 1..218: note = Hu22F10G3-3.3-LC

SEQ ID NO: 544 - protein, 218 aa, synthetic construct; identical to SEQ ID NO: 536
  source 1..218: note = Hu22F10G3-3.4-LC

SEQ ID NO: 545 - protein, 218 aa, synthetic construct; identical to SEQ ID NO: 533
  source 1..218: note = Hu22F10G3-4.1-LC

SEQ ID NO: 546 - protein, 218 aa, synthetic construct; identical to SEQ ID NO: 534
  source 1..218: note = Hu22F10G3-4.2-LC

SEQ ID NO: 547 - protein, 218 aa, synthetic construct; identical to SEQ ID NO: 535
  source 1..218: note = Hu22F10G3-4.3-LC

SEQ ID NO: 548 - protein, 218 aa, synthetic construct; identical to SEQ ID NO: 536
  source 1..218: note = Hu22F10G3-4.4-LC

SEQ ID NO: 549 - protein, 218 aa, synthetic construct
  source 1..218: note = Hu22F10G3-1.1-NA-LC
DIVMTQSPDSLAVSLGERATINCRASESVDVYGNAFMHWYQQKPGQPPKLLIYLAFNLKSGVPDRFSGSGSGTDFTLTISSLQAEDVAVYYCQQNNEDPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 550 - protein, 218 aa, synthetic construct
  source 1..218: note = Hu22F10G3-1.2-NA-LC
DIVMTQSPDSLAVSLGERATINCRASESVDVYGNAFMHWYQQKPGQPPKLLIYLAFNLKSGVPARFSGSGSGTDFTLTISSLQAEDVAVYYCQQNNEDPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 551 - protein, 218 aa, synthetic construct
  source 1..218: note = Hu22F10G3-1.3-NA-LC
DIVMTQSPDSLAVSLGERATISCRASESVDVYGNAFMHWYQQKPGQPPKLLIYLAFNLKSGVPDRFSGSGSGTDFTLTISSLQAEDVAVYYCQQNNEDPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 552 - protein, 218 aa, synthetic construct
  source 1..218: note = Hu22F10G3-1.4-NA-LC
DIVMTQSPDSLAVSLGERATISCRASESVDVYGNAFMHWYQQKPGQPPKLLIYLAFNLKSGVPARFSGSGSGTDFTLTISSLQAEDVATYYCQQNNEDPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 553 - protein, 218 aa, synthetic construct; identical to SEQ ID NO: 549
  source 1..218: note = Hu22F10G3-2.1-NA-LC

SEQ ID NO: 554 - protein, 218 aa, synthetic construct; identical to SEQ ID NO: 550
  source 1..218: note = Hu22F10G3-2.2-NA-LC

SEQ ID NO: 555 - protein, 218 aa, synthetic construct; identical to SEQ ID NO: 551
  source 1..218: note = Hu22F10G3-2.3-NA-LC

SEQ ID NO: 556 - protein, 218 aa, synthetic construct; identical to SEQ ID NO: 552
  source 1..218: note = Hu22F10G3-2.4-NA-LC

SEQ ID NO: 557 - protein, 218 aa, synthetic construct; identical to SEQ ID NO: 549
  source 1..218: note = Hu22F10G3-3.1-NA-LC

SEQ ID NO: 558 - protein, 218 aa, synthetic construct; identical to SEQ ID NO: 550
  source 1..218: note = Hu22F10G3-3.2-NA-LC

SEQ ID NO: 559 - protein, 218 aa, synthetic construct; identical to SEQ ID NO: 551
  source 1..218: note = Hu22F10G3-3.3-NA-LC

SEQ ID NO: 560 - protein, 218 aa, synthetic construct; identical to SEQ ID NO: 552
  source 1..218: note = Hu22F10G3-3.4-NA-LC

SEQ ID NO: 561 - protein, 218 aa, synthetic construct; identical to SEQ ID NO: 549
  source 1..218: note = Hu22F10G3-4.1-NA-LC

SEQ ID NO: 562 - protein, 218 aa, synthetic construct; identical to SEQ ID NO: 550
  source 1..218: note = Hu22F10G3-4.2-NA-LC

SEQ ID NO: 563 - protein, 218 aa, synthetic construct; identical to SEQ ID NO: 551
  source 1..218: note = Hu22F10G3-4.3-NA-LC

SEQ ID NO: 564 - protein, 218 aa, synthetic construct; identical to SEQ ID NO: 552
  source 1..218: note = Hu22F10G3-4.4-NA-LC

SEQ ID NO: 565 - protein, 218 aa, synthetic construct
  source 1..218: note = Hu22F10G3-1.1-QS-LC
DIVMTQSPDSLAVSLGERATINCRASESVDVYGQSFMHWYQQKPGQPPKLLIYLAFNLKSGVPDRFSGSGSGTDFTLTISSLQAEDVAVYYCQQNNEDPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 566 - protein, 218 aa, synthetic construct
  source 1..218: note = Hu22F10G3-1.2-QS-LC
DIVMTQSPDSLAVSLGERATINCRASESVDVYGQSFMHWYQQKPGQPPKLLIYLAFNLKSGVPARFSGSGSGTDFTLTISSLQAEDVAVYYCQQNNEDPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 567 - protein, 218 aa, synthetic construct
  source 1..218: note = Hu22F10G3-1.3-QS-LC
DIVMTQSPDSLAVSLGERATISCRASESVDVYGQSFMHWYQQKPGQPPKLLIYLAFNLKSGVPDRFSGSGSGTDFTLTISSLQAEDVAVYYCQQNNEDPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 568 - protein, 218 aa, synthetic construct
  source 1..218: note = Hu22F10G3-1.4-QS-LC
DIVMTQSPDSLAVSLGERATISCRASESVDVYGQSFMHWYQQKPGQPPKLLIYLAFNLKSGVPARFSGSGSGTDFTLTISSLQAEDVATYYCQQNNEDPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 569 - protein, 218 aa, synthetic construct; identical to SEQ ID NO: 565
  source 1..218: note = Hu22F10G3-2.1-QS-LC

SEQ ID NO: 570 - protein, 218 aa, synthetic construct; identical to SEQ ID NO: 566
  source 1..218: note = Hu22F10G3-2.2-QS-LC

SEQ ID NO: 571 - protein, 218 aa, synthetic construct; identical to SEQ ID NO: 567
  source 1..218: note = Hu22F10G3-2.3-QS-LC

SEQ ID NO: 572 - protein, 218 aa, synthetic construct; identical to SEQ ID NO: 568
  source 1..218: note = Hu22F10G3-2.4-QS-LC

SEQ ID NO: 573 - protein, 218 aa, synthetic construct; identical to SEQ ID NO: 565
  source 1..218: note = Hu22F10G3-3.1-QS-LC

SEQ ID NO: 574 - protein, 218 aa, synthetic construct; identical to SEQ ID NO: 566
  source 1..218: note = Hu22F10G3-3.2-QS-LC

SEQ ID NO: 575 - protein, 218 aa, synthetic construct; identical to SEQ ID NO: 567
  source 1..218: note = Hu22F10G3-3.3-QS-LC

SEQ ID NO: 576 - protein, 218 aa, synthetic construct; identical to SEQ ID NO: 568
  source 1..218: note = Hu22F10G3-3.4-QS-LC

SEQ ID NO: 577 - protein, 218 aa, synthetic construct; identical to SEQ ID NO: 565
  source 1..218: note = Hu22F10G3-4.1-QS-LC

SEQ ID NO: 578 - protein, 218 aa, synthetic construct; identical to SEQ ID NO: 566
  source 1..218: note = Hu22F10G3-4.2-QS-LC

SEQ ID NO: 579 - protein, 218 aa, synthetic construct; identical to SEQ ID NO: 567
  source 1..218: note = Hu22F10G3-4.3-QS-LC

SEQ ID NO: 580 - protein, 218 aa, synthetic construct; identical to SEQ ID NO: 568
  source 1..218: note = Hu22F10G3-4.4-QS-LC

SEQ ID NO: 581 - protein, 449 aa, synthetic construct
  source 1..449: note = Hu47A5H2-12-EN-HC
QVQLVQSGAEVKKPGASVKVSCKASGYSFTGYWMHWVRQAPGQGLEWMGMIHPSDSETRLNQKFKDRVTMTVDKSTSTVYMELSSLRSEDTAVYYCASEGLRRGSFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 582 - protein, 449 aa, synthetic construct
  source 1..449: note = Hu47A5H2-13-EN-HC
QVQLVQSGAEVKKPGASVKVSCKGSGYSFTGYWMHWVRQAPGQGLEWMGMIHPSDSETRLNQKFKDKVTMTVDKSTSTVYMELSSLRSEDTAVYYCASEGLRRGSFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 583 - protein, 449 aa, synthetic construct; identical to SEQ ID NO: 582
  source 1..449: note = Hu47A5H2-14-EN-HC

SEQ ID NO: 584 - protein, 449 aa, synthetic construct; identical to SEQ ID NO: 582
  source 1..449: note = Hu47A5H2-15-EN-HC

SEQ ID NO: 585 - protein, 449 aa, synthetic construct; identical to SEQ ID NO: 582
  source 1..449: note = Hu47A5H2-16-EN-HC

SEQ ID NO: 586 - protein, 9 aa, synthetic construct
SLLMWITQC